(19) United States
(12) Patent Application Publication
(10) Pub. No.: US 2018/0236103 A1
(43) Pub. Date: Aug. 23, 2018

(54) CRISPR/CAS-RELATED METHODS AND COMPOSITIONS FOR TREATING HEPATITIS B VIRUS

(71) Applicant: EDITAS MEDICINE, INC., Cambridge, MA (US)

(72) Inventors: Ari E. Friedland, Boston, MA (US); Penrose O'Donnell, Yarmouth, ME (US)

(73) Assignee: EDITAS MEDICINE, INC., Cambridge, MA (US)

(21) Appl. No.: 15/958,439

(22) Filed: Apr. 20, 2018

Related U.S. Application Data
(63) Continuation of application No. PCT/US2016/057810, filed on Oct. 20, 2016.
(60) Provisional application No. 62/244,724, filed on Oct. 21, 2015.
(60) Provisional application No. 62/294,834, filed on Feb. 12, 2016.

(51) Int. Cl. A61K 48/00; C12N 9/22; C12N 15/113; C12N 15/85; C12N 15/90; A61P 31/22

(52) U.S. Cl. CPC A61K 48/005; C12N 9/22; A61P 31/22; C12N 15/85; C12N 15/907; C12N 15/1131

(57) ABSTRACT

CRISPR/CAS-related genome editing systems, compositions and methods for preventing and/or treating HBV infection are disclosed.

### CROSS-REFERENCES TO RELATED APPLICATIONS

This application is a Continuation of International Patent Application No. PCT/US16/57810, filed Oct. 20, 2016, which claims priority to U.S. Provisional Application No. 62/244,724, filed Oct. 21, 2015, and U.S. Provisional Application No. 62/294,834, filed Feb. 12, 2016, the contents of each of which are hereby incorporated by reference in their entirety herein, and to each of which priority is claimed.

### SEQUENCE LISTING

The present specification makes reference to a Sequence Listing (submitted electronically as a .txt file named “084177_0170SEQ” on Apr. 20, 2018). The 084177_0170SEQ.txt file was generated on Apr. 20, 2018 and is 32,232,586 bytes in size. The entire contents of the Sequence Listing are hereby incorporated by reference.

### FIELD OF THE INVENTION

The disclosure relates to CRISPR/CAS-related methods, compositions and genome editing systems for editing of a target nucleic acid sequence, e.g., altering one or more of the hepatitis B virus (HBV) viral genes, e.g., one or more of PreC, C, X, PreS1, PreS2, S, P and/or SP gene(s), and applications thereof in connection with HBV.

### BACKGROUND

Hepatitis B is a viral disease that is a frequent cause of cirrhosis and mortality worldwide. Chronic hepatitis B affects more than 240 million individuals worldwide (Franco et al, World J. Hepatol. 2012, 4, 74; Schweitzer et. al., Lancet, 2015, 50140-6736(15)61412-X). Hepatitis B is responsible for approximately 1 million deaths every year worldwide (Hepatitis B Foundation accessed Aug. 15, 2015 at: www.hepb.org/hepb/statistics.htm). In the United States (U.S.), 1 million individuals are chronically infected with Hepatitis B (Hepatitis B Foundation, accessed Aug. 15, 2015 at: www.hepb.org/hepb/statistics.htm). 5,000 deaths per year in the U.S. are due to hepatitis B infection (Hepatitis B Foundation accessed Aug. 15, 2015 at: www.hepb.org/hepb/statistics.htm).

Certain areas of the world have high prevalence rates, including Sub-Saharan Africa, East Asia and Pacific Nations. In these areas, more than 8% of the population is chronically infected with HBV. In the U.S., 0.3% of the population is chronically infected with HBV.

Hepatitis B is caused by hepatitis B virus (HBV). HBV is transmitted through exposure to blood or bodily fluids, including through sexual contact or the sharing of needles by intravenous drug use. Infants may acquire the infection in the perinatal period from an infected mother.

Acute infection with HBV is often asymptomatic. Chronic hepatitis B (CHB) infection develops in some proportion of subjects infected, depending on age and immunologic status. Up to 90% of adults who are infected will clear the virus and not develop CHB. Approximately 10% of adults will not clear the infection and will develop chronic hepatitis B (CHB). The inverse is true for infants: up to 90% of infants infected will develop CHB and approximately 10% of those infected will clear the infection. Children who are infected with HBV are at a much higher risk of developing CHB than adults and, subsequently, severe disease sequelae. In particular, between 25% and 50% of children infected with HBV will develop CHB.

CHB causes cirrhosis and hepatocellular carcinoma (HCC) in a significant subset of subjects. Subjects with CHB have a 1-2% annual risk of developing cirrhosis, and a 2-5% annual risk of developing HCC (Liaw et al, Hepatology, 1988; 8:493-496; Fattovich et al, Gastroenterology, 2004; 127:S35-S50). Between 15% and 40% of subjects with CHB will develop cirrhosis, HCC or liver failure (Perz et al, J Hepatol, 2006; 45: 529-538). Furthermore, subjects with HBV are also at risk for developing superinfection with Hepatitis D virus (HDV). HDV requires the presence of infection with HBV, as HDV relies on HBsAg presence for assembly and infectivity. Co-infection with HDV leads to more severe disease and a higher risk of disease sequelae. Subjects have 2-3 times the risk of developing cirrhosis or hepatocellular carcinoma (HCC) and have 2-3 times the risk of dying from the disease.

Host immune defense is very important to combating HBV infection. CD4+ T-cells and CD8+ cells are responsible for recognizing and clearing the pathogen. Subjects with impaired T-cell responses, including those with HIV, those receiving immunosuppressants following organ transplants, and neonates with developing immune systems, are more likely to develop chronic hepatitis B and are therefore more likely to develop cirrhosis and/or HCC.

Interferons and antiviral therapies, including nucleoside and nucleotide inhibitors, are the approved therapies for the treatment of chronic hepatitis B. Interferons (IFNs) include interferon-alpha (IFN) and PEGylated interferon (PEG-IFN), and nucleoside and nucleotide analogues include tenofovir and entacavir. These therapies decrease viral replication rates. The World Health Organization guidelines for the treatment of Hepatitis B advise treatment with both interferons and nucleos(t)ide analogues. In the United States, first line treatment with nucleos(t)ide analogues is the generally accepted standard of care. However, in subjects with HBV-HDV co-infection, nucleos(t)ide analogues are not effective. IFN or PEG-IFN is therefore used in the setting of HBV-HDV coinfection.

Interferon therapy and antiviral therapies control HBV replication, as evidenced by decreases in HBV DNA counts in subjects on active therapy. However, the majority of subjects with CHB will not achieve a functional cure after treatment with currently available therapies. 8-10% of subjects with CHB who undergo antiviral and/or IFN-based therapy achieve a functional cure, as defined by a loss of Hepatitis B surface antigen (HBsAg) expression in the blood. In addition, there is concern that resistant HBV strains will develop following treatment with nucleos(t)ide analogues.

A vaccine against HBV is available and is recommended for health care workers and infants in the United States. The incidence of new cases in the U.S. has declined considerably since the introduction of the vaccine in the mid-1980s. In spite of the existence of a hepatitis B vaccine and the use of antiviral therapy, chronic hepatitis B rates in the U.S. have remained constant for the last 16 years. Since 1999, the prevalence of CHB in the U.S. has remained stable at 0.3% (Roberts et. al, Hepatology 2015; Aug. 6. doi: 10.1002/hep.28109). As such, CHB remains a considerable public health problem in the U.S. and worldwide and current treatment regimens do not cure the disease in the majority of subjects.

Therefore, new therapies are needed to control and treat HBV, especially CHB. Novel therapies targeting HBV genomic DNA could produce a functional cure of the disease, defined by a loss of HBs antigen positivity in serum assays. Such therapies could prevent the development of cirrhosis in subjects with CHB and may also decrease the risk of hepatocellular carcinoma in subjects with CHB.

### SUMMARY OF THE DISCLOSURE

The methods, genome editing systems, and compositions discussed herein, provide for the treatment, prevention and/or reduction of hepatitis B virus (HBV), by introducing one or more mutations in the HBV genome, or by modifying the expression of one or more HBV proteins. The HBV genome includes but is not limited to the coding sequences of the PreC, C, X, PreS1, PreS2, S, P and SP genes which encode the Hbe, Hbc, Hbx, LHBs, MHBs, SHBs, Pol and HBSP proteins, respectively.

HBV is a hepadnavirus that preferentially affects hepatocytes. Enveloped virions contain a 3.2 kB double-stranded DNA genome with four partially overlapping open reading frames (ORFs). During chronic HBV infection, HBV DNA resides in the nucleus of hepatocytes in covalently closed circular DNA (cccDNA) form. Current therapies approved for the treatment of chronic HBV do not target HBV cccDNA.

The methods, genome editing systems, and compositions discussed herein provide for treatment, prevention and/or reduction of HBV, or its symptoms, by altering (e.g., knocking out and/or knocking down) one or more of the HBV viral genes, e.g., by knocking out one or more of PreC, C, X, PreS1, PreS2, S, P and/or SP gene(s). The methods, genome editing systems, and compositions discussed herein provide for treatment, prevention and/or reduction of HBV, or its symptoms, by knocking out one or more of the HBV viral genes, e.g., by knocking out one or more of PreC, X, PreS1, PreS2, S, P and/or SP gene(s). The methods, genome editing systems, and compositions discussed herein provide for treatment, prevention and/or reduction of HBV, or its symptoms, by knocking down one or more of the HBV viral genes, e.g., by knocking down one or more of PreC, X, PreS1, PreS2, S, P and/or SP gene(s). Methods and compositions discussed herein provide for treatment, prevention and/or reduction of HBV, or its symptoms, by concomitantly knocking out one or more of the HBV viral genes and knocking down one or more of the HBV viral genes, e.g., by knocking out one or more of PreC, C, X, PreS1, PreS2, S, P and/or SP gene(s) and knocking down one or more of PreC, C, X, PreS1, PreS2, S, P and/or SP gene(s). The methods, genome editing systems, and compositions discussed herein provide for treatment, prevention and/or reduction of HBV or its symptoms, by alteration of one or more positions within HBV genomic DNA leading to its destruction and/or elimination from infected cells.

In one aspect, the methods, genome editing systems, and compositions discussed herein may be used to alter one or more of PreC, C, X, PreS1, PreS2, S, P and/or SP gene(s) to treat, prevent and/or reduce HBV by targeting the gene(s), e.g., the non-coding or coding regions, e.g., the promoter region, or a transcribed sequence of PreC, C, X, PreS1, PreS2, S, P and/or SP gene(s). In certain embodiments, coding sequence, e.g., a coding region, e.g., an early coding region, of one or more of PreC, X, PreS1, PreS2, S, P and/or SP gene(s), is targeted for alteration e.g., knockout or knockdown of expression. In certain embodiments, coding sequence, e.g., a coding region, e.g., an early coding region, of one or more of PreC, X, PreS1, PreS2, S, P and/or SP gene(s), is targeted for alteration, e.g., knockout or knockdown of expression.

In certain embodiments, coding sequence, e.g., a coding region, e.g., an early coding region, of two or more of PreC, C, X, PreS1, PreS2, S, P and/or SP gene(s), is targeted for alteration and concomitant knockout and knockdown of expression. In certain embodiments, a non-coding sequence, e.g., promoter, an enhancer, 3′UTR, and/or polyadenylation signal, of two or more of PreC, C, X, PreS1, PreS2, S, P and/or SP gene(s), is targeted for alteration and concomitant knockout and knockdown of expression.

In certain embodiments, altering (e.g., knocking out and/or knocking down) the PreC, C, X, PreS1, PreS2, S, P or SP gene refers to (1) reducing or eliminating PreC, C, X, PreS1, PreS2, S, P or SP gene expression, (2) interfering with Precore, Core, X protein, Long surface protein, middle surface protein, S protein (also known as HBs antigen and HBsAg), polymerase protein, and/or Hepatitis B spliced protein function (proteins abbreviated, respectively, as HBe, HBc, HBx, PreS1, PreS2, S, Pol, and/or HBSP), or (3) reducing or eliminating the intracellular, serum and/or intra-parenchymal levels of HBe, HBc, HBx, LHBs, MHBs, SHBs, Pol, and/or HBSP proteins.

In certain embodiments, any sequence within the HBV genome, e.g., a coding region, e.g., an early coding region, or a non-coding region, e.g., promoter, an enhancer, 3′UTR, and/or polyadenylation signal of two or more of PreC, C, X, PreS1, PreS2, S, P and/or SP gene(s) is targeted for alteration (e.g., targeted knockout or targeted knockdown).

In certain embodiments, the methods, genome editing systems and compositions provide an alteration that comprises disrupting the PreC, C, X, PreS1, PreS2, S, P and/or SP gene by the insertion or deletion of one or more nucleotides mediated by Cas9 (e.g., enzymatically active Cas9 (eaCas9), e.g., Cas9 nuclease or Cas9 nickase) as described below. This type of alteration is also referred to as “knocking out” the PreC, C, X, PreS1, PreS2, S, P and/or SP gene.

In certain embodiments, the methods, genome editing systems and compositions provide an alteration of the expression of one or more of the PreC, C, X, PreS1, PreS2, S, P and/or SP genes that does not comprise nucleotide insertion or deletion in the PreC, C, X, PreS1, PreS2, S, P and/or SP gene and is mediated by enzymatically inactive Cas9 (eiCas9) or an eiCas9-fusion protein, as described below. This type of alteration is also referred to as “knocking down” the expression of one of more of the PreC, C, X, PreS1, PreS2, S, P and/or SP gene.

Knocking out PreC, C, X, PreS1, PreS2, S, P or SP genes, individually or in combination, can reduce HBV protein expression, infectivity, replication, and/or packaging and can therefore reduce, prevent and/or treat HBV infection. Knock down of the PreC, C, X, PreS1, PreS2, S, P or SP genes, individually or in combination, can reduce HBV protein expression, infectivity, replication, and/or packaging and can therefore reduce, prevent and/or treat HBV infection. Knock down of the PreC, C, X, PreS1, PreS2, S, P or SP genes, individually or in combination, can reduce HBV protein expression, causing the reduction of HBV peptide presentation by MHC class I and II molecules and the reversal of T-cell failure, which can treat HBV infection. Concomitant knockout and knock down of the PreC, C, X PreS1, PreS2, S, P or SP genes, individually or in combination, can reduce HBV protein expression, infectivity, replication, and/or packaging and can therefore reduce, prevent and/or treat HBV infection.

Knockout, knockdown or concomitant knockout and knockdown of the expression of the PreC, C, X, PreS1, PreS2, S, P or SP gene, individually or in combination, may cause any of the following, singly or in combination: decreased HBV DNA production, decreased HBV cccDNA production, decreased viral infectivity, decreased packaging of viral particles, decreased production of production of viral proteins, e.g., HBe, HBc, HBx, LHBs, MHBs, SHBs, Pol, and/or HBSP proteins, decreased presentation of HBV peptides by MHC class I and class II molecules, reversal of T-cell exhaustion and/or T-cell failure, and/or reversal of B-cell dysfunction. Knockout, knockdown or concomitant knockout and knockdown of the PreC, C, X PreS1, PreS2, S, P or SP genes, individually or in combination, may cause a decline in viral protein production, e.g., HBs Ag, HBeAg, HBcAg, HBxAg, HB preS1Ag, HB preS2Ag, HBsAg, HBpolAg and/or HBspAg. In certain embodiments, a decline in viral protein production may cause the restoration of immune response to HBV and clearance of chronic and/or acute HBV infection.

A vigorous CD8⁺ T cell response is thought to be important in the clearance of HBV (Schmidt et. al, Emerging Microbes & Infections (2013) 2, e15; Published online 27 Mar. 2013). The development of chronic HBV infection and concomitant failure to clear HBV is thought to be due to an impaired CD8⁺ T cell response to HBV (Ferrari C, et al. J Immunol 1990; 145: 3442-3449). The ability to restore CD8⁺ T cell response to HBV is thought to lead to the clearance and resolution of chronic HBV. (Webster et al. J Virol 2004; 78: 5707-5719.)

In certain embodiments, the methods, genome editing systems and compositions induce a decline in HBV protein production, e.g., HBe, HBc, HBx, LHBs, MHBs, SHBs, Pol, and/or HBSP protein production, so that there is a corresponding decline in HBV peptide presentation, e.g., HBe-derived, HBc-derived, HBx-derived, LHBs-derived, MHBs-derived, SHBs-derived, Pol-derived, and/or HBSP-derived peptide presentation, by MHC Class I molecules. MHC Class I molecules present HBV-derived peptides on infected liver cells and antigen presenting cells. In certain embodiments, the methods, genome editing systems and compositions lead to reconstitution of functional CD8⁺ T cell-mediated toxicity against HBV-infected hepatocytes, including CD-8⁺ T-cell mediated cell killing and/or CD-8⁺ T cell-mediated interferon (IFN) secretion locally within the liver parenchyma. In certain embodiments, CD-8⁺ T cell-mediated IFN secretion locally, e.g., within the liver parenchyma and/or at or near the site of HBV infected hepatocytes, mediates cell killing and clearance of HBV-infected cells without the systemic side effects of systemic IFN therapy. In certain embodiments, CD-8⁺ T cell-mediated IFN secretion locally leads to the clearance of HBV-infected hepatocytes and to a functional cure of HBV infection. In certain embodiments, the methods, genome editing systems and compositions lead to a reconstitution of immune competence by restoring activation of T-cell mediated cytotoxicity in subjects. IFN therapy in chronic HBV infection attempts to boost the immune response to HBV infection. the methods, genome editing systems and compositions described herein induce a local IFN response to HBV infection. In certain embodiments, the methods, genome editing systems and compositions described herein are more effective and have fewer systemic side effects, e.g., fever, malaise, or muscle aches, than systemic IFN-based therapy.

In certain embodiments, the methods, genome editing systems and compositions induce a decline in certain HBV proteins, e.g., HBc, e.g., HBpol, e.g., HBx, whose expression is thought to be the cause of T-cell failure in chronic HBV (Feng et. al, J Biomed Sci. 2007 January; 14(1):43-57).

In certain embodiments, the methods, genome editing systems and compositions induce a decline in any and/or all HBV protein production, e.g., HBe, HBc, HBx, LHBs, MHBs, SHBs, Pol, and/or HBSP protein production, as a high viral load is thought to be the primary mechanism for the failure of HBV-specific CD8+ T-cell responses (Schmidt et. al, Emerging Microbes & Infections (2013) 2, e15; Published online 27 Mar. 2013).

In certain embodiments, the methods, genome editing systems and compositions induce a decline in HBV protein production, e.g., HBe, HBc, HBx, LHBs, MHBs, SHBs, Pol, and/or HBSP protein. In certain embodiments, a decline in HBV protein production gives rise to a reduction in the overwhelming presentation of antigens to the humoral (B-cell) mediated immune system. In certain embodiments, B-cell mediated antibody production is no longer overwhelmed by HBV antigen production and B-cell mediated antibody production is stoichiometrically equivalent to HBV antigen production, e.g., HBsAg production is decreased and anti-HBs antibody can mediate clearance of HBsAg. In certain embodiments, a reduction in the volume and presentation of HBV antigens, e.g., HBeAg, HBcAg, HBxAg, HBsAg, HBpolAg allows for effective humoral immunity, e.g., viral-specific neutralizing antibody production, e.g., anti-HBe Ag production, e.g., anti-HBcAg production, e.g., anti-HBxAg production, e.g., anti-HBsAg production, e.g., anti-HBpolAg production. In certain embodiments, a reduction in the presentation of HBV antigens, e.g., HBeAg, HBcAg, HBxAg, HBsAg, HBpolAg allows for B-cell mediated antibody clearance of HBV antigens and viral particles, including the Dane particle.

In certain embodiments, a reduction in viral protein production leads to the reversal of ‘immune exhaustion’, with return of functional B-cell and T-cell responses against hepatocytes infected with HBV. In certain embodiments, the methods, genome editing systems and compositions induce a decline in viral protein production that causes B and T cells to achieve clearance of hepatocytes infected with HBV. In certain embodiments, the methods, genome editing systems and compositions induce a decline in viral protein production that causes a subject to achieve a functional virologic cure of chronic HBV, which is defined by a lack of HBsAg positivity on a serum assay.

In another aspect, the methods, genome editing systems and compositions discussed herein may be used to alter one or more of PreC, C, X PreS1, PreS2, S, P and/or SP gene(s) to treat, prevent and/or reduce HBV infection by targeting the coding sequence of one or more of PreC, C, X PreS1, PreS2, S, P and/or SP gene(s). In certain embodiments, the gene(s), e.g., the coding sequence of one or more of the PreC, C, X PreS1, PreS2, S, P and/or SP gene(s), are targeted to knock out one or more of PreC, C, X PreS1, PreS2, S, P and/or SP gene(s), e.g., to eliminate expression of one or more of PreC, C, X PreS1, PreS2, S, P and/or SP gene(s), e.g., to knockout one or more copies of one or more of PreC, C, X PreS1, PreS2, S, P and/or SP gene(s), e.g., by induction of an alteration comprising a deletion or mutation in one or more of PreC, C, X PreS1, PreS2, S, P and/or SP gene(s). In certain embodiments, the methods, genome editing systems and compositions provide an alteration that comprises an insertion or deletion. As described herein, a targeted knockout approach is mediated by non-homologous end joining (NHEJ) using a CRISPR/Cas system comprising a Cas9 molecule, fusion-protein or polypeptide, e.g., an enzymatically active Cas9 (eaCas9) molecule. In certain embodiments, the Cas9 molecule, fusion-protein or polypeptide is an S. pyogenes Cas9 variant. In certain embodiments, the S. pyogenes Cas9 variant is the EQR variant. In certain embodiments, the S. pyogenes Cas9 variant is the VRER variant. In certain embodiments, the Cas9 molecule, fusion-protein or polypeptide is an S. aureus Cas9 variant. In certain embodiments, the S. aureus Cas9 variant is the KKH variant.

In certain embodiments, an early coding sequence of one or more of PreC, C, X, PreS1, PreS2, S, P and/or SP gene(s) are targeted to knockout one or more of PreC, C, X PreS1, PreS2, S, P and/or SP gene(s). In certain embodiments, targeting affects one or more copies of the PreC, C, X, PreS1, PreS2, S, P and/or SP gene(s). In certain embodiments, a targeted knockout approach reduces or eliminates expression of one or more functional PreC, C, X, PreS1, PreS2, S, P and/or SP gene product(s). In certain embodiments, the methods, genome editing systems and compositions provide an alteration that comprises an insertion or deletion.

In another aspect, the methods, genome editing systems and compositions discussed herein may be used to alter one or more of PreC, C, X, PreS1, PreS2, S, P and/or SP gene(s) to treat, prevent and/or reduce HBV by targeting non-coding sequence of the PreC, C, X, PreS1, PreS2, S, P and/or SP gene(s), e.g., promoter, an enhancer, 3′UTR, and/or polyadenylation signal. In certain embodiments, the gene(s), e.g., the non-coding sequence of one or more PreC, C, X, PreS1, PreS2, S, P and/or SP gene(s), is targeted to knockout the gene(s), e.g., to eliminate expression of the gene(s), e.g., to knockout one or more copies of the PreC, C, X, PreS1, PreS2, S, P and/or SP gene(s), e.g., by induction of an alteration comprising a deletion or mutation in the PreC, C, X, PreS1, PreS2, S, P and/or SP gene(s). In certain embodiments, the methods, genome editing systems and compositions provide an alteration that comprises an insertion or deletion. In another aspect, a transcriptional regulatory region, e.g., a promoter region (e.g., a promoter region that controls the transcription of one or more of the PreC, C, X, PreS1, PreS2, S, P or SP genes) is targeted to alter (e.g., knock down) the expression of one or more of the PreC, C, X, PreS1, PreS2, S, P and/or SP gene(s). This type of alteration of the expression is also sometimes referred to as “knocking down” the expression of one or more of the PreC, C, X, PreS1, PreS2, S, P and/or SP gene(s). In certain embodiments, a targeted knockdown approach is mediated by a CRISPR/Cas system comprising a Cas9 molecule, e.g., an enzymatically inactive Cas9 (eiCas9) molecule or an eiCas9 fusion protein (e.g., an eiCas9 fused to a transcription repressor domain or chromatin modifying protein), as described herein. In an eiCas9 fusion protein (e.g., an eiCas9 fused to a transcription repressor domain or chromatin modifying protein, one or more gRNA molecules comprising a targeting domain are configured to target an enzymatically inactive Cas9 (eiCas9) molecule or an eiCas9 fusion protein (e.g., an eiCas9 fused to a transcription repressor domain), sufficiently close to the transcriptional regulatory region, e.g., a promoter region (e.g., a promoter region that controls the transcription of one or more PreC, C, X, PreS1, PreS2, S, P or SP genes). In certain embodiments, the eiCas9 molecule, fusion-protein or polypeptide is an S. pyogenes Cas9 variant. In certain embodiments, the S. pyogenes Cas9 variant is the EQR variant. In certain embodiments, the S. pyogenes Cas9 variant is the VRER variant. In certain embodiments, the Cas9 molecule, fusion-protein or polypeptide is an S. aureus Cas9 variant. In certain embodiments, the S. aureus Cas9 variant is the KKH variant. In certain embodiments, this approach gives rise to reduction, decrease or repression of the expression of one or more of the PreC, C, X PreS1, PreS2, S, P or SP genes. In certain embodiments, a promoter region that controls the transcription of one or more PreC, C, X, PreS1, PreS2, S, P or SP genes is located within HBV cccDNA. In certain embodiments, a promoter region that controls the transcription of one or more PreC, C, X, PreS1, PreS2, S, P or SP genes is located within integrated HBV DNA.

In certain embodiments, knockdown of one or more of the PreC, C, X PreS1, PreS2, S, P and/or SP gene(s) is performed by targeting the gene(s) within HBV cccDNA and/or integrated HBV DNA. In certain embodiments, eiCas9 or an eiCas9 fusion protein is utilized to knock down one or more of the PreC, C, X PreS1, PreS2, S, P and/or SP gene(s) located within the HBV cccDNA residing in an infected hepatocyte. In certain embodiments, eiCas9 or an eiCas9 fusion protein is utilized to knock down one or more of the PreC, C, X PreS1, PreS2, S, P and/or SP gene(s) that are integrated within the human genome in an infected hepatocyte. In certain embodiments, knockdown one or more of the PreC, C, X, PreS1, PreS2, S, P and/or SP gene(s) (located on cccDNA and/or integrated HBV DNA) may decrease the production of HBV rcDNA, HBV linearized DNA, HBV RNA intermediates and/or HBV proteins, e.g., HBe, HBc, HBx, LHBs, MHBs, SHBs, Pol, and/or HBSP.

In certain embodiments, HBV protein expression, including HBsAg production, results from expression at integrated HBV DNA sites in the human genome. In certain embodiments, knockdown of HBV protein production, e.g., HBe, HBc, HBx, LHBs, MHBs, SHBs, Pol, and/or HBSP protein production, by eiCas9 or an eiCas9 fusion protein mediated knock down of HBV DNA in cccDNA form and/or HBV DNA in integrated form allows recovery of a subject's B-cell mediated antibody response to HBV. In certain embodiments, knockdown of HBV protein production, e.g., HBe, HBc, HBx, LHBs, MHBs, SHBs, Pol, and/or HBSP protein production, by eiCas9 or an eiCas9 fusion protein mediated knock down of HBV DNA in cccDNA form and/or HBV DNA in integrated form allows recovery of a subject's T-cell mediated response to HBV. The methods, genome editing systems and compositions described herein promote the recovery of B-cell and/or T-cell mediated response to HBV. In certain embodiments, the methods, genome editing systems and compositions described herein lead to the reversal of immune exhaustion in a subject. In certain embodiments, the methods, genome editing systems and compositions described herein lead to clearance of infected hepatocytes.

In certain embodiments, knockdown of HBV protein production, e.g., HBc (HB core protein), HBpol (HB polymerase protein), HBx (HB x protein) and/or HBs (HB s protein) by eiCas9 or an eiCas9 fusion protein mediated knockdown of integrated genomic HBV DNA, leads to reversal of immune exhaustion in a subject, restoration of T-cell mediated immunity and/or clearance of chronic HBV infection.

In certain embodiments, knockdown of HBc (HB core protein) production, by eiCas9 or an eiCas9 fusion protein mediated knock down of HBV cccDNA, leads to reversal of immune exhaustion in a subject, restoration of T-cell mediated immunity and/or clearance of chronic HBV infection. In certain embodiments, knockdown of HBx (HB x protein) production, by eiCas9 or an eiCas9 fusion protein mediated knock down of HBV cccDNA, leads to reversal of immune exhaustion in a subject, restoration of T-cell mediated immunity and/or clearance of chronic HBV infection. In certain embodiments, knockdown of HBpol (HB polymerase protein) production, by eiCas9 or an eiCas9 fusion protein mediated knock down of HBV cccDNA, leads to reversal of immune exhaustion in a subject, restoration of T-cell mediated immunity and/or clearance of chronic HBV infection. In certain embodiments, knockdown of HBs (HB s protein) production, by eiCas9 or an eiCas9 fusion protein mediated knock down of HBV cccDNA, leads to reversal of immune exhaustion in a subject, restoration of T-cell mediated immunity and/or clearance of chronic HBV infection.

In certain embodiments, knockdown of HB core protein production, by eiCas9 or an eiCas9 fusion protein mediated knockdown of both integrated genomic HBV DNA and HBV cccDNA, leads to reversal of immune exhaustion, restoration of T-cell mediated immunity and/or clearance of chronic HBV infection in a subject. In certain embodiments, knockdown of HB x protein production, by eiCas9 or an eiCas9 fusion protein mediated knockdown of both integrated genomic HBV DNA and HBV cccDNA, leads to reversal of immune exhaustion, restoration of T-cell mediated immunity and/or clearance of chronic HBV infection in a subject. In certain embodiments, knockdown of HB polymerase protein production, by eiCas9 or an eiCas9 fusion protein mediated knockdown of both integrated genomic HBV DNA and HBV cccDNA, leads to reversal of immune exhaustion, restoration of T-cell mediated immunity and/or clearance of chronic HBV infection in a subject.

In certain embodiments, knockdown of HBs protein production, by eiCas9 or an eiCas9 fusion protein mediated knockdown of both integrated genomic HBV DNA and HBV cccDNA, leads to reversal of immune exhaustion, restoration of T-cell mediated immunity and/or clearance of chronic HBV infection in a subject.

In certain embodiments, knockdown of one or more of HBV protein production, e.g., HBe, HBc, HBx, LHBs, MHBs, SHBs, Pol, and/or HBSP, by eiCas9 or an eiCas9 fusion protein mediated knock down of integrated genomic HBV DNA and/or HBV cccDNA, leads to reversal of immune exhaustion, restoration of T-cell mediated immunity and/or clearance of chronic HBV infection in a subject.

In certain embodiments, knockdown of one or more of the PreC, C, X PreS1, PreS2, S, P and/or SP gene(s) cures HBV infection. In certain embodiments, the knockdown of one or more of the PreC, C, X PreS1, PreS2, S, P and/or SP gene(s) provides a functional cure of the HBV infection. In certain embodiments, knockdown of one or more of the PreC, C, X PreS1, PreS2, S, P and/or SP gene(s) leads to a sustained virologic response to HBV infection. In certain embodiments, knockdown of one or more of the PreC, C, X PreS1, PreS2, S, P and/or SP gene(s) is an effective method of preventing the sequelae of chronic HBV, including fibrosis, cirrhosis, and hepatocellular carcinoma.

In certain embodiments, one or more of the PreC, C, X PreS1, PreS2, S, P and/or SP gene(s) that is known to be integrated into the subject genome is targeted for knockdown. In certain embodiments, one or more of the PreC, C, X, PreS1, PreS2, S, P and/or SP gene(s) or one or more of a region of the HBV genome, e.g., the DR1 region, e.g., the DR2 region, e.g., PreC, e.g., C, that is known not to be integrated into the subject genome, is targeted for knockout. The DR1 region is a 12 base pair direct repeat region near the 5′ end of the HBV genome. The DR2 region is a 12 base pair direct repeat region near the 3′ end of the HBV genome. The HBV genome has been demonstrated to integrate into the human genome using the DR1 and/or DR2 regions as the host-viral DNA junction (DeJean et. al, Proceedings of National Academy of Science, 1984: 81:5350-5354). A common 2 base pair deletion in each of the DR1 and DR2 regions has been identified in integrated HBV DNA. In certain embodiments, targeting of the full DR1 and/or DR2 sequence for knockout (e.g., non-deleted form), e.g., 5′ T-T-C-A-C-C-T-C-T-G-C, allows for specific knockout of a region that is known not to be integrated and/or is less commonly integrated into a subject's DNA. In certain embodiments, targeting of a partially deleted DR1 and/or DR2 sequence for knockdown, e.g., 5′ C-A-C-C-T-C-T-G-C, allows for specific knockdown of a region that is known to be integrated into a subject's DNA.

In certain embodiments, the methods, genome editing systems and compositions comprise knockdown of a region of the HBV genome, e.g., one or more of the PreC, C, X PreS1, PreS2, S, P and/or SP gene(s), that is integrated into the subject genome. In certain embodiments, the methods, genome editing systems and compositions comprise knockdown of a region of the HBV genome, e.g., one or more of the PreC, C, X PreS1, PreS2, S, P and/or SP gene(s), in a manner that targets both a region of HBV cccDNA and an integrated region of the HBV genome.

In certain embodiments, the methods, genome editing systems and compositions disclosed herein can comprise knockdown of a region of the HBV genome, e.g., the S gene, e.g., one or more of the PreC, C, X PreS1, PreS2, P and/or SP gene(s) that is integrated into the subject genome in order to decrease circulating HBV antigen levels, including but not limited to HBsAg. In a chimpanzee model, integrated DNA is implicated in the production of HBsAg and in circulating HBs antigen-emia (Wooddell et al., AASLD abstract #32, Hepatology, 2015: 222A-223A). In certain embodiments, the method comprises knockdown of a region of the HBV genome, e.g., the S gene, to induce a functional cure of HBV infection.

In certain embodiments, the methods, genome editing systems and compositions comprise knockout of a region of the HBV genome, e.g., one or more of the PreC, C, X PreS1, PreS2, S, P and/or SP gene(s), that is not integrated into the subject genome. In certain embodiments, the methods, genome editing systems and compositions comprise knockout of a region of the HBV genome, e.g., one or more of the PreC, C, X, PreS1, PreS2, S, P and/or SP gene(s), in a manner that targets both a region of HBV cccDNA and an integrated region of the HBV genome.

In certain embodiments, the methods, genome editing systems and compositions comprise concomitant 1) knockout and 2) knockdown of two distinct regions of the HBV genome, e.g., 1) knockdown of a region of the HBV genome that is integrated into the subject genome and 2) knockout of a different region of the HBV genome that is not integrated into the subject genome (e.g., on the HBV ccc DNA).

The methods, genome editing systems and compositions described herein may reduce the risk of hepatocellular carcinoma in a subject who has been exposed to HBV or who has chronic HBV. The methods, genome editing systems and compositions described herein may also reduce the risk of cirrhosis, fibrosis and end stage liver disease in a subject who has been exposed to HBV or who has chronic HBV.

In certain embodiments, the coding region of the PreC, C, X, PreS1, PreS2, S, P or SP gene, is targeted to alter the expression of the PreC, C, X, PreS1, PreS2, S, P or SP gene. In certain embodiments, a non-coding region (e.g., an enhancer region, a promoter region, 5′ UTR, 3′UTR, polyadenylation signal) of the PreC, C, X, PreS1, PreS2, S, P or SP gene is targeted to alter the expression of the PreC, C, X, PreS1, PreS2, S, P or SP gene. In certain embodiments, the promoter region of the PreC, C, X, PreS1, PreS2, S, P or SP gene is targeted to knock down the expression of one or more of the PreC, C, X, PreS1, PreS2, S, P or SP gene. A targeted knockdown approach alters, e.g., reduces or eliminates the expression of the PreC, C, X, PreS1, PreS2, S, P or SP gene. As described herein, in certain embodiments, a targeted knockdown is mediated by targeting an enzymatically inactive Cas9 (eiCas9) or an eiCas9 fused to a transcription repressor domain or chromatin modifying protein to alter transcription, e.g., to block, reduce, or decrease transcription, of the PreC, C, X, PreS1, PreS2, S, P or SP gene.

In certain embodiments, one or more gRNA molecules comprise a targeting domain configured to target an enzymatically inactive Cas9 (eiCas9) or an eiCas9 fusion protein (e.g., an eiCas9 fused to a transcription repressor domain), sufficiently close to an HBV target knockdown position to reduce, decrease or repress expression of the PreC, C, X, PreS1, PreS2, S, P or SP gene.

The presently disclosed subject matter provides a genome editing system, a composition or a vector comprising: a gRNA molecule comprising a targeting domain that is complementary with a target sequence of a Hepatitis B virus (HBV) viral gene selected from the group consisting of PreC gene, C gene, X gene, PreS1 gene, PreS2 gene, S gene, P gene and SP gene. In certain embodiments, the genome editing system, composition, or vector further comprises a Cas9 molecule. In certain embodiments, the targeting domain is configured to form a double strand break or a single strand break within about 500 bp, about 450 bp, about 400 bp, about 350 bp, about 300 bp, about 250 bp, about 200 bp, about 150 bp, about 100 bp, about 50 bp, about 25 bp, or about 10 bp of an HBV target position, thereby altering the HBV viral gene. Alteration of the HBV viral gene can include knockout of the HBV viral gene, knockdown of the HBV viral gene, or concomitant knockout and knockdown of the HBV viral gene.

In certain embodiments, the targeting domain comprises a nucleotide sequence that is identical to, or differs by no more than 1, 2, or 3 nucleotides from, a nucleotide sequence selected from SEQ ID NOS: 215-141071.

In certain embodiments, the Cas9 molecule is an S. pyogenes Cas9 molecule, and the targeting domain comprises a nucleotide sequence that is identical to, or differs by no more than 1, 2, or 3 nucleotides from, a nucleotide sequence selected from the group consisting of:

(a) SEQ ID NOS: 15389-16329;

(b) SEQ ID NOS: 31598-32518;

(c) SEQ ID NOS: 47978-48841;

(d) SEQ ID NOS: 62798-63714;

(e) SEQ ID NOS: 79221-80079;

(f) SEQ ID NOS: 94449-95356;

(g) SEQ ID NOS: 110120-111022; and

(h) SEQ ID NOS: 125842-126712.

In certain embodiments, the S. pyogenes Cas9 molecule recognizes a Protospacer Adjacent Motif (PAM) of NGG, the genome editing system, composition, or vector targets HBV genotype A (HBV-A), and the targeting domain comprises a nucleotide sequence that is identical to, or differs by no more than 1, 2, or 3 nucleotides from, a nucleotide sequence selected from SEQ ID NOS: 15389-16329.

In certain embodiments, the S. pyogenes Cas9 molecule recognizes a PAM of NGG, the genome editing system, composition, or vector targets HBV genotype B (HBV-B), and the targeting domain comprises a nucleotide sequence that is identical to, or differs by no more than 1, 2, or 3 nucleotides from, a nucleotide sequence selected from SEQ ID NOS: 31598-32518.

In certain embodiments, the S. pyogenes Cas9 molecule recognizes a PAM of NGG, the genome editing system, composition, or vector targets HBV genotype C (HBV-C), and the targeting domain comprises a nucleotide sequence that is identical to, or differs by no more than 1, 2, or 3 nucleotides from, a nucleotide sequence selected from SEQ ID NOS: 47978-48841.

In certain embodiments, the S. pyogenes Cas9 molecule recognizes a PAM of NGG, the genome editing system, composition, or vector targets HBV genotype D (HBV-D), and the targeting domain comprises a nucleotide sequence that is identical to, or differs by no more than 1, 2, or 3 nucleotides from, a nucleotide sequence selected from SEQ ID NOS: 62798-63714.

In certain embodiments, the Cas9 molecule is an S. pyogenes Cas9 EQR variant, and the targeting domain comprises a nucleotide sequence that is identical to, or differs by no more than 1, 2, or 3 nucleotides from, a nucleotide sequence selected from the group consisting of:

(a) SEQ ID NOS: 215-1565;

(b) SEQ ID NOS: 2225-3535;

(c) SEQ ID NOS: 4169-5381;

(d) SEQ ID NOS: 5977-7325;

(e) SEQ ID NOS: 7953-9213;

(f) SEQ ID NOS: 9830-11082;

(g) SEQ ID NOS: 11678-12954; and

(h) SEQ ID NOS: 13563-14791.

In certain embodiments, the S. pyogenes Cas9 EQR variant recognizes a PAM selected from the group consisting of NGAG, NGCG, NGGG, NGTG, NGAA, NGAT, and NGAC, the genome editing system, composition, or vector targets HBV genotype A (HBV-A), and the targeting domain comprises a nucleotide sequence that is identical to, or differs by no more than 1, 2, or 3 nucleotides from, a nucleotide sequence selected from SEQ ID NOS: 215-1565.

In certain embodiments, the S. pyogenes Cas9 EQR variant recognizes a PAM selected from the group consisting of NGAG, NGCG, NGGG, NGTG, NGAA, NGAT, and NGAC, the genome editing system, composition, or vector targets HBV genotype B (HBV-B), and the targeting domain comprises a nucleotide sequence that is identical to, or differs by no more than 1, 2, or 3 nucleotides from, a nucleotide sequence selected from SEQ ID NOS: 2225-3535.

In certain embodiments, the S. pyogenes Cas9 EQR variant recognizes a PAM selected from the group consisting of NGAG, NGCG, NGGG, NGTG, NGAA, NGAT, and NGAC, the genome editing system, composition, or vector targets HBV genotype C (HBV-C), and the targeting domain comprises a nucleotide sequence that is identical to, or differs by no more than 1, 2, or 3 nucleotides from, a nucleotide sequence selected from SEQ ID NOS: 4169-5381.

In certain embodiments, the S. pyogenes Cas9 EQR variant recognizes a PAM selected from the group consisting of NGAG, NGCG, NGGG, NGTG, NGAA, NGAT, and NGAC, the genome editing system, composition, or vector targets HBV genotype D (HBV-D), and the targeting domain comprises a nucleotide sequence that is identical to, or differs by no more than 1, 2, or 3 nucleotides from, a nucleotide sequence selected from SEQ ID NOS: 5977-7325.

In certain embodiments, the Cas9 molecule is an S. pyogenes Cas9 VRER variant, and the targeting domain comprises a nucleotide sequence that is identical to, or differs by no more than 1, 2, or 3 nucleotides from, a nucleotide sequence selected from the group consisting of:

(a) SEQ ID NOS: 1566-2224;

(b) SEQ ID NOS: 3536-4168;

(c) SEQ ID NOS: 5382-5976;

(d) SEQ ID NOS: 7326-7952;

(e) SEQ ID NOS: 9214-9829;

(f) SEQ ID NOS: 11083-11677;

(g) SEQ ID NOS: 12955-13562; and

(h) SEQ ID NOS: 14792-15388.

In certain embodiments, the S. pyogenes Cas9 VRER variant recognizes a PAM selected from the group consisting of NGCG, NGCA, NGCT, and NGCC, the genome editing system, composition, or vector targets HBV genotype A (HBV-A), and the targeting domain comprises a nucleotide sequence that is identical to, or differs by no more than 1, 2, or 3 nucleotides from, a nucleotide sequence selected from SEQ ID NOS: 1566-2224.

In certain embodiments, the S. pyogenes Cas9 VRER variant recognizes a PAM selected from the group consisting of NGCG, NGCA, NGCT, and NGCC, the genome editing system, composition, or vector targets HBV genotype B (HBV-B), and the targeting domain comprises a nucleotide sequence that is identical to, or differs by no more than 1, 2, or 3 nucleotides from, a nucleotide sequence selected from SEQ ID NOS: 3536-4168.

In certain embodiments, the S. pyogenes Cas9 VRER variant recognizes a PAM selected from the group consisting of NGCG, NGCA, NGCT, and NGCC, the genome editing system, composition, or vector targets HBV genotype C (HBV-C), and the targeting domain comprises a nucleotide sequence that is identical to, or differs by no more than 1, 2, or 3 nucleotides from, a nucleotide sequence selected from SEQ ID NOS: 5382-5976.

In certain embodiments, the S. pyogenes Cas9 VRER variant recognizes a PAM selected from the group consisting of NGCG, NGCA, NGCT, and NGCC, the genome editing system, composition, or vector targets HBV genotype D (HBV-D), and the targeting domain comprises a nucleotide sequence that is identical to, or differs by no more than 1, 2, or 3 nucleotides from, a nucleotide sequence selected from SEQ ID NOS: 7326-7952.

In certain embodiments, the Cas9 molecule is an S. aureus Cas9 molecule, and the targeting domain comprises a nucleotide sequence that is identical to, or differs by no more than 1, 2, or 3 nucleotides from, a nucleotide sequence selected from the group consisting of:

(a) SEQ ID NOS: 16330-19822;

(b) SEQ ID NOS: 32519-35976;

(c) SEQ ID NOS: 48842-51921;

(d) SEQ ID NOS: 63715-67224;

(e) SEQ ID NOS: 80080-83218;

(f) SEQ ID NOS: 95357-98663;

(g) SEQ ID NOS: 111023-114350; and

(h) SEQ ID NOS: 126713-129862.

In certain embodiments, the S. aureus Cas9 molecule recognizes a PAM of either NNNRRT or NNNRRV, the genome editing system, composition, or vector targets HBV genotype A (HBV-A), and the targeting domain comprises a nucleotide sequence that is identical to, or differs by no more than 1, 2, or 3 nucleotides from, a nucleotide sequence selected from SEQ ID NOS: 16330-19822.

In certain embodiments, the S. aureus Cas9 molecule recognizes a PAM of either NNNRRT or NNNRRV, the genome editing system, composition, or vector targets HBV genotype B (HBV-B), and the targeting domain comprises a nucleotide sequence that is identical to, or differs by no more than 1, 2, or 3 nucleotides from, a nucleotide sequence selected from SEQ ID NOS: 32519-35976.

In certain embodiments, the S. aureus Cas9 molecule recognizes a PAM of either NNNRRT or NNNRRV, the genome editing system, composition, or vector targets HBV genotype C (HBV-C), and the targeting domain comprises a nucleotide sequence that is identical to, or differs by no more than 1, 2, or 3 nucleotides from, a nucleotide sequence selected from SEQ ID NOS: 48842-51921.

In certain embodiments, the S. aureus Cas9 molecule recognizes a PAM of either NNNRRT or NNNRRV, the genome editing system, composition, or vector targets HBV genotype D (HBV-D), and the targeting domain comprises a nucleotide sequence that is identical to, or differs by no more than 1, 2, or 3 nucleotides from, a nucleotide sequence selected from SEQ ID NOS: 63715-67224.

In certain embodiments, the Cas9 molecule is an S. aureus Cas9 KKH variant, and the targeting domain comprises a nucleotide sequence that is identical to, or differs by no more than 1, 2, or 3 nucleotides from, a nucleotide sequence selected from the group consisting of:

(a) SEQ ID NOS: 19823-31597;

(b) SEQ ID NOS: 35977-47977;

(c) SEQ ID NOS: 51922-62797;

(d) SEQ ID NOS: 67225-79220;

(e) SEQ ID NOS: 83219-94448;

(f) SEQ ID NOS: 98664-110119;

(g) SEQ ID NOS: 114351-125841; and

(h) SEQ ID NOS: 129863-141071.

In certain embodiments, the S. aureus Cas9 KKH variant recognizes a PAM of either NNNRRT or NNNRRV, the genome editing system, composition, or vector targets HBV genotype A (HBV-A), and the targeting domain comprises a nucleotide sequence that is identical to, or differs by no more than 1, 2, or 3 nucleotides from, a nucleotide sequence selected from SEQ ID NOS: 19823-31597.

In certain embodiments, the S. aureus Cas9 KKH variant recognizes a PAM of either NNNRRT or NNNRRV, the genome editing system, composition, or vector targets HBV genotype B (HBV-B), and the targeting domain comprises a nucleotide sequence that is identical to, or differs by no more than 1, 2, or 3 nucleotides from, a nucleotide sequence selected from SEQ ID NOS: 35977-47977.

In certain embodiments, the S. aureus Cas9 KKH variant recognizes a PAM of either NNNRRT or NNNRRV, the genome editing system, composition, or vector targets HBV genotype C (HBV-C), and the targeting domain comprises a nucleotide sequence that is identical to, or differs by no more than 1, 2, or 3 nucleotides from, a nucleotide sequence selected from SEQ ID NOS: 51922-62797.

In certain embodiments, the S. aureus Cas9 KKH variant recognizes a PAM of either NNNRRT or NNNRRV, the genome editing system, composition, or vector targets HBV genotype D (HBV-D), and the targeting domain comprises a nucleotide sequence that is identical to, or differs by no more than 1, 2, or 3 nucleotides from, a nucleotide sequence selected from SEQ ID NOS: 67225-79220.

The presently disclosed subject matter further provides a gRNA molecule, e.g., an isolated or non-naturally occurring gRNA molecule, comprising a targeting domain which is complementary with a target sequence of a Hepatitis B virus (HBV) viral gene selected from the group consisting of PreC gene, C gene, X gene, PreS1 gene, PreS2 gene, S gene, P gene and SP gene.

In certain embodiments, the targeting domain of the gRNA molecule is configured to provide a cleavage event, e.g., a double strand break or a single strand break, sufficiently close to a HBV target position in the PreC, C, X, PreS1, PreS2, S, P or SP gene to allow alteration, e.g., alteration associated with NHEJ, of a HBV target position in the PreC, C, X, PreS1, PreS2, S, P or SP gene. In certain embodiments, the targeting domain is configured such that a cleavage event, e.g., a double strand or single strand break, is positioned within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of a HBV target position. The break, e.g., a double strand or single strand break, can be positioned upstream or downstream of a HBV target position in the PreC, C, X, PreS1, PreS2, S, P or SP gene. In certain embodiments, the targeting domain of the gRNA molecule is configured to provide a cleavage event selected from a double strand break and a single strand break, within 500 (e.g., within 500, 400, 300, 250, 200, 150, 100, 80, 60, 40, 20, or 10) nucleotides of a HBV target position.

In certain embodiments, a second gRNA molecule comprising a second targeting domain is configured to provide a cleavage event, e.g., a double strand break or a single strand break, sufficiently close to the HBV target position in the PreC, C, X PreS1, PreS2, S, P or SP gene, to allow alteration, e.g., alteration associated with NHEJ, of the HBV target position in the PreC, C, X PreS1, PreS2, S, P or SP gene, either alone or in combination with the break positioned by said first gRNA molecule. In certain embodiments, the targeting domains of the first and second gRNA molecules are configured such that a cleavage event, e.g., a double strand or single strand break, is positioned, independently for each of the gRNA molecules, within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position. In certain embodiments, the breaks, e.g., double strand or single strand breaks, are positioned on both sides of a nucleotide of a HBV target position in the PreC, C, X, PreS1, PreS2, S, P or SP gene. In certain embodiments, the breaks, e.g., double strand or single strand breaks, are positioned on one side, e.g., upstream or downstream, of a nucleotide of a HBV target position in the PreC, C, X, PreS1, PreS2, S, P or SP gene. In certain embodiments, the targeting domain of the first and/or second gRNA molecule is configured to provide a cleavage event selected from a double strand break and a single strand break, within about 500 (e.g., within about 500, about 400, about 300, about 250, about 200, about 150, about 100, about 80, about 60, about 40, about 20, or about 10) nucleotides of a HBV target position.

In certain embodiments, a single strand break is accompanied by an additional single strand break, positioned by a second gRNA molecule, as discussed below. For example, the targeting domains are configured such that a cleavage event, e.g., the two single strand breaks, are positioned within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of a HBV target position. In certain embodiments, the first and second gRNA molecules are configured such, that when guiding a Cas9 molecule, e.g., a Cas9 nickase, a single strand break will be accompanied by an additional single strand break, positioned by a second gRNA, sufficiently close to one another to result in alteration of a HBV target position in the PreC, C, X PreS1, PreS2, S, P or SP gene. In certain embodiments, the first and second gRNA molecules are configured such that a single strand break positioned by said second gRNA is within about 10, about 20, about 30, about 40, or about 50 nucleotides of the break positioned by said first gRNA molecule, e.g., when the Cas9 molecule is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second gRNA molecule, as is discussed below. For example, the targeting domain of a first gRNA molecule is configured such that a double strand break is positioned upstream of a HBV target position in the PreC, C, X PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position; and the targeting domain of a second gRNA molecule is configured such that a double strand break is positioned downstream of a HBV target position in the PreC, C, X PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position.

In certain embodiments, a double strand break can be accompanied by two additional single strand breaks, positioned by a second gRNA molecule and a third gRNA molecule. For example, the targeting domain of a first gRNA molecule is configured such that a double strand break is positioned upstream of a HBV target position in the PreC, C, X PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position; and the targeting domains of a second and third gRNA molecule are configured such that two single strand breaks are positioned downstream of a HBV target position in the PreC, C, X, PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position. In certain embodiments, the targeting domain of the first, second and third gRNA molecules are configured such that a cleavage event, e.g., a double strand or single strand break, is positioned, independently for each of the gRNA molecules.

In certain embodiments, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a HBV target position in the PreC, C, X, PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position; and the targeting domains of a third and fourth gRNA molecule are configured such that two single strand breaks are positioned downstream of a HBV target position in the PreC, C, X PreS1, PreS2, S, P or SP gene, e.g., within about 1, about 2, about 3, about 4, about 5, about 10, about 15, about 20, about 25, about 30, about 35, about 40, about 45, about 50, about 60, about 70, about 80, about 90, about 100, about 150 or about 200 nucleotides of the target position. In certain embodiments, the targeting domain of the first, second, third, and/or fourth gRNA molecule is configured to provide a cleavage event selected from a double strand break and a single strand break, within about 500 (e.g., within about 500, about 400, about 300, about 250, about 200, about 150, about 100, about 80, about 60, about 40, about 20, or about 10) nucleotides of a HBV target position.

In certain embodiments, when multiple gRNAs are used to generate (1) two single stranded breaks in close proximity, (2) two double stranded breaks, e.g., flanking a HBV target position (e.g., to remove a piece of DNA, e.g., to create a deletion mutation) or to create more than one indel in the gene, e.g., in a coding region, e.g., an early coding region, (3) one double stranded break and two paired nicks flanking a HBV target position (e.g., to remove a piece of DNA, e.g., to insert a deletion) or (4) four single stranded breaks, two on each side of a position, that they are targeting the same HBV target position. In certain embodiments, multiple gRNAs may be used to target more than one HBV target position in the same gene, e.g., one or more of PreC, X, PreS1, PreS2, S, P and/or SP gene(s).

In certain embodiments, the targeting domain of the first gRNA molecule and the targeting domain of the second gRNA molecules are complementary to opposite strands of the target nucleic acid molecule. In certain embodiments, the first gRNA molecule and the second gRNA molecule are configured such that the PAMs are oriented outward.

In certain embodiments, the targeting domain of a gRNA molecule is configured to avoid unwanted target chromosome elements, such as repeat elements, e.g., Alu repeats, in the target domain. The gRNA molecule may be a first, second, third and/or fourth gRNA molecule, as described herein.

In certain embodiments, the targeting domain of a gRNA molecule is configured to position a cleavage event sufficiently far from a preselected nucleotide, e.g., the nucleotide of a coding region, such that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

In certain embodiments, the targeting domain comprises a nucleotide sequence that is identical to, or differs by no more than 1, 2, 3, 4, or 5 nucleotides from, the nucleotide sequence selected the nucleotide sequence selected from SEQ ID NOS: 215 to 141071.

In certain embodiments, an HBV target position in the coding region, e.g., the early coding region, of the PreC, C, X, PreS1, PreS2, S, P or SP gene is targeted, e.g., for knockout. In certain embodiments, a HBV target position in the non-coding region, e.g., promoter, an enhancer, 3′UTR, and/or polyadenylation signal of the PreC, C, X, PreS1, PreS2, S, P or SP gene is targeted, e.g., for knockout. In certain embodiments, a HBV target position in a transcriptional regulatory region, e.g., a promoter region (e.g., a promoter region that controls the transcription of one or more of the PreC, C, X, PreS1, PreS2, S, P or SP genes) is targeted to alter (e.g., knock down) the expression of one or more of the PreC, C, X, PreS1, PreS2, S, P and/or SP gene(s).

In certain embodiments, when the HBV target position is the PreC, C, X, PreS1, PreS2, S, P or SP gene coding region, e.g., an early coding region, and more than one gRNA is used to position breaks, e.g., two single stranded breaks or two double stranded breaks, or a combination of single strand and double strand breaks, e.g., to create one or more indels, in the target nucleic acid sequence.

In certain embodiments, when the HBV target position is the PreC, C, X, PreS1, PreS2, S, P or SP gene non-coding region, e.g., promoter, an enhancer, 3′UTR, and/or polyadenylation signal, and more than one gRNA is used to position breaks, e.g., two single stranded breaks or two double stranded breaks, or a combination of single strand and double strand breaks, e.g., to create one or more indels, in the target nucleic acid sequence.

In certain embodiments, the gRNA is a modular gRNA or a chimeric gRNA. In certain embodiments, the targeting domain has a length of 16, 17, 18, 19, 20, 21, 22, 23, 24, 25, or 26 nucleotides in length.

A gRNA as described herein may comprise from 5′ to 3′: a targeting domain (comprising a “core domain”, and optionally a “secondary domain”); a first complementarity domain; a linking domain; a second complementarity domain; and a proximal domain. In certain embodiments, the gRNA molecule further comprises a tail domain. In certain embodiments, the proximal domain and tail domain are taken together as a single domain.

In certain embodiments, a gRNA molecule comprises a linking domain of no more than 25 nucleotides in length; a proximal and tail domain, that taken together, are at least 20, 25, 30, or 40 nucleotides in length; and a targeting domain equal to or greater than 16, 17, 18, 19, 20, 21, 22, 23, 24, 25 or 26 nucleotides in length.

A cleavage event, e.g., a double strand or single strand break, is generated by a Cas9 molecule. The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule). In certain embodiments, the eaCas9 molecule catalyzes a double strand break.

The Cas9 molecule can a wild-type Cas9 molecule, a mutant Cas9 molecule, or a combination thereof. In certain embodiments, the mutant Cas9 molecule comprises a mutation selected from the group consisting of D10, E762, D986, H840, N854, N863, and N580. In certain embodiments, the Cas9 molecule is an S. aureus Cas9 molecule or an S. pyogenes Cas9 molecule. The S. aureus Cas9 molecule can be an S. aureus Cas9 variant. In certain embodiments, the S. aureus Cas9 variant is an S. aureus Cas9 KKH variant. The S. pyogenes Cas9 molecule can be an S. pyogenes Cas9 variant. In certain embodiments, the S. pyogenes Cas9 variant is an S. pyogenes Cas9 EQR variant or an S. pyogenes Cas9 VRER variant.

In certain embodiments, the eaCas9 molecule comprises HNH-like domain cleavage activity but has no, or no significant, RuvC-like domain cleavage activity. In certain embodiments, the eaCas9 molecule is an HNH-like domain nickase, e.g., the eaCas9 molecule comprises a mutation at D10, e.g., D10A. In certain embodiments, the eaCas9 molecule comprises RuvC-like domain cleavage activity but has no, or no significant, HNH-like domain cleavage activity. In certain embodiments, the eaCas9 molecule is a RuvC-like domain nickase, e.g., the eaCas9 molecule comprises a mutation at H840, e.g., H840A. In certain embodiments, the eaCas9 molecule is a RuvC-like domain nickase, e.g., the eaCas9 molecule comprises a mutation at N863, e.g., N863A.

In certain embodiments, a single strand break is formed in the strand of the target nucleic acid to which the targeting domain of said gRNA is complementary. In certain embodiments, a single strand break is formed in the strand of the target nucleic acid other than the strand to which the targeting domain of said gRNA is complementary.

The presently disclosed subject matter further provides a composition comprising a presently disclosed gRNA molecule as described herein. In certain embodiments, the composition is a pharmaceutical composition. In certain embodiments, certain compositions described herein, e.g., pharmaceutical compositions described herein, can be used in the treatment, prevention and/or reduction of HBV infection in a subject.

Furthermore, the presently disclosed subject matter provides a vector comprising a presently disclosed gRNA molecule as described herein. In certain embodiments, the vector is a viral vector, which can be an adeno-associated virus (AAV) vector or a lentivirus (LV) vector.

Additionally, the presently disclosed subject matter provides a cell comprising a presently disclosed genome editing system, a presently disclosed composition, or a presently disclosed vector, as described herein. In certain embodiments, the cell is a cell expressing sodium taurocholate co-transporting polypeptide (NTCP) receptor. In certain embodiments, the cell is a hepatocyte.

The presently disclosed subject matter further provides a nucleic acid composition, e.g., an isolated or non-naturally occurring nucleic acid composition, e.g., DNA, that comprises (a) a nucleotide sequence that encodes a presently disclosed gRNA molecule as described herein. The nucleic acid disclosed herein may further comprise (b) a nucleotide sequence that encodes a Cas9 (e.g., an eaCas9 or an eiCas9) molecule, or an eiCas9-fusion protein molecule. The nucleic acid composition disclosed herein may further comprise (c)(i) a nucleotide sequence that encodes a second gRNA molecule having a second targeting domain that is complementary to a second target sequence of the PreC, C, X, PreS1, PreS2, S, P or SP gene. The nucleic acid composition disclosed herein may further comprise (c)(ii) a nucleotide sequence that encodes a third gRNA molecule described herein having a third targeting domain that is complementary to a third target sequence of the PreC, C, X, PreS1, PreS2, S, P or SP gene. The nucleic acid composition disclosed herein may further comprise (c)(iii) a nucleotide sequence that encodes a fourth gRNA molecule described herein having a fourth targeting domain that is complementary to a fourth target sequence of the PreC, C, X, PreS1, PreS2, S, P or SP gene.

In certain embodiments, a nucleic acid composition encodes a second gRNA molecule comprising a targeting domain configured to provide a cleavage event, e.g., a double strand break or a single strand break, sufficiently close to a HBV target position in the PreC, C, X, PreS1, PreS2, S, P or SP gene, to allow alteration, e.g., alteration associated with NHEJ, of a HBV target position in the PreC, C, X PreS1, PreS2, S, P or SP gene, either alone or in combination with the break positioned by said first gRNA molecule.

In certain embodiments, a nucleic acid composition encodes a third gRNA molecule comprising a targeting domain configured to provide a cleavage event, e.g., a double strand break or a single strand break, sufficiently close to a HBV target position in the PreC, C, X, PreS1, PreS2, S, P or SP gene to allow alteration, e.g., alteration associated with NHEJ, of a HBV target position in the PreC, C, X PreS1, PreS2, S, P or SP gene, either alone or in combination with the break positioned by the first and/or second gRNA molecule.

In certain embodiments, a nucleic acid composition encodes a fourth gRNA molecule comprising a targeting domain configured to provide a cleavage event, e.g., a double strand break or a single strand break, sufficiently close to a HBV target position in the PreC, C, X, PreS1, PreS2, S, P or SP gene to allow alteration, e.g., alteration associated with NHEJ, of a HBV target position in the PreC, C, X PreS1, PreS2, S, P or SP gene, either alone or in combination with the break positioned by the first gRNA molecule, the second gRNA molecule and/or the third gRNA molecule.

In certain embodiments, the second gRNA is selected to target the same HBV target position as the first gRNA molecule. In certain embodiments, the third gRNA molecule and the fourth gRNA molecule are selected to target the same HBV target position as the first and second gRNA molecules.

In certain embodiments, the second, the third or the fourth gRNA molecule comprises a targeting domain comprising the nucleotide sequence selected from SEQ ID NOS: 215 to 141071.

In certain embodiments, (a) and (b) are present on one nucleic acid molecule, e.g., one vector, e.g., one viral vector. In certain embodiments, the nucleic acid molecule is an AAV vector. Exemplary AAV vectors that may be used in any of the described compositions and methods include an AAV2 vector, a modified AAV2 vector, an AAV3 vector, a modified AAV3 vector, an AAV6 vector, a modified AAV6 vector, an AAV8 vector and an AAV9 vector. In certain embodiments, the nucleic acid molecule is an LV vector.

In certain embodiments, (a) is present on a first nucleic acid molecule, e.g., a first vector, e.g., a first viral vector (e.g., a first AAV vector or a first LV vector); and (b) is present on a second nucleic acid molecule, e.g., a second vector, e.g., a second vector (e.g., a second AAV vector or a second LV vector). The first and second nucleic acid molecules may be AAV vectors. The first and second nucleic acid molecules may be LV vectors

Each of (a) and (c)(i) may be present on one nucleic acid molecule, e.g., one vector, e.g., one viral vector, e.g., the same AAV or LV vector. In certain embodiments, (a) and (c)(i) are on different vectors. For example, (a) may be present on a first nucleic acid molecule, e.g., a first vector, e.g., a first viral vector (e.g., a first AAV vector or a first LV vector); and (c)(i) may be present on a second nucleic acid molecule, e.g., a second vector, e.g., a second vector (e.g., a second AAV vector or a second LV vector).

In certain embodiments, (a), (b), and (c)(i) are present on one nucleic acid molecule, e.g., one vector, e.g., one viral vector (e.g., an AAV vector or a LV vector). In certain embodiments, one of (a), (b), and (c)(i) is encoded on a first nucleic acid molecule, e.g., a first vector, e.g., a first viral vector (e.g., a first AAV vector or a first LV vector); and a second and third of (a), (b), and (c)(i) is encoded on a second nucleic acid molecule, e.g., a second vector, e.g., a second vector (e.g., a second AAV vector or a second LV vector).

In certain embodiments, (a) is present on a first nucleic acid molecule, e.g., a first vector, e.g., a first viral vector (e.g., a first AAV vector or a first LV vector); and (b) and (c)(i) are present on a second nucleic acid molecule, e.g., a second vector, e.g., a second vector (e.g., a second AAV vector or a second LV vector).

In certain embodiments, (b) is present on a first nucleic acid molecule, e.g., a first vector, e.g., a first viral vector (e.g., a first AAV vector or a first LV vector); and (a) and (c)(i) are present on a second nucleic acid molecule, e.g., a second vector, e.g., a second vector (e.g., a second AAV vector or a second LV vector).

In certain embodiments, (c)(i) is present on a first nucleic acid molecule, e.g., a first vector, e.g., a first viral vector (e.g., a first AAV vector or a first LV vector); and (b) and (a) are present on a second nucleic acid molecule, e.g., a second vector, e.g., a second vector (e.g., a second AAV vector or a second LV vector).

In certain embodiments, each of (a), (b) and (c)(i) are present on different nucleic acid molecules, e.g., different vectors, e.g., different viral vectors (e.g., different AAV vectors or different LV vectors). For example, (a) may be on a first nucleic acid molecule, (b) on a second nucleic acid molecule, and (c)(i) on a third nucleic acid molecule (e.g., a third AAV vector or a third LV vector).

In certain embodiments, when a third and/or fourth gRNA molecule are present, (a), (b), (c)(i), (c)(ii) and (c)(iii) are present on one nucleic acid molecule, e.g., one vector, e.g., one viral vector (e.g., an AAV vector or a LV vector). In certain embodiments, each of (a), (b), (c)(i), (c)(ii) and (c)(iii) are present on the different nucleic acid molecules, e.g., different vectors, e.g., the different viral vectors (e.g., different AAV vectors or different LV vectors). In certain embodiments, (a), (b), (c)(i), (c) (ii) and (c)(iii) re present on more than one nucleic acid molecule, but fewer than five nucleic acid molecules, e.g., AAV vectors or LV vectors.

In certain embodiments, certain nucleic acid compositions described herein may comprise a promoter operably linked to the nucleotide sequence that encodes the gRNA molecule of (a), e.g., a promoter described herein. Such nucleic acid compositions may further comprise a second promoter operably linked to the sequence that encodes the second, third and/or fourth gRNA molecule of (c), e.g., a promoter described herein. The promoter and second promoter can differ from one another. In certain embodiments, the promoter and second promoter are the same.

In certain embodiments, certain nucleic acid compositions described herein may further comprise a promoter operably linked to the sequence that encodes the Cas9 molecule of (b), e.g., a promoter described herein.

The presently disclosed subject matter further provides methods of altering a HBV viral gene selected from the group consisting of PreC gene, C gene, Xgene, PreS1 gene, PreS2 gene, S gene, P gene and SP gene in a cell. In certain embodiments, the method comprises administering to said cell one of: (i) a genome editing system comprising a gRNA molecule comprising a targeting domain that is complementary with a target sequence of the HBV viral gene, and at least a Cas9 molecule; (ii) a vector comprising a polynucleotide encoding a gRNA molecule comprising a targeting domain that is complementary with a target sequence of the HBV viral gene, and a polynucleotide encoding a Cas9 molecule; or (iii) a composition comprising a gRNA molecule comprising a targeting domain that that is complementary with a target sequence of the HBV viral gene, and at least a Cas9 molecule. In certain embodiments, the alteration comprises knockout of the HBV viral gene, knockdown of the HBV viral gene, or concomitant knockout and knockdown of the HBV viral gene.

In certain embodiments, the presently disclosed subject matter provides methods of altering cells, e.g., altering the structure, e.g., altering the sequence, of a target nucleic acid of a cell, comprising contacting said cell with: (a) a gRNA that targets the PreC, C, X, PreS1, PreS2, S, P or SP gene, e.g., a gRNA as described herein; (b) a Cas9 (e.g., an eaCas9 or eiCas9) molecule or a Cas9 fusion protein; and optionally, (c) a second, third and/or fourth gRNA that targets PreC, C, X, PreS1, PreS2, S, P or SP gene, e.g., a second, third and/or fourth gRNA, as described herein. In certain embodiments, the methods disclosed herein comprise contacting said cell with (a) and (b). In certain embodiments, the methods disclosed herein comprise contacting said cell with (a), (b), and (c).

In certain embodiments, the cell is from a subject suffering from or likely to develop HBV infection. In certain embodiments, the cell is from a subject that would benefit from having a mutation at a HBV target position. In certain embodiments, the contacting step is performed in vivo.

In certain embodiments, the contacting step of the method comprises contacting the cell with a nucleic acid composition, e.g., a vector, e.g., an AAV vector or a LV vector, that expresses each of (a), (b), and (c). In certain embodiments, the contacting step of the method comprises delivering to the cell a Cas9 molecule or Cas9-fusion protein of (b) and a nucleic acid composition which encodes a gRNA of (a) and optionally, a second gRNA (c)(i) and further optionally, a third gRNA (c)(ii) and/or fourth gRNA (c)(iii).

The presently disclosed subject matter further provides methods of reducing, treating and/or preventing HBV infection in a subject. In certain embodiments, the method comprises administering to the subject one of: (i) a genome editing system comprising a gRNA molecule comprising a targeting domain that is complementary with a target sequence of a HBV viral gene selected from the group consisting of PreC gene, C gene, Xgene, PreS1 gene, PreS2 gene, S gene, P gene and SP gene, and at least a Cas9 molecule; (ii) a vector comprising a polynucleotide encoding a gRNA molecule comprising a targeting domain that is complementary with a target sequence of a HBV viral gene selected from the group consisting of PreC gene, C gene, Xgene, PreS1 gene, PreS2 gene, S gene, P gene and SP gene, and a polynucleotide encoding a Cas9 molecule; or (iii) a composition comprising a gRNA molecule comprising a targeting domain that that is complementary with a target sequence of a HBV viral gene selected from the group consisting of PreC gene, C gene, X gene, PreS1 gene, PreS2 gene, S gene, P gene and SP gene, and at least a Cas9 molecule.

In certain embodiments, disclosed herein is a method of treating a subject suffering from or likely to develop HBV, e.g., altering the structure, e.g., sequence, of a target nucleic acid of the subject, comprising contacting the subject (or a cell from the subject) with: (a) a gRNA that targets the PreC, C, X, PreS1, PreS2, S, P or SP gene, e.g., a gRNA disclosed herein; (b) a Cas9 molecule, e.g., a Cas9 molecule disclosed herein (e.g., an eaCas9 or eiCas9); and optionally, (c)(i) a second gRNA that targets the PreC, C, X, PreS1, PreS2, S, P or SP gene, e.g., a second gRNA disclosed herein, and further optionally, (c)(ii) a third gRNA, and still further optionally, (c)(iii) a fourth gRNA that target the PreC, C, X, PreS1, PreS2, S, P or SP gene, e.g., a third and fourth gRNA disclosed herein.

In certain embodiments, contacting comprises contacting with (a) and (b). In certain embodiments, contacting comprises contacting with (a), (b), and (c)(i). In certain embodiments, contacting comprises contacting with (a), (b), (c)(i) and (c)(ii). In certain embodiments, contacting comprises contacting with (a), (b), (c)(i), (c)(ii) and (c)(iii).

In certain embodiments, the method comprises acquiring knowledge of the sequence at a HBV target position in said subject. In certain embodiments, acquiring knowledge of the sequence at a HBV target position in said subject comprises sequencing one or more of the PreC, C, X, PreS1, PreS2, S, P and/or SP gene(s) or a portion of the PreC, C, X, PreS1, PreS2, S, P and/or SP gene.

In certain embodiments, the method comprises introducing a mutation at a HBV target position. In certain embodiments, the method comprises introducing a mutation at a HBV target position by NHEJ.

In certain embodiments, a cell of the subject is contacted is in vivo with (a), (b) and optionally (c)(i), further optionally (c)(ii), and still further optionally (c)(iii).

In certain embodiments, the cell of the subject is contacted in vivo by intravenous delivery of (a), (b), and optionally (c)(i), further optionally (c)(ii), and still further optionally (c)(iii).

In certain embodiments, the contacting step comprises contacting the subject with a nucleic acid composition, e.g., a vector, e.g., an AAV vector or a LV vector, described herein, e.g., a nucleic acid that encodes at least one of (a), (b), and optionally (c)(i), further optionally (c)(ii), and still further optionally (c)(iii).

In certain embodiments, the contacting step comprises delivering to said subject said Cas9 molecule of (b), as a protein or mRNA, and a nucleic acid composition which encodes (a), and optionally (c)(i), further optionally (c)(ii), and still further optionally (c)(iii).

In certain embodiments, the contacting step comprises delivering to the subject the Cas9 molecule of (b), as a protein or mRNA, the gRNA of (a), as an RNA, and optionally the second gRNA of (c)(i), further optionally said third gRNA of (c)(ii), and still further optionally said fourth gRNA of (c)(iii), as an RNA.

In certain embodiments, the contacting step comprises delivering to the subject the gRNA of (a), as an RNA, optionally said second gRNA of (c)(i), further optionally said third gRNA of (c)(ii), and still further optionally said fourth gRNA of (c)(iii), as an RNA, a nucleic acid that encodes the Cas9 molecule of (b).

When the method comprises (1) introducing a mutation at a HBV target position by NHEJ or (2) knocking down expression of one or more of the PreC, C, X, PreS1, PreS2, S, P and/or SP gene(s), e.g., by targeting the promoter region, a Cas9 molecule of (b) and at least one guide RNA, e.g., a guide RNA of (a) are included in the contacting step.

In certain embodiments, a cell of the subject is contacted is in vivo with (a), (b) and optionally (c)(i), further optionally (c)(ii), and still further optionally (c)(iii). In certain embodiments, the cell of the subject is contacted in vivo by intravenous delivery of (a), (b) and optionally (c)(i), further optionally (c)(ii), and still further optionally (c)(iii).

In certain embodiments, the contacting step comprises contacting the subject with a nucleic acid composition, e.g., a vector, e.g., an AAV vector or a LV vector, described herein, e.g., a nucleic acid that encodes at least one of (a), (b), and optionally (c)(i), further optionally (c)(ii), and still further optionally (c)(iii).

In certain embodiments, the contacting step comprises delivering to said subject said Cas9 molecule of (b), as a protein or mRNA, and a nucleic acid composition which encodes (a) and optionally (c)(i), further optionally (c)(ii), and still further optionally (c)(iii).

In certain embodiments, the contacting step comprises delivering to the subject the Cas9 molecule of (b), as a protein or mRNA, the gRNA of (a), as an RNA, and optionally the second gRNA of (c)(i), further optionally said third gRNA of (c)(ii), and still further optionally said fourth gRNA of (c)(iii), as an RNA.

In certain embodiments, the contacting step comprises delivering to the subject the gRNA of (a), as an RNA, optionally said second gRNA of (c)(i), further optionally said third gRNA of (c)(ii), and still further optionally said fourth gRNA of (c)(iii), as an RNA, and a nucleic acid that encodes the Cas9 molecule of (b).

In certain embodiments, disclosed herein is a reaction mixture comprising a gRNA molecule, a nucleic acid composition, or a composition described herein, and a cell, e.g., a cell from a subject having, or likely to develop HBV, or a subject which would benefit from a mutation at a HBV target position.

In certain embodiments, disclosed herein is a kit comprising, (a) a gRNA molecule described herein, or nucleic acid composition that encodes the gRNA, and one or more of the following: (b) a Cas9 molecule, e.g., a Cas9 molecule described herein (e.g., an eaCas9 or eiCas9), or a nucleic acid composition or mRNA that encodes the Cas9; (c)(i) a second gRNA molecule, e.g., a second gRNA molecule described herein or a nucleic acid composition that encodes (c)(i); (c)(ii) a third gRNA molecule, e.g., a second gRNA molecule described herein or a nucleic acid composition that encodes (c)(ii); (c)(iii) a fourth gRNA molecule, e.g., a second gRNA molecule described herein or a nucleic acid composition that encodes (c)(iii).

In certain embodiments, the kit comprises a nucleic acid composition, e.g., an AAV vector, that encodes one or more of (a), (b), (c)(i), (c)(ii), and (c)(iii).

In yet another aspect, disclosed herein is a gRNA molecule, e.g., a gRNA molecule described herein, for use in treating, or delaying the onset or progression of HBV infection in a subject, e.g., in accordance with a method of treating, or delaying the onset or progression of HBV infection as described herein.

In certain embodiments, the gRNA molecule is used in combination with a Cas9 molecule, e.g., a Cas9 molecule described herein (e.g., an eaCas9 or eiCas9). For example, and not by way of limitation, the Cas9 molecule, fusion-protein or polypeptide is an S. pyogenes Cas9 variant, e.g., the EQR variant or the VRER variant. In certain embodiments, the Cas9 molecule, fusion-protein or polypeptide is an S. aureus Cas9 variant, e.g., the KKH variant. Additionally or alternatively, in certain embodiments, the gRNA molecule is used in combination with a second, third and/or fourth gRNA molecule, e.g., a second, third and/or fourth gRNA molecule described herein.

In still another aspect, disclosed herein is use of a gRNA molecule, e.g., a gRNA molecule described herein, in the manufacture of a medicament for treating, or delaying the onset or progression of HBV in a subject, e.g., in accordance with a method of treating, or delaying the onset or progression of HBV as described herein.

In certain embodiments, the medicament comprises a Cas9 molecule, e.g., a Cas9 molecule described herein, e.g., the S. pyogenes Cas9 EQR variant, the S. pyogenes Cas9 VRER variant or the S. aureus KKH variant. Additionally or alternatively, in certain embodiments, the medicament comprises a second, third and/or fourth gRNA molecule, e.g., a second, third and/or fourth gRNA molecule described herein.

Other features and advantages of the subject matter disclosed herein will be apparent from the detailed description, drawings, and from the claims.

### BRIEF DESCRIPTION OF THE DRAWINGS

FIGS. 1A-1I are representations of several exemplary gRNAs. FIG. 1A depicts a modular gRNA molecule derived in part (or modeled on a sequence in part) from Streptococcus pyogenes (S. pyogenes) as a duplexed structure (SEQ ID NOs:39 and 40, respectively, in order of appearance); FIG. 1B depicts a unimolecular gRNA molecule derived in part from S. pyogenes as a duplexed structure (SEQ ID NO:41); FIG. 1C depicts a unimolecular gRNA molecule derived in part from S. pyogenes as a duplexed structure (SEQ ID NO:42); FIG. 1D depicts a unimolecular gRNA molecule derived in part from S. pyogenes as a duplexed structure (SEQ ID NO:43); FIG. 1E depicts a unimolecular gRNA molecule derived in part from S. pyogenes as a duplexed structure (SEQ ID NO:44); FIG. 1F depicts a modular gRNA molecule derived in part from Streptococcus thermophilus (S. thermophilus) as a duplexed structure (SEQ ID NOs:45 and 46, respectively, in order of appearance); and FIG. 1G depicts an alignment of modular gRNA molecules of S. pyogenes and S. thermophilus (SEQ ID NOs:39, 45, 47, and 46, respectively, in order of appearance). FIGS. 1H-1I depicts additional exemplary structures of unimolecular gRNA molecules. FIG. 1H shows an exemplary structure of a unimolecular gRNA molecule derived in part from S. pyogenes as a duplexed structure (SEQ ID NO:42). FIG. 1I shows an exemplary structure of a unimolecular gRNA molecule derived in part from S. aureus as a duplexed structure (SEQ ID NO:38).

FIGS. 2A-2G depict an alignment of Cas9 sequences (Chylinski 2013). The N-terminal RuvC-like domain is boxed and indicated with a “Y.” The other two RuvC-like domains are boxed and indicated with a “B.” The HNH-like domain is boxed and indicated by a “G.” Sm: S. mutans (SEQ ID NO:1); Sp: S. pyogenes (SEQ ID NO:2); St: S. thermophilus (SEQ ID NO:4); and Li: L. innocua (SEQ ID NO:5). “Motif” (SEQ ID NO:14) is a consensus sequence based on the four sequences. Residues conserved in all four sequences are indicated by single letter amino acid abbreviation; “*” indicates any amino acid found in the corresponding position of any of the four sequences; and “-” indicates absent.

FIGS. 3A-3B show an alignment of the N-terminal RuvC-like domain from the Cas9 molecules disclosed in Chylinski 2013 (SEQ ID NOs:52-95, 120-123). The last line of FIG. 3B identifies 4 highly conserved residues.

FIGS. 4A-4B show an alignment of the N-terminal RuvC-like domain from the Cas9 molecules disclosed in Chylinski 2013 with sequence outliers removed (SEQ ID NOs:52-123). The last line of FIG. 4B identifies 3 highly conserved residues.

FIGS. 5A-5C show an alignment of the HNH-like domain from the Cas9 molecules disclosed in Chylinski 2013 (SEQ ID NOs:124-198). The last line of FIG. 5C identifies conserved residues.

FIGS. 6A-6B show an alignment of the HNH-like domain from the Cas9 molecules disclosed in Chylinski 2013 with sequence outliers removed (SEQ ID NOs:124-141, 148, 149, 151-153, 162, 163, 166-174, 177-187, 194-198). The last line of FIG. 6B identifies 3 highly conserved residues.

FIG. 7 illustrates gRNA domain nomenclature using an exemplary gRNA sequence (SEQ ID NO:42).

FIGS. 8A and 8B provide schematic representations of the domain organization of S. pyogenes Cas9. FIG. 8A shows the organization of the Cas9 domains, including amino acid positions, in reference to the two lobes of Cas9 (recognition (REC) and nuclease (NUC) lobes). FIG. 8B shows the percent homology of each domain across 83 Cas9 orthologs.

FIG. 9 shows the plasmid map for pAF196.

FIG. 10 shows the plasmid map for pAF197.

FIG. 11 shows the plasmid map for pAF198.

FIG. 12 shows the plasmid map for pAF199.

FIG. 13 shows the plasmid map for pDRmini004.

FIG. 14 shows the reduction in GFP expression of the transfected cell population due to Cas9-mediated cleavage of the HBV target sequences in plasmids pAF196-199.

### DETAILED DESCRIPTION

For purposes of clarity of disclosure and not by way of limitation, the detailed description is divided into the following subsections:

1. Definitions

2. Hepatitis B virus (HBV)

3. Methods to Treat, Prevent and/or Reduce Hepatitis B virus Infection

4. Methods of Altering the HBV genome, including PreC, C, X, PreS1, PreS2, S, P and/or SP gene(s)

5. Guide RNA (gRNA) Molecules

6. Methods for Designing gRNAs

7. Cas9 Molecules

8. Functional Analysis of Candidate Molecules

9. Genome Editing Approaches

10. Target Cells

11. Delivery, Formulations and Routes of Administration

12. Modified Nucleosides, Nucleotides, and Nucleic Acids

### 1. Definitions

As used herein, the term “about” or “approximately” means within an acceptable error range for the particular value as determined by one of ordinary skill in the art, which can depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, a “genome editing system” refers to a system that is capable of editing (e.g., modifying or altering) one or more target genes in a cell, for example by means of Cas9-mediated single or double strand breaks. Genome editing systems may comprise, in various embodiments, (a) one or more Cas9/gRNA complexes, and (b) separate Cas9 molecules and gRNAs that are capable of associating in a cell to form one or more Cas9/gRNA complexes. A genome editing system according to the present disclosure may be encoded by one or more nucleotides (e.g. RNA, DNA) comprising coding sequences for Cas9 and/or gRNAs that can associate to form a Cas9/gRNA complex, and the one or more nucleotides encoding the gene editing system may be carried by a vector as described herein.

In certain embodiments, the genome editing system targets one or more (e.g., two, three, four, five, six, seven or eight) HBV viral gene selected from the group consisting of PreC gene, C gene, X gene, PreS1 gene, PreS2 gene, S gene, P gene and SP gene.

In certain embodiments, the genome editing system that targets a PreC gene comprises a gRNA molecule comprising a targeting domain complementary to a target domain (also referred to as “target sequence”) of the PreC gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the PreC gene, or a polynucleotide encoding thereof. The genome editing system that targets a PreC gene may further comprise a third and a fourth gRNA molecules that target the PreC gene.

In certain embodiments, the genome editing system that targets a C gene comprises a gRNA molecule comprising a targeting domain complementary to a target domain (also referred to as “target sequence”) of the C gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the C gene, or a polynucleotide encoding thereof. The genome editing system that targets a C gene may further comprise a third and a fourth gRNA molecules that target the C gene.

In certain embodiments, the genome editing system that targets a X gene comprises a gRNA molecule comprising a targeting domain complementary to a target domain (also referred to as “target sequence”) of the Xgene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the Xgene, or a polynucleotide encoding thereof. The genome editing system that targets a X gene may further comprise a third and a fourth gRNA molecules that target the Xgene.

In certain embodiments, the genome editing system that targets a PreS1 gene comprises a gRNA molecule comprising a targeting domain complementary to a target domain (also referred to as “target sequence”) of the PreS1 gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the PreS1 gene, or a polynucleotide encoding thereof. The genome editing system that targets a PreS1 gene may further comprise a third and a fourth gRNA molecules that target the PreS1 gene.

In certain embodiments, the genome editing system that targets a PreS2 gene comprises a gRNA molecule comprising a targeting domain complementary to a target domain (also referred to as “target sequence”) of the PreS2 gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the PreS2 gene, or a polynucleotide encoding thereof. The genome editing system that targets a PreS2 gene may further comprise a third and a fourth gRNA molecules that target the PreS2 gene.

In certain embodiments, the genome editing system that targets a S gene comprises a gRNA molecule comprising a targeting domain complementary to a target domain (also referred to as “target sequence”) of the S gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the S gene, or a polynucleotide encoding thereof. The genome editing system that targets a S gene may further comprise a third and a fourth gRNA molecules that target the S gene.

In certain embodiments, the genome editing system that targets a P gene comprises a gRNA molecule comprising a targeting domain complementary to a target domain (also referred to as “target sequence”) of the P gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the P gene, or a polynucleotide encoding thereof. The genome editing system that targets a P gene may further comprise a third and a fourth gRNA molecules that target the P gene.

In certain embodiments, the genome editing system that targets a SP gene comprises a gRNA molecule comprising a targeting domain complementary to a target domain (also referred to as “target sequence”) of the SP gene, or a polynucleotide encoding thereof, and at least one Cas9 molecule or polynucleotide(s) encoding thereof. In certain embodiments, the genome editing system further comprises a second gRNA molecule comprising a targeting domain complementary to a second target sequence in the SP gene, or a polynucleotide encoding thereof. The genome editing system that targets a SP gene may further comprise a third and a fourth gRNA molecules that target the SP gene.

In certain embodiments, the genome editing system is implemented in a cell or in an in vitro contact. In certain embodiments, the genome editing system is used in a medicament, e.g., a medicament for modifying one or more HBV viral gene selected from the group consisting of PreC gene, C gene, X gene, PreS1 gene, PreS2 gene, S gene, P gene and SP gene, or a medicament for treating HBV infection. In certain embodiments, the genome editing system is used in therapy.

“HBV target knockdown position”, as used herein, refers to a position, e.g., in the PreC, C, X, PreS1, PreS2, S, P or SP gene, which if targeted by an eiCas9 or an eiCas9 fusion described herein, results in reduction or elimination of expression of functional PreC, C, X, PreS1, PreS2, S, P or SP gene product. In certain embodiments, transcription is reduced or eliminated. In certain embodiments, the position is in the PreC, C, X PreS1, PreS2, S, P or SP promoter sequence. In certain embodiments, a position in the promoter sequence of the PreC, C, X PreS1, PreS2, S, P or SP gene is targeted by an enzymatically inactive Cas9 (eiCas9) or an eiCas9-fusion protein, as described herein.

“PreC target knockout position”, as used herein, refers to a position in the PreC gene, e.g., disrupted by insertion or deletion of one or more nucleotides, e.g., disrupted by insertion or deletion of one or more nucleotides results in reduction or elimination of expression of functional PreC gene product. In certain embodiments, the position is in the PreC gene coding region, e.g., an early coding region. In certain embodiments, the position is in the PreC gene non-coding region. In certain embodiments, the non-coding region of the PreC gene is within the coding region of another HBV gene, such as the C, X PreS1, PreS2, S, P and/or SP gene. Because of the overlapping reading frames of the HBV genome, the use of “PreC gene non-coding region” is not, in the strictest sense, a non-transcribed region, but refers to the non-coding region the PreC gene, which may be the coding region of another gene. In certain embodiments, the non-coding region of the PreC gene may be a region within the subject genome, in the case of integration of the PreC gene (along with other HBV genes) within the human genome.

“C target knockout position”, as used herein, refers to a position in the C gene, e.g., disrupted by insertion or deletion of one or more nucleotides, results in reduction or elimination of expression of functional C gene product. In certain embodiments, the position is in the C gene coding region, e.g., an early coding region. In certain embodiments, the position is in the C gene non-coding region. In certain embodiments, the non-coding region of the C gene is within the coding region of another HBV gene, such as the PreC, X PreS1, PreS2, S, P and/or SP gene. Because of the overlapping reading frames of the HBV genome, the use of “C gene non-coding region” is not, in the strictest sense, a non-transcribed region, but refers to the non-coding region the C gene, which may be the coding region of another gene. In certain embodiments, the non-coding region of the C gene may be a region within the subject genome, in the case of integration of the C gene (along with other HBV genes) within the human genome.

“X target knockout position”, as used herein, refers to a position in the Xgene, e.g., disrupted by insertion or deletion of one or more nucleotides, results in reduction or elimination of expression of functional X gene product. In certain embodiments, the position is in the Xgene coding region, e.g., an early coding region. In certain embodiments, the position is in the Xgene non-coding region. In certain embodiments, the non-coding region of the Xgene is within the coding region of another HBV gene, such as the PreC, C, PreS1, PreS2, S, P and/or SP gene. Because of the overlapping reading frames of the HBV genome, the use of “Xgene non-coding region” is not, in the strictest sense, a non-transcribed region, but refers to the non-coding region the Xgene, which may be the coding region of another gene. In certain embodiments, the non-coding region of the Xgene may be a region within the subject genome, in the case of integration of the Xgene (along with other HBV genes) within the human genome.

“PreS1 target knockout position”, as used herein, refers to a position in the PreS1 gene, e.g., disrupted by insertion or deletion of one or more nucleotides, results in reduction or elimination of expression of functional PreS1 gene product. In certain embodiments, the position is in the PreS1 gene coding region, e.g., an early coding region. In certain embodiments, the position is in the PreS1 gene non-coding region. In certain embodiments, the non-coding region of the PreS1 gene is within the coding region of another HBV gene, such as the PreC, C, X, PreS2, S, P and/or SP gene. Because of the overlapping reading frames of the HBV genome, the use of “PreS1 gene non-coding region” is not, in the strictest sense, a non-transcribed region, but refers to the non-coding region the PreS1 gene, which may be the coding region of another gene. In certain embodiments, the non-coding region of the PreS1 gene may be a region within the subject genome, in the case of integration of the PreS1 gene (along with other HBV genes) within the human genome.

“PreS2 target knockout position”, as used herein, refers to a position in the PreS2 gene, e.g., disrupted by insertion or deletion of one or more nucleotides, results in reduction or elimination of expression of functional PreS2 gene product. In certain embodiments, the position is in the PreS2 gene coding region, e.g., an early coding region. In certain embodiments, the position is in the PreS2 gene non-coding region. In certain embodiments, the non-coding region of the PreS2 gene is within the coding region of another HBV gene, such as the PreC, C, X, PreS1, S, P and/or SP gene. Because of the overlapping reading frames of the HBV genome, the use of “PreS2 gene non-coding region” is not, in the strictest sense, a non-transcribed region, but refers to the non-coding region the PreS2 gene, which may be the coding region of another gene. In certain embodiments, the non-coding region of the PreS2 gene may be a region within the subject genome, in the case of integration of the PreS2 gene (along with other HBV genes) within the human genome.

“S target knockout position”, as used herein, refers to a position in the S gene, e.g., disrupted by insertion or deletion of one or more nucleotides, results in reduction or elimination of expression of functional S gene product. In certain embodiments, the position is in the S gene coding region, e.g., an early coding region. In certain embodiments, the position is in the S gene non-coding region. In certain embodiments, the non-coding region of the S gene is within the coding region of another HBV gene, such as the PreC, C, X, PreS1, PreS2, P and/or SP gene. Because of the overlapping reading frames of the HBV genome, the use of “S gene non-coding region” is not, in the strictest sense, a non-transcribed region, but refers to the non-coding region the S gene, which may be the coding region of another gene. In certain embodiments, the non-coding region of the S gene may be a region within the subject genome, in the case of integration of the S gene (along with other HBV genes) within the human genome.

“P target knockout position”, as used herein, refers to a position in the P gene, e.g., disrupted by insertion or deletion of one or more nucleotides, results in reduction or elimination of expression of functional P gene product. In certain embodiments, the position is in the P gene coding region, e.g., an early coding region. In certain embodiments, the position is in the P gene non-coding region. In certain embodiments, the non-coding region of the P gene is within the coding region of another HBV gene, such as the PreC, C, X, PreS1, PreS2, S and/or SP gene. Because of the overlapping reading frames of the HBV genome, the use of “P gene non-coding region” is not, in the strictest sense, a non-transcribed region, but refers to the non-coding region the P gene, which may be the coding region of another gene. In certain embodiments, the non-coding region of the P gene may be a region within the subject genome, in the case of integration of the P gene (along with other HBV genes) within the human genome.

“SP target knockout position”, as used herein, refers to a position in the SP gene, e.g., disrupted by insertion or deletion of one or more nucleotides, results in reduction or elimination of expression of functional SP gene product. In certain embodiments, the position is in the SP gene coding region, e.g., an early coding region. In certain embodiments, the position is in the SP gene non-coding region. In certain embodiments, the non-coding region of the SP gene is within the coding region of another HBV gene, such as the PreC, C, X, PreS1, PreS2, S and/or P gene. Because of the overlapping reading frames of the HBV genome, the use of “SP gene non-coding region” is not, in the strictest sense, a non-transcribed region, but refers to the non-coding region the SP gene, which may be the coding region of another gene. In certain embodiments, the non-coding region of the SP gene may be a region within the subject genome, in the case of integration of the SP gene (along with other HBV genes) within the human genome.

“Domain”, as used herein, is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Governing gRNA molecule”, as used herein, refers to a gRNA molecule that comprises a targeting domain that is complementary to a target domain on a nucleic acid composition that comprises a sequence that encodes a component of the CRISPR/Cas system that is introduced into a cell or subject. In certain embodiments, a governing gRNA does not target an endogenous cell or subject sequence. In certain embodiments, a governing gRNA molecule comprises a targeting domain that is complementary with a target sequence on: (a) a nucleic acid composition that encodes a Cas9 molecule; (b) a nucleic acid composition that encodes a gRNA molecule which comprises a targeting domain that targets a position in the HBV genome (e.g., PreC gene, C gene, X gene, PreS1 gene, PreS2 gene, S gene, P gene and SP gene) (a target gene gRNA); or on more than one nucleic acid that encodes a CRISPR/Cas component, e.g., both (a) and (b). In certain embodiments, a nucleic acid molecule that encodes a CRISPR/Cas component, e.g., that encodes a Cas9 molecule or a target gene gRNA, comprises more than one target domain that is complementary with a governing gRNA targeting domain. In certain embodiments, a governing gRNA molecule complexes with a Cas9 molecule and results in Cas9 mediated inactivation of the targeted nucleic acid, e.g., by cleavage or by binding to the nucleic acid, and results in cessation or reduction of the production of a CRISPR/Cas system component. In certain embodiments, the Cas9 molecule forms two complexes: a complex comprising a Cas9 molecule with a target gene gRNA, which complex will alter the PreC gene, C gene, X gene, PreS1 gene, PreS2 gene, S gene, P gene and/or SP gene; and a complex comprising a Cas9 molecule with a governing gRNA molecule, which complex will act to prevent further production of a CRISPR/Cas system component, e.g., a Cas9 molecule or a target gene gRNA molecule. In certain embodiments, a governing gRNA molecule/Cas9 molecule complex binds to or promotes cleavage of a control region sequence, e.g., a promoter, operably linked to a sequence that encodes a Cas9 molecule, a sequence that encodes a transcribed region, an exon, or an intron, for the Cas9 molecule. In certain embodiments, a governing gRNA molecule/Cas9 molecule complex binds to or promotes cleavage of a control region sequence, e.g., a promoter, operably linked to a gRNA molecule, or a sequence that encodes the gRNA molecule. In certain embodiments, the governing gRNA, e.g., a Cas9-targeting governing gRNA molecule, or a target gene gRNA-targeting governing gRNA molecule, limits the effect of the Cas9 molecule/target gene gRNA molecule complex-mediated gene targeting. In certain embodiments, a governing gRNA places temporal, level of expression, or other limits, on activity of the Cas9 molecule/target gene gRNA molecule complex. In certain embodiments, a governing gRNA reduces off-target or other unwanted activity. In certain embodiments, a governing gRNA molecule inhibits, e.g., entirely or substantially entirely inhibits, the production of a component of the Cas9 system and thereby limits, or governs, its activity.

“Modulator”, as used herein, refers to an entity, e.g., a drug, that can alter the activity (e.g., enzymatic activity, transcriptional activity, or translational activity), amount, distribution, or structure of a subject molecule or genetic sequence. In certain embodiments, modulation comprises cleavage, e.g., breaking of a covalent or non-covalent bond, or the forming of a covalent or non-covalent bond, e.g., the attachment of a moiety, to the subject molecule. In certain embodiments, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In certain embodiments, it has less than 50, 20, or 10 amino acid residues.

A “Cas9 molecule” or “Cas9 polypeptide” as used herein refers to a molecule or polypeptide, respectively, that can interact with a gRNA molecule and, in concert with the gRNA molecule, localize to a site comprising a target domain (also referred to as “target sequence”) and, in certain embodiments, a PAM sequence. Cas9 molecules and Cas9 polypeptides include both naturally occurring Cas9 molecules and Cas9 polypeptides and engineered, altered, or modified Cas9 molecules or Cas9 polypeptides that differ, e.g., by at least one amino acid residue, from a reference sequence, e.g., the most similar naturally occurring Cas9 molecule. In certain embodiments, the Cas9 molecule is a wild-type S. pyogenes Cas9, which recognizes a NGG PAM sequence. In certain embodiments, the Cas9 molecule is an S. pyogenes Cas9 EQR variant, which recognizes a NGAG PAM sequence, A NGCG PAM sequence, a NGGG PAM sequence, a NGTG PAM sequence, a NGAA PAM sequence, a NGAT PAM sequence or a NGAC PAM sequence. In certain embodiments, the Cas9 molecule is an S. pyogenes Cas9 VRER variant, which recognizes a NGCG PAM sequence, a NGCA PAM sequence, a NGCT PAM sequence, or a NGCC PAM sequence. In certain embodiments, the Cas9 molecule is a wild-type S. aureus Cas9, which recognizes a NNNRRT PAM sequence, or a NNNRRV PAM sequence. In certain embodiments, the Cas9 molecule is an S. aureus Cas9 KKH variant, which recognizes a NNNRRT PAM sequence or a NNNRRV PAM sequence.

A “reference molecule” as used herein refers to a molecule to which a modified or candidate molecule is compared. For example, a reference Cas9 molecule refers to a Cas9 molecule to which a modified or candidate Cas9 molecule is compared. Likewise, a reference gRNA refers to a gRNA molecule to which a modified or candidate gRNA molecule is compared. The modified or candidate molecule may be compared to the reference molecule on the basis of sequence (e.g., the modified or candidate molecule may have X % sequence identity or homology with the reference molecule) or activity (e.g., the modified or candidate molecule may have X % of the activity of the reference molecule). For example, where the reference molecule is a Cas9 molecule, a modified or candidate molecule may be characterized as having no more than 10% of the nuclease activity of the reference Cas9 molecule. Examples of reference Cas9 molecules include naturally occurring unmodified Cas9 molecules, e.g., a naturally occurring Cas9 molecule from S. pyogenes, S. aureus, or N. meningitidis. In certain embodiments, the reference Cas9 molecule is the naturally occurring Cas9 molecule having the closest sequence identity or homology with the modified or candidate Cas9 molecule to which it is being compared. In certain embodiments, the reference Cas9 molecule is a parental molecule having a naturally occurring or known sequence on which a mutation has been made to arrive at the modified or candidate Cas9 molecule.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In certain embodiments, the subject is a human. In certain embodiments, the subject is poultry.

“Treat”, “treating” and “treatment”, as used herein, mean the treatment of a disease in a mammal, e.g., in a human, including (a) inhibiting the disease, i.e., arresting or preventing its development or progression; (b) relieving the disease, i.e., causing regression of the disease state; (c) relieving one or more symptoms of the disease; and (d) curing the disease.

“Prevent,” “preventing,” and “prevention” as used herein means the prevention of a disease in a mammal, e.g., in a human, including (a) avoiding or precluding the disease; (b) affecting the predisposition toward the disease; (c) preventing or delaying the onset of at least one symptom of the disease.

“X” as used herein in the context of an amino acid sequence, refers to any amino acid (e.g., any of the twenty natural amino acids) unless otherwise specified.

### 2. Hepatitis B Virus (HBV)

HBV is a hepadnavirus that preferentially affects hepatocytes. Enveloped virions contain a 3.2 kB double-stranded DNA genome with four partially overlapping open reading frames (ORFs). The ORFs encode the envelope, core, polymerase and X proteins. HBV enters hepatocytes by binding to the sodium taurocholate co-transporting polypeptide (NTCP) receptor. Inside hepatocytes, the virus uncoats and is transported into the nucleus, where the relaxed circular DNA (rcDNA) of the capsid is repaired to generate covalently closed circular DNA (cccDNA). The cccDNA is transcribed into viral pregenomic RNA (pgRNA) and viral mRNA using host RNA polymerase II. Viral pgRNA and mRNA is transported from the nucleus to the cytoplasm, where it is translated into viral proteins, including viral reverse transcriptase, HBsAg and HBeAg. In the cytoplasm, viral pgRNA is reverse transcribed by viral reverse transcriptase to generate rcDNA that is ready for packaging. The virus is then packaged and secreted from the hepatocyte.

### 3. Methods to Treat, Prevent and/or Reduce Hepatitis B Virus Infection

Methods and compositions described herein provide for a therapy, e.g., a one-time therapy, or a multi-dose therapy, that reduces, prevents and/or treats HBV infection.

The methods described herein involve targeted knockout and/or knockdown of the viral HBV genome, including HBV DNA in the form of cccDNA, HBV DNA in the form of rcDNA, linearized DNA within the nucleus and/or DNA intermediates in the cytoplasm. The method described herein involves targeted knockout and/or knock down of integrated viral HBV, including HBV DNA which has integrated into the subject's genome. Currently available methods to treat HBV do not target HBV cccDNA and have no effect on the presence of intra-nuclear DNA. Current methods to treat HBV also do not target integrated HBV DNA and have no effect on the production of viral proteins produced by integrated or ccc HBV DNA. The method described herein fulfills a need that is unmet in current approaches to the treatment of HBV. Such an approach will be effective as a stand-alone therapy or may be given concomitantly with current therapies to eliminate the virus and produce a cure or improved control of Hepatitis B.

HBV relies on viral genes, e.g., PreC, C, X PreS1, PreS2, S, P and/or SP for infection, proliferation and assembly. In certain embodiments, altering, e.g., knocking out or knocking down PreC, C, X, PreS1, PreS2, S, P or SP individually or in combination can reduce, prevent and/or treat HBV infections. In certain embodiments, altering, e.g., knocking down PreC, C, X, PreS1, PreS2, S, P or SP individually or in combination can reduce, prevent and/or treat HBV infections. As the HBV virus establishes chronic and/or latent infection in hepatocytes, local delivery that delivers a treatment in the region of chronic infection can be used. Targeting knockout and/or knock down to a discrete region or regions (e.g., hepatocytes, e.g., the liver) can reduce or eliminate latent infection by disabling the HBV virus.

Described herein are methods to reduce, prevent and/or treat HBV by knocking out or knocking down viral genes, or by causing destruction of HBV viral genomic DNA. In certain embodiments, methods described herein comprise knockout or knockdown of a HBV viral gene, e.g., HBV encoded open reading frames (ORFs), e.g., ORF C, ORF P, ORF S, or ORF X. In certain embodiments, methods described herein comprise knockout or knockdown of any region of the HBV genome, e.g., HBV encoded genes, e.g., PreC, C, X PreS1, PreS2, S, P or SP. In certain embodiments, methods described herein comprise knockout or knockdown of any one of or a combination of (e.g., any two, any three, four, five, six, seven or all of the) the genes, e.g., PreC, C, X PreS1, PreS2, S, P or SP. In certain embodiments, methods described herein comprise knockout or knockdown of one or a combination (e.g., any two, three, four, five, six, seven or all of) the HBV encoded genes, e.g., PreC, C, X PreS1, PreS2, S, or P.

When there are two alterations events (e.g., knocking down or knocking out the expression of genes, e.g., PreC, C, X, PreS1, PreS2, S, P or SP), the two alteration events may occur sequentially or simultaneously. In certain embodiments, the knocking out of a gene occurs prior to knocking down of a gene. In certain embodiments, the knockout of a gene is concurrent with the knockdown of a gene. In certain embodiments, the knockout of a gene is subsequent to the knockdown of a gene. In certain embodiments, the effect of the alterations is synergistic.

In certain embodiments, the methods described herein reduce, prevent and/or treat HBV by knocking out of at least one HBV viral gene, e.g., HBV encoded open reading frames (ORFs), e.g., ORF C, ORF P, ORF S, or ORF X. In certain embodiments, the methods described herein comprise knockout of any region of the HBV genome, e.g., HBV encoded genes, e.g PreC, C, X, PreS1, PreS2, S, P or SP. In certain embodiments, the methods described herein comprise knockout of any one of or a combination of (e.g., any two, any three, four, five, six, seven or all of the) the genes, e.g., PreC, C, X, PreS1, PreS2, S, P or SP. In certain embodiments, the methods described herein comprise knockout of any region of the HBV genome that contains the coding region of a gene that encodes an HBV protein, e.g., LHBs, MHBs, SHBs, HBe, HBc, polymerase/reverse transcriptase (pol), HBx or HBSP. In certain embodiments, the methods described herein comprise knockout of any one of or a combination of (e.g., any two, any three, four, five, six, seven or all of the) the genes that encode HBV proteins, e.g., LHBs, MHBs, SHBs, HBe, HBc, polymerase/reverse transcriptase (Pol), HBx or HBSP.

In certain embodiments, the methods described herein reduce, prevent and/or treat HBV by knocking down viral gene expression (e.g., knocking down the expression of one or more of the PreC, C, X, PreS1, PreS2, S, P or SP genes). In certain embodiments, the methods described herein comprise knockdown of the expression of one or more of the PreC, C, X, PreS1, PreS2, S, P or SP genes, e.g., knocking down HBV encoded open reading frames (ORFs): ORF C, ORF P, ORF S, ORF X. In certain embodiments, the methods described herein comprise knockdown of any region of the HBV genome, e.g., HBV encoded genes, e.g., PreC, C, X, PreS1, PreS2, S, P or SP. In certain embodiments, the methods described herein comprise knockdown of any one of or a combination of (e.g., any two, any three, four, five, six, seven or all of the) the genes, e.g., PreC, C, X, PreS1, PreS2, S, P or SP. Methods described herein comprise knocking down a HBV gene or genes residing on any form of the HBV genome in the nucleus of hepatocytes, including but not limited to knocking down of a gene or genes residing on cccDNA and/or knocking down of a gene or genes residing on integrated HBV DNA within the subject genome.

In certain embodiments, the methods described herein comprise knocking down any region of the HBV genome that contains the coding region of a gene that encodes a HBV protein, e.g., LHBs, MHBs, SHBs, HBe, HBc, polymerase/reverse transcriptase (pol), HBx or HBSP. In certain embodiments, the methods described herein comprise knocking down any one of or a combination of (e.g., any two, any three, four, five, six, seven or all of the) the genes that encode HBV proteins, e.g., LHBs, MHBs, SHBs, HBe, HBc, polymerase/reverse transcriptase (pol), HBx or HBSP.

In certain embodiments, the knockout of genes encoded on the HBV genome include, but are not limited to, those found on integrated HBV DNA and/or intra-nuclear HBV DNA, e.g., intra-nuclear cccDNA, e.g., intra-nuclear HBV relaxed circular DNA (rcDNA), e.g., intra-nuclear linearized HBV DNA, and/or those found on intra-cytoplasmic DNA, e.g., intra-cytoplasmic HBV DNA intermediates, e.g., intra-cytoplasmic plus-strand DNA, e.g., intra-cytoplasmic minus-strand DNA, prevents the transcription of genes vital to the proliferation, assembly and/or infectivity of HBV. Altering (e.g., knocking out or knocking down) the genes encoded on the HBV genome or on integrated HBV DNA may prevent the transcription of genes vital to the proliferation, assembly and/or infectivity of HBV. In certain embodiments, the methods described herein eliminate and/or decrease the levels of HBV DNA, HBV cccDNA, and/or HBV rcDNA in infected hepatocytes. In certain embodiments, the methods can described herein can be used to eliminate and/or decrease the levels of HBV DNA, HBV cccDNA, and/or HBV rcDNA in infected liver cells, kupfer cell, a sinusoidal epithelial cells, a stellate cells, renal tubular epithelial cells or lymphocytes, including but not limited to CD4⁺ T-cells and/or CD8⁺ T cells. In certain embodiments, the methods described herein prevent, cure or decrease the severity of HBV infection and/or chronic HBV. The methods described herein eliminate and/or decrease the levels of HBV proteins produced by HBV DNA, HBV cccDNA, integrated HBV DNA, and/or HBV rcDNA. In certain embodiments, the methods described herein decrease the levels of circulating HBsAg and HBeAg, permitting a reversal of ‘immune exhaustion’ in a subject and the effective mounting of an immunologic response to HBV. There is evidence that reduction in viral load and circulating viral proteins leads to a stoichiometric reversal in the ratio of HBsAg to anti-HBs, which allows anti-HBs to clear HBsAg and HBV Dane particles.

In certain embodiments, the knockout methods described herein cause the permanent destruction of HBV cccDNA in a large enough percent of hepatocytes to allow for immune reconstitution and subsequent clearance of infected hepatocytes via T- and B-cell mediated mechanisms. In certain embodiments, the knockout methods described herein are administered on a recurring basis (e.g., repeated administration) to allow for additive knock out of HBV DNA. In certain embodiments, the knockout methods described herein are administered weekly or monthly over the course of 1, 2, 3, 4, 6, 9 and/or 12 months.

HBV integration events into the genome are ubiquitous and random. The virus integrates throughout the genome at intronic, exonic and promoter regions. The risk of HCC is higher in subjects who have greater than 3 integration events per hepatocyte and in subjects in whom integration occurs more often in promoter and/or exonic regions. Furthermore, these subjects develop HCC at younger ages and without first developing cirrhosis and fibrosis (Sung et al, Nature Genetics 2012; 44(7):765-770). Subjects at high risk for developing HCC may be identified via liver biopsy and sequencing of HBV integration events and locations. The eiCas-9 mediated knockdown of HBV genes that have been integrated into the genome, particularly in subjects who are at high risk for HCC, decreases the likelihood of a subject developing HCC.

In certain embodiments, any HBV-infected hepatocyte treated with the methods described herein may undergo natural apoptosis within 1-2 years. For example, and not by way of limitation, within one to two years of treatment, partial or substantially all treated HBV-infected hepatocytes may undergo T-cell mediated cytotoxic cell death. For example, within one to two years of treatment, partial or substantially all treated HBV-infected hepatocytes may naturally apoptose, leaving new, uninfected hepatocytes to re-populate the liver. In certain embodiments, the methods described herein lead to the clearance of HBV from and the clearance of chronic HBV infection in hepatocytes. In certain embodiments, the methods described herein prevent, cure or decrease the severity of sequelae of HBV infection, including cirrhosis, end-stage liver disease and hepatocellular carcinoma.

ORF P includes the nucleotide coding sequence (CDS) P. The CDS P encodes the HBV polymerase/reverse transcriptase (Pol) protein. The HBV genome is replicated from an RNA template in the cytoplasm. Minus strand DNA is synthesized using RNA as a template, and plus strand DNA is then synthesized from the minus strand template. Pol is involved in the priming of minus-strand DNA synthesis, reverse transcriptase activity to synthesize the minus strand from RNA, and polymerase activity to synthesize plus strand DNA. Pol is also involved in capsid formation. Pol is integral to the HBV life cycle. In certain embodiments, the methods described herein knock down and/or knock out Pol expression. In certain embodiments, the knock down and/or knock out of Pol expression can lead to the clearance of HBV infection.

ORF C includes the nucleotide coding sequence (CDS) C. The CDS C encodes the capsid protein, also known as the viral core protein, as well as the HBe antigen (HBeAg). The capsid protein is involved in the structure of the viral nucleocapsid. The function of HBeAg is unknown. HBV core protein is integral to the HBV life cycle. Methods described herein knock down and/or knock out core protein expression. In certain embodiments, the knockdown and/or knockout of core protein expression can lead to the clearance of HBV infection.

ORF S includes the nucleotide coding sequence (CDS) S. The CDS S encodes the PreS1, PreS2 and S regions, which encode, respectively, the long surface protein, middle surface protein, S protein (also known as small surface protein and/or HBs antigen (HBsAg)). The long-surface protein contributes to receptor binding and initiation of infection. S protein is another viral surface glycoprotein that is present in the blood of infected subjects. HBsAg loss (meaning undetectable blood levels) indicates a functional cure of HBV infection. HBV S protein is integral to the HBV life cycle. In certain embodiments, the methods described herein knock down and/or knock out S protein expression. In certain embodiments, the knockdown and/or knockout of S protein expression can lead to the clearance of HBV infection.

ORF X includes the nucleotide coding sequence (CDS) X. The CDS X encodes the X protein, which has an unknown function.

In certain embodiments, altering (e.g., knocking out or knocking down) the expression of the genes, e.g., PreC, C, X, PreS1, PreS2, S, P or SP, individually or in combination, can reduce HBV protein expression, infectivity, replication, packaging and can therefore reduce, prevent and/or treat HBV infection.

In certain embodiments, highly conserved regions of the HBV genome are targeted in order to protect from causing viral escape. Highly conserved regions of the HBV genome are less likely to tolerate mutations, so targeting these regions will make it less likely that escape mutants will arise.

In certain embodiments, one or more regions of the HBV genome, e.g., the DR1 region or the DR2 region, that is known not to be integrated into the subject's genome is targeted for knock out. For example, and not by way of limitation, a method disclosed herein can knock out the DR1 region and/or the DR2 region. The DR1 region is a 12 base pair direct repeat region near the 5′ end of the HBV genome. The DR2 region is a 12 base pair direct repeat region near the 3′ end of the HBV genome.

In certain embodiments, altering (e.g., knocking out or knocking down) the expression of the HBV genes, e.g., PreC, C, X PreS1, PreS2, S, P or SP, individually or in combination, can make HBV more susceptible to antiviral therapy. Mutations in certain genes can render HBV and other viruses more susceptible to treatment with antivirals (Zhou et al., Journal of Virology 2014; 88(19): 11121-11129). In certain embodiments, altering (e.g., knocking out or knocking down HBV genes, e.g., PreC, C, X, PreS1, PreS2, S, P or SP, individually or in combination, may be combined with antiviral therapy to reduce, prevent and/or treat HBV infection. In certain embodiments, the compositions and methods described herein can be used in combination with another antiviral therapy, e.g., tenofovir, e.g., entecavir, e.g., another anti-HBV therapy described herein, to reduce, prevent and/or treat HBV infection. In certain embodiments, the compositions and methods described herein can be used in combination with another therapy, e.g., interferon, e.g., pegylated-interferon, e.g., PD-1 inhibition, e.g., another anti-HBV therapy, to reduce, prevent and/or treat HBV infection.

In certain embodiments, one or more of the PreC, C, X, PreS1, PreS2, S, P and/or SP gene(s) is targeted as a targeted knockout, e.g., to inhibit essential viral functions, including, e.g., viral gene transcription, viral genome replication and viral capsid formation. In certain embodiments, said approach comprises knocking out one HBV gene (e.g., PreC, C, X, PreS1, PreS2, S, P or SP gene). In certain embodiments, said approach comprises knocking out two HBV genes, e.g., two of PreC, C, X, PreS1, PreS2, S, P or SP gene(s). In certain embodiments, said approach comprises knocking out three HBV genes, e.g., three of PreC, C, X, PreS1, PreS2, S, P or SP gene(s). In certain embodiments, said approach comprises knocking out four HBV genes, e.g., four of PreC, C, X, PreS1, PreS2, S, P and SP genes. In certain embodiments, said approach comprises knocking out five HBV genes, e.g., five of PreC, C, X, PreS1, PreS2, S, P and SP genes. In certain embodiments, said approach comprises knocking out six HBV genes, e.g., six of PreC, C, X, PreS1, PreS2, S, P and SP genes. In certain embodiments, said approach comprises knocking out seven HBV genes, e.g., seven of PreC, C, X, PreS1, PreS2, S, P and SP genes. In certain embodiments, said approach comprises knocking out eight HBV genes, e.g., each of PreC, C, X, PreS1, PreS2, S, P and SP genes.

In certain embodiments, one or more of the PreC, C, X, PreS1, PreS2, S, P and/or SP gene(s) is targeted as a targeted knockdown, e.g., to inhibit essential viral functions, including, e.g., viral gene transcription, viral genome replication and viral capsid formation. In certain embodiments, said approach comprises knocking down the expression of one HBV gene (e.g., one of the PreC, C, X, PreS1, PreS2, S, P or SP gene). In certain embodiments, said approach comprises knocking down the expression of two HBV genes, e.g., two of PreC, C, X, PreS1, PreS2, S, P or SP gene(s). In certain embodiments, said approach comprises knocking down the expression of three HBV genes, e.g., three of PreC, C, X, PreS1, PreS2, S, P or SP gene(s). In certain embodiments, said approach comprises knocking down the expression of four HBV genes, e.g., four of PreC, C, X, PreS1, PreS2, S, P and SP genes. In certain embodiments, said approach comprises knocking down the expression of five HBV genes, e.g., five of PreC, C, X, PreS1, PreS2, S, P and SP genes. In certain embodiments, said approach comprises knocking down the expression of six HBV genes, e.g., six of PreC, C, X, PreS1, PreS2, S, P and SP genes. In certain embodiments, said approach comprises knocking down the expression of seven HBV genes, e.g., seven of PreC, C, X, PreS1, PreS2, S, P and SP genes. In certain embodiments, said approach comprises knocking down the expression of eight HBV genes, e.g., each of PreC, C, X, PreS1, PreS2, S, P and SP genes.

In certain embodiments, two or more of the PreC, C, X, PreS1, PreS2, S, P and/or SP gene(s) are targeted as a targeted knockout and/or knockdown, e.g., to inhibit essential viral functions, including, e.g., viral gene transcription, viral genome replication and viral capsid formation. In certain embodiments, said approach comprises knocking out the expression of one HBV gene (e.g., PreC, C, X, PreS1, PreS2, S, P or SP gene) and knocking down the expression of one HBV gene (e.g., PreC, C, X, PreS1, PreS2, S, P or SP gene) that is different from the gene targeted by the knockout approach. In certain embodiments, said approach comprises knocking out the expression of one or more HBV genes, e.g., one or more of PreC, C, X, PreS1, PreS2, S, P or SP gene(s) and knocking down the expression of one or more HBV genes, e.g., one or more of PreC, C, X, PreS1, PreS2, S, P or SP gene(s) that are different from the target gene(s) targeted by the knockout approach.

Inhibiting essential viral functions, e.g., viral gene transcription, viral genome replication and viral capsid formation, may decrease the duration and/or severity of HBV infection, including but not limited to acute, occult, latent and/or chronic infection, and/or decreases shedding of viral particles. Subjects also experience shorter duration(s) of illness, decreased risk of cirrhosis, decreased risk of hepatitis, decreased risk of end stage liver disease, decreased risk of hepatocellular carcinoma, decreased risk of transmission to sexual partners, decreased risk of transmission to the fetus in the case of pregnancy and/or the potential for full clearance of HBV (cure).

In certain embodiments, altering (e.g., knocking out or knocking down) the expression of the PreC, C, X, PreS1, PreS2, S, P or SP genes, individually or in combination, can reduce HBV protein expression. In certain embodiments, the reduction in HBV protein expression can cause the reduction of HBV peptide presentation by MEW class I and II molecules and the reversal of T-cell failure, which can treat HBV infection. In certain embodiments, a reduction in viral protein production can lead to the reversal of immune exhaustion and a return of functional B-cell and T-cell responses against hepatocytes infected with HBV.

In certain embodiments, the methods disclosed herein can cause the decline in HBe, HBc, HBx, LHBs, MHBs, SHBs, Pol, and/or HBSP protein production. For example, and not by way of limitation, the methods disclosed herein can comprise inducing a decline in certain HBV proteins, e.g., HBc, e.g., HBpol, e.g., HBx, whose expression is thought to be the cause of T-cell failure in chronic HBV (Feng et. al, J Biomed Sci. 2007 January; 14(1):43-57). In certain embodiments, the method comprises inducing a decline in any and/or all HBV protein production, e.g., HBe, HBc, HBx, LHBs, MHBs, SHBs, Pol, and/or HBSP protein production, as a high viral load is thought to be the primary mechanism for the failure of HBV-specific CD8+ T-cell responses (Schmidt et. al, Emerging Microbes & Infections (2013) 2, e15; Published online 27 Mar. 2013).

In certain embodiments, a decline in HBV protein production, e.g., a decline in HBe, HBc, HBx, LHBs, MHBs, SHBs, Pol, and/or HBSP protein production, gives rise to a reduction in the overwhelming presentation of antigens to the humoral (B-cell) mediated immune system. In certain embodiments, B-cell mediated antibody production is no longer overwhelmed by HBV antigen production and B-cell mediated antibody production is stoichiometrically equivalent to HBV antigen production, e.g., HBsAg production is decreased and anti-HBs antibody can mediate clearance of HbsAg. In certain embodiments, a reduction in the volume and presentation of HBV antigens, e.g., HBeAg, HBcAg, HBxAg, HBsAg, HBpolAg allows for effective humoral immunity, e.g., viral-specific neutralizing antibody production, e.g., anti-HBe Ag production, e.g., anti-HBcAg production, e.g., anti-HBxAg production, e.g., anti-HBsAg production, e.g., anti-HBpolAg production. In certain embodiments, a reduction in the presentation of HBV antigens, e.g., HBeAg, HBcAg, HBxAg, HBsAg, HBpolAg allows for B-cell mediated antibody clearance of HBV antigens and viral particles, including the Dane particle.

In certain embodiments, knockdown of HBV protein production, e.g., HBc (HB core protein), HBpol (HB polymerase protein), HBx (HB x protein) and/or HBs (HB s protein) leads to reversal of immune exhaustion in a subject, restoration of T-cell mediated immunity and/or clearance of chronic HBV infection. For example, and not by way of limitation, knock down of HBV protein production can be performed by eiCas9 or an eiCas9 fusion protein mediated knock down of integrated genomic HBV DNA.

In certain embodiments, knockdown of HBc (HB core protein) production, e.g., by eiCas9 or an eiCas9 fusion protein mediated knock down of HBV cccDNA, leads to reversal of immune exhaustion in a subject, restoration of T-cell mediated immunity and/or clearance of chronic HBV infection. In certain embodiments, knockdown of HBc production, by eiCas9 or an eiCas9 fusion protein mediated knock down of both integrated genomic HBV DNA and HBV cccDNA, leads to reversal of immune exhaustion, restoration of T-cell mediated immunity and/or clearance of chronic HBV infection in a subject.

In certain embodiments, knockdown of HBx (HB x protein) production, by eiCas9 or an eiCas9 fusion protein mediated knockdown of HBV cccDNA, leads to reversal of immune exhaustion in a subject, restoration of T-cell mediated immunity and/or clearance of chronic HBV infection. In certain embodiments, knockdown of HBx production, by eiCas9 or an eiCas9 fusion protein mediated knockdown of both integrated genomic HBV DNA and HBV cccDNA, leads to reversal of immune exhaustion, restoration of T-cell mediated immunity and/or clearance of chronic HBV infection in a subject.

In certain embodiments, knockdown of HBpol (HB polymerase protein) production, by eiCas9 or an eiCas9 fusion protein mediated knock down of HBV cccDNA, leads to reversal of immune exhaustion in a subject, restoration of T-cell mediated immunity and/or clearance of chronic HBV infection. In certain embodiments, knockdown of HBpol production, by eiCas9 or an eiCas9 fusion protein mediated knock down of both integrated genomic HBV DNA and HBV cccDNA, leads to reversal of immune exhaustion, restoration of T-cell mediated immunity and/or clearance of chronic HBV infection in a subject.

In certain embodiments, knockdown of HBs (HB S protein) production, by eiCas9 or an eiCas9 fusion protein mediated knock down of HBV cccDNA, leads to reversal of immune exhaustion in a subject, restoration of T-cell mediated immunity and/or clearance of chronic HBV infection.

In certain embodiments, the methods described herein eliminate and/or decrease the levels of circulating HBsAg, HBeAg and other HBV proteins (e.g., HBpreC, HBc, HBpreS1, HBpreS2, HBp, HBsp) to a degree that permits T-cell and/or B-cell recovery, including T-cell mediated cytotoxic clearance of infected hepatocytes and B-cell mediated clearance of HBsAg and/or Dane particles thereby producing a functional or virologic cure of HBV infection based on immunologic clearance of infected cells.

In certain embodiments, the knockdown methods described herein cause the continued transient knockdown of circulating HBV proteins, e.g., HBs, HBe, HBpreC, HBc, HBpreS1, HBpreS2, HBp, HBsp for long enough (e.g., 1 month, 3 months, 6 months, 1 year, 2 years) to allow for immune reconstitution and subsequent clearance of infected hepatocytes via T- and B-cell mediated mechanisms. In certain embodiments, the knockdown methods described herein are administered on a recurring basis (repeated administration) to allow for continued knockdown of circulating HBV proteins. In certain embodiments, the knock down methods described herein are administered weekly or monthly over the course of 1, 2, 3, 4, 6, 9 and/or 12 months. In certain embodiments, the knockdown methods described herein are given concomitantly with immune activating therapies such as, but not limited to, IFN and PD-1 inhibitors.

Knocking out and/or knocking down one or more copies (e.g., 1, 2, 3, 4, 5, 6, 7, 8, 9, 10, 15, 20, 25 or 50 copies) of one or more target genes (e.g., PreC, C, X, PreS1, PreS2, S, P or SP gene) may be performed prior to disease onset or after disease onset, but preferably early in the disease course.

In certain embodiments, the method comprises initiating treatment of a subject prior to disease onset. In certain embodiments, the method comprises initiating treatment of a subject after disease onset.

In certain embodiments, the method comprises initiating treatment of a subject well after disease onset, e.g., 1, 2, 3, 4, 5, 6, 7, 8, 9, 10, 12, 16, 24, 36, 48 or more months after onset of HBV infection. In certain embodiments, the method comprises initiating treatment of a subject well after disease onset, e.g., 1, 2, 3, 4, 5, 10, 15, 20, 25, 40, 50 or 60 years after onset of HBV infection. This may be effective as disease progression is slow in some cases and a subject may present well into the course of illness.

In certain embodiments, the method comprises initiating treatment of a subject in an advanced stage of disease, e.g., during immune-tolerant phase, e.g., during immune-active phase, e.g., during inactive carrier phase. In certain embodiments, the method comprises initiating treatment of a subject in the case of acute disease. In certain embodiments, the method comprises initiating treatment of a subject in the case of severe disease exacerbation, e.g., during acute hepatitis. In certain embodiments, the method comprises initiating treatment of a subject in the case of asymptomatic disease, e.g., during latent infection, e.g., during chronic infection with low ALT levels and/or low HBV DNA levels and/or absence of cirrhosis.

In certain embodiments, the method comprises initiating treatment of a subject in the case of occult hepatitis B infection (OBI), including but not limited to subjects testing negative for HBsAG and positive for HBV DNA.

In certain embodiments, the method comprises initiating treatment of a subject at risk for hepatocellular carcinoma secondary to exposure to acute HBV. In certain embodiments, the method comprises initiating treatment of a subject at risk for hepatocellular carcinoma due to chronic HBV. In certain embodiments, the method comprises initiating treatment of a subject at risk for hepatocellular carcinoma due to exposure to HBV, including but not limited to subjects with increased HBV integration events, subjects with HBV integration events in known oncogenes, subjects with HBV integration events in exonic and/or promoter regions.

Overall, initiation of treatment for subjects at all stages of disease is expected to improve healing, decrease duration of disease and be of benefit to subjects.

In certain embodiments, the method comprises initiating treatment of a subject prior to disease expression. In certain embodiments, the method comprises initiating treatment of a subject in an early stage of disease, e.g., when a subject has been exposed to HBV or is thought to have been exposed to HBV.

In certain embodiments, the method comprises initiating treatment of a subject prior to disease expression. In certain embodiments, the method comprises initiating treatment of a subject in an early stage of disease, e.g., when a subject has tested positive for HBV infection but has no signs or symptoms.

In certain embodiments, the method comprises initiating treatment of a subject at the appearance of elevated liver enzymes, e.g., elevated AST, e.g., elevated ALT.

In certain embodiments, the method comprises initiating treatment at the appearance of any of the following symptoms consistent or associated with HBV hepatitis: jaundice, nausea and vomiting, weakness, dark urine, fever, abdominal pain, loss of appetite, confusion and changes in mental status, and joint pain.

In certain embodiments, the method comprises initiating treatment of a subject at the appearance of laboratory evidence consistent with acute or chronic HBV infection, including but not limited to: presence of HBV DNA in the blood, presence of HBsAg in the blood, presence of HBeAg in the blood, presence of HBxAg in the blood, elevated HBV DNA levels in the blood, elevated HBsAg levels in the blood, elevated HBeAg levels in the blood, elevated HBxAg levels in the blood, presence of anti-HBs in the blood, presence of anti-HBc in the blood, presence of anti-HBe in the blood, presence of anti-HBx in the blood.

In certain embodiments, the method comprises initiating treatment of a subject with evidence of HBV infection on liver biopsy, including but not limited to: presence of HBV DNA, presence of HBsAg, presence of HBeAg, presence of HBxAg, presence of hepatitis delta virus.

In certain embodiments, the method comprises initiating treatment of a subject with evidence of hepatitis delta virus (HDV) infection, including but not limited to: presence of HDV DNA on blood test, presence of HDV DNA on liver biopsy.

In certain embodiments, the method comprises initiating treatment of a subject with evidence of HBV infection, including but not limited to: hepatic fibrosis on ultrasound, increased liver stiffness on Fibroscan.

In certain embodiments, the method comprises initiating treatment at the appearance of any of the following signs consistent with or associated with HBV cirrhosis: spider angioma, palmar erythema, hepatomegaly, jaundice, splenomegaly, easy bruising and bleeding, hepatic encephalopathy, or portal hypertension.

In certain embodiments, the method comprises initiating treatment in a patient with signs consistent with HBV cirrhosis and/or hepatitis on ultrasound, fibroscan, liver biopsy, blood test, CT scan and/or MRI.

In certain embodiments, the method comprises initiating treatment in utero in case of high risk of maternal-to-fetal transmission.

In certain embodiments, the method comprises initiating treatment during pregnancy in case of mother who has active HBV infection or has recent primary HBV infection or who has chronic HBV infection or who has occult HBV infection.

In certain embodiments, the method comprises initiating treatment of a subject who has received a HBV vaccine. In certain embodiments, the method comprises initiating treatment of a subject who has evidence of, who is at risk for, or who is a member of a population at risk for a “vaccine escape” mutation, including but not limited to HBV-G145R mutants.

In certain embodiments, the method comprises initiating treatment prior to organ transplantation or immediately following organ transplantation. In certain embodiments, the method comprises initiating treatment prior to hematopoietic stem cell transplantation (HSCT) or immediately following HSCT. In certain embodiments, the method comprises initiating treatment prior to chemotherapy or immediately following chemotherapy. In certain embodiments, the method comprises initiating treatment prior to or immediately following immunosuppressant therapy.

In certain embodiments, the method comprises initiating treatment in case of suspected exposure to HBV.

In certain embodiments, the method comprises initiating treatment prophylactically, especially in case of suspected exposure of infants, children or immune suppressed subjects.

In certain embodiments, the method comprises initiating treatment prophylactically, especially in case of suspected exposure of health care workers.

In certain embodiments, the method comprises initiating treatment of a subject who suffers from or is at risk of developing severe manifestations of HBV infections, e.g., neonates, infants, children, subjects with HIV, subjects who are on immunosuppressant therapy following organ transplantation, subjects who have cancer, subjects who are undergoing chemotherapy, subjects who will undergo chemotherapy, subjects who are undergoing radiation therapy, subjects who will undergo radiation therapy.

Both HIV positive subjects and post-transplant subjects may experience chronic HBV, and have a high risk of developing HBV-related cirrhosis and/or HBV-related hepatocellular carcinoma. Neonates are also at risk for chronic HBV. Inhibiting essential viral functions, e.g., viral gene transcription, viral genome replication and viral capsid formation, may provide superior protection to said populations at risk for chronic HBV infections. Subjects treated with the treatment described herein may experience lower rates of chronic HBV, lower rates of cirrhosis and lower rates of hepatocellular carcinoma, which will profoundly improve quality of life.

In certain embodiments, the method comprises initiating treatment of a subject who has tested positive for HBV. In certain embodiments, the method comprises initiating treatment of a subject who has tested positive for HDV.

In certain embodiments, a cell is manipulated by editing (e.g., introducing a mutation in) one or more target genes, e.g., PreC, C, X, PreS1, PreS2, S, P or SP gene(s). In certain embodiments, the expression of one or more target genes (e.g., one or more PreC, C, X, PreS1, PreS2, S, P or SP gene(s) described herein) is modulated, e.g., in vivo.

In certain embodiments, the method comprises delivery of gRNA by an AAV. In certain embodiments, the method comprises delivery of gRNA by a lentivirus. In certain embodiments, the method comprises delivery of gRNA by a nanoparticle, e.g., lipid nanoparticle.

In certain embodiments, the method further comprising treating the subject with a second antiviral therapy, e.g., an anti-HBV therapy described herein. In certain embodiments, the method further comprising treating the subject with a second therapy that stimulates the immune system, e.g., PEG-interferon, a PD-1 inhibitor, a vaccine. The compositions described herein can be administered concurrently with, prior to, or subsequent to, one or more additional therapies or therapeutic agents. The composition and the other therapy or therapeutic agent can be administered in any order. In certain embodiments, the effect of the two treatments is synergistic. Exemplary anti-HBV therapies include, but are not limited to, interferon, PEG-interferon, entacavir, tenofovir, a therapeutic vaccine, or an immune-stimulatory therapy, e.g., a PD-1 inhibitor.

When two or more genes (e.g., PreC, C, X PreS1, PreS2, S, P or SP) are targeted for alteration, the two or more genes (e.g., PreC, C, X, PreS1, PreS2, S, P or SP) may be altered sequentially or simultaneously. In certain embodiments, the effect of the alterations is synergistic.

### 4. Methods of Altering the HBV Genome, Including PreC, C, X, PreS1, PreS2, S, P and/or SP Gene(s)

As disclosed herein, a position in the HBV genome (e.g., any location on the HBV genome) can by altered by gene editing, e.g., using CRISPR-Cas9 mediated methods as described herein. In certain embodiments, a position in the HBV genome, e.g., a HBV target position in the PreC, C, X, PreS1, PreS2, S, P or SP gene(s), can be altered alone or in combination by gene editing, e.g., using CRISPR-Cas9 mediated methods as described herein.

The methods, genome editing systems and compositions discussed herein provide for altering a HBV genome, e.g., a target position in the HBV genome, including but not limited to a target position in one or more of the PreC, C, X PreS1, PreS2, S, P and/or SP gene(s). In certain embodiments, a HBV target position can be altered by gene editing, e.g., using CRISPR-Cas9 mediated methods to alter a position in the HBV genome, e.g., by a presently disclosed genome editing system. In certain embodiments, a HBV target position can be altered by a presently disclosed genome editing system to alter one or more of the PreC, C, X, PreS1, PreS2, S, P and/or SP gene(s).

Disclosed herein are methods, genome editing systems and compositions for altering (e.g., knocking out or knocking down) a HBV target position in the PreC, C, X, PreS1, PreS2, S, P and/or SP gene(s). Altering (e.g., knocking out or knocking down) the HBV target position is achieved, e.g., by: (1) knocking out one or more of the PreC, C, X PreS1, PreS2, S, P and/or SP gene(s): (a) insertion or deletion (e.g., NHEJ-mediated insertion or deletion) of one or more nucleotides in close proximity to or within the early coding region of the PreC, C, X, PreS1, PreS2, S, P and/or SP gene(s), or (b) deletion (e.g., NHEJ-mediated deletion) of a genomic sequence or multiple genomic sequences including at least a portion or portions of the PreC, C, X, PreS1, PreS2, S, P and/or SP gene(s), or (2) knocking down one or more of the PreC, C, X, PreS1, PreS2, S, P and/or SP gene(s) mediated by enzymatically inactive Cas9 (eiCas9) molecule or an eiCas9-fusion protein by targeting non-coding region, e.g., a promoter region, of the gene. In certain embodiments, eiCas9 or an eiCas9-fusion protein mediated knockdown of one or more of the PreC, C, X, PreS1, PreS2, S, P and/or SP gene(s) knocks down a gene or genes located on HBV cccDNA. In certain embodiments, eiCas9 or an eiCas9-fusion protein mediated knockdown of one or more of the PreC, C, X, PreS1, PreS2, S, P and/or SP gene(s) knocks down a gene or genes located on HBV rcDNA. In certain embodiments, eiCas9 or an eiCas9-fusion protein mediated knockdown of one or more of the PreC, C, X, PreS1, PreS2, S, P and/or SP gene(s) knocks down a gene or genes located on HBV linearized DNA. In certain embodiments, eiCas9 mediated or eiCas9-fusion protein mediated knockdown of one or more of the PreC, C, X, PreS1, PreS2, S, P and/or SP gene(s) knocks down a gene or genes that is located within the human genome, because the HBV genome has been integrated into a subject's genome.

All approaches give rise to altering (e.g., knocking out or knocking down) the HBV genome (e.g., one or more of the PreC, C, X, PreS1, PreS2, S, P or SP genes.)

In certain embodiments, the methods, genome editing systems and compositions described herein introduce one or more breaks near the early coding region in one or more of the PreC, C, X, PreS1, PreS2, S, P and/or SP gene(s). In certain embodiments, the methods, genome editing systems and compositions described herein introduce two or more breaks to flank at least a portion of one or more of the PreC, C, X, PreS1, PreS2, S, P and/or SP gene(s). The two or more breaks remove (e.g., delete) a genomic sequence including at least a portion of one or more of the PreC, C, X, PreS1, PreS2, S, P and/or SP gene(s). In certain embodiments, the methods, genome editing systems and compositions described herein comprise knockdown of one or more of the PreC, C, X, PreS1, PreS2, S, P and/or SP gene(s) mediated by enzymatically inactive Cas9 (eiCas9) molecule or an eiCas9-fusion protein by targeting the promoter region of HBV target knockdown position. The methods, genome editing systems and compositions described herein result in altering (e.g., knocking out or knocking down) the HBV genome (e.g., HBV cccDNA, linearized HBV DNA, HBV rcDNA and/or integrated HBV DNA), and/or altering (e.g., knocking out or knocking down) one or more of the PreC, C, X, PreS1, PreS2, S, P and/or SP gene(s).

An alteration of one or more of the PreC, C, X, PreS1, PreS2, S, P and/or SP gene(s) can be mediated by any mechanism. Exemplary mechanisms that can be associated with an alteration of one or more of the PreC, C, X, PreS1, PreS2, S, P and/or SP gene(s) include, but are not limited to, non-homologous end joining (e.g., classical or alternative), microhomology-mediated end joining (MMEJ), homology-directed repair (e.g., endogenous donor template mediated), SDSA (synthesis dependent strand annealing), single strand annealing or single strand invasion.

4.1. Knocking Out One or More of the PreC, C, X, PreS1, PreS2, S, P and/or SP Gene(s) by Introducing an Indel or a Deletion in One or More HBV Gene(s)

In certain embodiments, the method comprises introducing an insertion or deletion of one or more nucleotides in close proximity to the HBV target knockout position (e.g., the early coding region) of the PreC, C, X PreS1, PreS2, S, P and/or SP gene(s). As described herein, in certain embodiments, the method comprises the introduction of one or more breaks (e.g., single strand breaks or double strand breaks) sufficiently close to (e.g., either 5′ or 3′ to) the early coding region of the HBV target knockout position, such that the break-induced indel could be reasonably expected to span the HBV target knockout position (e.g., the early coding region). NHEJ-mediated repair of the break(s) allows for the NHEJ-mediated introduction of an indel in close proximity to within the early coding region of the HBV target knockout position.

In certain embodiments, the method comprises introducing a deletion of a genomic sequence comprising at least a portion of one or more of the HBV gene(s) PreC, C, X PreS1, PreS2, S, P and/or SP. As described herein, in certain embodiments, the method comprises the introduction of two double stand breaks—one 5′ and the other 3′ to (i.e., flanking) the HBV target position. In certain embodiments, two gRNAs, e.g., unimolecular (or chimeric) or modular gRNA molecules, are configured to position the two double strand breaks on opposite sides of the HBV target knockout position in the PreC, C, X PreS1, PreS2, S, P and/or SP gene(s).

In certain embodiments, a single strand break is introduced (e.g., positioned by one gRNA molecule) at or in close proximity to a HBV target position in the PreC, C, X PreS1, PreS2, S, P and/or SP gene. In certain embodiments, a single gRNA molecule (e.g., with a Cas9 nickase) is used to create a single strand break at or in close proximity to the HBV target position, e.g., the gRNA is configured such that the single strand break is positioned either upstream or downstream of the HBV target position. In certain embodiments, the break is positioned to avoid unwanted target chromosome elements, such as repeat elements, e.g., an Alu repeat.

In certain embodiments, a double strand break is introduced (e.g., positioned by one gRNA molecule) at or in close proximity to a HBV target position in the PreC, C, X PreS1, PreS2, S, P and/or SP gene. In certain embodiments, a single gRNA molecule (e.g., with a Cas9 nuclease other than a Cas9 nickase) is used to create a double strand break at or in close proximity to the HBV target position, e.g., the gRNA molecule is configured such that the double strand break is positioned either upstream or downstream of a HBV target position. In certain embodiments, the break is positioned to avoid unwanted target chromosome elements, such as repeat elements, e.g., an Alu repeat.

In certain embodiments, two single strand breaks are introduced (e.g., positioned by two gRNA molecules) at or in close proximity to a HBV target position in the PreC, C, X PreS1, PreS2, S, P and/or SP gene. In certain embodiments, two gRNA molecules (e.g., with one or two Cas9 nickcases) are used to create two single strand breaks at or in close proximity to the HBV target position, e.g., the gRNAs molecules are configured such that both of the single strand breaks are positioned upstream or downstream of the HBV target position. In certain embodiments, two gRNA molecules (e.g., with two Cas9 nickcases) are used to create two single strand breaks at or in close proximity to the HBV target position, e.g., the gRNAs molecules are configured such that one single strand break is positioned upstream and a second single strand break is positioned downstream of the HBV target position. In certain embodiments, the breaks are positioned to avoid unwanted target chromosome elements, such as repeat elements, e.g., an Alu repeat.

In certain embodiments, two double strand breaks are introduced (e.g., positioned by two gRNA molecules) at or in close proximity to a HBV target position in the PreC, C, X PreS1, PreS2, S, P and/or SP gene. In certain embodiments, two gRNA molecules (e.g., with one or two Cas9 nucleases that are not Cas9 nickases) are used to create two double strand breaks to flank a HBV target position, e.g., the gRNA molecules are configured such that one double strand break is positioned upstream and a second double strand break is positioned downstream of the HBV target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a HBV target position in the PreC, C, X, PreS1, PreS2, S, P and/or SP gene. In certain embodiments, three gRNA molecules (e.g., with a Cas9 nuclease other than a Cas9 nickase and one or two Cas9 nickases) to create one double strand break and two single strand breaks to flank a HBV target position, e.g., the gRNA molecules are configured such that the double strand break is positioned upstream or downstream of of the HBV target position, and the two single strand breaks are positioned at the opposite site, e.g., downstream or upstream, of the HBV target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a HBV target position in the PreC, C, X PreS1, PreS2, S, P and/or SP gene. In certain embodiments, four gRNA molecule (e.g., with one or more Cas9 nickases are used to create four single strand breaks to flank a HBV target position in the PreC, C, X PreS1, PreS2, S, P and/or SP gene, e.g., the gRNA molecules are configured such that a first and second single strand breaks are positioned upstream of the HBV target position, and a third and a fourth single stranded breaks are positioned downstream of the HBV target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two or more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

4.2 Knocking Out One or More PreC, C, X, PreS1, PreS2, S, P or SP Genes by Deleting (e.g., NHEJ-Mediated Deletion) a Genomic Sequence PreC, C, X, PreS1, PreS2, S, P or SP Genes or Multiple Genomic Sequences Including at Least a Portion or Portions of the PreC, C, X, PreS1, PreS2, S, P and/or SP Gene(s).

In certain embodiments, the method comprises deleting (e.g., NHEJ-mediated deletion) a genomic sequence including at least a portion of the PreC, C, X, PreS1, PreS2, S, P or SP genes or multiple genomic sequences including at least a portion or portions of the PreC, C, X PreS1, PreS2, S, P and/or SP gene(s). In certain embodiments, deleting (e.g., NHEJ-mediated deletion) a genomic sequence or multiple genomic sequences including at least a portion or portions of the PreC, C, X PreS1, PreS2, S, P and/or SP gene(s) gives rise to destruction of the genomic DNA and/or clearance of the DNA from infected cells. In certain embodiments, deleting (e.g., NHEJ-mediated deletion) a genomic sequence or multiple genomic sequences within the HBV genome gives rise to destruction of the genomic DNA which causes reduction and/or cessation of transcription of HBV RNA. In certain embodiments, deleting a genomic sequence or multiple genomic sequences within the HBV genome gives rise to destruction of the genomic DNA and the cessation of translation of HBV proteins, e.g., HBe, HBc, HBx, LHBs, MHBs, SHBs, Pol, and/or HBSP proteins. In certain embodiments, deleting (e.g., NHEJ-mediated deletion) a genomic sequence or multiple genomic sequences within the HBV genome gives rise to destruction of the genomic DNA which causes any of the following, singly or in combination: decreased HBV DNA production, decreased HBV cccDNA production, decreased viral infectivity, decreased packaging of viral particles, decreased production of production of viral proteins, e.g., HBe, HBc, HBx, LHBs, MHBs, SHBs, Pol, and/or HBSP proteins. In certain embodiments, deleting (e.g., NHEJ-mediated deletion) a genomic sequence or multiple genomic sequences within the HBV genome gives rise to destruction of the genomic DNA which causes a decline in HBsAg production to such a point that anti-HBsAg production is no longer overwhelmed by HBsAg production, such that a subject is capable of mounting a functional immune response to HBV, e.g., a subject reverses ‘immune exhaustion’, and a subject can achieve a functional virologic cure of chronic HBV.

As described herein, in certain embodiments, the method comprises the introduction two sets of breaks (e.g., a pair of double strand breaks, one double strand break or a pair of single strand breaks, or two pairs of single strand breaks) to flank a region of the PreC, C, X PreS1, PreS2, S, P or SP genes (e.g., a coding region, e.g., an early coding region, or a non-coding region, e.g., a non-coding sequence of the PreC, C, X PreS1, PreS2, S, P or SP genes, e.g., a promoter, an enhancer, an intron, a 3′UTR, and/or a polyadenylation signal). NHEJ-mediated repair of the break(s) may allow for alteration of the PreC, C, X, PreS1, PreS2, S, P or SP genes as described herein, which reduces or eliminates expression of the gene, e.g., to knock out one or both alleles of the PreC, C, X PreS1, PreS2, S, P or SP genes.

In certain embodiments, two double strand breaks are introduced (e.g., positioned by two gRNA molecules) at or in close proximity to a HBV target position in the PreC, C, X PreS1, PreS2, S, P or SP genes. In certain embodiments, two gRNA molecules (e.g., with one or two Cas9 nucleases that are not Cas9 nickases) are used to create two double strand breaks to flank a HBV target position, e.g., the gRNA molecules are configured such that one double strand break is positioned upstream and a second double strand break is positioned downstream of the HBV target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a HBV target position in the PreC, C, X, PreS1, PreS2, S, P or SP genes. In certain embodiments, three gRNA molecules (e.g., with a Cas9 nuclease other than a Cas9 nickase and one or two Cas9 nickases) to create one double strand break and two single strand breaks to flank a HBV target position, e.g., the gRNA molecules are configured such that the double strand break is positioned upstream or downstream of the HBV target position, and the two single strand breaks are positioned at the opposite site, e.g., downstream or upstream, of the HBV target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a HBV target position in the PreC, C, X PreS1, PreS2, S, P or SP genes. In certain embodiments, four gRNA molecule (e.g., with one or more Cas9 nickases are used to create four single strand breaks to flank a HBV target position in the PreC, C, X PreS1, PreS2, S, P or SP genes, e.g., the gRNA molecules are configured such that a first and second single strand breaks are positioned upstream of the HBV target position, and a third and a fourth single stranded breaks are positioned of the HBV target position. In certain embodiments, the breaks are positioned to avoid unwanted target chromosome elements, such as repeat elements, e.g., an Alu repeat.

In certain embodiments, multiple (e.g., three four, five, six, seven, eight or more) gRNA molecules are used with one or more (e.g., two, three, four or more) Cas9 molecule. In certain embodiments, when the multiple (e.g., three four, five, six, seven, eight or more) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

4.3 Knocking Down One or More of the PreC, C, X, PreS1, PreS2, S, P and/or SP Gene(s) Mediated by an Enzymatically Inactive Cas9 (eiCas9) Molecule

A targeted knockdown approach reduces or eliminates expression of functional PreC, C, X PreS1, PreS2, S, P and/or SP genes product. As described herein, in certain embodiments, a targeted knockdown is mediated by targeting an enzymatically inactive Cas9 (eiCas9) molecule or an eiCas9 fused to a transcription repressor domain or chromatin modifying protein to PreC, C, X PreS1, PreS2, S, P and/or SP genes. For example, and not by way of limitation, one or more genes (e.g., PreC, C, X, PreS1, PreS2, S, P and/or SP) can be knocked down using the methods disclosed herein.

Methods and compositions discussed herein may be used to alter the expression of one or more of the PreC, C, X, PreS1, PreS2, S, P and SP genes to reduce, prevent and/or treat HBV infection by targeting a transcriptional regulatory region, e.g., a promoter region (e.g., a promoter region that controls the transcription of one or more of the PreC, C, X, PreS1, PreS2, S, P or SP genes). In certain embodiments, the promoter region is targeted to knock down expression of one or more of the PreC, C, X, PreS1, PreS2, S, P or SP genes. A targeted knockdown approach reduces or eliminates expression of functional PreC, C, X, PreS1, PreS2, S, P or SP genes product. As described herein, in certain embodiments, a targeted knockdown is mediated by targeting an enzymatically inactive Cas9 (eiCas9) or an eiCas9 fused to a transcription repressor domain or chromatin modifying protein to PreC, C, X, PreS1, PreS2, S, P or SP genes.

In certain embodiments, one or more eiCas9s may be used to block binding of one or more endogenous transcription factors. In certain embodiments, an eiCas9 can be fused to a chromatin modifying protein. Altering chromatin status can result in decreased expression of the target gene. One or more eiCas9s fused to one or more chromatin modifying proteins may be used to alter chromatin status.

In certain embodiments, eiCas9 mediated reduction in the expression of one or more of the PreC, C, X, PreS1, PreS2, S, P or SP genes causes the reduction and/or cessation of transcription of HBV RNA. In certain embodiments, eiCas9 mediated reduction in the expression of one or more of the PreC, C, X, PreS1, PreS2, S, P or SP genes leads to reduction and/or cessation of translation of HBV proteins, e.g., HBe, HBc, HBx, LHBs, MHBs, SHBs, Pol, and/or HBSP proteins. In certain embodiments, eiCas9 mediated reduction in the expression of one or more of the PreC, C, X, PreS1, PreS2, S, P or SP genes gives rise to any of the following, singly or in combination: decreased HBV DNA production, decreased HBV replication, decreased viral infectivity, decreased packaging of viral particles, decreased production of viral proteins, e.g., HBe, HBc, HBx, LHBs, MHBs, SHBs, Pol, and/or HBSP proteins. In certain embodiments, eiCas9 mediated reduction in the expression of one or more of the PreC, C, X, PreS1, PreS2, S, P or SP genes gives rise to a decline in HBsAg production to such a point that anti-HBsAg production in a subject is no longer overwhelmed by HBsAg production, such that a subject is capable of mounting a functional immune response to HBV, e.g., a subject reverses ‘immune exhaustion’, and a subject can achieve a functional virologic cure of chronic HBV.

In certain embodiments, knockdown of one or more of the PreC, C, X PreS1, PreS2, S, P and/or SP gene(s) cures HBV infection. In certain embodiments, knockdown of one or more of the PreC, C, X, PreS1, PreS2, S, P and/or SP gene(s) leads to a functional cure of HBV infection. In certain embodiments, knockdown of one or more of the PreC, C, X PreS1, PreS2, S, P and/or SP gene(s) leads to a sustained virologic response to HBV infection. In certain embodiments, knockdown of one or more of the PreC, C, X PreS1, PreS2, S, P and/or SP gene(s) is an effective method of preventing the sequelae of chronic HBV, including fibrosis, cirrhosis, and hepatocellular carcinoma.

In certain embodiments, a targeted knockdown approach induces a decline in HBV protein production, e.g., HBe, HBc, HBx, LHBs, MHBs, SHBs, Pol, and/or HBSP protein production. For example, and not by way of limitation, a targeted knockdown approach induces a decline in the protein production of one or more HBV protein such as HBe, HBc, HBx, LHBs, MHBs, SHBs, Pol, and/or HBSP protein. In certain embodiments, a targeted knockdown approach comprises inducing a decline in certain HBV proteins, e.g., HBc, e.g., HBpol, e.g., HBx, whose expression is thought to be the cause of T-cell failure in chronic HBV (Feng et. al, J Biomed Sci. 2007 January; 14(1):43-57). In certain embodiments a targeted knockdown approach comprises inducing a decline in any and/or all HBV protein production, e.g., HBe, HBc, HBx, LHBs, MHBs, SHBs, Pol, and/or HBSP protein production, and the restoration of a subject's immune response to HBV, as a high viral load is thought to be the primary mechanism for the failure of HBV-specific CD8+ T-cell responses (Schmidt et. al, Emerging Microbes & Infections (2013) 2, e15; Published online 27 Mar. 2013).

In certain embodiments, a targeted knockdown approach induces a decline in HBV protein production, e.g., HBe, HBc, HBx, LHBs, MHBs, SHBs, Pol, and/or HBSP protein production, so that there is a corresponding decline in HBV peptide presentation, e.g., HBe-derived, HBc-derived, HBx-derived, LHBs-derived, MHBs-derived, SHBs-derived, Pol-derived, and/or HBSP-derived peptide presentation, by MHC Class I molecules. MHC Class I molecules present HBV-derived peptides on infected liver cells and antigen presenting cells. In certain embodiments, a targeted knockdown approach leads to reconstitution of functional CD8+ T cell-mediated toxicity against HBV-infected hepatocytes, including CD-8+ T-cell mediated cell killing and/or CD-8+ T cell-mediated interferon (IFN) secretion locally within the liver parenchyma. In certain embodiments, CD-8+ T cell-mediated IFN secretion locally, e.g., within the liver parenchyma and/or at or near the site of HBV infected hepatocytes, mediates cell killing and clearance of HBV-infected cells without the systemic side effects of systemic IFN therapy. For example, and not by way of limitation, the methods described herein are more effective and have fewer systemic side effects, e.g., fever, malaise, or muscle aches, than systemic IFN-based therapy. In certain embodiments, CD-8+ T cell-mediated IFN secretion locally leads to the clearance of HBV-infected hepatocytes and to a functional cure of HBV infection. In certain embodiments, a targeted knockdown approach induces a reconstitution of immune competence by restoring activation of T-cell mediated cytotoxicity in subjects. In certain embodiments, a targeted knockdown approach comprises inducing a local IFN response to HBV infection.

In certain embodiments, the method comprises knocking down a region of the HBV genome, e.g., the S gene, e.g., one or more of the PreC, C, X, PreS1, PreS2, P and/or SP gene(s) that is integrated into the subject genome in order to decrease circulating HBV antigen levels, including but not limited to HBsAg. In a chimpanzee model, integrated DNA is implicated in the production of HBsAg and in circulating HBs antigen-emia (Wooddell et al., AASLD abstract #32, Hepatology, 2015: 222A-223A). In certain embodiments, the method comprises knocking down a region of the HBV genome, e.g., the S gene, to induce a functional cure of HBV infection.

In certain embodiments, the method comprises knockdown of a region of the HBV genome, e.g., one or more of the PreC, C, X, PreS1, PreS2, P and/or SP gene(s) that is integrated into the subject genome. In certain embodiments, the method does not comprise knocking down and/or knocking out the S gene. In certain embodiments, the method can further include analyzing the levels of HBsAg to indicate whether the method resulted in a functional cure of the HBV infection. For example, and not by way of limitation, HBsAg can be used as a marker to determine if a method disclosed herein resulted in a functional cure of the HBV infection. In certain embodiments, minimal detection of HBsAg indicates that the patient subjected to a method disclosed herein achieved a functional virologic cure of chronic HBV.

### 5. Guide RNA (gRNA) Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule) (e.g., chimeric), or modular (comprising more than one, and typically two, separate RNA molecules). The gRNA molecules provided herein comprise a targeting domain comprising, consisting of, or consisting essentially of a nucleic acid sequence fully or partially complementary to a target domain (also referred to as “target sequence”). In certain embodiments, the gRNA molecule further comprises one or more additional domains, including for example a first complementarity domain, a linking domain, a second complementarity domain, a proximal domain, a tail domain, and a 5′ extension domain. Each of these domains is discussed in detail below. In certain embodiments, one or more of the domains in the gRNA molecule comprises a nucleotide sequence identical to or sharing sequence homology with a naturally occurring sequence, e.g., from S. pyogenes, S. aureus, or S. thermophilus. In certain embodiments, one or more of the domains in the gRNA molecule comprises a nucleotide sequence identical to or sharing sequence homology with a naturally occurring sequence, e.g., from S. pyogenes or S. aureus,

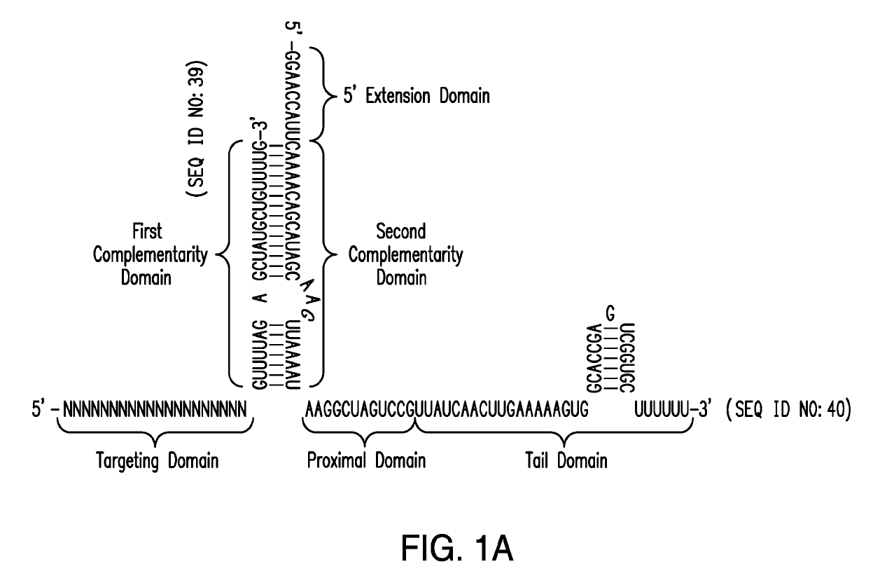

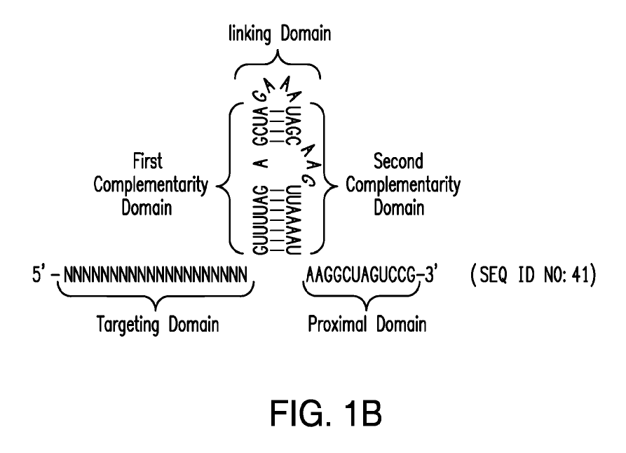

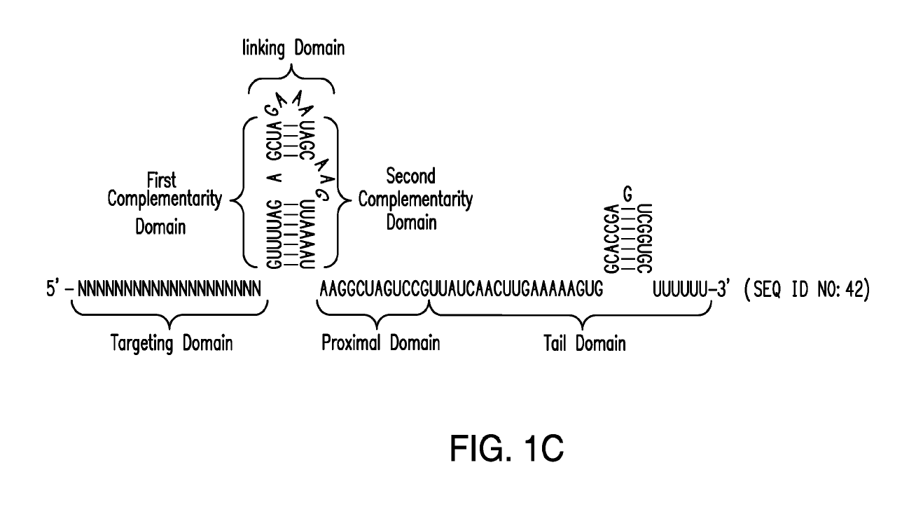

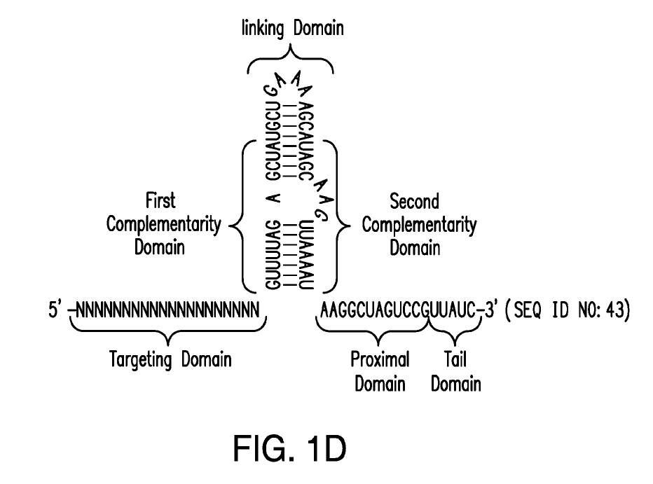

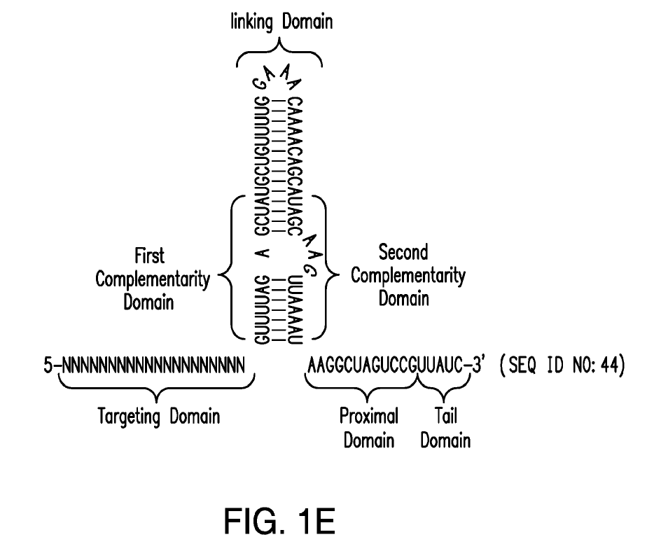

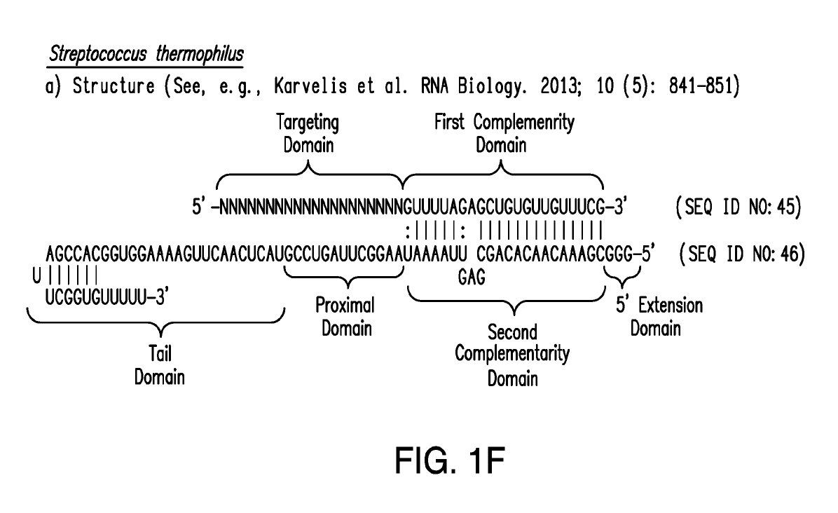

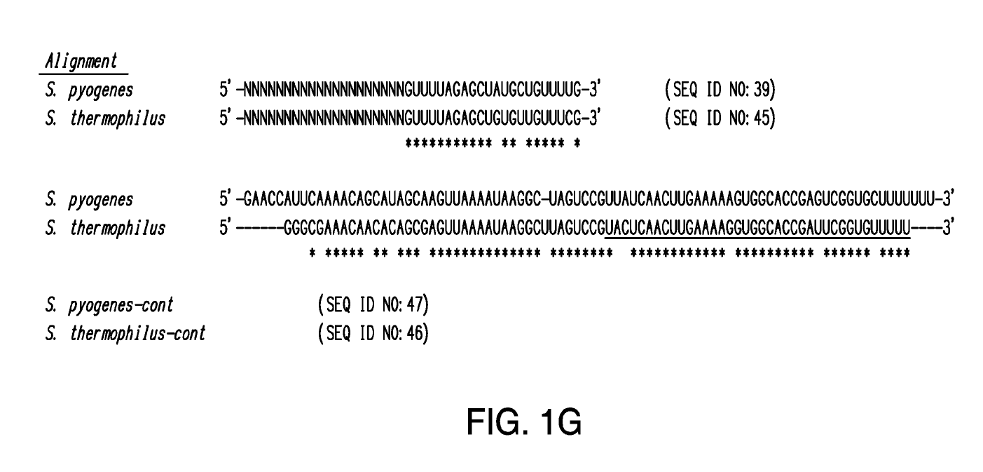

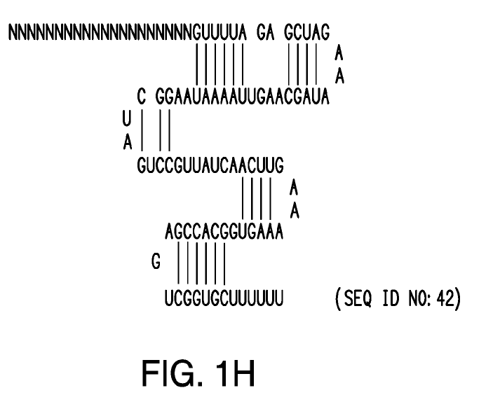

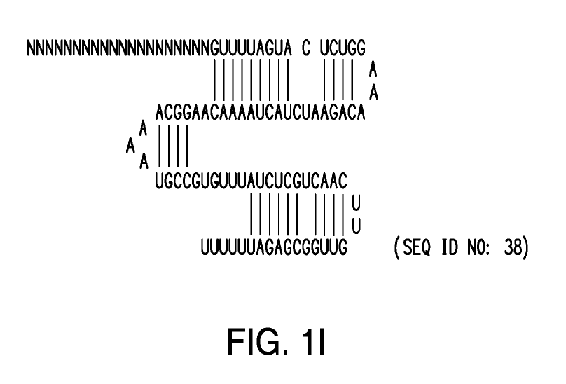

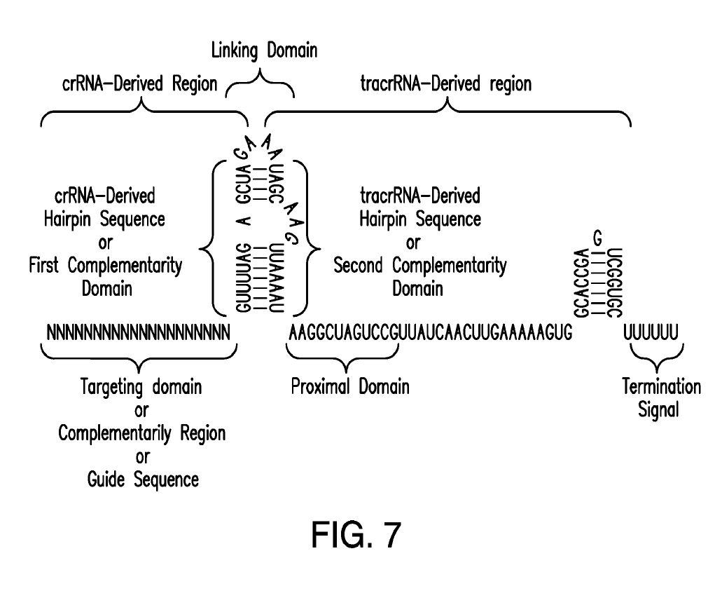

Several exemplary gRNA structures are provided in FIGS. 1A-1I. With regard to the three-dimensional form, or intra- or inter-strand interactions of an active form of a gRNA, regions of high complementarity are sometimes shown as duplexes in FIGS. 1A-1I and other depictions provided herein. FIG. 7 illustrates gRNA domain nomenclature using the gRNA sequence of SEQ ID NO:42, which contains one hairpin loop in the tracrRNA-derived region. In certain embodiments, a gRNA may contain more than one (e.g., two, three, or more) hairpin loops in this region (see, e.g., FIGS. 1H-1I).

In certain embodiments, a unimolecular, or chimeric, gRNA comprises, preferably from 5′ to 3′:

a targeting domain complementary to a target domain in a HBV viral gene selected from the group consisting of PreC gene, C gene, Xgene, PreS1 gene, PreS2 gene, S gene, P gene and SP gene, e.g., a targeting domain comprising a nucleotide sequence selected from SEQ ID NOs: 215 to 141071;

a first complementarity domain;

a linking domain;

a second complementarity domain (which is complementary to the first complementarity domain);

a proximal domain; and

optionally, a tail domain.

In certain embodiments, a modular gRNA comprises:

a first strand comprising, preferably from 5′ to 3′:

a targeting domain complementary to a target domain in a HBV viral gene, e.g., a targeting domain comprising a nucleotide sequence selected from SEQ ID NOs: 215 to 141071; and

a first complementarity domain; and

a second strand, comprising, preferably from 5′ to 3′:

optionally, a 5′ extension domain;

a second complementarity domain;

a proximal domain; and

optionally, a tail domain.

5.1 Targeting Domain

The targeting domain (sometimes referred to alternatively as the guide sequence) comprises, consists of, or consists essentially of a nucleic acid sequence that is complementary or partially complementary to a target nucleic acid sequence in a HBV viral gene selected from the group consisting of PreC gene, C gene, Xgene, PreS1 gene, PreS2 gene, S gene, P gene and SP gene. The nucleic acid sequence in a HBV viral gene selected from the group consisting of PreC gene, C gene, X gene, PreS1 gene, PreS2 gene, S gene, P gene and SP gene to which all or a portion of the targeting domain is complementary or partially complementary is referred to herein as the target domain.

Methods for selecting targeting domains are known in the art (see, e.g., Fu 2014; Sternberg 2014). Examples of suitable targeting domains for use in the methods, compositions, and kits described herein comprise nucleotide sequences set forth in SEQ ID NOs: 215 to 8407.

The strand of the target nucleic acid comprising the target domain is referred to herein as the complementary strand because it is complementary to the targeting domain sequence. Since the targeting domain is part of a gRNA molecule, it comprises the base uracil (U) rather than thymine (T); conversely, any DNA molecule encoding the gRNA molecule can comprise thymine rather than uracil. In a targeting domain/target domain pair, the uracil bases in the targeting domain will pair with the adenine bases in the target domain. In certain embodiments, the degree of complementarity between the targeting domain and target domain is sufficient to allow targeting of a Cas9 molecule to the target nucleic acid.

In certain embodiments, the targeting domain comprises a core domain and an optional secondary domain. In certain of these embodiments, the core domain is located 3′ to the secondary domain, and in certain of these embodiments the core domain is located at or near the 3′ end of the targeting domain. In certain of these embodiments, the core domain consists of or consists essentially of about 8 to about 13 nucleotides at the 3′ end of the targeting domain. In certain embodiments, only the core domain is complementary or partially complementary to the corresponding portion of the target domain, and in certain of these embodiments the core domain is fully complementary to the corresponding portion of the target domain. In certain embodiments, the secondary domain is also complementary or partially complementary to a portion of the target domain. In certain embodiments, the core domain is complementary or partially complementary to a core domain target in the target domain, while the secondary domain is complementary or partially complementary to a secondary domain target in the target domain. In certain embodiments, the core domain and secondary domain have the same degree of complementarity with their respective corresponding portions of the target domain. In certain embodiments, the degree of complementarity between the core domain and its target and the degree of complementarity between the secondary domain and its target may differ. In certain of these embodiments, the core domain may have a higher degree of complementarity for its target than the secondary domain, whereas in other embodiments the secondary domain may have a higher degree of complementarity than the core domain.

In certain embodiments, the targeting domain and/or the core domain within the targeting domain is 3 to 100, 5 to 100, 10 to 100, or 20 to 100 nucleotides in length, and in certain of these embodiments the targeting domain or core domain is 3 to 15, 3 to 20, 5 to 20, 10 to 20, 15 to 20, 5 to 50, 10 to 50, or 20 to 50 nucleotides in length. In certain embodiments, the targeting domain and/or the core domain within the targeting domain is 6, 7, 8, 9, 10, 11, 12, 13, 14, 15, 16, 17, 18, 19, 20, 21, 22, 23, 24, 25, or 26 nucleotides in length. In certain embodiments, the targeting domain and/or the core domain within the targeting domain is 6+/−2, 7+/−2, 8+/−2, 9+/−2, 10+/−2, 10+/−4, 10+/−5, 11+/−2, 12+/−2, 13+/−2, 14+/−2, 15+/−2, or 16+−2, 20+/−5, 30+/−5, 40+/−5, 50+/−5, 60+/−5, 70+/−5, 80+/−5, 90+/−5, or 100+/−5 nucleotides in length.

In certain embodiments wherein the targeting domain includes a core domain, the core domain is 3 to 20 nucleotides in length, and in certain of these embodiments the core domain 5 to 15 or 8 to 13 nucleotides in length. In certain embodiments wherein the targeting domain includes a secondary domain, the secondary domain is 0, 1, 2, 3, 4, 5, 6, 7, 8, 9, 10, 11, 12, 13, 14 or 15 nucleotides in length. In certain embodiments wherein the targeting domain comprises a core domain that is 8 to 13 nucleotides in length, the targeting domain is 26, 25, 24, 23, 22, 21, 20, 19, 18, 17, or 16 nucleotides in length, and the secondary domain is 13 to 18, 12 to 17, 11 to 16, 10 to 15, 9 to 14, 8 to 13, 7 to 12, 6 to 11, 5 to 10, 4 to 9, or 3 to 8 nucleotides in length, respectively.

In certain embodiments, the targeting domain is fully complementary to the target domain. Likewise, where the targeting domain comprises a core domain and/or a secondary domain, in certain embodiments one or both of the core domain and the secondary domain are fully complementary to the corresponding portions of the target domain. In certain embodiments, the targeting domain is partially complementary to the target domain, and in certain of these embodiments where the targeting domain comprises a core domain and/or a secondary domain, one or both of the core domain and the secondary domain are partially complementary to the corresponding portions of the target domain. In certain of these embodiments, the nucleic acid sequence of the targeting domain, or the core domain or targeting domain within the targeting domain, is at least about 80%, about 85%, about 90%, or about 95% complementary to the target domain or to the corresponding portion of the target domain. In certain embodiments, the targeting domain and/or the core or secondary domains within the targeting domain include one or more nucleotides that are not complementary with the target domain or a portion thereof, and in certain of these embodiments the targeting domain and/or the core or secondary domains within the targeting domain include 1, 2, 3, 4, 5, 6, 7, or 8 nucleotides that are not complementary with the target domain. In certain embodiments, the core domain includes 1, 2, 3, 4, or 5 nucleotides that are not complementary with the corresponding portion of the target domain. In certain embodiments wherein the targeting domain includes one or more nucleotides that are not complementary with the target domain, one or more of said non-complementary nucleotides are located within five nucleotides of the 5′ or 3′ end of the targeting domain. In certain of these embodiments, the targeting domain includes 1, 2, 3, 4, or 5 nucleotides within five nucleotides of its 5′ end, 3′ end, or both its 5′ and 3′ ends that are not complementary to the target domain. In certain embodiments wherein the targeting domain includes two or more nucleotides that are not complementary to the target domain, two or more of said non-complementary nucleotides are adjacent to one another, and in certain of these embodiments the two or more consecutive non-complementary nucleotides are located within five nucleotides of the 5′ or 3′ end of the targeting domain. In certain embodiments, the two or more consecutive non-complementary nucleotides are both located more than five nucleotides from the 5′ and 3′ ends of the targeting domain.

In certain embodiments, the targeting domain, core domain, and/or secondary domain do not comprise any modifications. In certain embodiments, the targeting domain, core domain, and/or secondary domain, or one or more nucleotides therein, have a modification, including but not limited to the modifications set forth below. In certain embodiments, one or more nucleotides of the targeting domain, core domain, and/or secondary domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the targeting domain, core domain, and/or secondary domain render the targeting domain and/or the gRNA comprising the targeting domain less susceptible to degradation or more bio-compatible, e.g., less immunogenic. In certain embodiments, the targeting domain and/or the core or secondary domains include 1, 2, 3, 4, 5, 6, 7, or 8 or more modifications, and in certain of these embodiments the targeting domain and/or core or secondary domains include 1, 2, 3, or 4 modifications within five nucleotides of their respective 5′ ends and/or 1, 2, 3, or 4 modifications within five nucleotides of their respective 3′ ends. In certain embodiments, the targeting domain and/or the core or secondary domains comprise modifications at two or more consecutive nucleotides.

In certain embodiments wherein the targeting domain includes core and secondary domains, the core and secondary domains contain the same number of modifications. In certain of these embodiments, both domains are free of modifications. In other embodiments, the core domain includes more modifications than the secondary domain, or vice versa.

In certain embodiments, modifications to one or more nucleotides in the targeting domain, including in the core or secondary domains, are selected to not interfere with targeting efficacy, which can be evaluated by testing a candidate modification using a system as set forth below. gRNAs having a candidate targeting domain having a selected length, sequence, degree of complementarity, or degree of modification can be evaluated using a system as set forth below. The candidate targeting domain can be placed, either alone or with one or more other candidate changes in a gRNA molecule/Cas9 molecule system known to be functional with a selected target, and evaluated.

In certain embodiments, all of the modified nucleotides are complementary to and capable of hybridizing to corresponding nucleotides present in the target domain. In certain embodiments, 1, 2, 3, 4, 5, 6, 7 or 8 or more modified nucleotides are not complementary to or capable of hybridizing to corresponding nucleotides present in the target domain.

5.2 First and Second Complementarity Domains

The first and second complementarity (sometimes referred to alternatively as the crRNA-derived hairpin sequence and tracrRNA-derived hairpin sequences, respectively) domains are fully or partially complementary to one another. In certain embodiments, the degree of complementarity is sufficient for the two domains to form a duplexed region under at least some physiological conditions. In certain embodiments, the degree of complementarity between the first and second complementarity domains, together with other properties of the gRNA, is sufficient to allow targeting of a Cas9 molecule to a target nucleic acid. Examples of first and second complementary domains are set forth in FIGS. 1A-1G.

In certain embodiments (see, e.g., FIGS. 1A-1B) the first and/or second complementarity domain includes one or more nucleotides that lack complementarity with the corresponding complementarity domain. In certain embodiments, the first and/or second complementarity domain includes 1, 2, 3, 4, 5, or 6 nucleotides that do not complement with the corresponding complementarity domain. For example, the second complementarity domain may contain 1, 2, 3, 4, 5, or 6 nucleotides that do not pair with corresponding nucleotides in the first complementarity domain. In certain embodiments, the nucleotides on the first or second complementarity domain that do not complement with the corresponding complementarity domain loop out from the duplex formed between the first and second complementarity domains. In certain of these embodiments, the unpaired loop-out is located on the second complementarity domain, and in certain of these embodiments the unpaired region begins 1, 2, 3, 4, 5, or 6 nucleotides from the 5′ end of the second complementarity domain.

In certain embodiments, the first complementarity domain is 5 to 30, 5 to 25, 7 to 25, 5 to 24, 5 to 23, 7 to 22, 5 to 22, 5 to 21, 5 to 20, 7 to 18, 7 to 15, 9 to 16, or 10 to 14 nucleotides in length, and in certain of these embodiments the first complementarity domain is 5, 6, 7, 8, 9, 10, 11, 12, 13, 14, 15, 16, 17, 18, 19, 20, 21, 22, 23, 24, or 25 nucleotides in length. In certain embodiments, the second complementarity domain is 5 to 27, 7 to 27, 7 to 25, 5 to 24, 5 to 23, 5 to 22, 5 to 21, 7 to 20, 5 to 20, 7 to 18, 7 to 17, 9 to 16, or 10 to 14 nucleotides in length, and in certain of these embodiments the second complementarity domain is 5, 6, 7, 8, 9, 10, 11, 12, 13, 14, 15, 16, 17, 18, 19, 20, 21, 22, 23, 24, 25, or 26 nucleotides in length. In certain embodiments, the first and second complementarity domains are each independently 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length. In certain embodiments, the second complementarity domain is longer than the first complementarity domain, e.g., 2, 3, 4, 5, or 6 nucleotides longer.

In certain embodiments, the first and/or second complementarity domains each independently comprise three subdomains, which, in the 5′ to 3′ direction are: a 5′ subdomain, a central subdomain, and a 3′ subdomain. In certain embodiments, the 5′ subdomain and 3′ subdomain of the first complementarity domain are fully or partially complementary to the 3′ subdomain and 5′ subdomain, respectively, of the second complementarity domain.

In certain embodiments, the 5′ subdomain of the first complementarity domain is 4 to 9 nucleotides in length, and in certain of these embodiments the 5′ domain is 4, 5, 6, 7, 8, or 9 nucleotides in length. In certain embodiments, the 5′ subdomain of the second complementarity domain is 3 to 25, 4 to 22, 4 to 18, or 4 to 10 nucleotides in length, and in certain of these embodiments the 5′ domain is 3, 4, 5, 6, 7, 8, 9, 10, 11, 12, 13, 14, 15, 16, 17, 18, 19, 20, 21, 22, 23, 24, or 25 nucleotides in length. In certain embodiments, the central subdomain of the first complementarity domain is 1, 2, or 3 nucleotides in length. In certain embodiments, the central subdomain of the second complementarity domain is 1, 2, 3, 4, or 5 nucleotides in length. In certain embodiments, the 3′ subdomain of the first complementarity domain is 3 to 25, 4 to 22, 4 to 18, or 4 to 10 nucleotides in length, and in certain of these embodiments the 3′ subdomain is 3, 4, 5, 6, 7, 8, 9, 10, 11, 12, 13, 14, 15, 16, 17, 18, 19, 20, 21, 22, 23, 24, or 25 nucleotides in length. In certain embodiments, the 3′ subdomain of the second complementarity domain is 4 to 9, e.g., 4, 5, 6, 7, 8 or 9 nucleotides in length.

The first and/or second complementarity domains can share homology with, or be derived from, naturally occurring or reference first and/or second complementarity domain. In certain of these embodiments, the first and/or second complementarity domains have at least about 50%, about 60%, about 70%, about 80%, about 85%, about 90%, or about 95% homology with, or differ by no more than 1, 2, 3, 4, 5, or 6 nucleotides from, the naturally occurring or reference first and/or second complementarity domain. In certain of these embodiments, the first and/or second complementarity domains may have at least about 50%, about 60%, about 70%, about 80%, about 85%, about 90%, or about 95% homology with homology with a first and/or second complementarity domain from S. pyogenes or S. aureus.

In certain embodiments, the first and/or second complementarity domains do not comprise any modifications. In other embodiments, the first and/or second complementarity domains or one or more nucleotides therein have a modification, including but not limited to a modification set forth below. In certain embodiments, one or more nucleotides of the first and/or second complementarity domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the first and/or second complementarity domain render the first and/or second complementarity domain and/or the gRNA comprising the first and/or second complementarity less susceptible to degradation or more bio-compatible, e.g., less immunogenic. In certain embodiments, the first and/or second complementarity domains each independently include 1, 2, 3, 4, 5, 6, 7, or 8 or more modifications, and in certain of these embodiments the first and/or second complementarity domains each independently include 1, 2, 3, or 4 modifications within five nucleotides of their respective 5′ ends, 3′ ends, or both their 5′ and 3′ ends. In certain embodiments, the first and/or second complementarity domains each independently contain no modifications within five nucleotides of their respective 5′ ends, 3′ ends, or both their 5′ and 3′ ends. In certain embodiments, one or both of the first and second complementarity domains comprise modifications at two or more consecutive nucleotides.

In certain embodiments, modifications to one or more nucleotides in the first and/or second complementarity domains are selected to not interfere with targeting efficacy, which can be evaluated by testing a candidate modification in a system as set forth below. gRNAs having a candidate first or second complementarity domain having a selected length, sequence, degree of complementarity, or degree of modification can be evaluated in a system as set forth below. The candidate complementarity domain can be placed, either alone or with one or more other candidate changes in a gRNA molecule/Cas9 molecule system known to be functional with a selected target, and evaluated.

In certain embodiments, the duplexed region formed by the first and second complementarity domains is, for example, 6, 7, 8, 9, 10, 11, 12, 13, 14, 15, 16, 17, 18, 19, 20, 21, or 22 bp in length, excluding any looped out or unpaired nucleotides.

In certain embodiments, the first and second complementarity domains, when duplexed, comprise 11 paired nucleotides (see, for e.g., gRNA of SEQ ID NO:48). In certain embodiments, the first and second complementarity domains, when duplexed, comprise 15 paired nucleotides (see, e.g., gRNA of SEQ ID NO:50). In certain embodiments, the first and second complementarity domains, when duplexed, comprise 16 paired nucleotides (see, e.g., gRNA of SEQ ID NO:51). In certain embodiments, the first and second complementarity domains, when duplexed, comprise 21 paired nucleotides (see, e.g., gRNA of SEQ ID NO:29).

In certain embodiments, one or more nucleotides are exchanged between the first and second complementarity domains to remove poly-U tracts. For example, nucleotides 23 and 48 or nucleotides 26 and 45 of the gRNA of SEQ ID NO:48 may be exchanged to generate the gRNA of SEQ ID NOs:49 or 31, respectively. Similarly, nucleotides 23 and 39 of the gRNA of SEQ ID NO:29 may be exchanged with nucleotides 50 and 68 to generate the gRNA of SEQ ID NO:30.

5.3 Linking Domain

The linking domain is disposed between and serves to link the first and second complementarity domains in a unimolecular or chimeric gRNA. FIGS. 1B-1E provide examples of linking domains. In certain embodiments, part of the linking domain is from a crRNA-derived region, and another part is from a tracrRNA-derived region.

In certain embodiments, the linking domain links the first and second complementarity domains covalently. In certain of these embodiments, the linking domain consists of or comprises a covalent bond. In other embodiments, the linking domain links the first and second complementarity domains non-covalently. In certain embodiments, the linking domain is ten or fewer nucleotides in length, e.g., 1, 2, 3, 4, 5, 6, 7, 8, 9, or 10 nucleotides. In other embodiments, the linking domain is greater than 10 nucleotides in length, e.g., 11, 12, 13, 14, 15, 16, 17, 18, 19, 20, 21, 22, 23, 24, or 25 or more nucleotides. In certain embodiments, the linking domain is 2 to 50, 2 to 40, 2 to 30, 2 to 20, 2 to 10, 2 to 5, 10 to 100, 10 to 90, 10 to 80, 10 to 70, 10 to 60, 10 to 50, 10 to 40, 10 to 30, 10 to 20, 10 to 15, 20 to 100, 20 to 90, 20 to 80, 20 to 70, 20 to 60, 20 to 50, 20 to 40, 20 to 30, or 20 to 25 nucleotides in length. In certain embodiments, the linking domain is 10+/−5, 20+/−5, 20+/−10, 30+/−5, 30+/−10, 40+/−5, 40+/−10, 50+/−5, 50+/−10, 60+/−5, 60+/−10, 70+/−5, 70+/−10, 80+/−5, 80+/−10, 90+/−5, 90+/−10, 100+/−5, or 100+/−10 nucleotides in length.

In certain embodiments, the linking domain shares homology with, or is derived from, a naturally occurring sequence, e.g., the sequence of a tracrRNA that is 5′ to the second complementarity domain. In certain embodiments, the linking domain has at least about 50%, about 60%, about 70%, about 80%, about 90%, or about 95% homology with or differs by no more than 1, 2, 3, 4, 5, or 6 nucleotides from a linking domain disclosed herein, e.g., the linking domains of FIGS. 1B-1E.

In certain embodiments, the linking domain does not comprise any modifications. In other embodiments, the linking domain or one or more nucleotides therein have a modification, including but not limited to the modifications set forth below. In certain embodiments, one or more nucleotides of the linking domain may comprise a 2′ modification (e.g., a modification at the 2′ position on ribose), e.g., a 2-acetylation, e.g., a 2′ methylation. In certain embodiments, the backbone of the linking domain can be modified with a phosphorothioate. In certain embodiments, modifications to one or more nucleotides of the linking domain render the linking domain and/or the gRNA comprising the linking domain less susceptible to degradation or more bio-compatible, e.g., less immunogenic. In certain embodiments, the linking domain includes 1, 2, 3, 4, 5, 6, 7, or 8 or more modifications, and in certain of these embodiments the linking domain includes 1, 2, 3, or 4 modifications within five nucleotides of its 5′ and/or 3′ end. In certain embodiments, the linking domain comprises modifications at two or more consecutive nucleotides.

In certain embodiments, modifications to one or more nucleotides in the linking domain are selected to not interfere with targeting efficacy, which can be evaluated by testing a candidate modification in a system as set forth below. gRNAs having a candidate linking domain having a selected length, sequence, degree of complementarity, or degree of modification can be evaluated in a system as set forth below. The candidate linking domain can be placed, either alone or with one or more other candidate changes in a gRNA molecule/Cas9 molecule system known to be functional with a selected target, and evaluated.

In certain embodiments, the linking domain comprises a duplexed region, typically adjacent to or within 1, 2, or 3 nucleotides of the 3′ end of the first complementarity domain and/or the 5′ end of the second complementarity domain. In certain of these embodiments, the duplexed region of the linking region is 10+/−5, 15+/−5, 20+/−5, 20+/−10, or 30+/−5 bp in length. In certain embodiments, the duplexed region of the linking domain is 1, 2, 3, 4, 5, 6, 7, 8, 9, 10, 11, 12, 13, 14, or 15 bp in length. In certain embodiments, the sequences forming the duplexed region of the linking domain are fully complementarity. In other embodiments, one or both of the sequences forming the duplexed region contain one or more nucleotides (e.g., 1, 2, 3, 4, 5, 6, 7, or 8 nucleotides) that are not complementary with the other duplex sequence.

5.4 5′ Extension Domain

In certain embodiments, a modular gRNA as disclosed herein comprises a 5′ extension domain, i.e., one or more additional nucleotides 5′ to the second complementarity domain (see, e.g., FIG. 1A). In certain embodiments, the 5′ extension domain is 2 to 10 or more, 2 to 9, 2 to 8, 2 to 7, 2 to 6, 2 to 5, or 2 to 4 nucleotides in length, and in certain of these embodiments the 5′ extension domain is 2, 3, 4, 5, 6, 7, 8, 9, or 10 or more nucleotides in length.

In certain embodiments, the 5′ extension domain nucleotides do not comprise modifications, e.g., modifications of the type provided below. However, in certain embodiments, the 5′ extension domain comprises one or more modifications, e.g., modifications that it renders it less susceptible to degradation or more bio-compatible, e.g., less immunogenic. By way of example, the backbone of the 5′ extension domain can be modified with a phosphorothioate, or other modification(s) as set forth below. In certain embodiments, a nucleotide of the 5′ extension domain can comprise a 2′ modification (e.g., a modification at the 2′ position on ribose), e.g., a 2-acetylation, e.g., a 2′ methylation, or other modification(s) as set forth below.

In certain embodiments, the 5′ extension domain can comprise as many as 1, 2, 3, 4, 5, 6, 7, or 8 modifications. In certain embodiments, the 5′ extension domain comprises as many as 1, 2, 3, or 4 modifications within 5 nucleotides of its 5′ end, e.g., in a modular gRNA molecule. In certain embodiments, the 5′ extension domain comprises as many as 1, 2, 3, or 4 modifications within 5 nucleotides of its 3′ end, e.g., in a modular gRNA molecule.

In certain embodiments, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain. In certain embodiments, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In certain embodiments, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected so as to not interfere with gRNA molecule efficacy, which can be evaluated by testing a candidate modification in a system as set forth below. gRNAs having a candidate 5′ extension domain having a selected length, sequence, degree of complementarity, or degree of modification, can be evaluated in a system as set forth below. The candidate 5′ extension domain can be placed, either alone, or with one or more other candidate changes in a gRNA molecule/Cas9 molecule system known to be functional with a selected target and evaluated.

In certain embodiments, the 5′ extension domain has at least about 60%, about 70%, about 80%, about 85%, about 90%, or about 95% homology with, or differs by no more than 1, 2, 3, 4, 5, or 6 nucleotides from, a reference 5′ extension domain, e.g., a naturally occurring, e.g., an S. pyogenes, S. aureus, or S. thermophilus, 5′ extension domain, or a 5′ extension domain described herein, e.g., from FIGS. 1A-1G.

5.5 Proximal Domain

FIGS. 1A-1G provide examples of proximal domains.

In certain embodiments, the proximal domain is 5 to 20 or more nucleotides in length, e.g., 5, 6, 7, 8, 9, 10, 11, 12, 13, 14, 15, 16, 17, 18, 19, 20, 21, 22, 23, 24, 25, or 26 nucleotides in length. In certain of these embodiments, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length. In certain embodiments, the proximal domain is 5 to 20, 7, to 18, 9 to 16, or 10 to 14 nucleotides in length.

In certain embodiments, the proximal domain can share homology with or be derived from a naturally occurring proximal domain. In certain of these embodiments, the proximal domain has at least about 50%, about 60%, about 70%, about 80%, about 85%, about 90%, or about 95% homology with or differs by no more than 1, 2, 3, 4, 5, or 6 nucleotides from a proximal domain disclosed herein, e.g., an S. pyogenes, S. aureus, or S. thermophilus proximal domain, including those set forth in FIGS. 1A-1G.

In certain embodiments, the proximal domain does not comprise any modifications. In other embodiments, the proximal domain or one or more nucleotides therein have a modification, including but not limited to the modifications set forth in herein. In certain embodiments, one or more nucleotides of the proximal domain may comprise a 2′ modification (e.g., a modification at the 2′ position on ribose), e.g., a 2-acetylation, e.g., a 2′ methylation. In certain embodiments, the backbone of the proximal domain can be modified with a phosphorothioate. In certain embodiments, modifications to one or more nucleotides of the proximal domain render the proximal domain and/or the gRNA comprising the proximal domain less susceptible to degradation or more bio-compatible, e.g., less immunogenic. In certain embodiments, the proximal domain includes 1, 2, 3, 4, 5, 6, 7, or 8 or more modifications, and in certain of these embodiments the proximal domain includes 1, 2, 3, or 4 modifications within five nucleotides of its 5′ and/or 3′ end. In certain embodiments, the proximal domain comprises modifications at two or more consecutive nucleotides.

In certain embodiments, modifications to one or more nucleotides in the proximal domain are selected to not interfere with targeting efficacy, which can be evaluated by testing a candidate modification in a system as set forth below. gRNAs having a candidate proximal domain having a selected length, sequence, degree of complementarity, or degree of modification can be evaluated in a system as set forth below. The candidate proximal domain can be placed, either alone or with one or more other candidate changes in a gRNA molecule/Cas9 molecule system known to be functional with a selected target, and evaluated.

5.6 Tail Domain

A broad spectrum of tail domains are suitable for use in the gRNA molecules disclosed herein. FIGS. 1A and 1C-1G provide examples of such tail domains.

In certain embodiments, the tail domain is absent. In other embodiments, the tail domain is 1 to 100 or more nucleotides in length, e.g., 1, 2, 3, 4, 5, 6, 7, 8, 9, 10, 20, 30, 40, 50, 60, 70, 80, 90, or 100 nucleotides in length. In certain embodiments, the tail domain is 1 to 5, 1 to 10, 1 to 15, 1 to 20, 1 to 50, 10 to 100, 20 to 100, 10 to 90, 20 to 90, 10 to 80, 20 to 80, 10 to 70, 20 to 70, 10 to 60, 20 to 60, 10 to 50, 20 to 50, 10 to 40, 20 to 40, 10 to 30, 20 to 30, 20 to 25, 10 to 20, or 10 to 15 nucleotides in length. In certain embodiments, the tail domain is 5+/−5, 10+/−5, 20+/−10, 20+/−5, 25+/−10, 30+/−10, 30+/−5, 40+/−10, 40+/−5, 50+/−10, 50+/−5, 60+/−10, 60+/−5, 70+/−10, 70+/−5, 80+/−10, 80+/−5, 90+/−10, 90+/−5, 100+/−10, or 100+/−5 nucleotides in length,

In certain embodiments, the tail domain can share homology with or be derived from a naturally occurring tail domain or the 5′ end of a naturally occurring tail domain. In certain of these embodiments, the proximal domain has at least about 50%, about 60%, about 70%, about 80%, about 85%, about 90%, or about 95% homology with or differs by no more than 1, 2, 3, 4, 5, or 6 nucleotides from a naturally occurring tail domain disclosed herein, e.g., an S. pyogenes, S. aureus, or S. thermophilus tail domain, including those set forth in FIGS. 1A and 1C-1G.

In certain embodiments, the tail domain includes sequences that are complementary to each other and which, under at least some physiological conditions, form a duplexed region. In certain of these embodiments, the tail domain comprises a tail duplex domain which can form a tail duplexed region. In certain embodiments, the tail duplexed region is 3, 4, 5, 6, 7, 8, 9, 10, 11, or 12 bp in length. In certain embodiments, the tail domain comprises a single stranded domain 3′ to the tail duplex domain that does not form a duplex. In certain of these embodiments, the single stranded domain is 3 to 10 nucleotides in length, e.g., 3, 4, 5, 6, 7, 8, 9, 10, or 4 to 6 nucleotides in length.

In certain embodiments, the tail domain does not comprise any modifications. In other embodiments, the tail domain or one or more nucleotides therein have a modification, including but not limited to the modifications set forth herein. In certain embodiments, one or more nucleotides of the tail domain may comprise a 2′ modification (e.g., a modification at the 2′ position on ribose), e.g., a 2-acetylation, e.g., a 2′ methylation. In certain embodiments, the backbone of the tail domain can be modified with a phosphorothioate. In certain embodiments, modifications to one or more nucleotides of the tail domain render the tail domain and/or the gRNA comprising the tail domain less susceptible to degradation or more bio-compatible, e.g., less immunogenic. In certain embodiments, the tail domain includes 1, 2, 3, 4, 5, 6, 7, or 8 or more modifications, and in certain of these embodiments the tail domain includes 1, 2, 3, or 4 modifications within five nucleotides of its 5′ and/or 3′ end. In certain embodiments, the tail domain comprises modifications at two or more consecutive nucleotides.

In certain embodiments, modifications to one or more nucleotides in the tail domain are selected to not interfere with targeting efficacy, which can be evaluated by testing a candidate modification as set forth below. gRNAs having a candidate tail domain having a selected length, sequence, degree of complementarity, or degree of modification can be evaluated using a system as set forth below. The candidate tail domain can be placed, either alone or with one or more other candidate changes in a gRNA molecule/Cas9 molecule system known to be functional with a selected target, and evaluated.

In certain embodiments, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. In certain embodiments, the gRNA molecule includes a 3′ polyA tail that is prepared by in vitro transcription from a DNA template. In certain embodiments, the 5′ nucleotide of the targeting domain of the gRNA molecule is a guanine nucleotide, the DNA template comprises a T7 promoter sequence located immediately upstream of the sequence that corresponds to the targeting domain, and the 3′ nucleotide of the T7 promoter sequence is not a guanine nucleotide. In certain embodiments, the 5′ nucleotide of the targeting domain of the gRNA molecule is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

When a U6 promoter is used for in vivo transcription, these nucleotides may be the sequence UUUUUU. When an H1 promoter is used for transcription, these nucleotides may be the sequence UUUU. When alternate pol-III promoters are used, these nucleotides may be various numbers of uracil bases depending on, e.g., the termination signal of the pol-III promoter, or they may include alternate bases.

In certain embodiments, the proximal and tail domain taken together comprise, consist of, or consist essentially of the sequence set forth in SEQ ID NOs:32, 33, 34, 35, 36, or 37.

5.7 Exemplary Unimolecular/Chimeric gRNAs

In certain embodiments, a gRNA as disclosed herein has the structure: 5′ [targeting domain]-[first complementarity domain]-[linking domain]-[second complementarity domain]-[proximal domain]-[tail domain]-3′, wherein:

the targeting domain comprises a core domain and optionally a secondary domain, and is 10 to 50 nucleotides in length;

the first complementarity domain is 5 to 25 nucleotides in length and, in certain embodiments has at least about 50%, about 60%, about 70%, about 80%, about 85%, about 90%, or about 95% homology with a reference first complementarity domain disclosed herein;

the linking domain is 1 to 5 nucleotides in length;

the second complementarity domain is 5 to 27 nucleotides in length and, in certain embodiments has at least about 50%, about 60%, about 70%, about 80%, about 85%, about 90%, or about 95% homology with a reference second complementarity domain disclosed herein; the proximal domain is 5 to 20 nucleotides in length and, in certain embodiments has at least about 50%, about 60%, about 70%, about 80%, about 85%, about 90%, or about 95% homology with a reference proximal domain disclosed herein; and

the tail domain is absent or a nucleotide sequence is 1 to 50 nucleotides in length and, in certain embodiments has at least about 50%, about 60%, about 70%, about 80%, about 85%, about 90%, or about 95% homology with a reference tail domain disclosed herein.

In certain embodiments, a unimolecular gRNA as disclosed herein comprises, preferably from 5′ to 3′:

a targeting domain, e.g., comprising 10-50 nucleotides;

a first complementarity domain, e.g., comprising 15, 16, 17, 18, 19, 20, 21, 22, 23, 24, 25, or 26 nucleotides;

a linking domain;

a second complementarity domain;

a proximal domain; and

a tail domain,

wherein,

(a) the proximal and tail domain, when taken together, comprise at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), and/or (c) has at least about 50%, about 60%, about 70%, about 75%, about 60%, about 70%, about 80%, about 85%, about 90%, about 95%, or about 99% homology with the corresponding sequence of a naturally occurring gRNA, or with a gRNA described herein.

In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain.

In certain embodiments, there are at least 16, 19, 21, 26, 31, 32, 36, 41, 46, 50, 51, or 54 nucleotides 3′ to the last nucleotide of the second complementarity domain that are complementary to the corresponding nucleotides of the first complementarity domain.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary or partially complementary to the target domain or a portion thereof, e.g., the targeting domain is 16, 17, 18, 19, 20, 21, 22, 23, 24, 25, or 26 nucleotides in length. In certain of these embodiments, the targeting domain is complementary to the target domain over the entire length of the targeting domain, the entire length of the target domain, or both.

In certain embodiments, a unimolecular or chimeric gRNA molecule disclosed herein (comprising a targeting domain, a first complementary domain, a linking domain, a second complementary domain, a proximal domain and, optionally, a tail domain) comprises the amino acid sequence set forth in SEQ ID NO:42, wherein the targeting domain is listed as 20 N's (residues 1-20) but may range in length from 16 to 26 nucleotides, and wherein the final six residues (residues 97-102) represent a termination signal for the U6 promoter buy may be absent or fewer in number. In certain embodiments, the unimolecular, or chimeric, gRNA molecule is an S. pyogenes gRNA molecule.

In certain embodiments, a unimolecular or chimeric gRNA molecule disclosed herein (comprising a targeting domain, a first complementary domain, a linking domain, a second complementary domain, a proximal domain and, optionally, a tail domain) comprises the amino acid sequence set forth in SEQ ID NO:38, wherein the targeting domain is listed as 20 Ns (residues 1-20) but may range in length from 16 to 26 nucleotides, and wherein the final six residues (residues 97-102) represent a termination signal for the U6 promoter but may be absent or fewer in number. In certain embodiments, the unimolecular or chimeric gRNA molecule is an S. aureus gRNA molecule.

The sequences and structures of exemplary chimeric gRNAs are also shown in FIGS. 1H-1I.

5.8 Exemplary Modular gRNAs

In certain embodiments, a modular gRNA disclosed herein comprises:

a first strand comprising, preferably from 5′ to 3′;

a targeting domain, e.g., comprising 15, 16, 17, 18, 19, 20, 21, 22, 23, 24, 25, or 26 nucleotides;

a first complementarity domain; and

a second strand, comprising, preferably from 5′ to 3′:

optionally a 5′ extension domain;

a second complementarity domain;

a proximal domain; and

a tail domain,

wherein:

(a) the proximal and tail domain, when taken together, comprise at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), or (c), has at least about 50%, about 60%, about 70%, about 80%, about 85%, about 90%, about 95%, or about 99% homology with the corresponding sequence of a naturally occurring gRNA, or with a gRNA described herein.

In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain.

In certain embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 16, 17, 18, 19, 20, 21, 22, 23, 24, 25, or 26 nucleotides (e.g., 16, 17, 18, 19, 20, 21, 22, 23, 24, 25, or 26 consecutive nucleotides) having complementarity with the target domain, e.g., the targeting domain is 16, 17, 18, 19, 20, 21, 22, 23, 24, 25, or 26 nucleotides in length.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary to the target domain or a portion thereof. In certain of these embodiments, the targeting domain is complementary to the target domain over the entire length of the targeting domain, the entire length of the target domain, or both.

In certain embodiments, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In certain embodiments, the targeting domain has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In certain embodiments, the targeting domain has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

5.9 gRNA Delivery

In certain embodiments of the methods provided herein, the methods comprise delivery of one or more (e.g., two, three, or four) gRNA molecules as described herein. In certain of these embodiments, the gRNA molecules are delivered by intravenous injection, intramuscular injection, subcutaneous injection, or inhalation. In certain embodiments, the gRNA molecules are delivered with a Cas9 molecule in a genome editing system.

### 6. Methods for Designing gRNAs

Methods for selecting, designing, and validating targeting domains for use in the gRNAs described herein are provided. Exemplary targeting domains for incorporation into gRNAs are also provided herein.

Methods for selection and validation of target sequences as well as off-target analyses have been described previously (see, e.g., Mali 2013; Hsu 2013; Fu 2014; Heigwer 2014; Bae 2014; Xiao 2014). For example, a software tool can be used to optimize the choice of potential targeting domains corresponding to a user's target sequence, e.g., to minimize total off-target activity across the genome. Off-target activity may be other than cleavage. For each possible targeting domain choice using S. pyogenes Cas9, the tool can identify all off-target sequences (preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible targeting domain is then ranked according to its total predicted off-target cleavage; the top-ranked targeting domains represent those that are likely to have the greatest on-target cleavage and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate targeting domains and gRNAs comprising those targeting domains can be functionally evaluated using methods known in the art and/or as set forth herein.

HBV genomes have vast variants. The gRNAs were designed to provide maximal coverage of the conserved HBV genome. To optimize the choice of gRNA, eight different types of HBV consensus sequences (according to the database found at hbvdb.ibcp.fr/HBVdb/), e.g., HBV-A, HBV-B, HBV-C, HBV-D, HBV-E, HBV-F, HBV-G and HBV-H were selected as target sequences. The eight different types of HBV consensus sequences (e.g., HBV-A, HBV-B, HBV-C, HBV-D, HBV-E, HBV-F, HBV-G and HBV-H) represent significant genomic conservation between HBV subtypes and strain variants. The Targeting Domains discussed herein can be incorporated into the gRNAs described herein.

As a non-limiting example, guide RNAs (gRNAs) for use with an S. pyogenes Cas9, e.g., Cas9 EQR or VRER variant, or an S. aureus Cas9, e.g., Cas9 KKH variant, can be identified using a DNA sequence searching algorithm. Guide RNA design can be carried out using a custom guide RNA design software based on the public tool cas-offinder (reference: Cas-OFFinder: a fast and versatile algorithm that searches for potential off-target sites of Cas9 RNA-guided endonucleases, Bioinformatics. 2014 Feb. 17. Bae S, Park J, Kim J S. PMID: 24463181). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene can be obtained from the UCSC Genome browser and sequences can be screened for repeat elements using the publically available RepeatMasker program. RepeatMasker searches input DNA sequences for repeated elements and regions of low complexity. The output is a detailed annotation of the repeats present in a given query sequence.

Following identification, gRNAs can be ranked into tiers based on their distance to the target site, their orthogonality and presence of a 5′ G (based on identification of close matches in the human genome containing a relevant PAM). In certain embodiments, for a wild-type S. pyogenes Cas9, the PAM may be a NGG PAM. In certain embodiments, for an S. pyogenes Cas9 EQR variant, the PAM may be a NGAG PAM, A NGCG PAM, a NGGG PAM, a NGTG PAM, a NGAA PAM, a NGAT PAM or a NGAC PAM. In certain embodiments, for an S. pyogenes Cas9 VRER variant, the PAM may be a NGCG PAM, A NGCA PAM, a NGCT PAM, or a NGCC PAM. In certain embodiments, for a wild-type S. aureus Cas9, the PAM may be a NNNRRT PAM or a NNNRRV PAM. In certain embodiments, for an S. aureus Cas9 KKH variant, the PAM may be a NNNRRT PAM or a NNNRRV PAM. Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

In the case of knock out approach, gRNAs were identified for both single-gRNA nuclease cleavage and for a dual-gRNA paired “nickase” strategy. Criteria for selecting gRNAs and the determination for which gRNAs can be used for the dual-gRNA paired “nickase” strategy is based on two considerations:

1. gRNA pairs should be oriented on the DNA such that PAMs are facing out and cutting with the D10A Cas9 nickase will result in 5′ overhangs.

2. An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the site of one gRNA.

The targeting domains discussed herein can be incorporated into the gRNAs described herein.

gRNAs designed to be used with an S. pyogenes Cas9 can be identified and ranked into 4 tiers. The targeting domain for tier 1 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) a high level of orthogonality and (3) the presence of 5′G and (4) wherein the PAM is NGG. The targeting domain for tier 2 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) a high level of orthogonality and (3) wherein the PAM is NGG. The targeting domain for tier 3 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) the presence of 5′G and (3) wherein the PAM is NGG. The targeting domain for tier 4 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) wherein the PAM is NGG. Exemplary gRNAs (referred to by SEQ ID NO) designed to be used with an S. pyogenes Cas9 identified using this tiered-based approach with respect to knocking down the expression of one or more of HBV genes (e.g., PreC, C, X, PreS1, PreS2, S, P or SP genes) of the HBV-A, HBV-B, HBV-C, HBV-D, HBV-E, HBV-F, HBV-G, or HBV-H consensus sequences are provided in Table 1. In certain embodiments, the targeting domain hybridizes to the target domain through complementary base pairing. Any of the targeting domains set forth in the SEQ ID NOs of Table 1 can be used with an S. pyogenes eiCas9 molecule to reduce, decrease or repress the expression of one or more of the PreC, C, X PreS1, PreS2, S, P or SP genes.

TABLE 1
SEQ ID NOs of Exemplary gRNAs (S. pyogenes Cas9)

| Tier | HBV-A | HBV-B | HBV-C | HBV-D | HBV-E | HBV-F | HBV-G | HBV-H |
|---|---|---|---|---|---|---|---|---|
| 1 | 15389- | 31598- | 47978- | 62798- | 79221- | 94449- | 110120- | 125842- |
| | 15440 | 31662 | 48016 | 62855 | 79271 | 94494 | 110168 | 125890 |
| 2 | 15441- | 31663- | 48017- | 62856- | 79272- | 94495- | 110169- | 125891- |
| | 15631 | 31832 | 48127 | 62993 | 79402 | 94624 | 110314 | 125996 |
| 3 | 15632- | 31833- | 48128- | 62994- | 79403- | 94625- | 110315- | 125997- |
| | 15809 | 32002 | 48288 | 63154 | 79563 | 94790 | 110480 | 126154 |
| 4 | 15810- | 32003- | 48289- | 63155- | 79564- | 94791- | 110481- | 126155- |
| | 16329 | 32518 | 48841 | 63714 | 80079 | 95356 | 111022 | 126712 |

gRNAs designed to be used with an S. pyogenes Cas9 EQR variant can be identified and ranked into 5 tiers. The targeting domain for tier 1 gRNA molecules can be selected based on (1) distance to a target site, e.g., within the HBV genome (e.g., targeting the entire HBV-A sequence), (2) a high level of orthogonality and (3) the presence of 5′G and (4) wherein the PAM is NGAG. The targeting domain for tier 2 gRNA molecules can be selected based on (1) distance to a target site, e.g., within the HBV genome (e.g., targeting the entire HBV-A sequence), (2) a high level of orthogonality and (3) wherein the PAM is NGAG. The targeting domain for tier 3 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) the presence of 5′G and (3) wherein the PAM is NGAG. The targeting domain for tier 4 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) wherein the PAM is NGAG. The targeting domain for tier 5 gRNA molecules can be selected based (1) distance to a target site, e.g., within the HBV genome (e.g., targeting the entire HBV-A, HBV-B, HBV-C, HBV-D, HBV-E, HBV-F, HBV-G, or HBV-H sequence) and (2) wherein the PAM is NGCG, NGGG, NGTG, NGAA, NGAT or NGAC. Exemplary gRNAs (referred to by SEQ ID NO) designed to be used with an S. pyogenes Cas9 EQR variant identified using this tiered-based approach with respect to knocking out and knocking down the expression of one or more of HBV genes (e.g., PreC, C, X, PreS1, PreS2, S, P or SP genes) of the HBV-A, HBV-B, HBV-C, HBV-D, HBV-E, HBV-F, HBV-G, or HBV-H consensus sequences are provided in Table 2. In certain embodiments, the targeting domain hybridizes to the target domain through complementary base pairing. Any of the targeting domains set forth in the SEQ ID NOs of Table 2 can be used with an S. pyogenes Cas9 EQR molecule to reduce, decrease or repress the expression of one or more of the PreC, C, X, PreS1, PreS2, S, P or SP genes. Any of the targeting domains set forth in the SEQ ID NOs of Table 2 can be used with an S. pyogenes Cas9 EQR molecule to reduce, decrease or repress the expression of one or more of the PreC, C, X, PreS1, PreS2, S, P or SP genes.

TABLE 2
SEQ ID NOs of Exemplary gRNAs (S. pyogenes Cas9 EQR variant)

| Tier | HBV-A | HBV-B | HBV-C | HBV-D | HBV-E | HBV-F | HBV-G | HBV-H |
|---|---|---|---|---|---|---|---|---|
| 1 | 215- | 2225- | 4169- | 5977- | 7953- | 9830- | 11678- | 13563- |
| | 235 | 2254 | 4181 | 6001 | 7974 | 9852 | 11700 | 13580 |
| 2 | 236- | 2255- | 4182- | 6002- | 7975- | 9853- | 11701- | 13581- |
| | 275 | 2297 | 4206 | 6043 | 8008 | 9890 | 11739 | 13615 |
| 3 | 276- | 2298- | 4207- | 6044- | 8009- | 9891- | 11740- | 13616- |
| | 326 | 2339 | 4242 | 6087 | 8056 | 9941 | 11783 | 13670 |
| 4 | 327- | 2340- | 4243- | 6088- | 8057- | 9942- | 11784- | 13671- |
| | 456 | 2456 | 4364 | 6206 | 8174 | 10056 | 11901 | 13784 |
| 5 | 457- | 2457- | 4365- | 6207- | 8175- | 10057- | 11902- | 13785- |
| | 1565 | 3535 | 5381 | 7325 | 9213 | 11082 | 12954 | 14791 |

gRNAs designed to be used with an S. pyogenes Cas9 VRER variant can be identified and ranked into 5 tiers. The targeting domain for tier 1 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) a high level of orthogonality and (3) the presence of 5′G and (4) wherein the PAM is NGCG. The targeting domain for tier 2 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) (2) a high level of orthogonality and (3) wherein the PAM is NGCG. The targeting domain for tier 3 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) the presence of 5′G and (3) wherein the PAM is NGCG. The targeting domain for tier 4 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) wherein the PAM is NGCG. The targeting domain for tier 5 gRNA molecules can be selected based (1) distance to a target site, e.g., within the HBV genome (e.g., targeting the entire HBV-A, HBV-B, HBV-C, HBV-D, HBV-E, HBV-F, HBV-G, or HBV-H sequence) and (2) wherein the PAM is NGCA, NGCT or NGCC. Exemplary gRNAs (referred to by SEQ ID NO) designed to be used with an S. pyogenes Cas9 VRER variant identified using this tiered-based approach with respect to knocking out and knocking down the expression of one or more of HBV genes (e.g., PreC, C, X PreS1, PreS2, S, P or SP genes) of the HBV-A, HBV-B, HBV-C, HBV-D, HBV-E, HBV-F, HBV-G, or HBV-H consensus sequences are provided in Table 3. In certain embodiments, the targeting domain hybridizes to the target domain through complementary base pairing. Any of the targeting domains set forth in the SEQ ID NOs of Table 3 can be used with an S. pyogenes Cas9 VRER variant to reduce, decrease or repress the expression of one or more of the PreC, C, X, PreS1, PreS2, S, P or SP genes.

TABLE 3
SEQ ID NOs of Exemplary gRNAs (S. pyogenes Cas9 VRER variant)

| Tier | HBV-A | HBV-B | HBV-C | HBV-D | HBV-E | HBV-F | HBV-G | HBV-H |
|---|---|---|---|---|---|---|---|---|
| 1 | 1566- | 3536- | 5382- | 7326- | 9214- | 11083- | 12955- | 14792- |
| | 1587 | 3556 | 5402 | 7346 | 9239 | 11102 | 12978 | 14809 |
| 2 | 1588- | 3557- | 5403- | 7347- | 9240- | 11103- | 12979- | 14810- |
| | 1624 | 3594 | 5433 | 7379 | 9277 | 11131 | 13012 | 14843 |
| 3 | 1625- | 3595- | 5434- | 7380- | 9278- | 11132- | 13013- | 14844- |
| | 1637 | 3603 | 5445 | 7388 | 9287 | 11136 | 13022 | 14855 |
| 4 | 1638- | 3604- | 5446- | 7389- | 9288- | 11137- | 13023- | 14856- |
| | 1661 | 3617 | 5463 | 7407 | 9315 | 11154 | 13048 | 14875 |
| 5 | 1662- | 3618- | 5464- | 7408- | 9316- | 11155- | 13049- | 14876- |
| | 2224 | 4168 | 5976 | 7952 | 9829 | 11677 | 13562 | 15388 |

gRNAs designed to be used with an S. aureus Cas9 can be identified and ranked into 4 tiers. The targeting domain for tier 1 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H consensus sequence), (2) a high level of orthogonality and (3) the presence of 5′G and (4) PAM is NNNRRT. The targeting domain for tier 2 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) a high level of orthogonality and (3) PAM is NNNRRT. The targeting domain for tier 3 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) PAM is NNNRRT. The targeting domain for tier 4 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) PAM is NNNRRV. Exemplary gRNAs (referred to by SEQ ID NO) designed to be used with an S. aureus Cas9 identified using this tiered-based approach with respect to knocking down the expression of one or more of HBV genes (e.g., PreC, C, X, PreS1, PreS2, S, P or SP genes) of the HBV-A, HBV-B, HBV-C, HBV-D, HBV-E, HBV-F, HBV-G, or HBV-H consensus sequences are provided in Table 4. In certain embodiments, the targeting domain hybridizes to the target domain through complementary base pairing. Any of the targeting domains set forth in the SEQ ID NOs of Table 4 can be used with an S. aureus Cas9 molecule to reduce, decrease or repress the expression of one or more of the PreC, C, X, PreS1, PreS2, S, P or SP genes.

TABLE 4
SEQ ID NOs of Exemplary gRNAs (S. aureus Cas9)

| Tier | HBV-A | HBV-B | HBV-C | HBV-D | HBV-E | HBV-F | HBV-G | HBV-H |
|---|---|---|---|---|---|---|---|---|
| 1 | 16330- | 32519- | 48842- | 63715- | 80080- | 95357- | 111023- | 126713- |
| | 16465 | 32670 | 48938 | 63837 | 80196 | 95463 | 111153 | 126813 |
| 2 | 16466- | 32671- | 48939- | 63838- | 80197- | 95464- | 111154- | 126814- |
| | 16860 | 33102 | 49203 | 64239 | 80526 | 95766 | 111512 | 127115 |
| 3 | 16861- | 33103- | 49204- | 64240- | 80527- | 95767- | 111513- | 127116- |
| | 17036 | 33288 | 49380 | 64417 | 80733 | 95947 | 111683 | 127335 |
| 4 | 17037- | 33289- | 49381- | 64418- | 80734- | 95948- | 111684- | 127336- |
| | 19822 | 35976 | 51921 | 67224 | 83218 | 98663 | 114350 | 129862 |

gRNAs designed to be used with an S. aureus Cas9 KKH variant can be identified and ranked into 5 tiers. The targeting domain for tier 1 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H consensus sequence), (2) a high level of orthogonality and (3) the presence of 5′G and (4) PAM is NNNRRT. The targeting domain for tier 2 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) a high level of orthogonality and (3) PAM is NNNRRT. The targeting domain for tier 3 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence), (2) the presence of 5′G and (3) PAM is NNNRRT. The targeting domain for tier 4 gRNA molecules can be selected based on (1) distance to a target site, e.g., within the HBV genome (e.g., targeting the entire HBV-A, HBV-B, HBV-C, HBV-D, HBV-E, HBV-F, HBV-G, or HBV-H sequence) and (2) PAM is NNNRRT. The targeting domain for tier 5 gRNA molecules can be selected based (1) distance to a target site, e.g., within the HBV genome (e.g., targeting the entire HBV-A, HBV-B, HBV-C, HBV-D, HBV-E, HBV-F, HBV-G, or HBV-H sequence) and (2) PAM is NNNRRV. Exemplary gRNAs (referred to by SEQ ID NO) designed to be used with an S. aureus Cas9 KKH variant identified using this tiered-based approach with respect to knocking out and knocking down the expression of one or more of HBV genes (e.g., PreC, C, X, PreS1, PreS2, S, P or SP genes) of the HBV-A, HBV-B, HBV-C, HBV-D, HBV-E, HBV-F, HBV-G, or HBV-H consensus sequences are provided in Table 5. In certain embodiments, the targeting domain hybridizes to the target domain through complementary base pairing. Any of the targeting domains set forth in the SEQ ID NOs of Table 5 can be used with an S. aureus Cas9 KKH molecule to reduce, decrease or repress the expression of one or more of the PreC, C, X, PreS1, PreS2, S, P or SP genes.

TABLE 5
SEQ ID NOs of Exemplary gRNAs (S. aureus Cas9 KKH variant)

| Tier | HBV-A | HBV-B | HBV-C | HBV-D | HBV-E | HBV-F | HBV-G | HBV-H |
|---|---|---|---|---|---|---|---|---|
| 1 | 19823- | 35977- | 51922- | 67225- | 83219- | 98664- | 114351- | 129863- |
| | 20028 | 36242 | 52034 | 67439 | 83379 | 98816 | 114522 | 130008 |
| 2 | 20029- | 36243- | 52035- | 67440- | 83380- | 98817- | 114523- | 130009- |
| | 20625 | 36951 | 52350 | 68040 | 83813 | 99265 | 115019 | 130409 |
| 3 | 20626- | 36952- | 52351- | 68041- | 83814- | 99266- | 115020- | 130410- |
| | 20949 | 37327 | 52720 | 68423 | 84212 | 100864 | 115407 | 130793 |
| 4 | 20950- | 37328- | 52721- | 68424- | 84213- | 100865- | 115408- | 130794- |
| | 22289 | 38594 | 53126 | 69719 | 85477 | 100867 | 116642 | 132225 |
| 5 | 22290- | 38595- | 53127- | 69720- | 85478- | 100868- | 116643- | 132226- |
| | 31597 | 47977 | 62797 | 79220 | 94448 | 110119 | 125841 | 141071 |

Any of the targeting domains in the tables described herein can be used with a Cas9 nickase molecule to generate a single strand break.

Any of the targeting domains in the tables described herein can be used with a Cas9 nuclease molecule to generate a double strand break.

When two gRNAs designed for use to target two Cas9 molecules, one Cas9 can be one species, the second Cas9 can be from a different species. Both Cas9 species are used to generate a single or double-strand break, as desired.

One or more of the gRNA molecules described herein, e.g., those comprising the targeting domains described in Tables 1-5 can be used with at least one Cas9 molecule (e.g., an S. pyogenes Cas9 molecule and/or an S. aureus Cas9 molecule) to form a single or a double stranded cleavage. In certain embodiments, dual targeting is used to create two double strand breaks (e.g., by using at least one Cas9 nuclease, e.g., an S. pyogenes Cas9 nuclease and/or an S. aureus Cas9 nuclease) or two nicks (e.g., by using at least one Cas9 nickase, e.g., an S. pyogenes Cas9 nickase and/or an S. aureus Cas9 nickase) on opposite DNA strands with two gRNA molecules. In certain embodiments, a presently disclosed composition or genome editing system comprises two gRNA molecules comprising targeting domains that are complementary to opposite DNA strands, e.g., a gRNA molecule comprising any minus strand targeting domain that can be paired with a gRNA molecule comprising a plus strand targeting domain provided that the two gRNA molecules are oriented on the DNA such that PAMs face outward. In certain embodiments, two gRNA molecules are used to target two Cas9 nucleases (e.g., two S. pyogenes Cas9 nucleases, two S. aureus Cas9 nucleases, or one S. aureus Cas9 nuclease and one S. pyogenes Cas9 nuclease) or two Cas9 nickases (e.g., two S. pyogenes Cas9 nickases, two S. aureus Cas9 nickases, or one S. aureus Cas9 nickase and one Cas9 nickase). One or more of the gRNA molecules described herein, e.g., those comprising the targeting domains described in Tables 1-5 can be used with at least one Cas9 molecule to mediate the alteration of a HBV viral gene selected from the group consisting of PreC, C, X, PreS1, PreS2, S, P and SP genes, described in Section 4.

### 7. Cas9 Molecules

Cas9 molecules of a variety of species can be used in the methods and compositions described herein. While the S. pyogenes and S. aureus Cas9 molecules are the subject of much of the disclosure herein, Cas9 molecules, derived from, or based on the Cas9 proteins of other species listed herein can be used as well. These include, for example, Cas9 molecules from Acidovorax avenae, Actinobacillus pleuropneumonias, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitides, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

7.1 Cas9 Domains

Crystal structures have been determined for two different naturally occurring bacterial Cas9 molecules (Jinek 2014) and for S. pyogenes Cas9 with a guide RNA (e.g., a synthetic fusion of crRNA and tracrRNA) (Nishimasu 2014; Anders 2014).

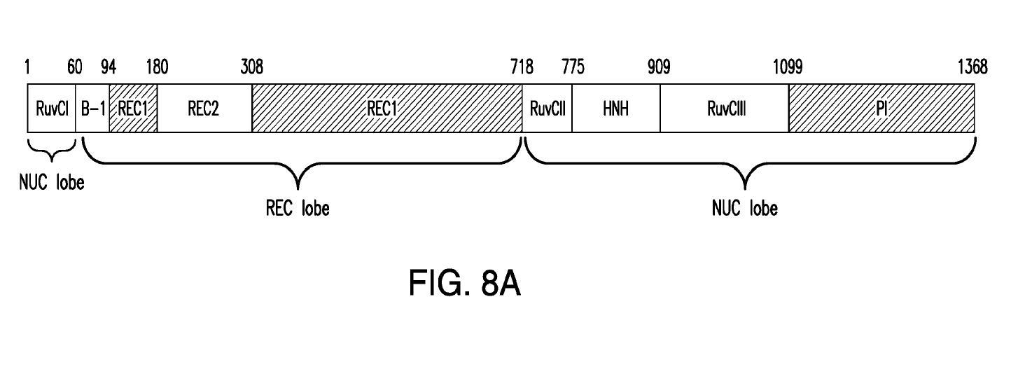

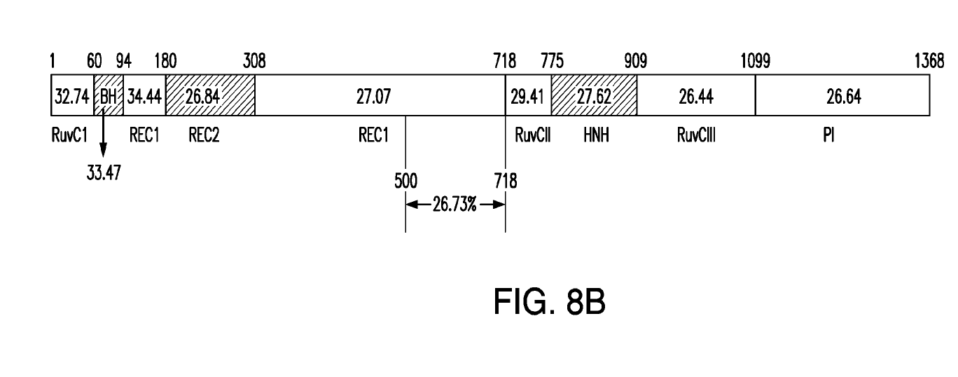

A naturally occurring Cas9 molecule comprises two lobes: a recognition (REC) lobe and a nuclease (NUC) lobe; each of which further comprise domains described herein. FIGS. 8A-8B provide a schematic of the organization of important Cas9 domains in the primary structure. The domain nomenclature and the numbering of the amino acid residues encompassed by each domain used throughout this disclosure is as described previously (Nishimasu 2014). The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long a helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain, the HNH domain, and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

7.1.1 RuvC-Like Domain and HNH-Like Domain

In certain embodiments, a Cas9 molecule or Cas9 polypeptide comprises an HNH-like domain and a RuvC-like domain, and in certain of these embodiments cleavage activity is dependent on the RuvC-like domain and the HNH-like domain. A Cas9 molecule or Cas9 polypeptide can comprise one or more of a RuvC-like domain and an HNH-like domain. In certain embodiments, a Cas9 molecule or Cas9 polypeptide comprises a RuvC-like domain, e.g., a RuvC-like domain described below, and/or an HNH-like domain, e.g., an HNH-like domain described below.

### RuvC-Like Domains

In certain embodiments, a RuvC-like domain cleaves a single strand, e.g., the non-complementary strand of the target nucleic acid molecule. The Cas9 molecule or Cas9 polypeptide can include more than one RuvC-like domain (e.g., one, two, three or more RuvC-like domains). In certain embodiments, a RuvC-like domain is at least 5, 6, 7, 8 amino acids in length but not more than 20, 19, 18, 17, 16 or 15 amino acids in length. In certain embodiments, the Cas9 molecule or Cas9 polypeptide comprises an N-terminal RuvC-like domain of about 10 to 20 amino acids, e.g., about 15 amino acids in length.

7.1.2 N-Terminal RuvC-Like Domains

Some naturally occurring Cas9 molecules comprise more than one RuvC-like domain with cleavage being dependent on the N-terminal RuvC-like domain. Accordingly, a Cas9 molecule or Cas9 polypeptide can comprise an N-terminal RuvC-like domain. Exemplary N-terminal RuvC-like domains are described below.

In certain embodiments, a Cas9 molecule or Cas9 polypeptide comprises an N-terminal RuvC-like domain comprising an amino acid sequence of Formula I:

(SEQ ID NO: 20)
D-X₁-G-X₂-X₃-X₄-X₅-G-X₆-X₇-X₈-X₉,

wherein,

X₁ is selected from I, V, M, L, and T (e.g., selected from I, V, and L);

X₂ is selected from T, I, V, S, N, Y, E, and L (e.g., selected from T, V, and I);

X₃ is selected from N, S, G, A, D, T, R, M, and F (e.g., A or N);

X₄ is selected from S, Y, N, and F (e.g., S);

X₅ is selected from V, I, L, C, T, and F (e.g., selected from V, I and L);

X₆ is selected from W, F, V, Y, S, and L (e.g., W);

X₇ is selected from A, S, C, V, and G (e.g., selected from A and S);

X₈ is selected from V, I, L, A, M, and H (e.g., selected from V, I, M and L); and

X₉ is selected from any amino acid or is absent (e.g., selected from T, V, I, L, A, F, S, A, Y, M, and R, or, e.g., selected from T, V, I, L, and A).

In certain embodiments, the N-terminal RuvC-like domain differs from a sequence of SEQ ID NO:20 by as many as 1 but no more than 2, 3, 4, or 5 residues.

In certain embodiments, the N-terminal RuvC-like domain is cleavage competent. In other embodiments, the N-terminal RuvC-like domain is cleavage incompetent.

In certain embodiments, a Cas9 molecule or Cas9 polypeptide comprises an N-terminal RuvC-like domain comprising an amino acid sequence of Formula II:

(SEQ ID NO: 21)
D-X₁-G-X2-X₃-S-X₅-G-X₆-X₇-X₈-X₉,,

wherein

X₁ is selected from I, V, M, L, and T (e.g., selected from I, V, and L);

X₂ is selected from T, I, V, S, N, Y, E, and L (e.g., selected from T, V, and I);

X₃ is selected from N, S, G, A, D, T, R, M and F (e.g., A or N);

X₅ is selected from V, I, L, C, T, and F (e.g., selected from V, I and L);

X₆ is selected from W, F, V, Y, S, and L (e.g., W);

X₇ is selected from A, S, C, V, and G (e.g., selected from A and S);

X₈ is selected from V, I, L, A, M, and H (e.g., selected from V, I, M and L); and

X₉ is selected from any amino acid or is absent (e.g., selected from T, V, I, L, A, F, S, A, Y, M, and R or selected from e.g., T, V, I, L, and A).

In certain embodiments, the N-terminal RuvC-like domain differs from a sequence of SEQ ID NO:21 by as many as 1 but not more than 2, 3, 4, or 5 residues.

In certain embodiments, the N-terminal RuvC-like domain comprises an amino acid sequence of Formula III:

(SEQ ID NO: 22)
D-I-G-X₂-X₃-S-V-G-W-A-X₈-X₉,

wherein

X₂ is selected from T, I, V, S, N, Y, E, and L (e.g., selected from T, V, and I);

X₃ is selected from N, S, G, A, D, T, R, M, and F (e.g., A or N);

X₈ is selected from V, I, L, A, M, and H (e.g., selected from V, I, M and L); and

X₉ is selected from any amino acid or is absent (e.g., selected from T, V, I, L, A, F, S, A, Y, M, and R or selected from e.g., T, V, I, L, and A).

In certain embodiments, the N-terminal RuvC-like domain differs from a sequence of SEQ ID NO:22 by as many as 1 but not more than, 2, 3, 4, or 5 residues.

In certain embodiments, the N-terminal RuvC-like domain comprises an amino acid sequence of Formula IV:

(SEQ ID NO: 23)
D-I-G-T-N-S-V-G-W-A-V-X,

wherein

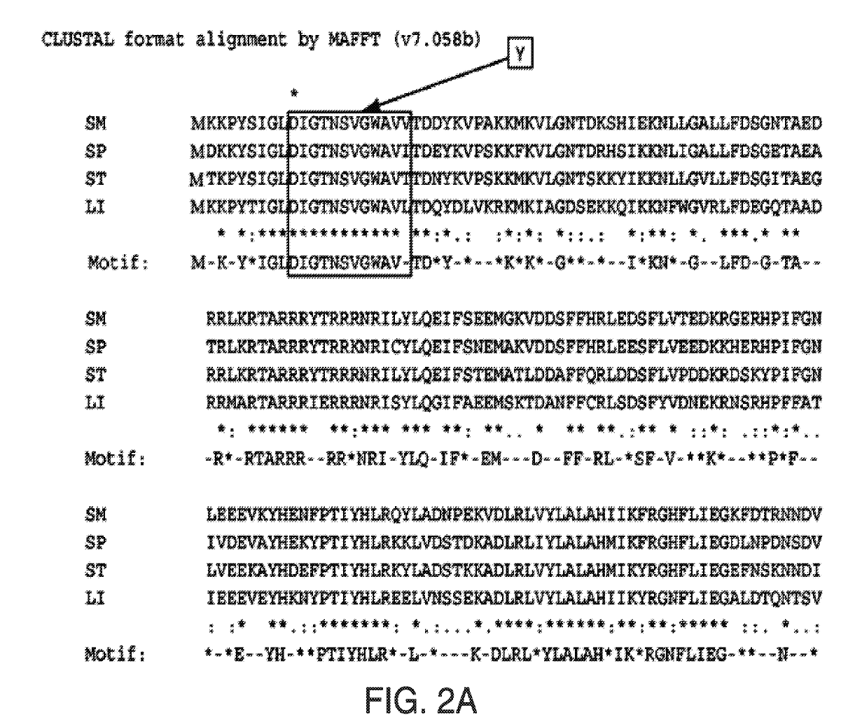

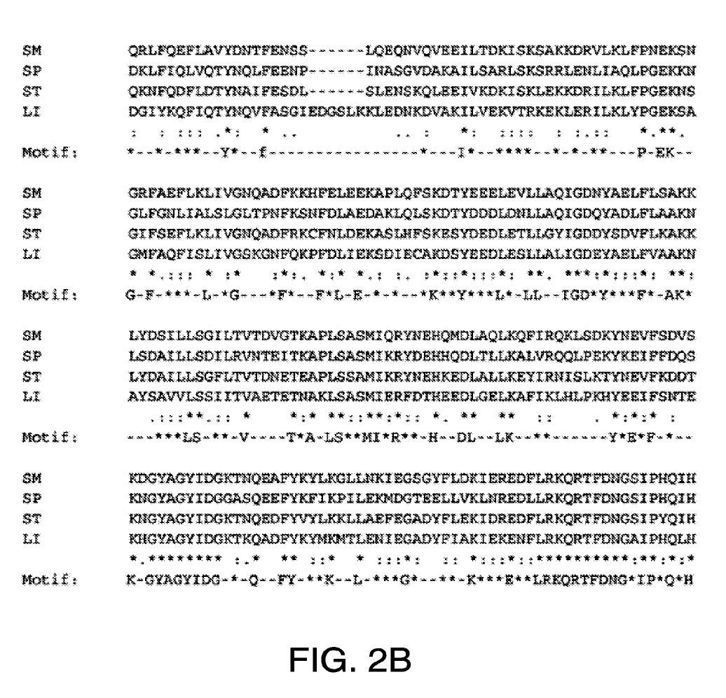

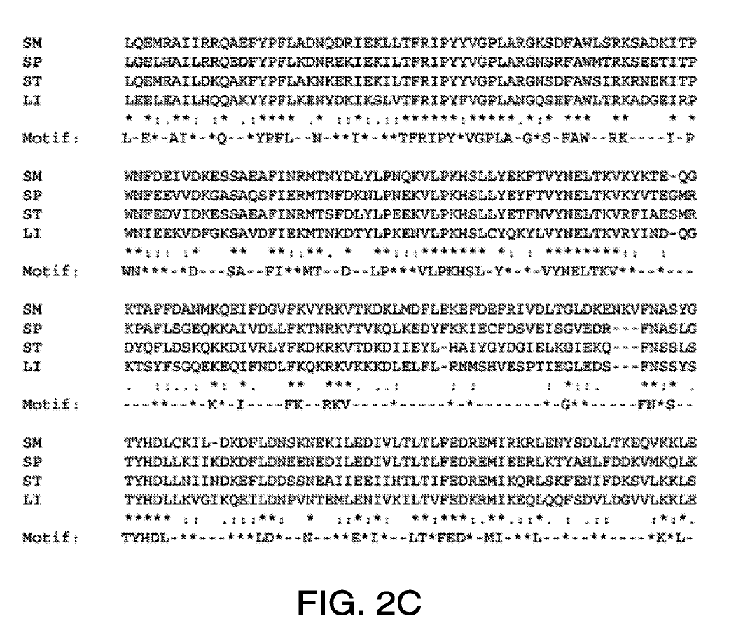

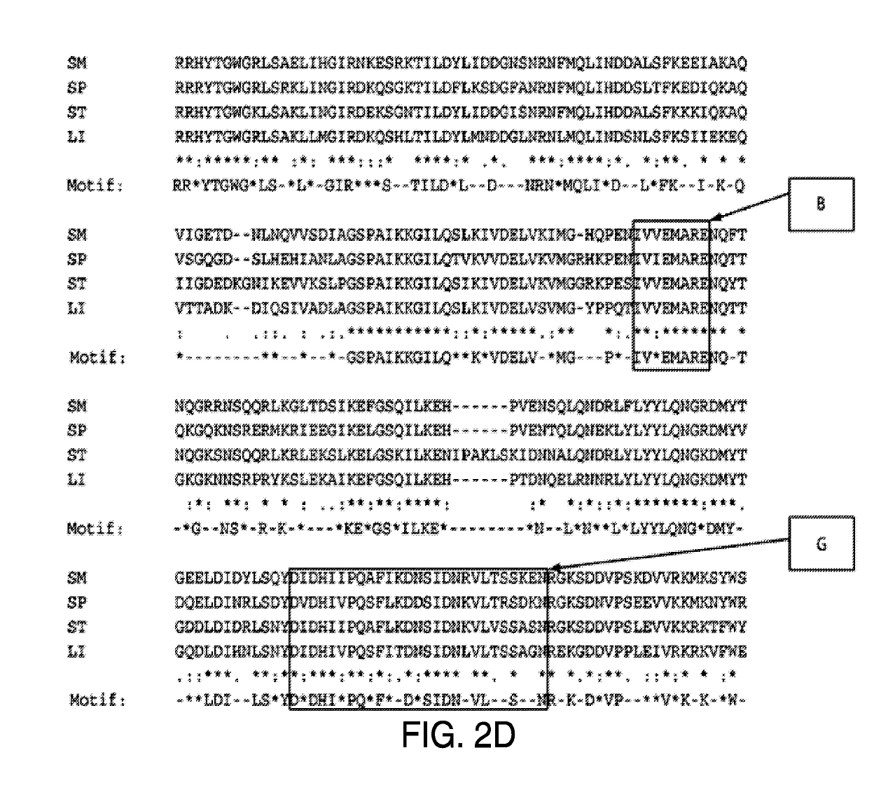

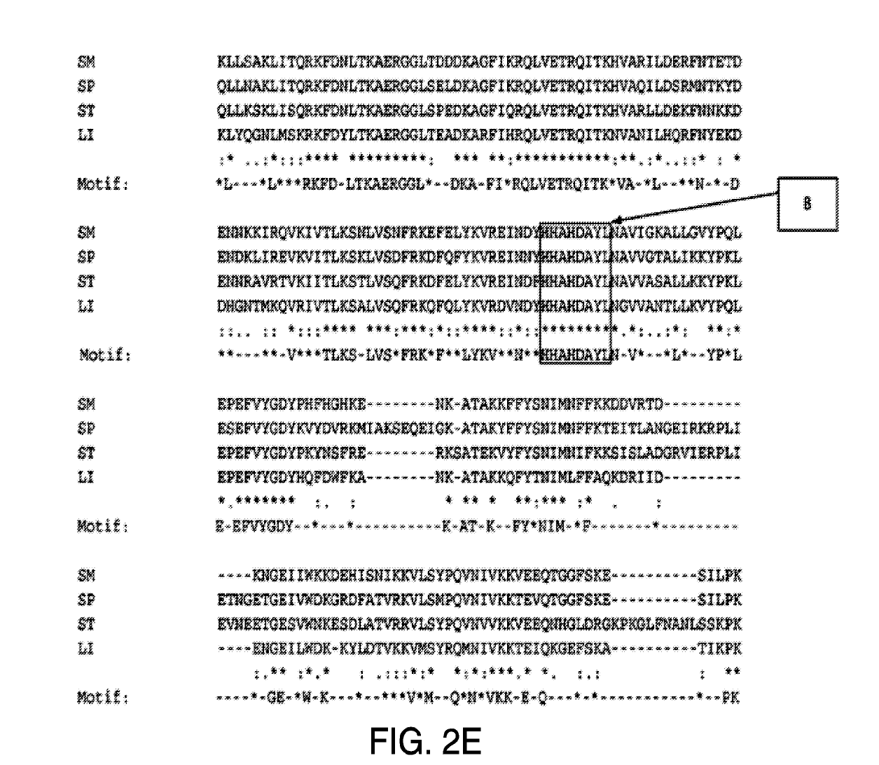

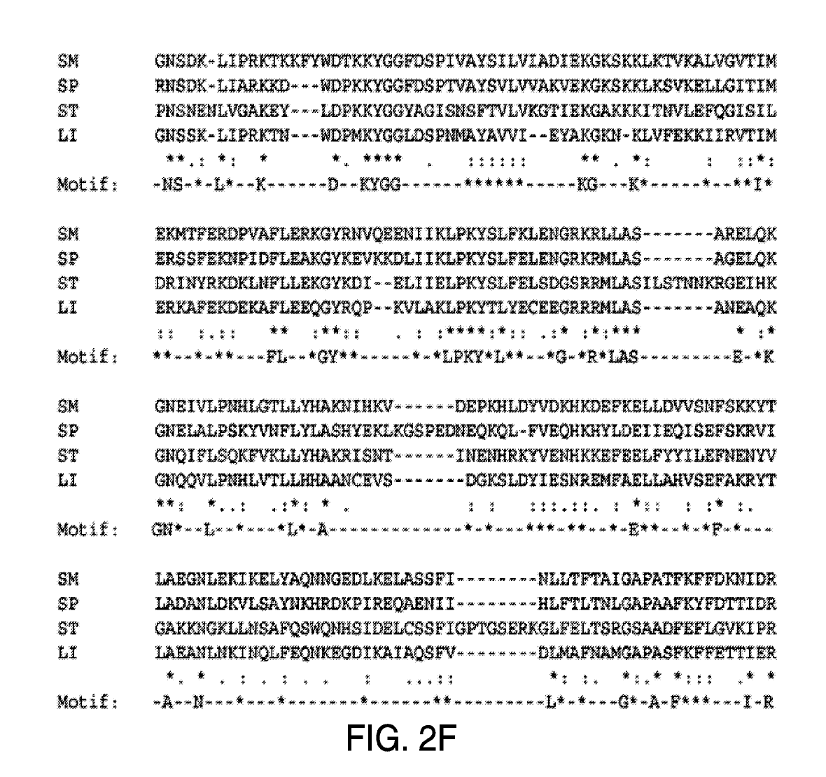

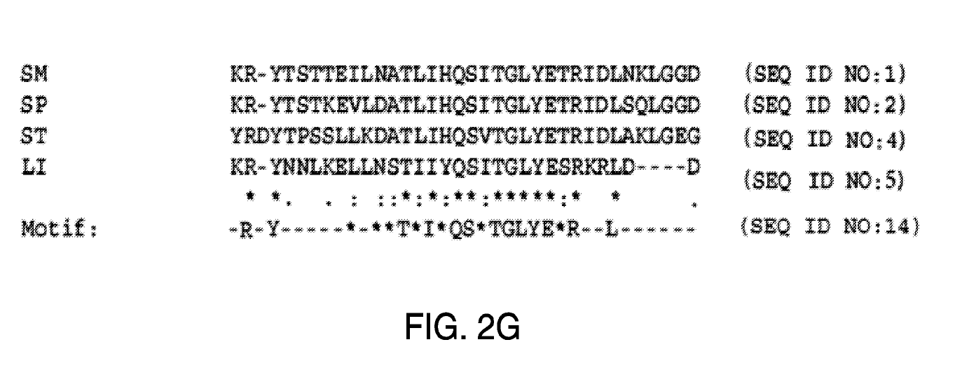

X is a non-polar alkyl amino acid or a hydroxyl amino acid, e.g., X is selected from V, I, L, and T (e.g., the Cas9 molecule can comprise an N-terminal RuvC-like domain shown in FIGS. 2A-2G (depicted as Y)).

In certain embodiments, the N-terminal RuvC-like domain differs from a sequence of SEQ ID NO:23 by as many as 1 but not more than, 2, 3, 4, or 5 residues.

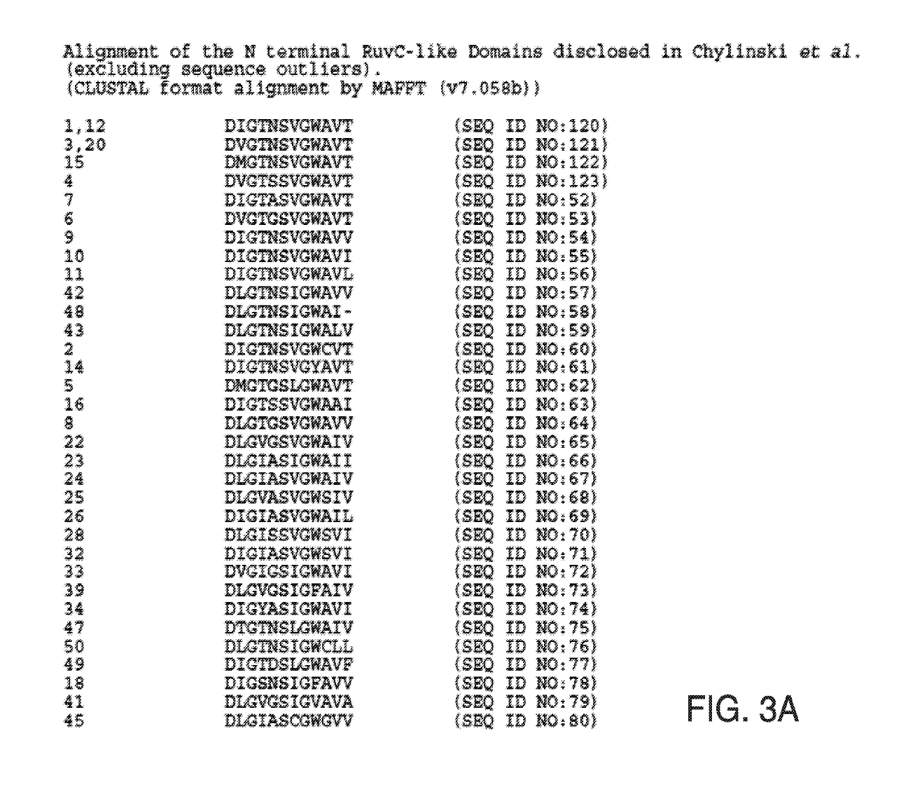

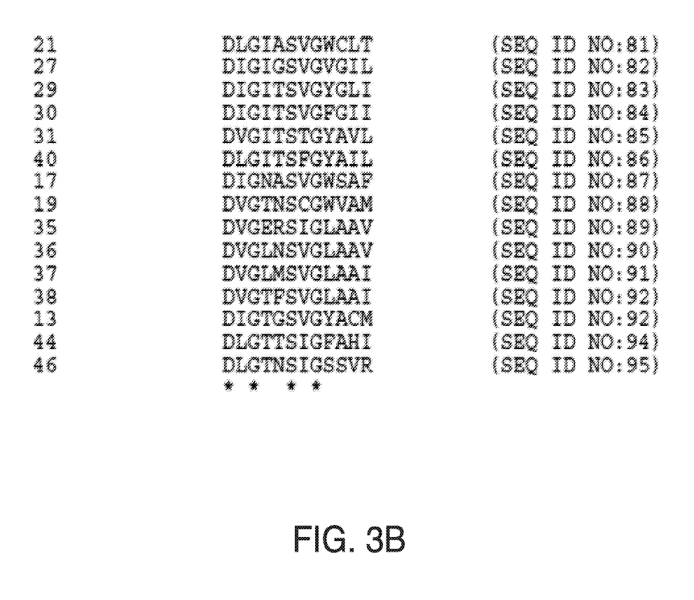

In certain embodiments, the N-terminal RuvC-like domain differs from a sequence of an N-terminal RuvC like domain disclosed herein, e.g., in FIGS. 3A-3B, as many as 1 but no more than 2, 3, 4, or 5 residues. In certain embodiments, 1, 2, 3 or all of the highly conserved residues identified in FIGS. 3A-3B are present.

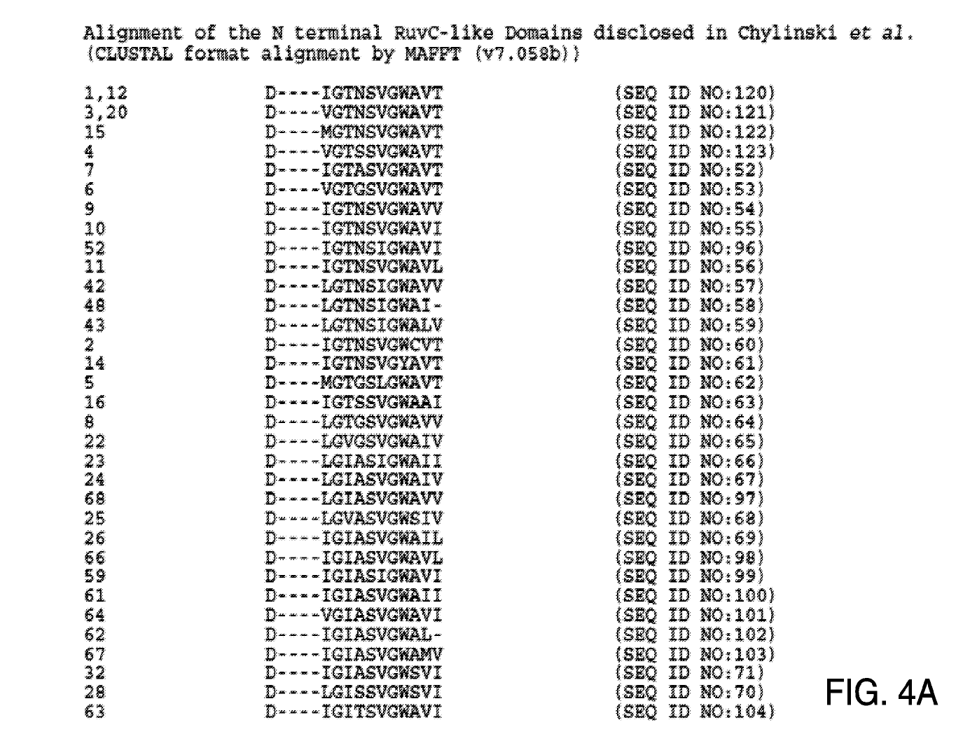

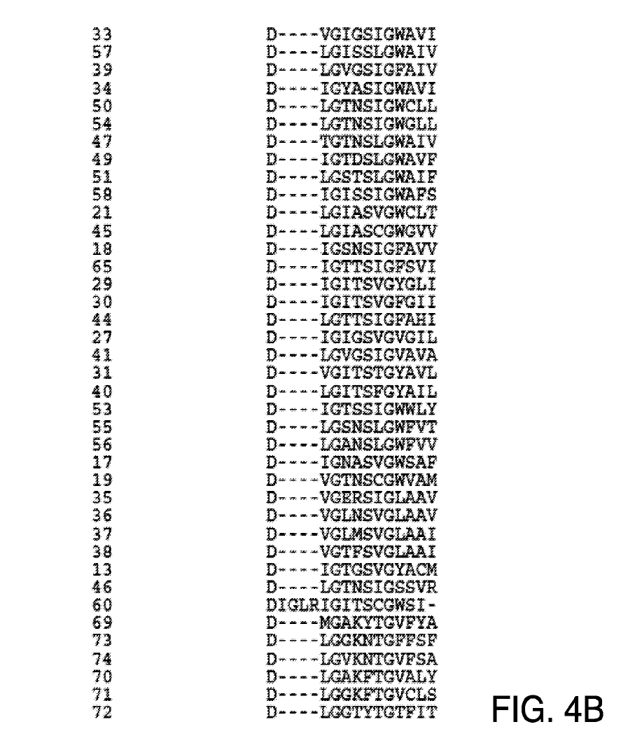

In certain embodiments, the N-terminal RuvC-like domain differs from a sequence of an N-terminal RuvC-like domain disclosed herein, e.g., in FIGS. 4A-4B, as many as 1 but no more than 2, 3, 4, or 5 residues. In certain embodiments, 1, 2, or all of the highly conserved residues identified in FIGS. 4A-4B are present.

7.1.3 Additional RuvC-Like Domains

In addition to the N-terminal RuvC-like domain, the Cas9 molecule or Cas9 polypeptide can comprise one or more additional RuvC-like domains. In certain embodiments, the Cas9 molecule or Cas9 polypeptide comprises two additional RuvC-like domains. In certain embodiments, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence of Formula V:

(SEQ ID NO: 15)
I-X₁-X₂-E-X₃-A-R-E

wherein,

X₁ is V or H;

X₂ is I, L or V (e.g., I or V); and

X₃ is M or T.

In certain embodiments, the additional RuvC-like domain comprises an amino acid sequence of Formula VI:

(SEQ ID NO: 16)
I-V-X₂-E-M-A-R-E,

wherein

X₂ is I, L or V (e.g., I or V) (e.g., the Cas9 molecule or Cas9 polypeptide can comprise an additional RuvC-like domain shown in FIG. 2A-2G (depicted as B)).

An additional RuvC-like domain can comprise an amino acid sequence of Formula VII:

(SEQ ID NO: 17)
H-H-A-X₁-D-A-X₂-X₃,

wherein

X₁ is H or L;

X₂ is R or V; and

X₃ is E or V.

In certain embodiments, the additional RuvC-like domain comprises the amino acid sequence:

(SEQ ID NO: 18)
H-H-A-H-D-A-Y-L.

In certain embodiments, the additional RuvC-like domain differs from a sequence of SEQ ID NOs:15-18 by as many as 1 but not more than 2, 3, 4, or 5 residues.

In certain embodiments, the sequence flanking the N-terminal RuvC-like domain has the amino acid sequence of Formula VIII:

(SEQ ID NO: 19)
K-X₁′-Y-X₂′-X₃′-X₄′-Z-T-D-X₉′-Y,

wherein

X₁′ is selected from K and P;

X₂′ is selected from V, L, I, and F (e.g., V, I and L);

X₃′ is selected from G, A and S (e.g., G);

X₄′ is selected from L, I, V, and F (e.g., L);

X₉′ is selected from D, E, N, and Q; and

Z is an N-terminal RuvC-like domain, e.g., as described above, e.g., having 5 to 20 amino acids.

7.1.4 HNH-Like Domains

In certain embodiments, an HNH-like domain cleaves a single stranded complementary domain, e.g., a complementary strand of a double stranded nucleic acid molecule. In certain embodiments, an HNH-like domain is at least 15, 20, or 25 amino acids in length but not more than 40, 35, or 30 amino acids in length, e.g., 20 to 35 amino acids in length, e.g., 25 to 30 amino acids in length. Exemplary HNH-like domains are described below.

In certain embodiments, a Cas9 molecule or Cas9 polypeptide comprises an HNH-like domain having an amino acid sequence of Formula IX:

(SEQ ID NO: 25)
X₁-X₂-X₃-H-X₄-X₅-P-X₆-X₇-X₈-X⁹-X¹⁰-X¹¹-X¹²-X¹³-X¹⁴-X¹⁵-N-
X¹⁶-X¹⁷-X¹⁸-X¹⁹-X₂₀-X₂₁-X₂₂-X₂₃-N,

wherein

X₁ is selected from D, E, Q and N (e.g., D and E);

X₂ is selected from L, I, R, Q, V, M, and K;

X₃ is selected from D and E;

X₄ is selected from I, V, T, A, and L (e.g., A, I and V);

X₅ is selected from V, Y, I, L, F, and W (e.g., V, I and L);

X₆ is selected from Q, H, R, K, Y, I, L, F, and W;

X₇ is selected from S, A, D, T, and K (e.g., S and A);

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₁ is selected from D, S, N, R, L, and T (e.g., D);

X₁₂ is selected from D, N and S;

X₁₃ is selected from S, A, T, G, and R (e.g., S);

X₁₄ is selected from I, L, F, S, R, Y, Q, W, D, K, and H (e.g., I, L and F);

X₁₅ is selected from D, S, I, N, E, A, H, F, L, Q, M, G, Y, and V;

X₁₆ is selected from K, L, R, M, T, and F (e.g., L, R and K);

X₁₇ is selected from V, L, I, A and T;

X₁₈ is selected from L, I, V, and A (e.g., L and I);

X₁₉ is selected from T, V, C, E, S, and A (e.g., T and V);

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiments, a HNH-like domain differs from a sequence of SEQ ID NO:25 by at least one but not more than, 2, 3, 4, or 5 residues.

In certain embodiments, the HNH-like domain is cleavage competent. In certain embodiments, the HNH-like domain is cleavage incompetent.

In certain embodiments, a Cas9 molecule or Cas9 polypeptide comprises an HNH-like domain comprising an amino acid sequence of Formula X:

(SEQ ID NO: 26)
X₁-X₂-X₃-H-X₄-X₅-P-X₆-S-X₈-X₉-X₁₀-D-D-S-X₁₄-X₁₅-N-K-V-
L-X₁₉-X₂₀-X₂₁-X₂₂-X₂₃-N,

wherein

X₁ is selected from D and E;

X₂ is selected from L, I, R, Q, V, M, and K;

X₃ is selected from D and E;

X₄ is selected from I, V, T, A, and L (e.g., A, I and V);

X₅ is selected from V, Y, I, L, F, and W (e.g., V, I and L);

X₆ is selected from Q, H, R, K, Y, I, L, F, and W;

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₄ is selected from I, L, F, S, R, Y, Q, W, D, K and H (e.g., I, L and F);

X₁₅ is selected from D, S, I, N, E, A, H, F, L, Q, M, G, Y, and V;

X₁₉ is selected from T, V, C, E, S, and A (e.g., T and V);

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiment, the HNH-like domain differs from a sequence of SEQ ID NO:26 by 1, 2, 3, 4, or 5 residues.

In certain embodiments, a Cas9 molecule or Cas9 polypeptide comprises an HNH-like domain comprising an amino acid sequence of Formula XI:

(SEQ ID NO: 27)
X₁-V-X₃-H-I-V-P-X₆-S-X₈-X₉-X₁₀-D-D-S-X₁₄-X₁₅-N-K-V-
L-T-X₂₀-X₂₁-X₂₂-X₂₃-N,

wherein

X₁ is selected from D and E;

X₃ is selected from D and E;

X₆ is selected from Q, H, R, K, Y, I, L, and W;

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₄ is selected from I, L, F, S, R, Y, Q, W, D, K, and H (e.g., I, L and F);

X₁₅ is selected from D, S, I, N, E, A, H, F, L, Q, M, G, Y, and V;

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiments, the HNH-like domain differs from a sequence of SEQ ID NO:27 by 1, 2, 3, 4, or 5 residues.

In certain embodiments, a Cas9 molecule or Cas9 polypeptide comprises an HNH-like domain having an amino acid sequence of Formula XII:

(SEQ ID NO: 28)
D-X₂-D-H-I-X₅-P-Q-X7-F-X₉-X₁₀-D-X₁₂-S-I-D-N-X₁₆-V-L-
X₁₉-X₂₀-S-X₂₂-X₂₃-N,

wherein

X₂ is selected from I and V;

X₅ is selected from I and V;

X₇ is selected from A and S;

X₉ is selected from I and L;

X₁₀ is selected from K and T;

X₁₂ is selected from D and N;

X₁₆ is selected from R, K, and L;

X₁₉ is selected from T and V;

X₂₀ is selected from S, and R;

X₂₂ is selected from K, D, and A; and

X₂₃ is selected from E, K, G, and N (e.g., the Cas9 molecule or Cas9 polypeptide can comprise an HNH-like domain as described herein).

In certain embodiments, the HNH-like domain differs from a sequence of SEQ ID NO:28 by as many as 1 but no more than 2, 3, 4, or 5 residues.

In certain embodiments, a Cas9 molecule or Cas9 polypeptide comprises the amino acid sequence of Formula XIII:

(SEQ ID NO: 24)
L-Y-Y-L-Q-N-G-X₁′-D-M-Y-X₂′-X₃′-X₄′-X₅′-L-D-I-X₆′-X₇′-
L-S-X₈′-Y-Z-N-R-X₉′-K-X₁₀-D-X₁₁′-V-P,

wherein

X₁′ is selected from K and R;

X₂′ is selected from V and T;

X₃′ is selected from G and D;

X₄′ is selected from E, Q and D;

X₅′ is selected from E and D;

X₆′ is selected from D, N, and H;

X₇′ is selected from Y, R, and N;

X₈′ is selected from Q, D, and N;

X₉′ is selected from G and E;

X₁₀′ is selected from S and G;

X₁₁′ is selected from D and N; and

Z is an HNH-like domain, e.g., as described above.

In certain embodiments, the Cas9 molecule or Cas9 polypeptide comprises an amino acid sequence that differs from a sequence of SEQ ID NO:24 by as many as 1 but not more than 2, 3, 4, or 5 residues.

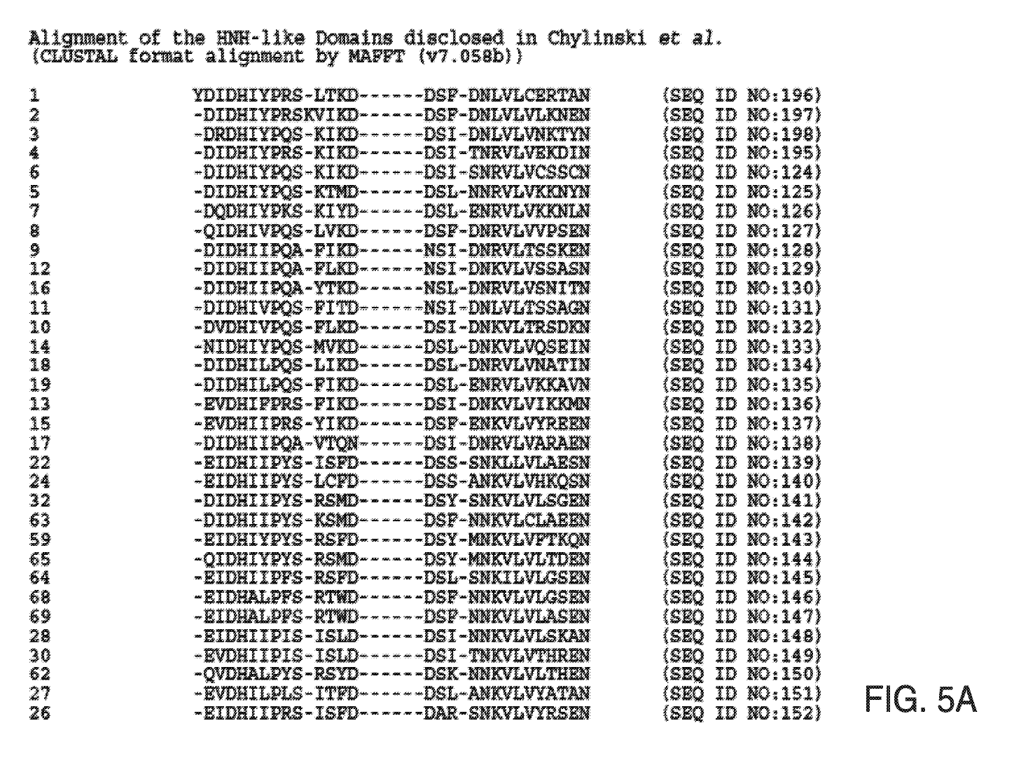

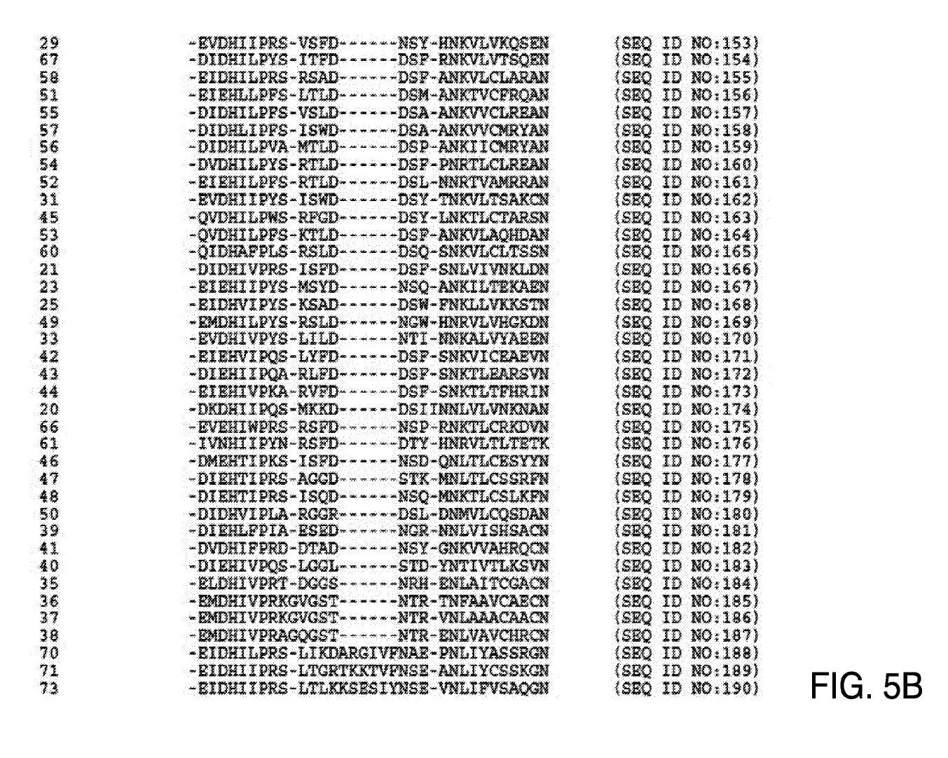

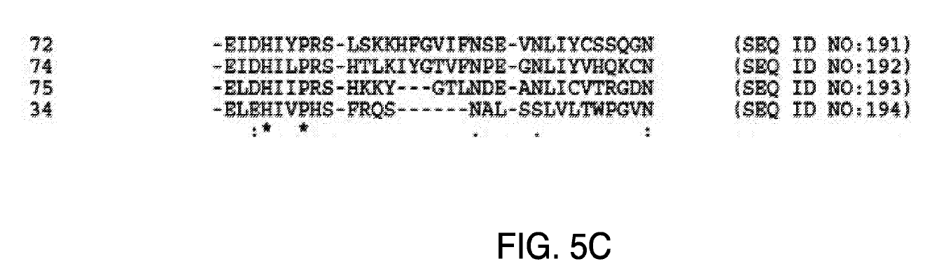

In certain embodiments, the HNH-like domain differs from a sequence of an HNH-like domain disclosed herein, e.g., in FIGS. 5A-5C, by as many as 1 but not more than 2, 3, 4, or 5 residues. In certain embodiments, 1 or both of the highly conserved residues identified in FIGS. 5A-5C are present.

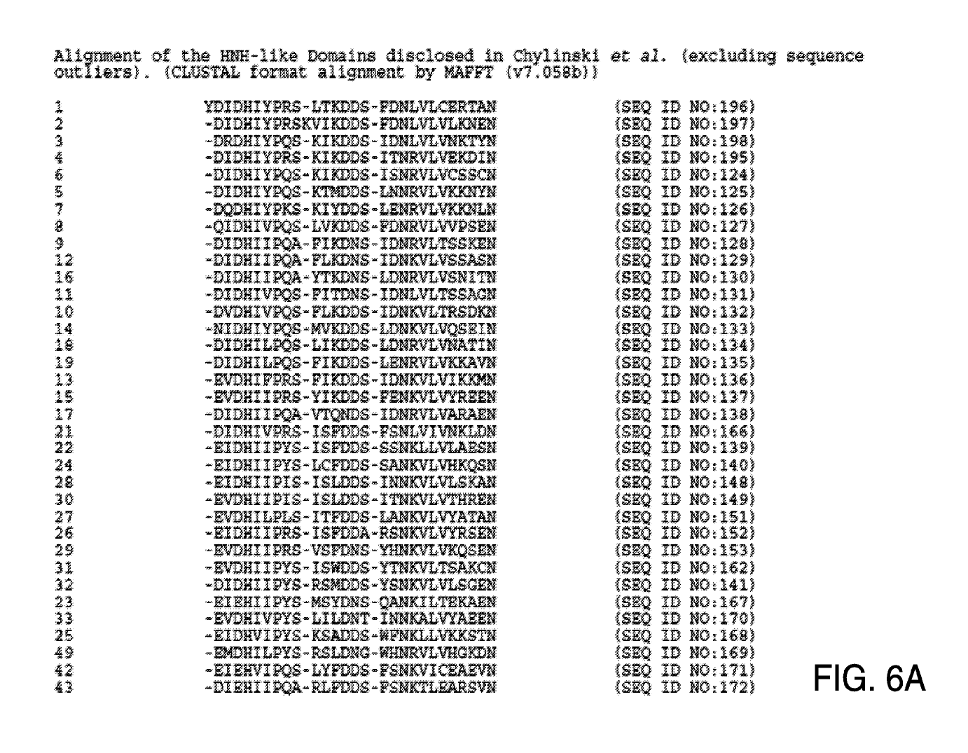

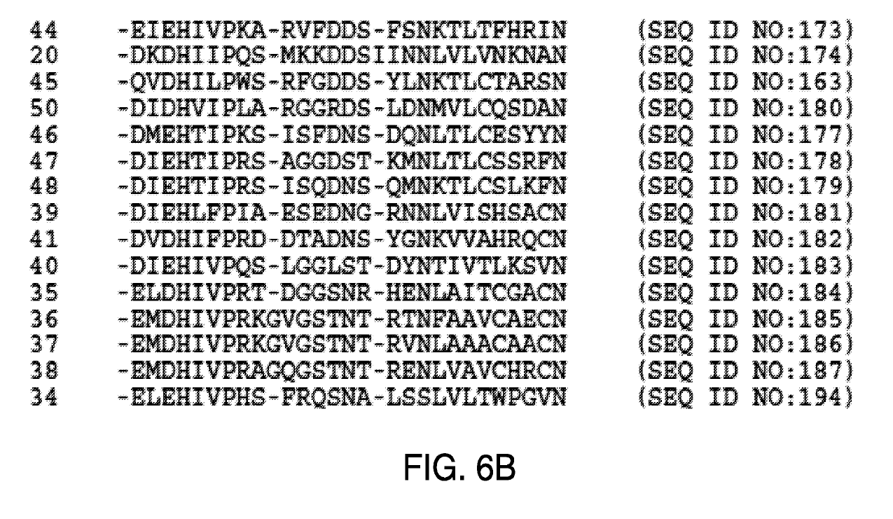

In certain embodiments, the HNH-like domain differs from a sequence of an HNH-like domain disclosed herein, e.g., in FIGS. 6A-6B, by as many as 1 but not more than 2, 3, 4, or 5 residues. In certain embodiments, 1, 2, or all 3 of the highly conserved residues identified in FIGS. 6A-6B are present.

7.2 Cas9 Activities

In certain embodiments, the Cas9 molecule or Cas9 polypeptide is capable of cleaving a target nucleic acid molecule. Typically wild-type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 (an enzymatically active Cas9) molecule or eaCas9 polypeptide.

In certain embodiments, an eaCas9 molecule or eaCas9 polypeptide comprises one or more of the following enzymatic activities:

a nickase activity, i.e., the ability to cleave a single strand, e.g., the non-complementary strand or the complementary strand, of a nucleic acid molecule;

a double stranded nuclease activity, i.e., the ability to cleave both strands of a double stranded nucleic acid and create a double stranded break, which in certain embodiments is the presence of two nickase activities;

an endonuclease activity;

an exonuclease activity; and

a helicase activity, i.e., the ability to unwind the helical structure of a double stranded nucleic acid.

In certain embodiments, an enzymatically active Cas9 (“eaCas9”) molecule or eaCas9 polypeptide cleaves both DNA strands and results in a double stranded break. In certain embodiments, an eaCas9 molecule or eaCas9 polypeptide cleaves only one strand, e.g., the strand to which the gRNA hybridizes to, or the strand complementary to the strand the gRNA hybridizes with. In certain embodiments, an eaCas9 molecule or eaCas9 polypeptide comprises cleavage activity associated with an HNH domain. In certain embodiments, an eaCas9 molecule or eaCas9 polypeptide comprises cleavage activity associated with a RuvC domain. In certain embodiments, an eaCas9 molecule or eaCas9 polypeptide comprises cleavage activity associated with an HNH domain and cleavage activity associated with a RuvC domain. In certain embodiments, an eaCas9 molecule or eaCas9 polypeptide comprises an active, or cleavage competent, HNH domain and an inactive, or cleavage incompetent, RuvC domain. In certain embodiments, an eaCas9 molecule or eaCas9 polypeptide comprises an inactive, or cleavage incompetent, HNH domain and an active, or cleavage competent, RuvC domain.

In certain embodiments, the Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an enzymatically inactive Cas9 (“eiCas9”) molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

7.3 Targeting and PAMs

A Cas9 molecule or Cas9 polypeptide can interact with a gRNA molecule and, in concert with the gRNA molecule, localizes to a site which comprises a target domain, and in certain embodiments, a PAM sequence. In certain embodiments, the Cas9 molecules or Cas9 polypeptides of the present disclosure (e.g., an eaCas9 or eiCas9) can be targeted using the gRNAs disclosed in WO 2015/089465, which is incorporated by reference herein in its entirety. In certain embodiments, the Cas9 molecule or Cas9 polypeptide targeted using the gRNAs disclosed in WO 2015/089465 is an S. pyogenes Cas9. In certain embodiments, the Cas9 molecule or Cas9 polypeptide targeted using the gRNAs disclosed in WO 2015/089465 is an S. aureus Cas9.

In certain embodiments, the ability of an eaCas9 molecule or eaCas9 polypeptide to interact with and cleave a target nucleic acid is PAM sequence dependent. A PAM sequence is a sequence in the target nucleic acid. In certain embodiments, cleavage of the target nucleic acid occurs upstream from the PAM sequence. eaCas9 molecules from different bacterial species can recognize different sequence motifs (e.g., PAM sequences). In certain embodiments, an eaCas9 molecule of S. pyogenes recognizes the sequence motif NGG and directs cleavage of a target nucleic acid sequence 1 to 10, e.g., 3 to 5, bp upstream from that sequence (see, e.g., Mali 2013).

In certain embodiments, the Cas9 molecule is an S. pyogenes Cas9 EQR variant or an S. pyogenes Cas9 VRER variant.

In certain embodiments, an eaCas9 molecule of an S. pyogenes Cas9 EQR variant recognizes the sequence motif of NGAG, NGCG, NGGG, NGTG, NGAA, NGAT or NGAC and directs cleavage of a target nucleic acid sequence at 1 to 10, e.g., 3 to 5, base pairs upstream from that sequence. In certain embodiments, an eaCas9 molecule of an S. pyogenes Cas9 EQR variant recognizes the sequence motif of NGAG and directs cleavage of a target nucleic acid sequence at 1 to 10, e.g., 3 to 5, base pairs upstream from that sequence. See Kleinstiver et al., NATURE 2015; 523(7561):481-5.

In certain embodiments, an eaCas9 molecule of S. pyogenes Cas9 VRER variant recognizes the sequence motif of NGCG, NGCA, NGCT PAM, or NGCC and directs cleavage of a target nucleic acid sequence at 1 to 10, e.g., 3 to 5, base pairs upstream from that sequence. In certain embodiments, an eaCas9 molecule of an S. pyogenes Cas9 VRER variant recognizes the sequence motif of NGCG and directs cleavage of a target nucleic acid sequence 1 to 10, e.g., 3 to 5, base pairs upstream from that sequence. See Kleinstiver et al., NATURE 2015; 523(7561):481-5.

In certain embodiments, an eaCas9 molecule of S. thermophilus recognizes the sequence motif NGGNG (SEQ ID NO:199) and/or NNAGAAW (W=A or T) (SEQ ID NO:200) and directs cleavage of a target nucleic acid sequence 1 to 10, e.g., 3 to 5, bp upstream from these sequences (see, e.g., Horvath 2010; Deveau 2008). In certain embodiments, an eaCas9 molecule of S. mutans recognizes the sequence motif NGG and/or NAAR (R=A or G) (SEQ ID NO:201) and directs cleavage of a target nucleic acid sequence 1 to 10, e.g., 3 to 5 bp, upstream from this sequence (see, e.g., Deveau 2008). In certain embodiments, an eaCas9 molecule of S. aureus recognizes the sequence motif NNGRR (R=A or G) (SEQ ID NO:202) and directs cleavage of a target nucleic acid sequence 1 to 10, e.g., 3 to 5, bp upstream from that sequence.

In certain embodiments, an eaCas9 molecule of S. aureus recognizes the sequence motif NNGRRN (R=A or G) (SEQ ID NO:203) and directs cleavage of a target nucleic acid sequence 1 to 10, e.g., 3 to 5, bp upstream from that sequence. In certain embodiments, an eaCas9 molecule of S. aureus recognizes the sequence motif NNGRRT (R=A or G) (SEQ ID NO:204) and directs cleavage of a target nucleic acid sequence 1 to 10, e.g., 3 to 5, bp upstream from that sequence. In certain embodiments, an eaCas9 molecule of S. aureus recognizes the sequence motif NNGRRV (R=A or G) (SEQ ID NO:205) and directs cleavage of a target nucleic acid sequence 1 to 10, e.g., 3 to 5, bp upstream from that sequence.

In certain embodiments, the Cas9 molecule is an S. aureus Cas9 KKH variant. In certain embodiments, an eaCas9 molecule of an S. aureus Cas9 KKH variant recognizes the sequence motif of NNGRRT or NNGRRV and directs cleavage of a target nucleic acid sequence at 1 to 10, e.g., 3 to 5, base pairs upstream from that sequence. In certain embodiments, an eaCas9 molecule of an S. aureus Cas9 KKH variant recognizes the sequence motif of NNGRRT and directs cleavage of a target nucleic acid sequence at 1 to 10, e.g., 3 to 5, base pairs upstream from that sequence. See Kleinstiver et al. (2015) NAT. BIOTECHNOL. doi: 10.1038/nbt.3404.

In certain embodiments, an eaCas9 molecule of Neisseria meningitidis recognizes the sequence motif NNNNGATT (SEQ ID NO: 8408) or NNNGCTT (SEQ ID NO: 8409) and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay as described previously (Jinek 2012). In the aforementioned embodiments, N can be any nucleotide residue, e.g., any of A, G, C, or T.

As is discussed herein, Cas9 molecules can be engineered to alter the PAM specificity of the Cas9 molecule.

Exemplary naturally occurring Cas9 molecules have been described previously (see, e.g., Chylinski 2013). Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. aureus, S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LIVID-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408).

Additional exemplary Cas9 molecules are a Cas9 molecule of Neisseria meningitides (Hou et al., PNAS Early Edition 2013, 1-6) and an S. aureus cas9 molecule.

In certain embodiments, a Cas9 molecule or Cas9 polypeptide comprises an amino acid sequence:

having about 60%, about 65%, about 70%, about 75%, about 80%, about 85%, about 90%, about 95%, about 96%, about 97%, about 98% or about 99% homology with;

differs at no more than, about 2%, about 5%, about 10%, about 15%, about 20%, about 30%, or about 40% of the amino acid residues when compared with;

differs by at least 1, 2, 5, 10 or 20 amino acids, but by no more than 100, 80, 70, 60, 50, 40 or 30 amino acids from; or

identical to any Cas9 molecule sequence described herein, or to a naturally occurring Cas9 molecule sequence, e.g., a Cas9 molecule from a species listed herein (e.g., SEQ ID NOs:1, 2, 4-6, or 12) or described in Chylinski 2013. In certain embodiments, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to localize to a target nucleic acid.

In certain embodiments, a Cas9 molecule or Cas9 polypeptide comprises any of the amino acid sequence of the consensus sequence of FIGS. 2A-2G, wherein “*” indicates any amino acid found in the corresponding position in the amino acid sequence of a Cas9 molecule of S. pyogenes, S. thermophilus, S. mutans, or L. innocua, and “-” indicates absent. In certain embodiments, a Cas9 molecule or Cas9 polypeptide differs from the sequence of the consensus sequence disclosed in FIGS. 2A-2G by at least 1, but no more than 2, 3, 4, 5, 6, 7, 8, 9, or 10 amino acid residues. In certain embodiments, a Cas9 molecule or Cas9 polypeptide comprises the amino acid sequence of SEQ ID NO:2. In other embodiments, a Cas9 molecule or Cas9 polypeptide differs from the sequence of SEQ ID NO:2 by at least 1, but no more than 2, 3, 4, 5, 6, 7, 8, 9, or 10 amino acid residues.

A comparison of the sequence of a number of Cas9 molecules indicate that certain regions are conserved. These are identified below as:

region 1 (residues 1 to 180, or in the case of region 1′ residues 120 to 180)

region 2 (residues 360 to 480);

region 3 (residues 660 to 720);

region 4 (residues 817 to 900); and

region 5 (residues 900 to 960).

In certain embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In certain embodiments, each of regions 1-5, independently, have about 50%, about 60%, about 70%, about 80%, about 85%, about 90%, about 95%, about 96%, about 97%, about 98% or about 99% homology with the corresponding residues of a Cas9 molecule or Cas9 polypeptide described herein, e.g., a sequence from FIGS. 2A-2G.

In certain embodiments, a Cas9 molecule or Cas9 polypeptide comprises an amino acid sequence referred to as region 1:

having about 50%, about 60%, about 70%, about 80%, about 85%, about 90%, about 95%, about 96%, about 97%, about 98% or about 99% homology with amino acids 1-180 (the numbering is according to the motif sequence in FIG. 2; 52% of residues in the four Cas9 sequences in FIGS. 2A-2G are conserved) of the amino acid sequence of Cas9 of S. pyogenes;

differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans, or Listeria innocua; or

is identical to amino acids 1-180 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 1′:

having about 55%, about 60%, about 65%, about 70%, about 75%, about 80%, about 85%, about 90%, about 95%, about 96%, about 97%, about 98% or about 99% homology with amino acids 120-180 (55% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans, or L. innocua; or

is identical to amino acids 120-180 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 2:

having about 50%, about 55%, about 60%, about 65%, about 70%, about 75%, about 80%, about 85%, about 90%, about 95%, about 96%, about 97%, about 98% or about 99% homology with amino acids 360-480 (52% of residues in the four Cas9 sequences in FIG. 2 are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans, or L. innocua; or

is identical to amino acids 360-480 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 3:

having about 55%, about 60%, about 65%, about 70%, about 75%, about 80%, about 85%, about 90%, about 95%, about 96%, about 97%, about 98%, or about 99% homology with amino acids 660-720 (56% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or L. innocua; or

is identical to amino acids 660-720 of the amino acid sequence of Cas9 of S. pyogenes, S. thermophilus, S. mutans or L. innocua.

In certain embodiments, a Cas9 molecule or Cas9 polypeptide comprises an amino acid sequence referred to as region 4:

having about 50%, about 55%, about 60%, about 65%, about 70%, about 75%, about 80%, about 85%, about 90%, about 95%, about 96%, about 97%, about 98%, or about 99% homology with amino acids 817-900 (55% of residues in the four Cas9 sequences in FIGS. 2A-2G are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans, or L. innocua; or

is identical to amino acids 817-900 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 5:

having about 50%, about 55%, about 60%, about 65%, about 70%, about 75%, about 80%, about 85%, about 90%, about 95%, about 96%, about 97%, about 98%, or about 99% homology with amino acids 900-960 (60% of residues in the four Cas9 sequences in FIGS. 2A-2G are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans, or L. innocua; or is identical to amino acids 900-960 of the amino acid sequence of Cas9 of S. pyogenes, S. thermophilus, S. mutans, or L. innocua.

7.4 Engineered or Altered Cas9

Cas9 molecules and Cas9 polypeptides described herein can possess any of a number of properties, including nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In certain embodiments, a Cas9 molecule or Cas9 polypeptide can include all or a subset of these properties. In certain embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (engineered, as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In certain embodiments, an engineered Cas9 molecule or Cas9 polypeptide can have an alteration that alters its size, e.g., a deletion of amino acid sequence that reduces its size, e.g., without significant effect on one or more, or any Cas9 activity. In certain embodiments, an engineered Cas9 molecule or Cas9 polypeptide can comprise an alteration that affects PAM recognition. In certain embodiments, an engineered Cas9 molecule is altered to recognize a PAM sequence other than that recognized by the endogenous wild-type PI domain. In certain embodiments, a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In certain embodiments, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In certain embodiments, a mutation or mutations do not have a substantial effect on a Cas9 activity, e.g. a Cas9 activity described herein. In certain embodiments, a mutation or mutations have a substantial effect on a Cas9 activity, e.g. a Cas9 activity described herein.

7.5 Modified-Cleavage Cas9

In certain embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

In certain embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In certain embodiments, an eaCas9 molecule or eaCas9 polypeptide comprises an active, or cleavage competent, HNH-like domain (e.g., an HNH-like domain described herein, e.g., SEQ ID NOs:24-28) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIGS. 2A-2G or an aspartic acid at position 10 of SEQ ID NO:2, e.g., can be substituted with an alanine. In certain embodiments, the eaCas9 molecule or eaCas9 polypeptide differs from wild-type in the N-terminal RuvC-like domain and does not cleave the target nucleic acid, or cleaves with significantly less efficiency, e.g., less than about 20%, about 10%, about 5%, about 1% or about 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In certain embodiments, the reference Cas9 molecule is the naturally occurring Cas9 molecule having the closest sequence identity or homology.

In certain embodiments, an eaCas9 molecule or eaCas9 polypeptide comprises an inactive, or cleavage incompetent, HNH domain and an active, or cleavage competent, N-terminal RuvC-like domain (e.g., a RuvC-like domain described herein, e.g., SEQ ID NOs:15-23). Exemplary inactive, or cleavage incompetent HNH-like domains can have a mutation at one or more of: a histidine in an HNH-like domain, e.g., a histidine shown at position 856 of the consensus sequence disclosed in FIGS. 2A-2G, e.g., can be substituted with an alanine; and one or more asparagines in an HNH-like domain, e.g., an asparagine shown at position 870 of the consensus sequence disclosed in FIGS. 2A-2G and/or at position 879 of the consensus sequence disclosed in FIGS. 2A-2G, e.g., can be substituted with an alanine. In certain embodiments, the eaCas9 differs from wild-type in the HNH-like domain and does not cleave the target nucleic acid, or cleaves with significantly less efficiency, e.g., less than about 20%, about 10%, about 5%, about 1% or about 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In certain embodiments, the reference Cas9 molecule is the naturally occurring Cas9 molecule having the closest sequence identity or homology.

In certain embodiments, exemplary Cas9 activities comprise one or more of PAM specificity, cleavage activity, and helicase activity. A mutation(s) can be present, e.g., in: one or more RuvC domains, e.g., an N-terminal RuvC domain; an HNH domain; a region outside the RuvC domains and the HNH domain. In certain embodiments, a mutation(s) is present in a RuvC domain. In certain embodiments, a mutation(s) is present in an HNH domain. In certain embodiments, mutations are present in both a RuvC domain and an HNH domain.

Exemplary mutations that may be made in the RuvC domain with reference to the S. pyogenes Cas9 sequence include: D10A, E762A, and/or D986A. Exemplary mutations that may be made in the HNH domain with reference to the S. pyogenes Cas9 sequence include: H840A, N854A, and/or N863A. Exemplary mutations that may be made in the RuvC domain with reference to the S. aureus Cas9 sequence include: D10A (see, e.g., SEQ ID NO:10). Exemplary mutations that may be made in the HNH domain with reference to the S. aureus Cas9 sequence include: N580A (see, e.g., SEQ ID NO:11).

Whether or not a particular sequence, e.g., a substitution, may affect one or more activity, such as targeting activity, cleavage activity, etc., can be evaluated or predicted, e.g., by evaluating whether the mutation is conservative. In certain embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In certain embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S. aureus or S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. aureus or S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S. aureus or S. pyogenes); or the ability to cleave a nucleic acid molecule, e.g., a double stranded or single stranded nucleic acid molecule, can be eliminated. In certain embodiments, the nickase is S. aureus Cas9-derived nickase comprising the sequence of SEQ ID NO:10 (D10A) or SEQ ID NO:11 (N580A) (Friedland 2015).

In certain embodiments, the altered Cas9 molecule is an eaCas9 molecule comprising one or more of the following activities: cleavage activity associated with a RuvC domain; cleavage activity associated with an HNH domain; cleavage activity associated with an HNH domain and cleavage activity associated with a RuvC domain.

In certain embodiments, the altered Cas9 molecule or Cas9 polypeptide comprises a sequence in which:

the sequence corresponding to the fixed sequence of the consensus sequence disclosed in FIGS. 2A-2G differs at no more than about 1%, about 2%, about 3%, about 4%, about 5%, about 10%, about 15%, or about 20% of the fixed residues in the consensus sequence disclosed in FIGS. 2A-2G; and

the sequence corresponding to the residues identified by “*” in the consensus sequence disclosed in FIGS. 2A-2G differs at no more than about 1%, about 2%, about 3%, about 4%, about 5%, about 10%, about 15%, about 20%, about 25%, about 30%, about 35%, or about 40% of the “*” residues from the corresponding sequence of naturally occurring Cas9 molecule, e.g., an S. pyogenes, S. thermophilus, S. mutans, or L. innocua Cas9 molecule.

In certain embodiments, the altered Cas9 molecule or Cas9 polypeptide is an eaCas9 molecule or eaCas9 polypeptide comprising the amino acid sequence of S. pyogenes Cas9 disclosed in FIGS. 2A-2G with one or more amino acids that differ from the sequence of S. pyogenes (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of S. thermophilus Cas9 disclosed in FIGS. 2A-2G with one or more amino acids that differ from the sequence of S. thermophilus (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of S. mutans Cas9 disclosed in FIGS. 2A-2G with one or more amino acids that differ from the sequence of S. mutans (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of L. innocua Cas9 disclosed in FIGS. 2A-2G with one or more amino acids that differ from the sequence of L. innocua (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide, e.g., an eaCas9 molecule or eaCas9 polypeptide, can be a fusion, e.g., of two of more different Cas9 molecules, e.g., of two or more naturally occurring Cas9 molecules of different species. For example, a fragment of a naturally occurring Cas9 molecule of one species can be fused to a fragment of a Cas9 molecule of a second species. As an example, a fragment of a Cas9 molecule of S. pyogenes comprising an N-terminal RuvC-like domain can be fused to a fragment of Cas9 molecule of a species other than S. pyogenes (e.g., S. thermophilus) comprising an HNH-like domain.

7.6 Cas9 with Altered or No PAM Recognition

Naturally occurring Cas9 molecules can recognize specific PAM sequences, for example the PAM recognition sequences described above for, e.g., S. pyogenes, S. thermophilus, S. mutans, and S. aureus.

In certain embodiments, a Cas9 molecule or Cas9 polypeptide has the same PAM specificities as a naturally occurring Cas9 molecule. In certain embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes in order to decrease off-target sites and/or improve specificity; or eliminate a PAM recognition requirement. In certain embodiments, a Cas9 molecule or Cas9 polypeptide can be altered, e.g., to increase length of PAM recognition sequence and/or improve Cas9 specificity to high level of identity (e.g., about 98%, about 99% or about 100% match between gRNA and a PAM sequence), e.g., to decrease off-target sites and/or increase specificity. In certain embodiments, the length of the PAM recognition sequence is at least 4, 5, 6, 7, 8, 9, 10 or 15 amino acids in length. In certain embodiments, the Cas9 specificity requires at least about 90%, about 95%, about 96%, about 97%, about 98%, or about 99% homology between the gRNA and the PAM sequence. Cas9 molecules or Cas9 polypeptides that recognize different PAM sequences and/or have reduced off-target activity can be generated using directed evolution. Exemplary methods and systems that can be used for directed evolution of Cas9 molecules are described (see, e.g., Esvelt 2011). Candidate Cas9 molecules can be evaluated, e.g., by methods described below.

7.7 Size-Optimized Cas9

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent to one of ordinary skill in the art upon review of this document.

A Cas9 molecule, e.g., an S. aureus or S. pyogenes Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome editing. A Cas9 molecule can comprise one or more deletions that do not substantially affect or decrease the activity of the resultant Cas9 molecules described herein. Activities that are retained in the Cas9 molecules comprising a deletion as described herein include one or more of the following:

a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in certain embodiments is the presence of two nickase activities;

an endonuclease activity;

an exonuclease activity;

a helicase activity, i.e., the ability to unwind the helical structure of a double stranded nucleic acid;

and recognition activity of a nucleic acid molecule, e.g., a target nucleic acid or a gRNA.

Activity of the Cas9 molecules described herein can be assessed using the activity assays described herein or in the art.

7.8 Identifying Regions Suitable for Deletion

Suitable regions of Cas9 molecules for deletion can be identified by a variety of methods. Naturally-occurring orthologous Cas9 molecules from various bacterial species can be modeled onto the crystal structure of S. pyogenes Cas9 (Nishimasu 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

7.9 Nucleic Acids Encoding Cas9 Molecules

Nucleic acids encoding the Cas9 molecules or Cas9 polypeptides, e.g., an eaCas9 molecule or eaCas9 polypeptides are provided herein. Exemplary nucleic acids encoding Cas9 molecules or Cas9 polypeptides have been described previously (see, e.g., Cong 2013; Wang 2013; Mali 2013; Jinek 2012).

In certain embodiments, a nucleic acid encoding a Cas9 molecule or Cas9 polypeptide can be a synthetic nucleic acid sequence. For example, the synthetic nucleic acid molecule can be chemically modified, e.g., as described herein. In certain embodiments, the Cas9 mRNA has one or more (e.g., all of the following properties: it is capped, polyadenylated, substituted with 5-methylcytidine and/or pseudouridine.

Additionally or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

Additionally or alternatively, a nucleic acid encoding a Cas9 molecule or Cas9 polypeptide may comprise a nuclear localization sequence (NLS). Nuclear localization sequences are known in the art.

An exemplary codon optimized nucleic acid sequence encoding a Cas9 molecule of S. pyogenes is set forth in SEQ ID NO:3. The corresponding amino acid sequence of an S. pyogenes Cas9 molecule is set forth in SEQ ID NO:2. In certain embodiments, the S. pyogenes Cas9 molecule is an S. pyogenes Cas9 variant. In certain embodiments, the S. pyogenes Cas9 variant is a EQR variant that has a sequence set forth in SEQ ID NO: 208. In certain embodiments, the S. pyogenes Cas9 variant is a VRER variant that has a sequence set forth in SEQ ID NO: 209.

Exemplary codon optimized nucleic acid sequences encoding an S. aureus Cas9 molecule are set forth in SEQ ID NOs:7-9, 206 and 207. In certain embodiments, the Cas9 molecule is a mutant S. aureus Cas9 molecule comprising a D10A mutation. In certain embodiments, the mutant S. aureus Cas9 molecule comprising a D10A mutation has a sequence set forth in SEQ ID NO: 10. In certain embodiments, the Cas9 molecule is a mutant S. aureus Cas9 molecule comprising a N580 mutation. In certain embodiments, the mutant S. aureus Cas9 molecule comprising a N580 mutation has a sequence set forth in SEQ ID NO: 11. An amino acid sequence of an S. aureus Cas9 molecule is set forth in SEQ ID NO:6.

If any of the above Cas9 sequences are fused with a peptide or polypeptide at the C-terminus, it is understood that the stop codon can be removed.

7.10 Other Cas Molecules and Cas Polypeptides

Various types of Cas molecules or Cas polypeptides can be used to practice the inventions disclosed herein. In certain embodiments, Cas molecules of Type II Cas systems are used. In certain embodiments, Cas molecules of other Cas systems are used. For example, Type I or Type III Cas molecules may be used. Exemplary Cas molecules (and Cas systems) have been described previously (see, e.g., Haft 2005 and Makarova 2011). Exemplary Cas molecules (and Cas systems) are also shown in Table 6.

TABLE 6
Cas Systems

| | | | Structure | Families | |
|---|---|---|---|---|---|
| | | | of encoded | (and | |
| | System | | protein | superfamily) | |
| Gene | type or | Name from | (PDB | of encoded | Represen- |
| name^{‡} | subtype | Haft 2005^{§} | accessions) ^{¶} | protein^{#}** | tatives |
| cas1 | Type I | cas1 | 3GOD, | COG1518 | SERP2463, |
| | Type II | | 3LFX and | | SPy1047 and |
| | Type III | | 2YZS | | ygbT |
| cas2 | Type I | cas2 | 2IVY, 2I8E | COG1343 | SERP2462, |
| | Type II | | and 3EXC | and | SPy1048, |
| | Type III | | | COG3512 | SPy1723 (N- |
| | | | | | terminal |
| | | | | | domain) and |
| | | | | | ygbF |
| cas3′ | Type I^{‡‡} | cas3 | NA | COG1203 | APE1232 |
| | | | | | and ygcB |
| cas3″ | Subtype | NA | NA | COG2254 | APE1231 |
| | I-A | | | | and BH0336 |
| | Subtype | | | | |
| | I-B | | | | |
| cas4 | Subtype | cas4 and | NA | COG1468 | APE1239 |
| | I-A | csa1 | | | and BH0340 |
| | Subtype | | | | |
| | I-B | | | | |
| | Subtype | | | | |
| | I-C | | | | |
| | Subtype | | | | |
| | I-D | | | | |
| | Subtype | | | | |
| | II-B | | | | |
| cas5 | Subtype | cas5a, | 3KG4 | COG1688 | APE1234, |
| | I-A | cas5d, | | (RAMP) | BH0337, |
| | Subtype | cas5e, | | | devS and |
| | I-B | cas5h, | | | ygcI |
| | Subtype | cas5p, | | | |
| | I-C | cas5t and | | | |
| | Subtype | cmx5 | | | |
| | I-E | | | | |
| cas6 | Subtype | cas6 and | 3I4H | COG1583 | PF1131 and |
| | I-A | cmx6 | | and | slr7014 |
| | Subtype | | | COG5551 | |
| | I-B | | | (RAMP) | |
| | Subtype | | | | |
| | I-D | | | | |
| | Subtype | | | | |
| | III-A | | | | |
| | Subtype | | | | |
| | III-B | | | | |
| cas6e | Subtype | cse3 | 1WJ9 | (RAMP) | ygcH |
| | I-E | | | | |
| cas6f | Subtype | csy4 | 2XLJ | (RAMP) | y1727 |
| | I-F | | | | |
| cas7 | Subtype | csa2, csd2, | NA | COG1857 | devR and |
| | I-A | cse4, csh2, | | and | ygcJ |
| | Subtype | csp1 and | | COG3649 | |
| | I-B | cst2 | | (RAMP) | |
| | Subtype | | | | |
| | I-C | | | | |
| | Subtype | | | | |
| | I-E | | | | |
| cas8a1 | Subtype | cmx1, cst1, | NA | BH0338-like | LA3191^{§§} |
| | I-A^{‡‡} | csx8, csx13 | | | and |
| | | and | | | PG2018^{§§} |
| | | CXXC- | | | |
| | | CXXC | | | |
| cas8a2 | Subtype | csa4 and | NA | PH0918 | AF0070, |
| | I-A^{‡‡} | csx9 | | | AF1873, |
| | | | | | MJ0385, |
| | | | | | PF0637, |
| | | | | | PH0918 and |
| | | | | | SSO1401 |
| cas8b | Subtype | csh1 and | NA | BH0338-like | MTH1090 |
| | I-B^{‡‡} | TM1802 | | | and TM1802 |
| cas8c | Subtype | csd1 and | NA | BH0338-like | BH0338 |
| | I-C^{‡‡} | csp2 | | | |
| cas9 | Type | csn1 and | NA | COG3513 | FTN_0757 |
| | II^{‡‡} | csx12 | | | and SPy1046 |
| cas10 | Type | cmr2, csm1 | NA | COG1353 | MTH326, |
| | III^{‡‡} | and csx11 | | | Rv2823c^{§§} |
| | | | | | and |
| | | | | | TM1794^{§§} |
| cas10d | Subtype | csc3 | NA | COG1353 | slr7011 |
| | I-D^{‡‡} | | | | |
| csy1 | Subtype | csy1 | NA | y1724-like | y1724 |
| | I-F^{‡‡} | | | | |
| csy2 | Subtype | csy2 | NA | (RAMP) | y1725 |
| | I-F | | | | |
| csy3 | Subtype | csy3 | NA | (RAMP) | y1726 |
| | I-F | | | | |
| cse1 | Subtype | cse1 | NA | YgcL-like | ygcL |
| | I-E^{‡‡} | | | | |
| cse2 | Subtype | cse2 | 2ZCA | YgcK-like | ygcK |
| | I-E | | | | |
| csc1 | Subtype | csc1 | NA | alr1563-like | alr1563 |
| | I-D | | | (RAMP) | |
| csc2 | Subtype | csc1 and | NA | COG1337 | slr7012 |
| | I-D | csc2 | | (RAMP) | |
| csa5 | Subtype | csa5 | NA | AF1870 | AF1870, |
| | I-A | | | | MJ0380, |
| | | | | | PF0643 and |
| | | | | | SSO1398 |
| csn2 | Subtype | csn2 | NA | SPy1049- | SPy1049 |
| | II-A | | | like | |
| csm2 | Subtype | csm2 | NA | COG1421 | MTH1081 |
| | III-A^{‡‡} | | | | and |
| | | | | | SERP2460 |
| csm3 | Subtype | csc2 and | NA | COG1337 | MTH1080 |
| | III-A | csm3 | | (RAMP) | and |
| | | | | | SERP2459 |
| csm4 | Subtype | csm4 | NA | COG1567 | MTH1079 |
| | III-A | | | (RAMP) | and |
| | | | | | SERP2458 |
| csm5 | Subtype | csm5 | NA | COG1332 | MTH1078 |
| | III-A | | | (RAMP) | and |
| | | | | | SERP2457 |
| csm6 | Subtype | APE2256 | 2WTE | COG1517 | APE2256 |
| | III-A | and csm6 | | | and SSO1445 |
| cmr1 | Subtype | cmr1 | NA | COG1367 | PF1130 |
| | III-B | | | (RAMP) | |
| cmr3 | Subtype | cmr3 | NA | COG1769 | PF1128 |
| | III-B | | | (RAMP) | |
| cmr4 | Subtype | cmr4 | NA | COG1336 | PF1126 |
| | III-B | | | (RAMP) | |
| cmr5 | Subtype | cmr5 | 2ZOP and | COG3337 | MTH324 and |
| | III-B^{‡‡} | | 2OEB | | PF1125 |
| cmr6 | Subtype | cmr6 | NA | COG1604 | PF1124 |
| | III-B | | | (RAMP) | |
| csb1 | Subtype | GSU0053 | NA | (RAMP) | Balac_1306 |
| | I-U | | | | and |
| | | | | | GSU0053 |
| csb2 | Subtype | NA | NA | (RAMP) | Balac_1305 |
| | I-U^{§§} | | | | and |
| | | | | | GSU0054 |
| csb3 | Subtype | NA | NA | (RAMP) | Balac_1303^{§§} |
| | I-U | | | | |
| csx17 | Subtype | NA | NA | NA | Btus_2683 |
| | I-U | | | | |
| csx14 | Subtype | NA | NA | NA | GSU0052 |
| | I-U | | | | |
| csx10 | Subtype | csx10 | NA | (RAMP) | Caur_2274 |
| | I-U | | | | |
| csx16 | Subtype | VVA1548 | NA | NA | VVA1548 |
| | III-U | | | | |
| csaX | Subtype | csaX | NA | NA | SSO1438 |
| | III-U | | | | |
| csx3 | Subtype | csx3 | NA | NA | AF1864 |
| | III-U | | | | |
| csx1 | Subtype | csa3, csx1, | 1XMX and | COG1517 | MJ1666, |
| | III-U | csx2, | 2171 | and | NE0113, |
| | | DXTHG, | | COG4006 | PF1127 and |
| | | NE0113 | | | TM1812 |
| | | and | | | |
| | | TIGR02710 | | | |
| csx15 | Unknown | NA | NA | TTE2665 | TTE2665 |
| csf1 | Type U | csfl | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

### 8. Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule have been described previously (Jinek 2012).

8.1 Binding and Cleavage Assay: Testing Cas9 Endonuclease Activity

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-32P]-ATP in 1× T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 μL. Reactions are initiated by the addition of 1 μL target DNA (10 nM) and incubated for 1 h at 37° C. Reactions are quenched by the addition of 20 μL of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of a candidate gRNA molecule or candidate Cas9 molecule.

8.2 Binding Assay: Testing the Binding of Cas9 Molecule to Target DNA

Exemplary methods for evaluating the binding of Cas9 molecule to target DNA have been described previously, e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1×TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 μL. Cas9 protein molecule is programmed with equimolar amounts of pre-annealed gRNA molecule and titrated from 100 pM to 1 μM. Radiolabeled DNA is added to a final concentration of 20 pM. Samples are incubated for 1 h at 37° C. and resolved at 4° C. on an 8% native polyacrylamide gel containing 1×TBE and 5 mM MgCl₂. Gels are dried and DNA visualized by phosphorimaging.

8.3 Differential Scanning Flourimetry (DSF)

The thermostability of Cas9-gRNA ribonucleoprotein (RNP) complexes can be measured via DSF. This technique measures the thermostability of a protein, which can increase under favorable conditions such as the addition of a binding RNA molecule, e.g., a gRNA.

The assay is performed using two different protocols, one to test the best stoichiometric ratio of gRNA:Cas9 protein and another to determine the best solution conditions for RNP formation.

To determine the best solution to form RNP complexes, a 2 uM solution of Cas9 in water+10× SYPRO Orange® (Life Technologies cat#S-6650) and dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with 2 uM Cas9 in optimal buffer from assay 1 above and incubating at RT for 10′ in a 384 well plate. An equal volume of optimal buffer+10× SYPRO Orange® (Life Technologies cat#S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

### 9. Genome Editing Approaches

Described herein are methods for targeted knockout of one or more copies (e.g., 1, 2, 3, 4, 5, 6, 7, 8, 9, 10 or more copies) of one or more genes in the HBV genome (e.g., PreC gene, C gene, X gene, PreS1 gene, PreS2 gene, S gene, P gene and/or SP gene), e.g., using one or more of the approaches or pathways described herein, e.g., using NHEJ.

9.1 NHEJ Approaches for Gene Targeting

In certain embodiments of the methods provided herein, NHEJ-mediated alteration is used to target gene-specific knockouts. As described herein, nuclease-induced non-homologous end-joining (NHEJ) can be used to target gene-specific knockouts. Nuclease-induced NHEJ can also be used to remove (e.g., delete) (e.g., coding sequence, non-coding sequence, or sequence insertions) in a gene of interest.

In certain embodiments, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; they are most commonly in the 1-50 bp range, but can reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs (e.g., motifs less than or equal to 50 nucleotides in length) as long as the generation of a specific final sequence is not required. If a double-strand break is targeted near to a target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. In this way, DNA segments as large as several hundred kilobases can be deleted. Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

9.1.1 Placement of Double Strand or Single Strand Breaks Relative to the Target Position

In certain embodiments, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position, e.g, an HBV target position. In certain embodiments, the cleavage site is between 0-30 bp away from the target position (e.g., less than 30, 25, 20, 15, 10, 9, 8, 7, 6, 5, 4, 3, 2 or 1 bp from the target position).

In certain embodiments, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In certain embodiments, the gRNAs are configured to position cuts at the same position, or within a few nucleotides of one another, on different strands, essentially mimicking a double strand break. In certain embodiments, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In certain embodiments, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In certain embodiments, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In certain embodiments, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair can ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20, or 10 bp).

9.2 HDR Repair, HDR-Mediated Knock-in, and Template Nucleic Acids

In certain embodiments of the methods provided herein, HDR-mediated sequence alteration is used to alter the sequence of one or more nucleotides in a HBV viral gene using an exogenously provided template nucleic acid (also referred to herein as a donor construct). In certain embodiments, HDR-mediated alteration of a HBV target position occurs by HDR with an exogenously provided donor template or template nucleic acid. For example, the donor construct or template nucleic acid provides for alteration of a HBV target position. In certain embodiments, a plasmid donor is used as a template for homologous recombination. In certain embodiments, a single stranded donor template is used as a template for alteration of the HBV target position by alternate methods of HDR (e.g., single strand annealing) between the target sequence and the donor template. Donor template-effected alteration of a HBV target position depends on cleavage by a Cas9 molecule. Cleavage by Cas9 can comprise a double strand break or two single strand breaks.

In certain embodiments, HDR-mediated sequence alteration is used to alter the sequence of one or more nucleotides in a HBV viral gene without using an exogenously provided template nucleic acid. In certain embodiments, alteration of a HBV target position occurs by HDR with endogenous genomic donor sequence. For example, the endogenous genomic donor sequence provides for alteration of the HBV target position. In certain embodiments, the endogenous genomic donor sequence is located on the same chromosome as the target sequence. In certain embodiments, the endogenous genomic donor sequence is located on a different chromosome from the target sequence. Alteration of a HBV target position by endogenous genomic donor sequence depends on cleavage by a Cas9 molecule. Cleavage by Cas9 can comprise a double strand break or two single strand breaks.

In certain embodiments of the methods provided herein, HDR-mediated alteration is used to alter a single nucleotide in a HBV viral gene. These embodiments may utilize either one double-strand break or two single-strand breaks. In certain embodiments, a single nucleotide alteration is incorporated using (1) one double-strand break, (2) two single-strand breaks, (3) two double-strand breaks with a break occurring on each side of the target position, (4) one double-strand break and two single strand breaks with the double strand break and two single strand breaks occurring on each side of the target position, (5) four single-strand breaks with a pair of single-strand breaks occurring on each side of the target position, or (6) one single-strand break.

Donor template-effected alteration of a HBV target position depends on cleavage by a Cas9 molecule. Cleavage by Cas9 can comprise a nick, a double-strand break, or two single-strand breaks, e.g., one on each strand of the target nucleic acid. After introduction of the breaks on the target nucleic acid, resection occurs at the break ends resulting in single stranded overhanging DNA regions.

In canonical HDR, a double-stranded donor template is introduced, comprising homologous sequence to the target nucleic acid that can either be directly incorporated into the target nucleic acid or used as a template to change the sequence of the target nucleic acid. After resection at the break, repair can progress by different pathways, e.g., by the double Holliday junction model (or double-strand break repair, DSBR, pathway) or the synthesis-dependent strand annealing (SDSA) pathway. In the double Holliday junction model, strand invasion by the two single stranded overhangs of the target nucleic acid to the homologous sequences in the donor template occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in alteration of the target nucleic acid. Crossover with the donor template may occur upon resolution of the junctions. In the SDSA pathway, only one single stranded overhang invades the donor template and new DNA is synthesized from the end of the invading strand to fill the gap resulting from resection. The newly synthesized DNA then anneals to the remaining single stranded overhang, new DNA is synthesized to fill in the gap, and the strands are ligated to produce the altered DNA duplex.

In alternative HDR, a single strand donor template, e.g., template nucleic acid, is introduced. A nick, single strand break, or double strand break at the target nucleic acid, for altering a desired target position, is mediated by a Cas9 molecule, e.g., described herein, and resection at the break occurs to reveal single stranded overhangs. Incorporation of the sequence of the template nucleic acid to alter a HBV target position typically occurs by the SDSA pathway, as described above.

Additional details on template nucleic acids are provided in Section IV entitled “Template nucleic acids” in International Application PCT/US2014/057905.

In certain embodiments, double strand cleavage is effected by a Cas9 molecule having cleavage activity associated with an HNH-like domain and cleavage activity associated with a RuvC-like domain, e.g., an N-terminal RuvC-like domain, e.g., a wild-type Cas9. Such embodiments require only a single gRNA.

In certain embodiments, one single-strand break, or nick, is effected by a Cas9 molecule having nickase activity, e.g., a Cas9 nickase as described herein (such as a D10A Cas9 nickase). A nicked target nucleic acid can be a substrate for alt-HDR.

In certain embodiments, two single-strand breaks, or nicks, are effected by a Cas9 molecule having nickase activity, e.g., cleavage activity associated with an HNH-like domain or cleavage activity associated with an N-terminal RuvC-like domain. Such embodiments usually require two gRNAs, one for placement of each single-strand break. In certain embodiments, the Cas9 molecule having nickase activity cleaves the strand to which the gRNA hybridizes, but not the strand that is complementary to the strand to which the gRNA hybridizes. In certain embodiments, the Cas9 molecule having nickase activity does not cleave the strand to which the gRNA hybridizes, but rather cleaves the strand that is complementary to the strand to which the gRNA hybridizes.

In certain embodiments, the nickase has HNH activity, e.g., a Cas9 molecule having the RuvC activity inactivated, e.g., a Cas9 molecule having a mutation at D10, e.g., the D10A mutation (see, e.g., SEQ ID NO:10). D10A inactivates RuvC; therefore, the Cas9 nickase has (only) HNH activity and can cut on the strand to which the gRNA hybridizes (e.g., the complementary strand, which does not have the NGG PAM on it). In certain embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In certain embodiments, a Cas9 molecule having an N863 mutation, e.g., the N863A mutation, mutation can be used as a nickase. N863A inactivates HNH therefore the Cas9 nickase has (only) RuvC activity and cuts on the non-complementary strand (the strand that has the NGG PAM and whose sequence is identical to the gRNA). In certain embodiments, a Cas9 molecule having an N580 mutation, e.g., the N580A mutation, mutation can be used as a nickase. N580A inactivates HNH therefore the Cas9 nickase has (only) RuvC activity and cuts on the non-complementary strand (the strand that has the NGG PAM and whose sequence is identical to the gRNA).

In certain embodiments, in which a nickase and two gRNAs are used to position two single strand nicks, one nick is on the + strand and one nick is on the − strand of the target nucleic acid. The PAMs can be outwardly facing. The gRNAs can be selected such that the gRNAs are separated by, from about 0-50, 0-100, or 0-200 nucleotides. In certain embodiments, there is no overlap between the target sequences that are complementary to the targeting domains of the two gRNAs. In certain embodiments, the gRNAs do not overlap and are separated by as much as 50, 100, or 200 nucleotides. In certain embodiments, the use of two gRNAs can increase specificity, e.g., by decreasing off-target binding (Ran 2013).

In certain embodiments, a single nick can be used to induce HDR, e.g., alt-HDR. In certain embodiments, a single nick can be used to increase the ratio of HR to NHEJ at a given cleavage site. In certain embodiments, a single strand break is formed in the strand of the target nucleic acid to which the targeting domain of said gRNA is complementary. In certain embodiments, a single strand break is formed in the strand of the target nucleic acid other than the strand to which the targeting domain of said gRNA is complementary.

9.2.1 Placement of Double Strand or Single Strand Breaks Relative to the Target Position

A double strand break or single strand break in one of the strands should be sufficiently close to a HBV target position that an alteration is produced in the desired region. In certain embodiments, the distance is not more than 50, 100, 200, 300, 350 or 400 nucleotides. In certain embodiments, the break should be sufficiently close to target position such that the target position is within the region that is subject to exonuclease-mediated removal during end resection. If the distance between the HBV target position and a break is too great, the sequence desired to be altered may not be included in the end resection and, therefore, may not be altered, as donor sequence, either exogenously provided donor sequence or endogenous genomic donor sequence, in certain embodiments is only used to alter sequence within the end resection region.

In certain embodiments, the methods described herein introduce one or more breaks near a HBV target position. In certain of these embodiments, two or more breaks are introduced that flank a HBV target position. The two or more breaks remove (e.g., delete) a genomic sequence including a HBV target position. All methods described herein result in altering a HBV target position within a HBV viral gene.

In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, or 200 nucleotides of the region desired to be altered, e.g., a mutation. The break, e.g., a double strand or single strand break, can be positioned upstream or downstream of the region desired to be altered, e.g., a mutation. In certain embodiments, a break is positioned within the region desired to be altered, e.g., within a region defined by at least two mutant nucleotides. In certain embodiments, a break is positioned immediately adjacent to the region desired to be altered, e.g., immediately upstream or downstream of a mutation.

In certain embodiments, a single strand break is accompanied by an additional single strand break, positioned by a second gRNA molecule, as discussed below. For example, the targeting domains bind configured such that a cleavage event, e.g., the two single strand breaks, are positioned within 1, 2, 3, 4, 5, 10, 15, 20, 25, 30, 35, 40, 45, 50, 60, 70, 80, 90, 100, 150, or 200 nucleotides of a target position. In certain embodiments, the first and second gRNA molecules are configured such that, when guiding a Cas9 nickase, a single strand break can be accompanied by an additional single strand break, positioned by a second gRNA, sufficiently close to one another to result in alteration of the desired region. In certain embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments in which a gRNA (unimolecular (or chimeric) or modular gRNA) and Cas9 nuclease induce a double strand break for the purpose of inducing HDR-mediated sequence alteration, the cleavage site is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In certain embodiments, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In certain embodiments, one can promote HDR by using nickases to generate a break with overhangs. While not wishing to be bound by theory, the single stranded nature of the overhangs can enhance the cell's likelihood of repairing the break by HDR as opposed to, e.g., NHEJ. Specifically, in certain embodiments, HDR is promoted by selecting a first gRNA that targets a first nickase to a first target sequence, and a second gRNA that targets a second nickase to a second target sequence which is on the opposite DNA strand from the first target sequence and offset from the first nick.

In certain embodiments, the targeting domain of a gRNA molecule is configured to position a cleavage event sufficiently far from a preselected nucleotide that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

9.3.2. Placement of a First Break and a Second Break Relative to Each Other

In certain embodiments, a double strand break can be accompanied by an additional double strand break, positioned by a second gRNA molecule, as is discussed below.

In certain embodiments, a double strand break can be accompanied by two additional single strand breaks, positioned by a second gRNA molecule and a third gRNA molecule.

In certain embodiments, a first and second single strand breaks can be accompanied by two additional single strand breaks positioned by a third gRNA molecule and a fourth gRNA molecule.

When two or more gRNAs are used to position two or more cleavage events, e.g., double strand or single strand breaks, in a target nucleic acid, the two or more cleavage events may be made by the same or different Cas9 proteins. For example, when two gRNAs are used to position two double stranded breaks, a single Cas9 nuclease may be used to create both double stranded breaks. When two or more gRNAs are used to position two or more single stranded breaks (nicks), a single Cas9 nickase may be used to create the two or more nicks. When two or more gRNAs are used to position at least one double stranded break and at least one single stranded break, two Cas9 proteins may be used, e.g., one Cas9 nuclease and one Cas9 nickase. In certain embodiments, two or more Cas9 proteins are used, and the two or more Cas9 proteins may be delivered sequentially to control specificity of a double stranded versus a single stranded break at the desired position in the target nucleic acid.

In certain embodiments, the targeting domain of the first gRNA molecule and the targeting domain of the second gRNA molecules are complementary to opposite strands of the target nucleic acid molecule. In certain embodiments, the gRNA molecule and the second gRNA molecule are configured such that the PAMs are oriented outward.

In certain embodiments, two gRNA are selected to direct Cas9-mediated cleavage at two positions that are a preselected distance from each other. In certain embodiments, the two points of cleavage are on opposite strands of the target nucleic acid. In certain embodiments, the two cleavage points form a blunt ended break, and in other embodiments, they are offset so that the DNA ends comprise one or two overhangs (e.g., one or more 5′ overhangs and/or one or more 3′ overhangs). In certain embodiments, each cleavage event is a nick. In certain embodiments, the nicks are close enough together that they form a break that is recognized by the double stranded break machinery (as opposed to being recognized by, e.g., the SSBr machinery). In certain embodiments, the nicks are far enough apart that they create an overhang that is a substrate for HDR, i.e., the placement of the breaks mimics a DNA substrate that has experienced some resection. For instance, in certain embodiments the nicks are spaced to create an overhang that is a substrate for processive resection. In certain embodiments, the two breaks are spaced within 25-65 nucleotides of each other. The two breaks may be, e.g., about 25, 30, 35, 40, 45, 50, 55, 60, or 65 nucleotides of each other. The two breaks may be, e.g., at least about 25, 30, 35, 40, 45, 50, 55, 60, or 65 nucleotides of each other. The two breaks may be, e.g., at most about 30, 35, 40, 45, 50, 55, 60, or 65 nucleotides of each other. In certain embodiments, the two breaks are about 25-30, 30-35, 35-40, 40-45, 45-50, 50-55, 55-60, or 60-65 nucleotides of each other.

In certain embodiments, the break that mimics a resected break comprises a 3′ overhang (e.g., generated by a DSB and a nick, where the nick leaves a 3′ overhang), a 5′ overhang (e.g., generated by a DSB and a nick, where the nick leaves a 5′ overhang), a 3′ and a 5′ overhang (e.g., generated by three cuts), two 3′ overhangs (e.g., generated by two nicks that are offset from each other), or two 5′ overhangs (e.g., generated by two nicks that are offset from each other).

In certain embodiments in which two gRNAs (independently, unimolecular (or chimeric) or modular gRNA) complexing with Cas9 nickases induce two single strand breaks for the purpose of inducing HDR-mediated alteration, the closer nick is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, or 75 to 100 bp) away from the target position and the two nicks can ideally be within 25-65 bp of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, 20, 10, or 5 bp away from each other). In certain embodiments, the cleavage site is between 0-100 bp (e.g., 0 to 75, 0 to 50, 0 to 25, 25 to 100, 25 to 75, 25 to 50, 50 to 100, 50 to 75, or 75 to 100 bp) away from the target position.

In certain embodiments, two gRNAs, e.g., independently, unimolecular (or chimeric) or modular gRNA, are configured to position a double-strand break on both sides of a target position. In certain embodiments, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In certain embodiments, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair can ideally be within 0-500 bp of the target position (e.g., no more than 450, 400, 350, 300, 250, 200, 150, 100, 50 or 25 bp from the target position). When nickases are used, the two nicks in a pair are, in certain embodiments, within 25-65 bp of each other (e.g., between 25 to 55, 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, or 20 or 10 bp).

When two gRNAs are used to target Cas9 molecules to breaks, different combinations of Cas9 molecules are envisioned. In certain embodiments, a first gRNA is used to target a first Cas9 molecule to a first target position, and a second gRNA is used to target a second Cas9 molecule to a second target position. In certain embodiments, the first Cas9 molecule creates a nick on the first strand of the target nucleic acid, and the second Cas9 molecule creates a nick on the opposite strand, resulting in a double stranded break (e.g., a blunt ended cut or a cut with overhangs).

Different combinations of nickases can be chosen to target one single stranded break to one strand and a second single stranded break to the opposite strand. When choosing a combination, one can take into account that there are nickases having one active RuvC-like domain, and nickases having one active HNH domain. In certain embodiments, a RuvC-like domain cleaves the non-complementary strand of the target nucleic acid molecule. In certain embodiments, an HNH-like domain cleaves a single stranded complementary domain, e.g., a complementary strand of a double stranded nucleic acid molecule. Generally, if both Cas9 molecules have the same active domain (e.g., both have an active RuvC domain or both have an active HNH domain), one can choose two gRNAs that bind to opposite strands of the target. In more detail, in certain embodiments a first gRNA is complementary with a first strand of the target nucleic acid and binds a nickase having an active RuvC-like domain and causes that nickase to cleave the strand that is non-complementary to that first gRNA, i.e., a second strand of the target nucleic acid; and a second gRNA is complementary with a second strand of the target nucleic acid and binds a nickase having an active RuvC-like domain and causes that nickase to cleave the strand that is non-complementary to that second gRNA, i.e., the first strand of the target nucleic acid. Conversely, in certain embodiments, a first gRNA is complementary with a first strand of the target nucleic acid and binds a nickase having an active HNH domain and causes that nickase to cleave the strand that is complementary to that first gRNA, i.e., a first strand of the target nucleic acid; and a second gRNA is complementary with a second strand of the target nucleic acid and binds a nickase having an active HNH domain and causes that nickase to cleave the strand that is complementary to that second gRNA, i.e., the second strand of the target nucleic acid. In another arrangement, if one Cas9 molecule has an active RuvC-like domain and the other Cas9 molecule has an active HNH domain, the gRNAs for both Cas9 molecules can be complementary to the same strand of the target nucleic acid, so that the Cas9 molecule with the active RuvC-like domain can cleave the non-complementary strand and the Cas9 molecule with the HNH domain can cleave the complementary strand, resulting in a double stranded break.

9.3.3 Homology Arms of the Donor Template

A homology arm should extend at least as far as the region in which end resection may occur, e.g., in order to allow the resected single stranded overhang to find a complementary region within the donor template. The overall length could be limited by parameters such as plasmid size or viral packaging limits. In certain embodiments, a homology arm does not extend into repeated elements, e.g., Alu repeats or LINE repeats.

Exemplary homology arm lengths include at least 50, 100, 250, 500, 750, 1000, 2000, 3000, 4000, or 5000 nucleotides. In certain embodiments, the homology arm length is 50-100, 100-250, 250-500, 500-750, 750-1000, 1000-2000, 2000-3000, 3000-4000, or 4000-5000 nucleotides.

A template nucleic acid, as that term is used herein, refers to a nucleic acid sequence which can be used in conjunction with a Cas9 molecule and a gRNA molecule to alter the structure of a HBV target position. In certain embodiments, the HBV target position can be a site between two nucleotides, e.g., adjacent nucleotides, on the target nucleic acid into which one or more nucleotides is added. Alternatively, the HBV target position may comprise one or more nucleotides that are altered by a template nucleic acid.

In certain embodiments, the target nucleic acid is modified to have some or all of the sequence of the template nucleic acid, typically at or near cleavage site(s). In certain embodiments, the template nucleic acid is single stranded. In certain embodiments, the template nucleic acid is double stranded. In certain embodiments, the template nucleic acid is DNA, e.g., double stranded DNA. In certain embodiments, the template nucleic acid is single stranded DNA. In certain embodiments, the template nucleic acid is encoded on the same vector backbone, e.g. AAV genome, plasmid DNA, as the Cas9 and gRNA. In certain embodiments, the template nucleic acid is excised from a vector backbone in vivo, e.g., it is flanked by gRNA recognition sequences. In certain embodiments, the template nucleic acid comprises endogenous genomic sequence.

In certain embodiments, the template nucleic acid alters the structure of the target position by participating in an HDR event. In certain embodiments, the template nucleic acid alters the sequence of the target position. In certain embodiments, the template nucleic acid results in the incorporation of a modified, or non-naturally occurring base into the target nucleic acid.

Typically, the template sequence undergoes a breakage mediated or catalyzed recombination with the target sequence. In certain embodiments, the template nucleic acid includes sequence that corresponds to a site on the target sequence that is cleaved by an eaCas9 mediated cleavage event.

In certain embodiments, the template nucleic acid includes sequence that corresponds to both a first site on the target sequence that is cleaved in a first Cas9 mediated event, and a second site on the target sequence that is cleaved in a second Cas9 mediated event.

A template nucleic acid typically comprises the following components:

[5′ homology arm]-[replacement sequence]-[3′ homology arm].

The homology arms provide for recombination into the chromosome, thus replacing the undesired element, e.g., a mutation or signature, with the replacement sequence. In certain embodiments, the homology arms flank the most distal cleavage sites.

In certain embodiments, the 3′ end of the 5′ homology arm is the position next to the 5′ end of the replacement sequence. In certain embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the replacement sequence.

In certain embodiments, the 5′ end of the 3′ homology arm is the position next to the 3′ end of the replacement sequence. In certain embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the replacement sequence.

In certain embodiments, to alter one or more nucleotides at a HBV target position, the homology arms, e.g., the 5′ and 3′ homology arms, may each comprise about 1000 bp of sequence flanking the most distal gRNAs (e.g., 1000 bp of sequence on either side of the HBV target position).

In certain embodiments, one or both homology arms may be shortened to avoid including certain sequence repeat elements, e.g., Alu repeats or LINE elements. For example, a 5′ homology arm may be shortened to avoid a sequence repeat element. In certain embodiments, a 3′ homology arm may be shortened to avoid a sequence repeat element. In certain embodiments, both the 5′ and the 3′ homology arms may be shortened to avoid including certain sequence repeat elements.

In certain embodiments, template nucleic acids for altering the sequence of a HBV target position may be designed for use as a single-stranded oligonucleotide, e.g., a single-stranded oligodeoxynucleotide (ssODN). When using a ssODN, 5′ and 3′ homology arms may range up to about 200 bp in length, e.g., at least 25, 50, 75, 100, 125, 150, 175, or 200 bp in length. Longer homology arms can also be for ssODNs as improvements in oligonucleotide synthesis continue to be made. In certain embodiments, a longer homology arm is made by a method other than chemical synthesis, e.g., by denaturing a long double stranded nucleic acid and purifying one of the strands, e.g., by affinity for a strand-specific sequence anchored to a solid substrate.

In certain embodiments, alt-HDR proceeds more efficiently when the template nucleic acid has extended homology 5′ to the nick (i.e., in the 5′ direction of the nicked strand). Accordingly, in certain embodiments, the template nucleic acid has a longer homology arm and a shorter homology arm, wherein the longer homology arm can anneal 5′ of the nick. In certain embodiments, the arm that can anneal 5′ to the nick is at least 25, 50, 75, 100, 125, 150, 175, or 200, 300, 400, 500, 600, 700, 800, 900, 1000, 1500, 2000, 3000, 4000, or 5000 nucleotides from the nick or the 5′ or 3′ end of the replacement sequence. In certain embodiments, the arm that can anneal 5′ to the nick is at least about 10%, about 20%, about 30%, about 40%, or about 50% longer than the arm that can anneal 3′ to the nick. In certain embodiments, the arm that can anneal 5′ to the nick is at least 2×, 3×, 4×, or 5× longer than the arm that can anneal 3′ to the nick. Depending on whether a ssDNA template can anneal to the intact strand or the nicked strand, the homology arm that anneals 5′ to the nick may be at the 5′ end of the ssDNA template or the 3′ end of the ssDNA template, respectively.

Similarly, in certain embodiments, the template nucleic acid has a 5′ homology arm, a replacement sequence, and a 3′ homology arm, such that the template nucleic acid has extended homology to the 5′ of the nick. For example, the 5′ homology arm and 3′ homology arm may be substantially the same length, but the replacement sequence may extend farther 5′ of the nick than 3′ of the nick. In certain embodiments, the replacement sequence extends at least about 10%, about 20%, about 30%, about 40%, about 50%, 2×, 3×, 4×, or 5× further to the 5′ end of the nick than the 3′ end of the nick.

In certain embodiments, alt-HDR proceeds more efficiently when the template nucleic acid is centered on the nick. Accordingly, in certain embodiments, the template nucleic acid has two homology arms that are essentially the same size. For instance, the first homology arm of a template nucleic acid may have a length that is within about 10%, about 9%, about 8%, about 7%, about 6%, about 5%, about 4%, about 3%, about 2%, or about 1% of the second homology arm of the template nucleic acid.

Similarly, in certain embodiments, the template nucleic acid has a 5′ homology arm, a replacement sequence, and a 3′ homology arm, such that the template nucleic acid extends substantially the same distance on either side of the nick. For example, the homology arms may have different lengths, but the replacement sequence may be selected to compensate for this. For example, the replacement sequence may extend further 5′ from the nick than it does 3′ of the nick, but the homology arm 5′ of the nick is shorter than the homology arm 3′ of the nick, to compensate. The converse is also possible, e.g., that the replacement sequence may extend further 3′ from the nick than it does 5′ of the nick, but the homology arm 3′ of the nick is shorter than the homology arm 5′ of the nick, to compensate.

9.3.4. Template Nucleic Acids

In certain embodiments, the template nucleic acid is double stranded. In certain embodiments, the template nucleic acid is single stranded. In certain embodiments, the template nucleic acid comprises a single stranded portion and a double stranded portion. In certain embodiments, the template nucleic acid comprises about 50 to 100 bp, e.g., 55 to 95, 60 to 90, 65 to 85, or 70 to 80 bp, homology on either side of the nick and/or replacement sequence. In certain embodiments, the template nucleic acid comprises about 50, 55, 60, 65, 70, 75, 80, 85, 90, 95, or 100 bp homology 5′ of the nick or replacement sequence, 3′ of the nick or replacement sequence, or both 5′ and 3′ of the nick or replacement sequences.

In certain embodiments, the template nucleic acid comprises about 150 to 200 bp, e.g., 155 to 195, 160 to 190, 165 to 185, or 170 to 180 bp, homology 3′ of the nick and/or replacement sequence. In certain embodiments, the template nucleic acid comprises about 150, 155, 160, 165, 170, 175, 180, 185, 190, 195, or 200 bp homology 3′ of the nick or replacement sequence. In certain embodiments, the template nucleic acid comprises less than about 100, 90, 80, 70, 60, 50, 40, 30, 20, 15, or 10 bp homology 5′ of the nick or replacement sequence.

In certain embodiment, the template nucleic acid comprises about 150 to 200 bp, e.g., 155 to 195, 160 to 190, 165 to 185, or 170 to 180 bp, homology 5′ of the nick and/or replacement sequence. In certain embodiment, the template nucleic acid comprises about 150, 155, 160, 165, 170, 175, 180, 185, 190, 195, or 200 bp homology 5′ of the nick or replacement sequence. In certain embodiments, the template nucleic acid comprises less than about 100, 90, 80, 70, 60, 50, 40, 30, 20, 15, or 10 bp homology 3′ of the nick or replacement sequence.

In certain embodiments, the template nucleic acid comprises a nucleotide sequence, e.g., of one or more nucleotides, that can be added to or can template a change in the target nucleic acid. In other embodiments, the template nucleic acid comprises a nucleotide sequence that may be used to modify the target position.

The template nucleic acid may comprise a replacement sequence. In certain embodiments, the template nucleic acid comprises a 5′ homology arm. In certain embodiments, the template nucleic acid comprises a 3′ homology arm.

In certain embodiments, the template nucleic acid is linear double stranded DNA. The length may be, e.g., about 150-200 bp, e.g., about 150, 160, 170, 180, 190, or 200 bp. The length may be, e.g., at least 150, 160, 170, 180, 190, or 200 bp. In certain embodiments, the length is no greater than 150, 160, 170, 180, 190, or 200 bp. In certain embodiments, a double stranded template nucleic acid has a length of about 160 bp, e.g., about 155-165, 150-170, 140-180, 130-190, 120-200, 110-210, 100-220, 90-230, or 80-240 bp.

The template nucleic acid can be linear single stranded DNA. In certain embodiments, the template nucleic acid is (i) linear single stranded DNA that can anneal to the nicked strand of the target nucleic acid, (ii) linear single stranded DNA that can anneal to the intact strand of the target nucleic acid, (iii) linear single stranded DNA that can anneal to the plus strand of the target nucleic acid, (iv) linear single stranded DNA that can anneal to the minus strand of the target nucleic acid, or more than one of the preceding. The length may be, e.g., about 150-200 nucleotides, e.g., about 150, 160, 170, 180, 190, or 200 nucleotides. The length may be, e.g., at least 150, 160, 170, 180, 190, or 200 nucleotides. In certain embodiments, the length is no greater than 150, 160, 170, 180, 190, or 200 nucleotides. In certain embodiments, a single stranded template nucleic acid has a length of about 160 nucleotides, e.g., about 155-165, 150-170, 140-180, 130-190, 120-200, 110-210, 100-220, 90-230, or 80-240 nucleotides.

In certain embodiments, the template nucleic acid is circular double stranded DNA, e.g., a plasmid. In certain embodiments, the template nucleic acid comprises about 500 to 1000 bp of homology on either side of the replacement sequence and/or the nick. In certain embodiments, the template nucleic acid comprises about 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises at least 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises no more than 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence.

In certain embodiments, one or both homology arms may be shortened to avoid including certain sequence repeat elements, e.g., Alu repeats, LINE elements. For example, a 5′ homology arm may be shortened to avoid a sequence repeat element, while a 3′ homology arm may be shortened to avoid a sequence repeat element. In certain embodiments, both the 5′ and the 3′ homology arms may be shortened to avoid including certain sequence repeat elements.

In certain embodiments, the template nucleic acid is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In certain embodiments, the template nucleic acid comprises about 150 to 1000 nucleotides of homology on either side of the replacement sequence and/or the nick. In certain embodiments, the template nucleic acid comprises about 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence. In certain embodiments, the template nucleic acid comprises at least 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence. In certain embodiments, the template nucleic acid comprises at most 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence.

In certain embodiments, the template nucleic acid is a lentiviral vector, e.g., an IDLV (integration deficiency lentivirus). In certain embodiments, the template nucleic acid comprises about 500 to 1000 bp of homology on either side of the replacement sequence and/or the nick. In certain embodiments, the template nucleic acid comprises about 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises at least 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises no more than 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence.

In certain embodiments, the template nucleic acid comprises one or more mutations, e.g., silent mutations, that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered. In certain embodiments, the cDNA comprises one or more mutations, e.g., silent mutations that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered.

In certain embodiments, the 5′ and 3′ homology arms each comprise a length of sequence flanking the nucleotides corresponding to the replacement sequence. In certain embodiments, a template nucleic acid comprises a replacement sequence flanked by a 5′ homology arm and a 3′ homology arm each independently comprising 10 or more, 20 or more, 50 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 1000 or more, 1100 or more, 1200 or more, 1300 or more, 1400 or more, 1500 or more, 1600 or more, 1700 or more, 1800 or more, 1900 or more, or 2000 or more nucleotides. In certain embodiments, a template nucleic acid comprises a replacement sequence flanked by a 5′ homology arm and a 3′ homology arm each independently comprising at least 50, 100, or 150 nucleotides, but not long enough to include a repeated element. In certain embodiments, a template nucleic acid comprises a replacement sequence flanked by a 5′ homology arm and a 3′ homology arm each independently comprising 5 to 100, 10 to 150, or 20 to 150 nucleotides. In certain embodiments, the replacement sequence optionally comprises a promoter and/or polyA signal.

9.4 Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter a target nucleic acid sequence. Instead, the complementary repeat sequence is utilized.

9.5 Other DNA Repair Pathways

9.5.1 SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott 2008, and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. For instance, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged.’ End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonuclease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Polβ, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

9.5.2 MMR (Mismatch Repair)

Cells contain three excision repair pathways: MMR, BER, and NER. The excision repair pathways have a common feature in that they typically recognize a lesion on one strand of the DNA, then exo/endonucleases remove the lesion and leave a 1-30 nucleotide gap that is sub-sequentially filled in by DNA polymerase and finally sealed with ligase. A more complete picture is given in Li, Cell Research (2008) 18:85-98, and a summary is provided here.

Mismatch repair (MMR) operates on mispaired DNA bases.

The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutLα which possesses an ATPase activity and is important for multiple steps of MMR. It possesses a PCNA/replication factor C (RFC)-dependent endonuclease activity which plays an important role in 3′ nick-directed MMR involving EXO1. (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

9.5.3 Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott, Nature Reviews Genetics 9, 619-631 (August 2008), and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the desired nucleotide into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Polβ that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′ ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′ flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

9.5.4 Nucleotide Excision Repair (NER)

Nucleotide excision repair (NER) is an important excision mechanism that removes bulky helix-distorting lesions from DNA. Additional details about NER are given in Marteijn et al., Nature Reviews Molecular Cell Biology 15, 465-481 (2014), and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol ε and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

9.5.5 Interstrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev1), and the Fanconi anemia (FA) proteins, e.g., FancJ.

9.5.6 Other Pathways

Several other DNA repair pathways exist in mammals.

Translesion synthesis (TLS) is a pathway for repairing a single stranded break left after a defective replication event and involves translesion polymerases, e.g., DNA polβ and Rev1.

Error-free postreplication repair (PRR) is another pathway for repairing a single stranded break left after a defective replication event.

9.6 Targeted Knockdown

Unlike CRISPR/Cas-mediated gene knockout, which permanently eliminates expression by mutating the gene at the DNA level, CRISPR/Cas knockdown allows for temporary reduction of gene expression through the use of artificial transcription factors. Mutating key residues in both DNA cleavage domains of the Cas9 protein (e.g. the D10A and H840A mutations) results in the generation of a catalytically inactive Cas9 (eiCas9 which is also known as dead Cas9 or dCas9) molecule. A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. Although an enzymatically inactive (eiCas9) Cas9 molecule itself can block transcription when recruited to early regions in the coding sequence, more robust repression can be achieved by fusing a transcriptional repression domain (for example KRAB, SID or ERD) to the Cas9 and recruiting it to the target knockdown position, e.g., within 1000 bp of sequence 3′ of the start codon or within 500 bp of a promoter region 5′ of the start codon of a gene (e.g., a HBV viral gene). It is likely that targeting DNAseI hypersensitive sites (DHSs) of the promoter may yield more efficient gene repression or activation because these regions are more likely to be accessible to the Cas9 protein and are also more likely to harbor sites for endogenous transcription factors. Especially for gene repression, blocking the binding site of an endogenous transcription factor can aid in downregulating gene expression. In certain embodiments, one or more eiCas9 molecules may be used to block binding of one or more endogenous transcription factors. In certain embodiments, an eiCas9 molecule can be fused to a chromatin modifying protein. Altering chromatin status can result in decreased expression of the target gene. One or more eiCas9 molecules fused to one or more chromatin modifying proteins may be used to alter chromatin status.

In certain embodiments, a gRNA molecule can be targeted to a known transcription response elements (e.g., promoters, enhancers, etc.), a known upstream activating sequences (UAS), and/or sequences of unknown or known function that are suspected of being able to control expression of the target DNA.

CRISPR/Cas-mediated gene knockdown can be used to reduce expression of an unwanted allele or transcript. In certain embodiments, permanent destruction of the gene is not ideal. In these embodiments, site-specific repression may be used to temporarily reduce or eliminate expression. In certain embodiments, the off-target effects of a Cas-repressor may be less severe than those of a Cas-nuclease as a nuclease can cleave any DNA sequence and cause mutations whereas a Cas-repressor may only have an effect if it targets the promoter region of an actively transcribed gene. However, while nuclease-mediated knockout is permanent, repression may only persist as long as the Cas-repressor is present in the cells. Once the repressor is no longer present, it is likely that endogenous transcription factors and gene regulatory elements would restore expression to its natural state.

9.7 Examples of gRNAs in Genome Editing Methods

gRNA molecules as described herein can be used with Cas9 molecules that generate a double strand break or a single strand break to alter the sequence of a target nucleic acid, e.g., a target position or target genetic signature. gRNA molecules useful in these methods are described below.

In certain embodiments, the gRNA molecule, e.g., a chimeric gRNA, is configured such that it comprises one or more of the following properties;

(a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;

(b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5; 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N. meningitidis tail domain, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain.

In certain embodiments, the gRNA is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), or c(i); a(i), b(xi), and c(ii).

In certain embodiments, the gRNA, e.g., a chimeric gRNA, is configured such that it comprises one or more of the following properties;

(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and (c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5, 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N. meningitidis tail domain, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain.

In certain embodiments, the gRNA is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), and c(i); a(i), b(xi), and c(ii).

In certain embodiments, the gRNA is used with a Cas9 nickase molecule having HNH activity, e.g., a Cas9 molecule having the RuvC activity inactivated, e.g., a Cas9 molecule having a mutation at D10, e.g., the D10A mutation. In certain embodiments, the gRNA is used with a Cas9 nickase molecule having RuvC activity, e.g., a Cas9 molecule having the HNH activity inactivated, e.g., a Cas9 molecule having a mutation at 840, e.g., the H840A. In certain embodiments, the gRNAs are used with a Cas9 nickase molecule having RuvC activity, e.g., a Cas9 molecule having the HNH activity inactivated, e.g., a Cas9 molecule having a mutation at N863, e.g., the N863A mutation. In certain embodiments, the gRNAs are used with a Cas9 nickase molecule having RuvC activity, e.g., a Cas9 molecule having the HNH activity inactivated, e.g., a Cas9 molecule having a mutation at N580, e.g., the N580A mutation.

In certain embodiments, a pair of gRNAs, e.g., a pair of chimeric gRNAs, comprising a first and a second gRNA, is configured such that they comprises one or more of the following properties;

(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides;

(c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5; 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N. meningitidis tail domain; or, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain;

(d) the gRNAs are configured such that, when hybridized to target nucleic acid, they are separated by 0-50, 0-100, 0-200, at least 10, at least 20, at least 30 or at least 50 nucleotides;

(e) the breaks made by the first gRNA and second gRNA are on different strands; and

(f) the PAMs are facing outwards.

In certain embodiments, one or both of the gRNAs is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(i), c, and d; a(i), b(i), c, and e; a(i), b(i), c, d, and e; a(i), b(ii), and c(i); a(i), b(ii), and c(ii); a(i), b(ii), c, and d; a(i), b(ii), c, and e; a(i), b(ii), c, d, and e; a(i), b(iii), and c(i); a(i), b(iii), and c(ii); a(i), b(iii), c, and d; a(i), b(iii), c, and e; a(i), b(iii), c, d, and e; a(i), b(iv), and c(i); a(i), b(iv), and c(ii); a(i), b(iv), c, and d; a(i), b(iv), c, and e; a(i), b(iv), c, d, and e; a(i), b(v), and c(i); a(i), b(v), and c(ii); a(i), b(v), c, and d; a(i), b(v), c, and e; a(i), b(v), c, d, and e; a(i), b(vi), and c(i); a(i), b(vi), and c(ii); a(i), b(vi), c, and d; a(i), b(vi), c, and e; a(i), b(vi), c, d, and e; a(i), b(vii), and c(i); a(i), b(vii), and c(ii); a(i), b(vii), c, and d; a(i), b(vii), c, and e; a(i), b(vii), c, d, and e; a(i), b(viii), and c(i); a(i), b(viii), and c(ii); a(i), b(viii), c, and d; a(i), b(viii), c, and e; a(i), b(viii), c, d, and e; a(i), b(ix), and c(i); a(i), b(ix), and c(ii); a(i), b(ix), c, and d; a(i), b(ix), c, and e; a(i), b(ix), c, d, and e; a(i), b(x), and c(i); a(i), b(x), and c(ii); a(i), b(x), c, and d; a(i), b(x), c, and e; a(i), b(x), c, d, and e; a(i), b(xi), and c(i); a(i), b(xi), and c(ii); a(i), b(xi), c, and d; a(i), b(xi), c, and e; a(i), b(xi), c, d, and e.

In certain embodiments, the gRNAs are used with a Cas9 nickase molecule having HNH activity, e.g., a Cas9 molecule having the RuvC activity inactivated, e.g., a Cas9 molecule having a mutation at D10, e.g., the D10A mutation. In certain embodiments, the gRNAs are used with a Cas9 nickase molecule having RuvC activity, e.g., a Cas9 molecule having the HNH activity inactivated, e.g., a Cas9 molecule having a mutation at H840, e.g., the H840A mutation. In certain embodiments, the gRNAs are used with a Cas9 nickase molecule having RuvC activity, e.g., a Cas9 molecule having the HNH activity inactivated, e.g., a Cas9 molecule having a mutation at N863, e.g., the N863A mutation. In certain embodiments, the gRNAs are used with a Cas9 nickase molecule having RuvC activity, e.g., a Cas9 molecule having the HNH activity inactivated, e.g., a Cas9 molecule having a mutation at N580, e.g., the N580A mutation.

10. Target Cells

Cas9 molecules and gRNA molecules, e.g., a Cas9 molecule/gRNA molecule complex, can be used to manipulate a cell, e.g., to edit a target nucleic acid, in a wide variety of cells.

In certain embodiments, a cell is manipulated by editing (e.g., introducing one or more mutations in) one or more forms of Hepatitis B Virus (HBV) genomic DNA, e.g., covalently closed circular HBV DNA (cccDNA), relaxed circular HBV DNA (rcDNA) or linear HBV DNA, e.g., as described herein. In certain embodiments, the expression of one or more HBV genes is modulated, e.g., in vivo. In certain embodiments, the expression of one or more HBV genes residing within integrated HBV DNA (e.g., HBV DNA that has integrated into the subject genome) is modulated, e.g., in vivo. In certain embodiments, the expression of one or more genes is modulated, e.g., ex vivo. In certain embodiments, editing (e.g., introducing one or more mutations in) the HBV genomic DNA (e.g., cccDNA, rcDNA or linear DNA) leads to partial or complete destruction of the HBV genomic DNA e.g., cccDNA, rcDNA or linear DNA), e.g., in vivo. In yet certain embodiments, editing (e.g., introducing one or more mutations in) the HBV genomic DNA (e.g., cccDNA, rcDNA or linear DNA) leads to partial or complete destruction of the HBV genomic DNA e.g., cccDNA, rcDNA or linear DNA), e.g., ex vivo.

The Cas9 and gRNA molecules, genome editing systems, compositions, or vectors described herein can be delivered to a target cell. Non-limiting examples of target cells include liver cells (including but not limited to hepatocytes, kupfer cells, sinusoidal epithelial cells, stellate cells, renal tubular epithelial cells). In certain embodiments, the target cell is a cell infected by HBV, e.g., a cell expressing sodium taurocholate co-transporting polypeptide (NTCP) receptor, e.g., a hepatocyte. In certain embodiments, the target cell is a hepatocyte.

### 11. Delivery, Formulations and Routes of Administration

The components, e.g., a Cas9 molecule, one or more gRNA molecules (e.g., a Cas9 molecule/gRNA molecule complex), and a donor template nucleic acid, or all three, can be delivered, formulated, or administered in a variety of forms, see, e.g., Tables 7 and 8. In certain embodiments, the Cas9 molecule, one or more gRNA molecules (e.g., two gRNA molecules) are present together in a genome editing system. In certain embodiments, the sequence encoding the Cas9 molecule and the sequence(s) encoding the two or more (e.g., 2, 3, 4, or more) different gRNA molecules are present on the same nucleic acid molecule, e.g., an AAV vector or a lentivirus (LV) vector. In certain embodiments, two sequences encoding the Cas9 molecules and the sequences encoding the two or more (e.g., 2, 3, 4, or more) different gRNA molecules are present on the same nucleic acid molecule, e.g., an AAV vector. When a Cas9 or gRNA component is encoded as DNA for delivery, the DNA can typically include a control region, e.g., comprising a promoter, to effect expression. Useful promoters for Cas9 molecule sequences include CMV, EFS, EF-1a, MSCV, PGK, CAG, ALB, TBG, SERPINA1, the Skeletal Alpha Actin promoter, the Muscle Creatine Kinase promoter, the Dystrophin promoter, the Alpha Myosin Heavy Chain promoter, and the Smooth Muscle Actin promoter. In certain embodiments, the promoter is a constitutive promoter. In certain embodiments, the promoter is a tissue specific promoter. Useful promoters for gRNAs include T7.H1, EF-1a, 7SK, U6, U1 and tRNA promoters. Promoters with similar or dissimilar strengths can be selected to tune the expression of components. Sequences encoding a Cas9 molecule can comprise a nuclear localization signal (NLS), e.g., an SV40 NLS. In certain embodiments, the sequence encoding a Cas9 molecule comprise at least two nuclear localization signals. In certain embodiments a promoter for a Cas9 molecule or a gRNA molecule can be, independently, inducible, tissue specific, or cell specific. Table 7 provides examples of how the components can be formulated, delivered, or administered.

TABLE 7
Elements

| | | Donor | |
|---|---|---|---|
| | | Template | |
| Cas9 | gRNA | Nucleic | |
| Molecule(s) | Molecule(s) | Acid | Comments |
| DNA | DNA | DNA | In certain embodiments, a Cas9 |
| | | | molecule (e.g., an eaCas9 or eiCas9 |
| | | | molecule) and a gRNA are transcribed |
| | | | from DNA. In certain embodiments, |
| | | | they are encoded on separate |
| | | | molecules. In certain embodiments, the |
| | | | donor template is provided as a separate |
| | | | DNA molecule. |
| DNA | DNA | | In certain embodiments, a Cas9 |
| | | | molecule (e.g., an eaCas9 or eiCas9 |
| | | | molecule) and a gRNA are transcribed |
| | | | from DNA. In certain embodiments, |
| | | | they are encoded on separate |
| | | | molecules. In certain embodiments, the |
| | | | donor template is provided on the same |
| | | | DNA molecule that encodes the gRNA. |
| DNA | | DNA | In certain embodiments, a Cas9 |
| | | | molecule (e.g., an eaCas9 or eiCas9 |
| | | | molecule) and a gRNA are transcribed |
| | | | from DNA, here from a single |
| | | | molecule. In certain embodiments, the |
| | | | donor template is provided as a separate |
| | | | DNA molecule. |
| DNA | DNA | | In certain embodiments, a Cas9 |
| | | | molecule (e.g., an eaCas9 or eiCas9 |
| | | | molecule), and a gRNA are transcribed |
| | | | from DNA. In certain embodiments, |
| | | | they are encoded on separate |
| | | | molecules. In certain embodiments, the |
| | | | donor template is provided on the same |
| | | | DNA molecule that encodes the Cas9. |
| DNA | RNA | DNA | In certain embodiments, a Cas9 |
| | | | molecule (e.g., an eaCas9 or eiCas9 |
| | | | molecule) is transcribed from DNA, and |
| | | | a gRNA is provided as in vitro |
| | | | transcribed or synthesized RNA. In |
| | | | certain embodiments, the donor |
| | | | template is provided as a separate DNA |
| | | | molecule. |
| DNA | RNA | | In certain embodiments, a Cas9 |
| | | | molecule (e.g., an eaCas9 or eiCas9 |
| | | | molecule) is transcribed from DNA, and |
| | | | a gRNA is provided as in vitro |
| | | | transcribed or synthesized RNA. In |
| | | | certain embodiments, the donor |
| | | | template is provided on the same DNA |
| | | | molecule that encodes the Cas9. |
| mRNA | RNA | DNA | In certain embodiments, a Cas9 |
| | | | molecule (e.g., an eaCas9 or eiCas9 |
| | | | molecule) is translated from in vitro |
| | | | transcribed mRNA, and a gRNA is |
| | | | provided as in vitro transcribed or |
| | | | synthesized RNA. In certain |
| | | | embodiments, the donor template is |
| | | | provided as a DNA molecule. |
| mRNA | DNA | DNA | In certain embodiments, a Cas9 |
| | | | molecule (e.g., an eaCas9 or eiCas9 |
| | | | molecule) is translated from in vitro |
| | | | transcribed mRNA, and a gRNA is |
| | | | transcribed from DNA. In certain |
| | | | embodiments, the donor template is |
| | | | provided as a separate DNA molecule. |
| mRNA | DNA | | In certain embodiments, a Cas9 |
| | | | molecule (e.g., an eaCas9 or eiCas9 |
| | | | molecule) is translated from in vitro |
| | | | transcribed mRNA, and a gRNA is |
| | | | transcribed from DNA. In certain |
| | | | embodiments, the donor template is |
| | | | provided on the same DNA molecule |
| | | | that encodes the gRNA. |
| Protein | DNA | DNA | In certain embodiments, a Cas9 |
| | | | molecule (e.g., an eaCas9 or eiCas9 |
| | | | molecule) is provided as a protein, and a |
| | | | gRNA is transcribed from DNA. In |
| | | | certain embodiments, the donor |
| | | | template is provided as a separate DNA |
| | | | molecule. |
| Protein | DNA | | In certain embodiments, a Cas9 |
| | | | molecule (e.g., an eaCas9 or eiCas9 |
| | | | molecule) is provided as a protein, and a |
| | | | gRNA is transcribed from DNA. In |
| | | | certain embodiments, the donor |
| | | | template is provided on the same DNA |
| | | | molecule that encodes the gRNA. |
| Protein | RNA | DNA | In certain embodiments (e.g., an eaCas9 |
| | | | or eiCas9 molecule) is provided as a |
| | | | protein, and a gRNA is provided as |
| | | | transcribed or synthesized RNA. This |
| | | | delivery method is referred to as “RNP |
| | | | delivery”. In certain embodiments, the |
| | | | donor template is provided as a DNA |
| | | | molecule. |

Table 8 summarizes various delivery methods for the components of a Cas system, e.g., the Cas9 molecule component and the gRNA molecule component, as described herein.

TABLE 8

| | | Delivery | | | |
|---|---|---|---|---|---|
| | | into Non- | Duration | | Type of |
| | | Dividing | of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

11.1 DNA-Based Delivery of a Cas9 Molecule and or One or More gRNA Molecule

Nucleic acid compositions encoding Cas9 molecules (e.g., eaCas9 molecules or eiCas9 molecules), gRNA molecules, a donor template nucleic acid, or any combination (e.g., two or all) thereof can be administered to subjects or delivered into cells by art-known methods or as described herein. For example, Cas9-encoding and/or gRNA-encoding DNA, as well as donor template nucleic acids can be delivered, e.g., by vectors (e.g., viral or non-viral vectors), non-vector based methods (e.g., using naked DNA or DNA complexes), or a combination thereof.

Nucleic acid compositions encoding Cas9 molecules (e.g., eaCas9 molecules or eiCas9 molecules) and/or gRNA molecules can be conjugated to molecules (e.g., N-acetylgalactosamine) promoting uptake by the target cells (e.g., the target cells described herein). Donor template molecules can likewise be conjugated to molecules (e.g., N-acetylgalactosamine) promoting uptake by the target cells (e.g., the target cells described herein).

In certain embodiments, the Cas9- and/or gRNA-encoding DNA is delivered by a vector (e.g., viral vector/virus or plasmid).

Vectors can comprise a sequence that encodes a Cas9 molecule and/or a gRNA molecule, and/or a donor template with high homology to the region (e.g., target sequence) being targeted. In certain embodiments, the donor template comprises all or part of a target sequence. Exemplary donor templates are a repair template, e.g., a gene correction template, or a gene mutation template, e.g., point mutation (e.g., single nucleotide (nt) substitution) template).

A vector can also comprise a sequence encoding a signal peptide (e.g., for nuclear localization, nucleolar localization, or mitochondrial localization), fused, e.g., to a Cas9 molecule sequence. For example, the vectors can comprise a nuclear localization sequence (e.g., from SV40) fused to the sequence encoding the Cas9 molecule.

One or more regulatory/control elements, e.g., promoters, enhancers, introns, polyadenylation signals, a Kozak consensus sequences, internal ribosome entry sites (IRES), a 2A sequence, and splice acceptor or donor can be included in the vectors. In certain embodiments, the promoter is recognized by RNA polymerase II (e.g., a CMV promoter). In other embodiments, the promoter is recognized by RNA polymerase III (e.g., a U6 promoter). In certain embodiments, the promoter is a regulated promoter (e.g., inducible promoter). In certain embodiments, the promoter is a constitutive promoter. In certain embodiments, the promoter is a tissue specific promoter. In certain embodiments, the promoter is a viral promoter. In certain embodiments, the promoter is a non-viral promoter.

In certain embodiments, the vector or delivery vehicle is a viral vector (e.g., for generation of recombinant viruses). In certain embodiments, the virus is a DNA virus (e.g., dsDNA or ssDNA virus). In certain embodiments, the virus is an RNA virus (e.g., an ssRNA virus). In certain embodiments, the virus infects dividing cells. In other embodiments, the virus infects non-dividing cells. Exemplary viral vectors/viruses include, e.g., retroviruses, lentiviruses (LV), adenovirus, adeno-associated virus (AAV), vaccinia viruses, poxviruses, and herpes simplex viruses.

In certain embodiments, the virus infects dividing cells. In other embodiments, the virus infects non-dividing cells. In certain embodiments, the virus infects both dividing and non-dividing cells. In certain embodiments, the virus can integrate into the host genome. In certain embodiments, the virus is engineered to have reduced immunity, e.g., in human. In certain embodiments, the virus is replication-competent. In other embodiments, the virus is replication-defective, e.g., having one or more coding regions for the genes necessary for additional rounds of virion replication and/or packaging replaced with other genes or deleted. In certain embodiments, the virus causes transient expression of the Cas9 molecule or molecules and/or the gRNA molecule or molecules. In other embodiments, the virus causes long-lasting, e.g., at least 1 week, 2 weeks, 1 month, 2 months, 3 months, 6 months, 9 months, 1 year, 2 years, or permanent expression, of the Cas9 molecule or molecules and/or the gRNA molecule or molecules. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In certain embodiments, the viral vector recognizes a specific cell type or tissue. For example, the viral vector can be pseudotyped with a different/alternative viral envelope glycoprotein; engineered with a cell type-specific receptor (e.g., genetic modification(s) of one or more viral envelope glycoproteins to incorporate a targeting ligand such as a peptide ligand, a single chain antibody, or a growth factor); and/or engineered to have a molecular bridge with dual specificities with one end recognizing a viral glycoprotein and the other end recognizing a moiety of the target cell surface (e.g., a ligand-receptor, monoclonal antibody, avidin-biotin and chemical conjugation).

In certain embodiments, the Cas9- and/or gRNA-encoding sequence is delivered by a recombinant retrovirus. In certain embodiments, the retrovirus (e.g., Moloney murine leukemia virus) comprises a reverse transcriptase, e.g., that allows integration into the host genome. In certain embodiments, the retrovirus is replication-competent. In other embodiments, the retrovirus is replication-defective, e.g., having one of more coding regions for the genes necessary for additional rounds of virion replication and packaging replaced with other genes, or deleted.

In certain embodiments, the Cas9- and/or gRNA-encoding nucleic acid sequence is delivered by a recombinant lentivirus. In certain embodiments, the donor template nucleic acid is delivered by a recombinant retrovirus. For example, the lentivirus is replication-defective, e.g., does not comprise one or more genes required for viral replication.

In certain embodiments, the Cas9- and/or gRNA-encoding nucleic acid sequence is delivered by a recombinant adenovirus. In certain embodiments, the donor template nucleic acid is delivered by a recombinant adenovirus. In certain embodiments, the adenovirus is engineered to have reduced immunity in human.

In certain embodiments, the Cas9- and/or gRNA-encoding nucleic acid sequence is delivered by a recombinant AAV. In certain embodiments, the donor template nucleic acid is delivered by a recombinant AAV. In certain embodiments, the AAV does not incorporate its genome into that of a host cell, e.g., a target cell as describe herein. In certain embodiments, the AAV can incorporate at least part of its genome into that of a host cell, e.g., a target cell as described herein. In certain embodiments, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh10, and pseudotyped AAV, such as AAV2/8, AAV2/5 and AAV2/6 can also be used in the disclosed methods. In certain embodiments, an AAV capsid that can be used in the methods described herein is a capsid sequence from serotype AAV1, AAV2, AAV3, AAV4, AAV5, AAV6, AAV7, AAV8, AAV9, AAV.rh8, AAV.rh10, AAV.rh32/33, AAV.rh43, AAV.rh64R1, or AAV7m8.

In certain embodiments, the Cas9- and/or gRNA-encoding nucleic acid sequence is delivered in a re-engineered AAV capsid, e.g., with about 50% or greater, e.g., about 60% or greater, about 70% or greater, about 80% or greater, about 90% or greater, or about 95% or greater, sequence homology with a capsid sequence from serotypes AAV1, AAV2, AAV3, AAV4, AAV5, AAV6, AAV7, AAV8, AAV9, AAV.rh8, AAV.rh10, AAV.rh32/33, AAV.rh43, or AAV.rh64R1.

In certain embodiments, the Cas9- and/or gRNA-encoding nucleic acid sequence is delivered by a chimeric AAV capsid. In certain embodiments, the donor template nucleic acid is delivered by a chimeric AAV capsid. Exemplary chimeric AAV capsids include, but are not limited to, AAV9i1, AAV2i8, AAV-DJ, AAV2G9, AAV2i8G9, or AAV8G9.

In certain embodiments, the AAV is a self-complementary adeno-associated virus (scAAV), e.g., a scAAV that packages both strands which anneal together to form double stranded DNA.

In certain embodiments, the Cas9- and/or gRNA-encoding DNA is delivered by a hybrid virus, e.g., a hybrid of one or more of the viruses described herein. In certain embodiments, the hybrid virus is hybrid of an AAV (e.g., of any AAV serotype), with a Bocavirus, B19 virus, porcine AAV, goose AAV, feline AAV, canine AAV, or MVM.

A packaging cell is used to form a virus particle that is capable of infecting a target cell. Exemplary packaging cells include 293 cells, which can package adenovirus, and ψ2 or PA317 cells, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., components for a Cas9 molecule, e.g., two Cas9 components. For example, an AAV vector used in gene therapy typically only possesses inverted terminal repeat (ITR) sequences from the AAV genome which are required for packaging and gene expression in the host or target cell. The missing viral functions can be supplied in trans by the packaging cell line and/or plasmid containing E2A, E4, and VA genes from adenovirus, and plasmid encoding Rep and Cap genes from AAV, as described in “Triple Transfection Protocol.” Henceforth, the viral DNA is packaged in a cell line, which contains a helper plasmid encoding the other AAV genes, namely rep and cap, but lacking ITR sequences. In certain embodiments, the viral DNA is packaged in a producer cell line, which contains E1A and/or E1B genes from adenovirus. The cell line is also infected with adenovirus as a helper. The helper virus (e.g., adenovirus or HSV) or helper plasmid promotes replication of the AAV vector and expression of AAV genes from the helper plasmid with ITRs. The helper plasmid is not packaged in significant amounts due to a lack of ITR sequences. Contamination with adenovirus can be reduced by, e.g., heat treatment to which adenovirus is more sensitive than AAV.

In certain embodiments, the viral vector is capable of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as peptide ligands, single chain antibodies, growth factors); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In certain embodiments, the viral vector achieves cell type specific expression. For example, a tissue-specific promoter can be constructed to restrict expression of the transgene (Cas9 and gRNA) to only the target cell. The specificity of the vector can also be mediated by microRNA-dependent control of transgene expression. In certain embodiments, the viral vector has increased efficiency of fusion of the viral vector and a target cell membrane. For example, a fusion protein such as fusion-competent hemagglutin (HA) can be incorporated to increase viral uptake into cells. In certain embodiments, the viral vector has the ability of nuclear localization. For example, a virus that requires the breakdown of the nuclear envelope (during cell division) and therefore can not infect a non-diving cell can be altered to incorporate a nuclear localization peptide in the matrix protein of the virus thereby enabling the transduction of non-proliferating cells.

In certain embodiments, the Cas9- and/or gRNA-encoding nucleic acid sequence is delivered by a non-vector based method (e.g., using naked DNA or DNA complexes). For example, the DNA can be delivered, e.g., by organically modified silica or silicate (Ormosil), electroporation, transient cell compression or squeezing (e.g., as described in Lee, et al, 2012, Nano Lett 12: 6322-27), gene gun, sonoporation, magnetofection, lipid-mediated transfection, dendrimers, inorganic nanoparticles, calcium phosphates, or a combination thereof.

In certain embodiments, delivery via electroporation comprises mixing the cells with the Cas9- and/or gRNA-encoding DNA in a cartridge, chamber or cuvette and applying one or more electrical impulses of defined duration and amplitude. In certain embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In certain embodiments, the Cas9- and/or gRNA-encoding nucleic acid sequence is delivered by a combination of a vector and a non-vector based method. In certain embodiments, the donor template nucleic acid is delivered by a combination of a vector and a non-vector based method. For example, virosomes combine liposomes combined with an inactivated virus (e.g., HIV or influenza virus), which can result in more efficient gene transfer, e.g., in respiratory epithelial cells than either viral or liposomal methods alone.

In certain embodiments, the delivery vehicle is a non-viral vector. In certain embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In certain embodiments, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown below in Table 9.

TABLE 9
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N-[1-(2,3-Dioleyloxy)propyl]N,N,N- | DOTMA | Cationic |
| trimethylammonium chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3 - | GAP-DLRIE | Cationic |
| bis(dodecyloxy)-1-propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl-1-propanaminium | | |
| trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxy- | CLIP-1 | Cationic |
| ethyl)]-dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl- | CCS | Cationic |
| spermine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane-1,9- | CDAN | Cationic |
| diamine | | |
| 2-(3-[Bis(3-amino-propyl)-amino]propylamino)- | RPR209120 | Cationic |
| N-ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]- | DLin-KC2- | Cationic |
| dioxolane | DMA | |
| dilinoleyl-methyl-4-dimethylaminobutyrate | DLin-MC3- | Cationic |
| | DMA | |

Exemplary polymers for gene transfer are shown below in Table 10.

TABLE 10
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In certain embodiments, the vehicle has targeting modifications to increase target cell update of nanoparticles and liposomes, e.g., cell specific antigens, monoclonal antibodies, single chain antibodies, aptamers, polymers, sugars (e.g., N-acetylgalactosamine (GalNAc)), and cell penetrating peptides. In certain embodiments, the vehicle uses fusogenic and endosome-destabilizing peptides/polymers. In certain embodiments, the vehicle undergoes acid-triggered conformational changes (e.g., to accelerate endosomal escape of the cargo). In certain embodiments, a stimuli-cleavable polymer is used, e.g., for release in a cellular compartment. For example, disulfide-based cationic polymers that are cleaved in the reducing cellular environment can be used.

In certain embodiments, the delivery vehicle is a biological non-viral delivery vehicle. In certain embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In certain embodiments, the vehicle is a genetically modified bacteriophage (e.g., engineered phages having large packaging capacity, less immunogenic, containing mammalian plasmid maintenance sequences and having incorporated targeting ligands). In certain embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In certain embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30-100 nm) of endocytic origin (e.g., can be produced from various cell types and can therefore be taken up by cells without the need of for targeting ligands).

In certain embodiments, one or more nucleic acid molecules (e.g., DNA molecules) other than the components of a Cas system, e.g., the Cas9 molecule component or components and/or the gRNA molecule component or components described herein, are delivered. In certain embodiments, the nucleic acid molecule is delivered at the same time as one or more of the components of the Cas system are delivered. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In certain embodiments, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component or components and/or the gRNA molecule component or components can be delivered by a nanoparticle, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

11.2 Delivery of a RNA Encoding a Cas9 Molecule

RNA encoding Cas9 molecules (e.g., eaCas9 molecules or eiCas9 molecules) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, transient cell compression or squeezing (e.g., as described in Lee, et al., 2012, Nano Lett 12: 6322-27), lipid-mediated transfection, peptide-mediated delivery, or a combination thereof. Cas9-encoding and/or gRNA-encoding RNA can be conjugated to molecules to promote uptake by the target cells (e.g., target cells described herein).

In certain embodiments, delivery via electroporation comprises mixing the cells with the RNA encoding Cas9 molecules (e.g., eaCas9 molecules, eiCas9 molecules or eiCas9 fusion proteins) and/or gRNA molecules with or without donor template nucleic acid molecules, in a cartridge, chamber or cuvette and applying one or more electrical impulses of defined duration and amplitude. In certain embodiments, delivery via electroporation is performed using a system in which cells are mixed with the RNA encoding Cas9 molecules (e.g., eaCas9 molecules, eiCas9 molecules or eiCas9 fusion proteins) and/or gRNA molecules with or without donor template nucleic acid molecules, in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

11.3 Delivery of a Cas9 Molecule Protein

Cas9 molecules (e.g., eaCas9 molecules or eiCas9 molecules) can be delivered into cells by art-known methods or as described herein. For example, Cas9 protein molecules can be delivered, e.g., by microinjection, electroporation, transient cell compression or squeezing (e.g., as described in Lee, et al, 2012, Nano Lett 12: 6322-27), lipid-mediated transfection, peptide-mediated delivery, or a combination thereof. Delivery can be accompanied by DNA encoding a gRNA or by a gRNA. Cas9 protein can be conjugated to molecules promoting uptake by the target cells (e.g., target cells described herein).

In certain embodiments, delivery via electroporation comprises mixing the cells with the Cas9 molecules (e.g., eaCas9 molecules, eiCas9 molecules or eiCas9 fusion proteins) and/or gRNA molecules with or without donor nucleic acid, in a cartridge, chamber or cuvette and applying one or more electrical impulses of defined duration and amplitude. In certain embodiments, delivery via electroporation is performed using a system in which cells are mixed with the Cas9 molecules (e.g., eaCas9 molecules, eiCas9 molecules or eiCas9 fusion proteins) and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

11. 4 RNP Delivery of Cas9 Molecule Protein and gRNA

In certain embodiments, the Cas9 molecule and gRNA molecule are delivered to target cells via Ribonucleoprotein (RNP) delivery. In certain embodiments, the Cas9 molecule is provided as a protein, and the gRNA molecule is provided as transcribed or synthesized RNA. The gRNA molecule can be generated by chemical synthesis. In certain embodiments, the gRNA molecule forms a RNP complex with the Cas9 molecule protein under suitable condition prior to delivery to the target cells. The RNP complex can be delivered to the target cells by any suitable methods known in the art, e.g., by electroporation, lipid-mediated transfection, protein or DNA-based shuttle, mechanical force, or hydraulic force.

11.5 Route of Administration

Systemic modes of administration include oral and parenteral routes. Parenteral routes include, by way of example, intravenous, intrarterial, intramuscular, intradermal, subcutaneous, intranasal, and intraperitoneal routes. Components administered systemically may be modified or formulated to target hepatocytes, or to target HBV-infected hepatocytes.

Local modes of administration include, by way of example, intraparenchymal delivery to the liver, intrahepatic artery infusion and infusion into the portal vein. In certain embodiments, significantly smaller amounts of the components (compared with systemic approaches) may exert an effect when administered locally (for example, directly into the liver parenchyma) compared to when administered systemically (for example, intravenously). Local modes of administration can reduce or eliminate the incidence of potentially toxic side effects that may occur when therapeutically effective amounts of a component are administered systemically.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device implanted in the liver.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

11.6 Bi-Modal or Differential Delivery of Components

Separate delivery of the components of a Cas system, e.g., the Cas9 molecule component or components and the gRNA molecule component or components, and more particularly, delivery of the components by differing modes, can enhance performance, e.g., by improving tissue specificity and safety.

In certain embodiments, the Cas9 molecule or molecules and the gRNA molecule or molecules are delivered by different modes, or as sometimes referred to herein as differential modes. Different or differential modes, as used herein, refer modes of delivery that confer different pharmacodynamic or pharmacokinetic properties on the subject component molecule, e.g., a Cas9 molecule or molecules or gRNA molecule or molecules, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components, e.g., a Cas9 molecule and a gRNA molecule, can be delivered by modes that differ in terms of resulting half-life or persistence of the delivered component within the body, or in a particular compartment, tissue or organ. In certain embodiments, a gRNA molecule can be delivered by such modes. The Cas9 molecule component can be delivered by a mode which results in less persistence or less exposure to the body or a particular compartment or tissue or organ. In certain embodiments, two Cas9 molecules can by delivered by modes that differ in terms of resulting half-life or persistence of the delivered component within the body, or in a particular compartment, tissue or organ. In certain embodiments, two or more gRNA molecules can by delivered by modes that differ in terms of resulting half-life or persistence of the delivered component within the body, or in a particular compartment, tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

In certain embodiments, the second component, two Cas9 molecules, is delivered in a transient manner, for example as mRNA or as protein, ensuring that the full Cas9/gRNA complex is only present and active for a short period of time. In certain embodiments, the second components, two Cas9 molecules, are delivered at two separate time points, e.g. a first Cas9 molecule delivered at one time point and a second Cas9 molecule delivered at a second time point, for example as mRNA or as protein, ensuring that the full Cas9/gRNA complexes are only present and active for a short period of time. Furthermore, the components can be delivered in different molecular form or with different delivery vectors that complement one another to enhance safety and tissue specificity.

Use of differential delivery modes can enhance performance, safety and efficacy. E.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. Two distinct second components, e.g., two distinct Cas9 molecules, are delivered by two distinct delivery modes that result in a second and third spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. The third mode comprises a second element selected from the group. In certain embodiments, the first mode of delivery comprises a first targeting element, e.g., a cell specific receptor or an antibody, and the second mode of delivery does not include that element. In embodiment, the second mode of delivery comprises a second targeting element, e.g., a second cell specific receptor or second antibody. In embodiment, the third mode of delivery comprises a second targeting element, e.g., a second cell specific receptor or second antibody.

When the Cas9 molecule or molecules are delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule or molecules and the Cas9 molecule or molecules are packaged in separated delivery vehicles with distinct but overlapping tissue tropism, the fully functional complex is only be formed in the tissue that is targeted by both vectors.

11. 7 Ex Vivo Delivery

In certain embodiments, each component of the genome editing system described in Table 7 are introduced into a cell which is then introduced into the subject, e.g., cells are removed from a subject, manipulated ex vivo and then introduced into the subject. Methods of introducing the components can include, e.g., any of the delivery methods described in Table 8.

### 12. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein, “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:

(i) alteration, e.g., replacement, of one or both of the non-linking phosphate oxygens and/or of one or more of the linking phosphate oxygens in the phosphodiester backbone linkage;

(ii) alteration, e.g., replacement, of a constituent of the ribose sugar, e.g., of the 2′ hydroxyl on the ribose sugar;

(iii) wholesale replacement of the phosphate moiety with “dephospho” linkers;

(iv) modification or replacement of a naturally occurring nucleobase;

(v) replacement or modification of the ribose-phosphate backbone;

(vi) modification of the 3′ end or 5′ end of the oligonucleotide, e.g., removal, modification or replacement of a terminal phosphate group or conjugation of a moiety; and

(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In certain embodiments, every base of a gRNA is modified, e.g., all bases have a modified phosphate group, e.g., all are phosphorothioate groups. In certain embodiments, all, or substantially all, of the phosphate groups of a unimolecular or modular gRNA molecule are replaced with phosphorothioate groups.

In certain embodiments, modified nucleotides, e.g., nucleotides having modifications as described herein, can be incorporated into a nucleic acid, e.g., a “modified nucleic acid.” In certain embodiments, the modified nucleic acids comprise one, two, three or more modified nucleotides. In certain embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in certain embodiments, the modified nucleic acids described herein can contain one or more modified nucleosides or nucleotides, e.g., to introduce stability toward nucleases.

In certain embodiments, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In certain embodiments, the modified nucleosides, modified nucleotides, and modified nucleic acids described herein can disrupt binding of a major groove interacting partner with the nucleic acid. In certain embodiments, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

12.1 Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In certain embodiments, aryl groups have from 6 to about 20 carbon atoms.

As used herein, “alkenyl” refers to an aliphatic group containing at least one double bond.

As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

12.2 Phosphate Backbone Modifications

12.2.1 The Phosphate Group

In certain embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In certain embodiments, the modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In certain embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

12.2.2 Replacement of the Phosphate Group

The phosphate group can be replaced by non-phosphorus containing connectors. In certain embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methyl enedimethylhydrazo and methyleneoxymethylimino.

12.2.3 Replacement of the Ribophosphate Backbone

Scaffolds that can mimic nucleic acids can also be constructed wherein the phosphate linker and ribose sugar are replaced by nuclease resistant nucleoside or nucleotide surrogates. In certain embodiments, the nucleobases can be tethered by a surrogate backbone. Examples can include, without limitation, the morpholino, cyclobutyl, pyrrolidine and peptide nucleic acid (PNA) nucleoside surrogates.

12.3 Sugar Modifications

The modified nucleosides and modified nucleotides can include one or more modifications to the sugar group. For example, the 2′ hydroxyl group (OH) can be modified or replaced with a number of different “oxy” or “deoxy” substituents. In certain embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In certain embodiments, the “oxy”-2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In certain embodiments, the “oxy”-2′ hydroxyl group modification can include the methoxyethyl group (MOE), (OCH₂CH₂OCH₃, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In certain embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with α-L-threofuranosyl-(3′→2′)).

12.4 Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In certain embodiments, the nucleobase can include, for example, naturally-occurring and synthetic derivatives of a base.

12.4.1 Uracil

In certain embodiments, the modified nucleobase is a modified uracil. Exemplary nucleobases and nucleosides having a modified uracil include without limitation pseudouridine (w), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ψ), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τcm⁵U), 1-taurinomethyl-pseudouridine, 5-taurinomethyl-2-thio-uridine (τm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s2U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s2Um), 5-methoxycarbonylmethyl-2′-O-methyl-uridine (mcm ⁵Um), 5-carbamoylmethyl-2′-O-methyl-uridine (ncm ⁵Um), 5-carboxymethylaminomethyl-2′-O-methyl-uridine (cmnm ⁵Um), 3,2′-O-dimethyl-uridine (m³Um), 5-(isopentenylaminomethyl)-2′-O-methyl-uridine (inm ⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

12.4.2 Cytosine

In certain embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

12.4.3 Adenine

In certain embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenosine, 7-deaza-8-aza-adenosine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenosine (m²A), N6-methyl-adenosine (m⁶A), 2-methylthio-N6-methyl-adenosine (ms2 m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms2io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenosine, 2-methylthio-adenosine, 2-methoxy-adenosine, α-thio-adenosine, 2′-O-methyl-adenosine (Am), N⁶,2′-O-dimethyl-adenosine (m⁶Am), N⁶-Methyl-2′-deoxyadenosine, N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

12.4.4 Guanine

In certain embodiments, the modified nucleobase is a modified guanine. Exemplary nucleobases and nucleosides having a modified guanine include without limitation inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m′G), N2-methyl-guanosine (m²G), N2,N2-dimethyl-guanosine (m² ₂G), N2,7-dimethyl-guanosine (m²,7G), N2, N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m² 2 Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′Im), O⁶-phenyl-2′-deoxyinosine, 2′-O-ribosylguanosine (phosphate) (Gr(p)), 1-thio-guanosine, O⁶-methyl-guanosine, O⁶-Methyl-2′-deoxyguanosine, 2′-F-ara-guanosine, and 2′-F-guanosine.

12.5 Exemplary Modified gRNAs

In certain embodiments, the modified nucleic acids can be modified gRNAs. It is to be understood that any of the gRNAs described herein can be modified in accordance with this section, including any gRNA that comprises a targeting domain comprising a nucleotide sequence selected from SEQ ID NOS: 208 to 141071.

The presently disclosed subject matter encompasses the realization that the improvements observed with a 5′ capped gRNA can be extended to gRNAs that have been modified in other ways to achieve the same type of structural or functional result (e.g., by the inclusion of modified nucleosides or nucleotides, or when an in vitro transcribed gRNA is modified by treatment with a phosphatase such as calf intestinal alkaline phosphatase to remove the 5′ triphosphate group). In certain embodiments, the modified gRNAs described herein may contain one or more modifications (e.g., modified nucleosides or nucleotides) which introduce stability toward nucleases (e.g., by the inclusion of modified nucleosides or nucleotides and/or a 3′ polyA tract).

Thus, in one aspect, methods, genome editing system and compositions discussed herein provide methods, genome editing system and compositions for gene editing of certain cells (e.g., ex vivo gene editing) by using gRNAs which have been modified at or near their 5′ end (e.g., within 1-10, 1-5, or 1-2 nucleotides of their 5′ end). In certain embodiments, a gRNA comprises a modification at or near its 3′ end (e.g., within 1-10, 1-5, or 1-2 nucleotides of its 3′ end). In certain embodiments, a gRNA molecule comprises both a modification at or near its 5′ end and a modification at or near its 3′ end.

In certain embodiments, the 5′ end of the gRNA molecule lacks a 5′ triphosphate group. In certain embodiments, the 5′ end of the targeting domain lacks a 5′ triphosphate group. In certain embodiments, the 5′ end of the gRNA molecule includes a 5′ cap. In certain embodiments, the 5′ end of the targeting domain includes a 5′ cap. In certain embodiments, the gRNA molecule lacks a 5′ triphosphate group. In certain embodiments, the gRNA molecule comprises a targeting domain and the 5′ end of the targeting domain lacks a 5′ triphosphate group. In certain embodiments, gRNA molecule includes a 5′ cap. In certain embodiments, the gRNA molecule comprises a targeting domain and the 5′ end of the targeting domain includes a 5′ cap.

In certain embodiments, the 5′ end of a gRNA is modified by the inclusion of a eukaryotic mRNA cap structure or cap analog (e.g., without limitation, a G(5)ppp(5)G cap analog, a m7G(5)ppp(5)G cap analog, or a 3′-O-Me-m7G(5)ppp(5)G anti reverse cap analog (ARCA)). In certain embodiments, the 5′ cap comprises a modified guanine nucleotide that is linked to the remainder of the gRNA molecule via a 5′-5′ triphosphate linkage. In certain embodiments, the 5′ cap analog comprises two optionally modified guanine nucleotides that are linked via a 5′-5′ triphosphate linkage. In certain embodiments, the 5′ end of the gRNA molecule has the chemical formula:

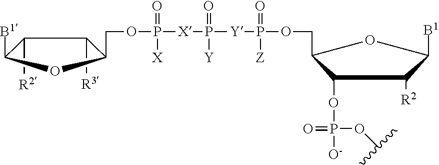

wherein:
each of B¹ and B^{1′} is independently

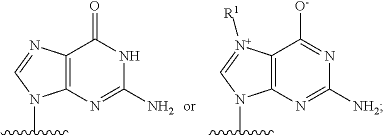

each R¹ is independently C₁₋₄ alkyl, optionally substituted by a phenyl or a 6-membered heteroaryl;
each of R², R^{2′}, and R^{3′} is independently H, F, OH, or O—C₁₋₄ alkyl;
each of X, Y, and Z is independently O or S; and
each of X′ and Y′ is independently O or CH₂.

In certain embodiments, each R¹ is independently —CH₃, —CH₂CH₃, or —CH₂C₆H₅.

In certain embodiments, R¹ is —CH₃.

In certain embodiments, B^{1′} is

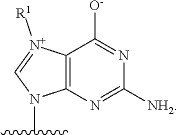

In certain embodiments, each of R², R^{2′}, and R^{3′} is independently H, OH, or O—CH₃.

In certain embodiments, each of X, Y, and Z is O.

In certain embodiments, X′ and Y′ are O.

In certain embodiments, the 5′ end of the gRNA molecule has the chemical formula:

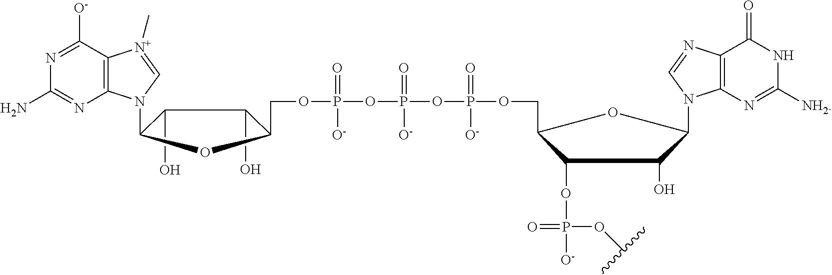

In certain embodiments, the 5′ end of the gRNA molecule has the chemical formula:

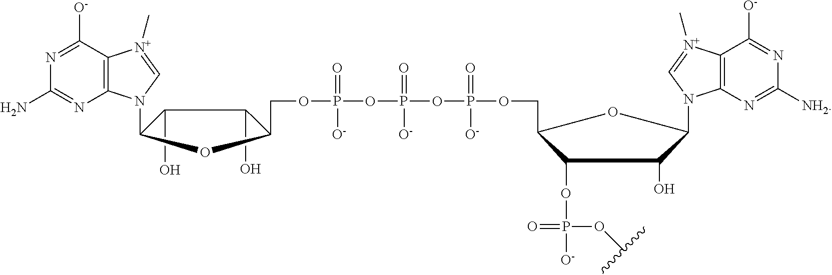

In certain embodiments, the 5′ end of the gRNA molecule has the chemical formula:

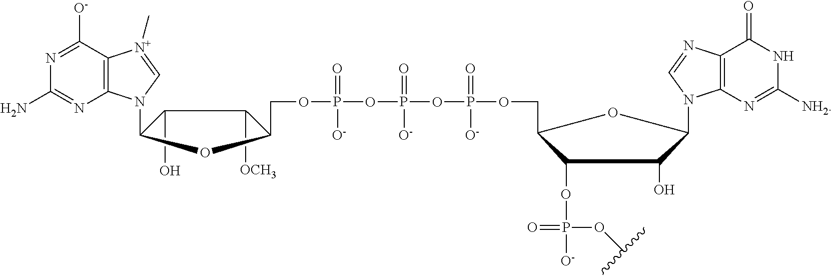

In certain embodiments, the 5′ end of the gRNA molecule has the chemical formula:

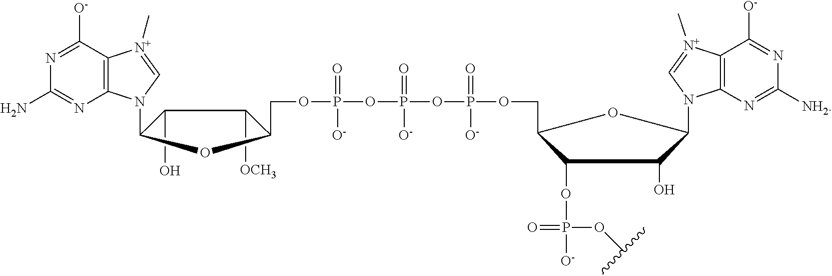

In certain embodiments, X is S, and Y and Z are O.

In certain embodiments, Y is S, and X and Z are O.

In certain embodiments, Z is S, and X and Y are O.

In certain embodiments, the phosphorothioate is the Sp diastereomer.

In certain embodiments, X′ is CH₂, and Y′ is O.

In certain embodiments, X′ is O, and Y′ is CH₂.

In certain embodiments, the 5′ cap comprises two optionally modified guanine nucleotides that are linked via an optionally modified 5′-5′ tetraphosphate linkage.

In certain embodiments, the 5′ end of the gRNA molecule has the chemical formula:

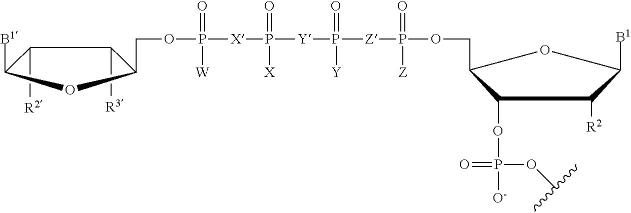

wherein:
each of B¹ and B^{1′} is independently

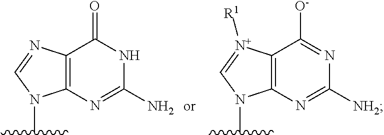

each R¹ is independently C₁₋₄ alkyl, optionally substituted by a phenyl or a 6-membered heteroaryl;
each of R², R^{2′}, and R^{3′} is independently H, F, OH, or O—C₁₋₄ alkyl;
each of W, X, Y, and Z is independently O or S; and
each of X′, Y′, and Z′ is independently O or CH₂.

In certain embodiments, each R¹ is independently —CH₃, —CH₂CH₃, or —CH₂C₆H₅.

In certain embodiments, R¹ is —CH₃.

In certain embodiments, B^{1′} is

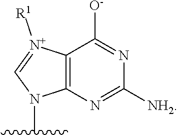

In certain embodiments, each of R², R^{2′}, and R^{3′} is independently H, OH, or O—CH₃.

In certain embodiments, each of W, X, Y, and Z is O.

In certain embodiments, each of X′, Y′, and Z′ are O.

In certain embodiments, X′ is CH₂, and Y′ and Z′ are O.

In certain embodiments, Y′ is CH₂, and X′ and Z′ are O.

In certain embodiments, Z′ is CH₂, and X′ and Y′ are O.

In certain embodiments, the 5′ cap comprises two optionally modified guanine nucleotides that are linked via an optionally modified 5′-5′ pentaphosphate linkage.

In certain embodiments, the 5′ end of the gRNA molecule has the chemical formula:

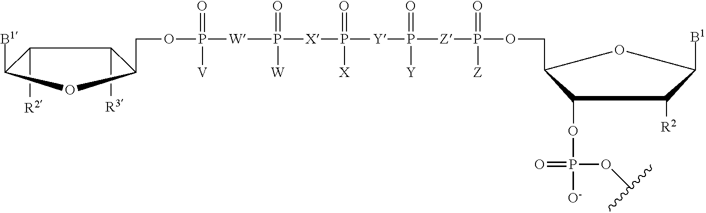

wherein:
each of B¹ and B^{1′} is independently

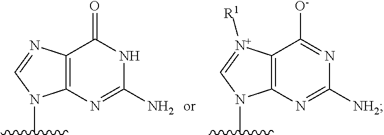

each R¹ is independently C1-4 alkyl, optionally substituted by a phenyl or a 6-membered heteroaryl;
each of R², R^{2′}, and R^{3′} is independently H, F, OH, or O—C₁₋₄ alkyl;
each of V, W, X, Y, and Z is independently O or S; and
each of W′, X′, Y′, and Z′ is independently O or CH₂.

In certain embodiments, each R¹ is independently —CH₃, —CH₂CH₃, or —CH₂C₆H₅.

In certain embodiments, R¹ is —CH₃.

In certain embodiments, B^{1′} is

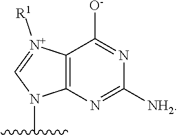

In certain embodiments, each of R², R^{2′}, and R^{3′} is independently H, OH, or O—CH₃.

In certain embodiments, each of V, W, X, Y, and Z is O.

In certain embodiments, each of W′, X′, Y′, and Z′ is O.

As used herein, the term “5′ cap” encompasses traditional mRNA 5′ cap structures but also analogs of these. For example, in addition to the 5′ cap structures that are encompassed by the chemical structures shown above, one may use, e.g., tetraphosphate analogs having a methylene-bis(phosphonate) moiety (e.g., see Rydzik, A M et al., (2009) Org Biomol Chem 7(22):4763-76), analogs having a sulfur substitution for a non-bridging oxygen (e.g., see Grudzien-Nogalska, E. et al, (2007) RNA 13(10): 1745-1755), N7-benzylated dinucleoside tetraphosphate analogs (e.g., see Grudzien, E. et al., (2004) RNA 10(9): 1479-1487), or anti-reverse cap analogs (e.g., see U.S. Pat. No. 7,074,596 and Jemielity, J. et al., (2003) RNA 9(9): 1 108-1 122 and Stepinski, J. et al., (2001) RNA 7(10):1486-1495). The present application also encompasses the use of cap analogs with halogen groups instead of OH or OMe (e.g., see U.S. Pat. No. 8,304,529); cap analogs with at least one phosphorothioate (PS) linkage (e.g., see U.S. Pat. No. 8,153,773 and Kowalska, J. et al., (2008) RNA 14(6): 1 1 19-1131); and cap analogs with at least one boranophosphate or phosphoroselenoate linkage (e.g., see U.S. Pat. No. 8,519,110); and alkynyl-derivatized 5′ cap analogs (e.g., see U.S. Pat. No. 8,969,545).

In general, the 5′ cap can be included during either chemical synthesis or in vitro transcription of the gRNA. In certain embodiments, a 5′ cap is not used and the gRNA (e.g., an in vitro transcribed gRNA) is instead modified by treatment with a phosphatase (e.g., calf intestinal alkaline phosphatase) to remove the 5′ triphosphate group.

The presently disclosed subject matter also provides for methods, genome editing system and compositions for gene editing by using gRNAs which comprise a 3′ polyA tail (also called a polyA tract herein). Such gRNAs may, for example, be prepared by adding a polyA tail to a gRNA molecule precursor using a polyadenosine polymerase following in vitro transcription of the gRNA molecule precursor. For example, in certain embodiments, a polyA tail may be added enzymatically using a polymerase such as E. coli polyA polymerase (E-PAP). gRNAs including a polyA tail may also be prepared by in vitro transcription from a DNA template. In certain embodiments, a polyA tail of defined length is encoded on a DNA template and transcribed with the gRNA via an RNA polymerase (such as T7 RNA polymerase). gRNAs with a polyA tail may also be prepared by ligating a polyA oligonucleotide to a gRNA molecule precursor following in vitro transcription using an RNA ligase or a DNA ligase with or without a splinted DNA oligonucleotide complementary to the gRNA molecule precursor and the polyA oligonucleotide. For example, in certain embodiments, a polyA tail of defined length is synthesized as a synthetic oligonucleotide and ligated on the 3′ end of the gRNA with either an RNA ligase or a DNA ligase with or without a splinted DNA oligonucleotide complementary to the guide RNA and the polyA oligonucleotide. gRNAs including the polyA tail may also be prepared synthetically, in one or several pieces that are ligated together by either an RNA ligase or a DNA ligase with or without one or more splinted DNA oligonucleotides.

In certain embodiments, the polyA tail is comprised of fewer than 50 adenine nucleotides, for example, fewer than 45 adenine nucleotides, fewer than 40 adenine nucleotides, fewer than 35 adenine nucleotides, fewer than 30 adenine nucleotides, fewer than 25 adenine nucleotides or fewer than 20 adenine nucleotides. In certain embodiments the polyA tail is comprised of between 5 and 50 adenine nucleotides, for example between 5 and 40 adenine nucleotides, between 5 and 30 adenine nucleotides, between 10 and 50 adenine nucleotides, or between 15 and 25 adenine nucleotides. In certain embodiments, the polyA tail is comprised of about 20 adenine nucleotides.

The presently disclosed subject matter also provides for methods, genome editing system and compositions for gene editing (e.g., ex vivo gene editing) by using gRNAs which include one or more modified nucleosides or nucleotides that are described herein.

While some of the exemplary modifications discussed in this section may be included at any position within the gRNA sequence, in certain embodiments, a gRNA comprises a modification at or near its 5′ end (e.g., within 1-10, 1-5, or 1-2 nucleotides of its 5′ end). In certain embodiments, a gRNA comprises a modification at or near its 3′ end (e.g., within 1-10, 1-5, or 1-2 nucleotides of its 3′ end). In certain embodiments, a gRNA comprises both a modification at or near its 5′ end and a modification at or near its 3′ end.

The presently disclosed subject matter also provides for methods, genome editing system and compositions for gene editing by using a gRNA molecule which comprises a polyA tail. In certain embodiments, a polyA tail of undefined length ranging from 1 to 1000 nucleotide is added enzymatically using a polymerase such as E. coli polyA polymerase (E-PAP). In certain embodiments, the polyA tail of a specified length (e.g., 1, 5, 10, 20, 30, 40, 50, 60, 100, or 150 nucleotides) is encoded on a DNA template and transcribed with the gRNA via an RNA polymerase (e.g., T7 RNA polymerase). In certain embodiments, a polyA tail of defined length (e.g., 1, 5, 10, 20, 30, 40, 50, 60, 100, or 150 nucleotides) is synthesized as a synthetic oligonucleotide and ligated on the 3′ end of the gRNA with either an RNA ligase or a DNA ligase with our without a splinted DNA oligonucleotide complementary to the guide RNA and the polyA oligonucleotide. In certain embodiments, the entire gRNA including a defined length of polyA tail is made synthetically, in one or several pieces, and ligated together by either an RNA ligase or a DNA ligase with or without a splinted DNA oligonucleotide.

In certain embodiments, a gRNA molecule (e.g., an in vitro transcribed gRNA) comprises a targeting domain which is complementary with a target domain from a gene expressed in a eukaryotic cell, wherein the gRNA molecule is modified at its 5′ end and comprises a 3′ polyA tail. The gRNA molecule may, for example, lack a 5′ triphosphate group (e.g., the 5′ end of the targeting domain lacks a 5′ triphosphate group). In certain embodiments, a gRNA (e.g., an in vitro transcribed gRNA) is modified by treatment with a phosphatase (e.g., calf intestinal alkaline phosphatase) to remove the 5′ triphosphate group and comprises a 3′ polyA tail as described herein. The gRNA molecule may alternatively include a 5′ cap (e.g., the 5′ end of the targeting domain includes a 5′ cap). In certain embodiments, a gRNA (e.g., an in vitro transcribed gRNA) contains both a 5′ cap structure or cap analog and a 3′ polyA tail as described herein. In certain embodiments, the 5′ cap comprises a modified guanine nucleotide that is linked to the remainder of the gRNA molecule via a 5′-5′ triphosphate linkage. In certain embodiments, the 5′ cap comprises two optionally modified guanine nucleotides that are linked via an optionally modified 5′-5′ triphosphate linkage (e.g., as described above). In certain embodiments, the polyA tail is comprised of between 5 and 50 adenine nucleotides, for example between 5 and 40 adenine nucleotides, between 5 and 30 adenine nucleotides, between 10 and 50 adenine nucleotides, between 15 and 25 adenine nucleotides, fewer than 30 adenine nucleotides, fewer than 25 adenine nucleotides or about 20 adenine nucleotides.

In certain embodiments, the presently disclosed subject matter provides for a gRNA molecule comprising a targeting domain which is complementary with a target domain from a gene expressed in a eukaryotic cell, wherein the gRNA molecule comprises a 3′ polyA tail which is comprised of fewer than 30 adenine nucleotides (e.g., fewer than 25 adenine nucleotides, between 15 and 25 adenine nucleotides, or about 20 adenine nucleotides). In certain embodiments, these gRNA molecules are further modified at their 5′ end (e.g., the gRNA molecule is modified by treatment with a phosphatase to remove the 5′ triphosphate group or modified to include a 5′ cap as described herein).

In certain embodiments, gRNAs can be modified at a 3′ terminal U ribose. In certain embodiments, the 5′ end and a 3′ terminal U ribose of the gRNA are modified (e.g., the gRNA is modified by treatment with a phosphatase to remove the 5′ triphosphate group or modified to include a 5′ cap as described herein).

For example, the two terminal hydroxyl groups of the U ribose can be oxidized to aldehyde groups and a concomitant opening of the ribose ring to afford a modified nucleoside as shown below:

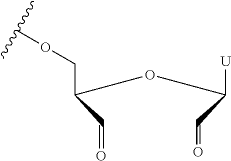

wherein “U” can be an unmodified or modified uridine.

In certain embodiments, the 3′ terminal U can be modified with a 2′3′ cyclic phosphate as shown below:

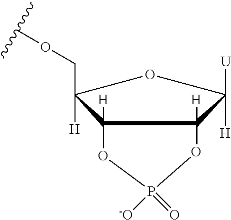

wherein “U” can be an unmodified or modified uridine.

In certain embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines, cytidines and guanosines can be replaced with modified adenosines, cytidines and guanosines, e.g., with modifications at the 8-position, e.g., 8-bromo guanosine, or with any of the modified adenosines, cytidines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclylamino, arylamino, diarylamino, heteroarylamino, diheteroarylamino, or amino acid); or cyano (—CN). In certain embodiments, the phosphate backbone can be modified as described herein, e.g., with a phosphothioate group. In certain embodiments, one or more of the nucleotides of the gRNA can each independently be a modified or unmodified nucleotide including, but not limited to 2′-sugar modified, such as, 2′-O-methyl, 2′O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In certain embodiments, a gRNA can include “locked” nucleic acids (LNA) in which the 2′ OH-group can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclylamino, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclylamino, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNA molecules include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA molecule are deoxynucleotides.

14.6 miRNA Binding Sites

microRNAs (or miRNAs) are naturally occurring cellular 19-25 nucleotide long noncoding RNAs. They bind to nucleic acid molecules having an appropriate miRNA binding site, e.g., in the 3′ UTR of an mRNA, and down-regulate gene expression. In certain embodiments, this down regulation occurs by either reducing nucleic acid molecule stability or inhibiting translation. An RNA species disclosed herein, e.g., an mRNA encoding Cas9 can comprise an miRNA binding site, e.g., in its 3′UTR. The miRNA binding site can be selected to promote down regulation of expression is a selected cell type. By way of example, the incorporation of a binding site for miR-122, a microRNA abundant in liver, can inhibit the expression of the gene of interest in the liver.

### EXAMPLES

The following Examples are merely illustrative and are not intended to limit the scope or content of the invention in any way.

### Example 1: Evaluation of Candidate Guide RNA Molecules (gRNA Molecules)

The suitability of candidate gRNAmolecules can be evaluated as described in this example. Although described for a chimeric gRNA molecule, the approach can also be used to evaluate modular gRNA molecules.

Cloning gRNA Molecules into Vectors

For each gRNA, a pair of overlapping oligonucleotides is designed and obtained. Oligonucleotides are annealed and ligated into a digested vector backbone containing an upstream U6 promoter and the remaining sequence of a long chimeric gRNA molecule. Plasmid is sequence-verified and prepped to generate sufficient amounts of transfection-quality DNA. Alternate promoters maybe used to drive in vivo transcription (e.g., H1 promoter) or for in vitro transcription (e.g., a T7 promoter).

Cloning gRNAs in Linear dsDNA Molecule (STITCHR)

For each gRNA, a single oligonucleotide is designed and obtained. The U6 promoter and the gRNA scaffold (e.g., including everything except the targeting domain, e.g., including sequences derived from the crRNA and tracrRNA, e.g., including a first complementarity domain; a linking domain; a second complementarity domain; a proximal domain; and a tail domain) are separately PCR amplified and purified as dsDNA molecules. The gRNA-specific oligonucleotide is used in a PCR reaction to stitch together the U6 and the gRNA scaffold, linked by the targeting domain specified in the oligonucleotide. Resulting dsDNA molecule (STITCHR product) is purified for transfection. Alternate promoters may be used to drive in vivo transcription (e.g., H1 promoter) or for in vitro transcription (e.g., T7 promoter). Any gRNA scaffold may be used to create gRNAs compatible with Cas9s from any bacterial species.

Initial gRNA Screen

Each gRNA to be tested is transfected, along with a plasmid expressing Cas9 and a small amount of a GFP-expressing plasmid into human cells. In preliminary experiments, these cells can be immortalized human cell lines such as 293T, K562 or U2OS. Alternatively, primary human cells may be used. In certain embodiments, cells may be relevant to the eventual therapeutic cell target (for example, an erythroid cell). The use of primary cells similar to the potential therapeutic target cell population may provide important information on gene targeting rates in the context of endogenous chromatin and gene expression.

Transfection may be performed using lipid transfection (such as Lipofectamine or Fugene) or by electroporation (such as Lonza Nucleofection). Following transfection, GFP expression can be determined either by fluorescence microscopy or by flow cytometry to confirm consistent and high levels of transfection. These preliminary transfections can comprise different gRNAs and different targeting approaches (17-mers, 20-mers, nuclease, dual-nickase, etc.) to determine which gRNAs/combinations of gRNAs give the greatest activity.

Efficiency of cleavage with each gRNA may be assessed by measuring NHEJ-induced indel formation at the target locus by a T7E1-type assay or by sequencing. Alternatively, other mismatch-sensitive enzymes, such as Cell/Surveyor nuclease, may also be used.

For the T7E1 assay, PCR amplicons are approximately 500-700 bp with the intended cut site placed asymmetrically in the amplicon. Following amplification, purification and size-verification of PCR products, DNA is denatured and re-hybridized by heating to 95° C. and then slowly cooling. Hybridized PCR products are then digested with T7 Endonuclease I (or other mismatch-sensitive enzyme) which recognizes and cleaves non-perfectly matched DNA. If indels are present in the original template DNA, when the amplicons are denatured and re-annealed, this results in the hybridization of DNA strands harboring different indels and therefore lead to double-stranded DNA that is not perfectly matched. Digestion products may be visualized by gel electrophoresis or by capillary electrophoresis. The fraction of DNA that is cleaved (density of cleavage products divided by the density of cleaved and uncleaved) may be used to estimate a percent NHEJ using the following equation: % NHEJ=(1−(1−fraction cleaved)^{1/2}). The T7E1 assay is sensitive down to about 2-5% NHEJ.

Sequencing may be used instead of, or in addition to, the T7E1 assay. For Sanger sequencing, purified PCR amplicons are cloned into a plasmid backbone, transformed, miniprepped and sequenced with a single primer. Sanger sequencing may be used for determining the exact nature of indels after determining the NHEJ rate by T7E1.

Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). This method allows for detection of very low NHEJ rates.

### Example 2: Assessment of Gene Targeting by NHEJ

The gRNAs that induce the greatest levels of NHEJ in initial tests can be selected for further evaluation of gene targeting efficiency. In this case, cells are derived from disease subjects and, therefore, harbor the relevant relevant target sequences.

Following transfection (usually 2-3 days post-transfection,) genomic DNA may be isolated from a bulk population of transfected cells and PCR may be used to amplify the target region. Following PCR, gene targeting efficiency to generate the desired mutations (either knockout of a target gene or removal of a target sequence motif) may be determined by sequencing. For Sanger sequencing, PCR amplicons may be 500-700 bp long. For next generation sequencing, PCR amplicons may be 300-500 bp long. If the goal is to knockout gene function, sequencing may be used to assess what percent of viral copies have undergone NHEJ-induced indels that result in a frameshift or large deletion or insertion that would be expected to destroy gene function. If the goal is to remove a specific sequence motif, sequencing may be used to assess what percent of viral copies have undergone NHEJ-induced deletions that span this sequence.

### Example 3: Assessment of Activity of Individual gRNAs Targeting Synthetic HBV Constructs

Four plasmids containing HBV sequences were constructed as reporters to measure Cas9-mediated cleavage of target DNA. These reporter plasmids, pAF196-199, encode a Green Fluorescent Protein (GFP) driven by a CMV promoter. The target HBV sequences were inserted in frame with the GFP, at its N-terminus, with a P2A self-cleaving peptide sequence between them.

gRNAs were identified using a custom guide RNA design software based on the public tool cas-offinder (Bae et al. Bioinformatics. 2014; 30(10): 1473-1475). Each gRNA to be tested was generated as a STITCHR product and co-transfected with a plasmid expressing the S. pyogenes Cas9 EQR variant (pDRmini004) into HEK293FT cells. The pDRmini004 plasmid encodes the S. pyogenes Cas9 EQR variant with a C-terminal nuclear localization signals (NLS) and a C-terminal triple flag tag, driven by a CMV promoter. gRNA and Cas9-encoding DNA was introduced into cells along with one of the target plasmids (pAF196, pAF197, pAF198, or pAF199) by Minis TransIT-293 transfection reagent. Two days post-transfection, cells were removed from their growth plates by trypsinization, washed in PBS buffer, and analyzed with a BD Accuri Flow Cytometer.

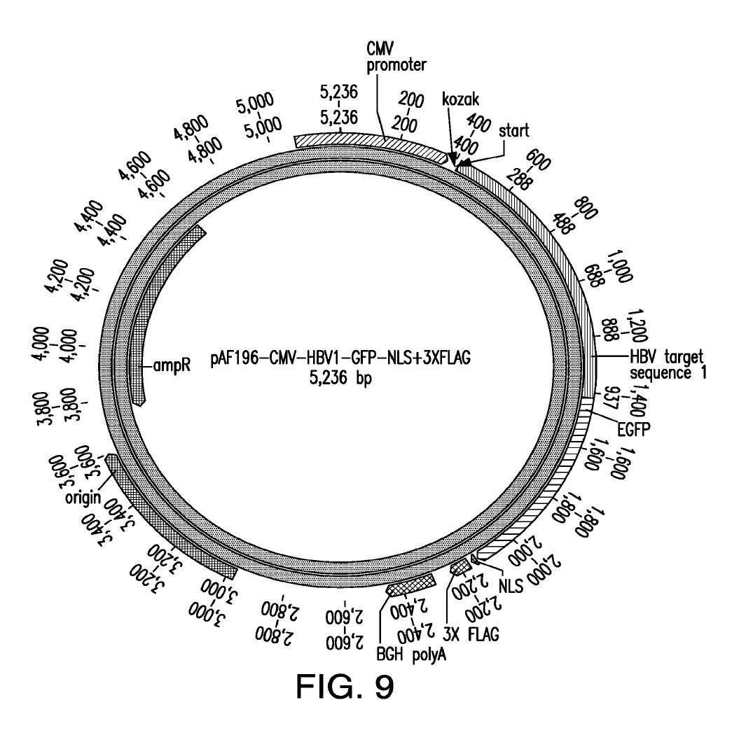

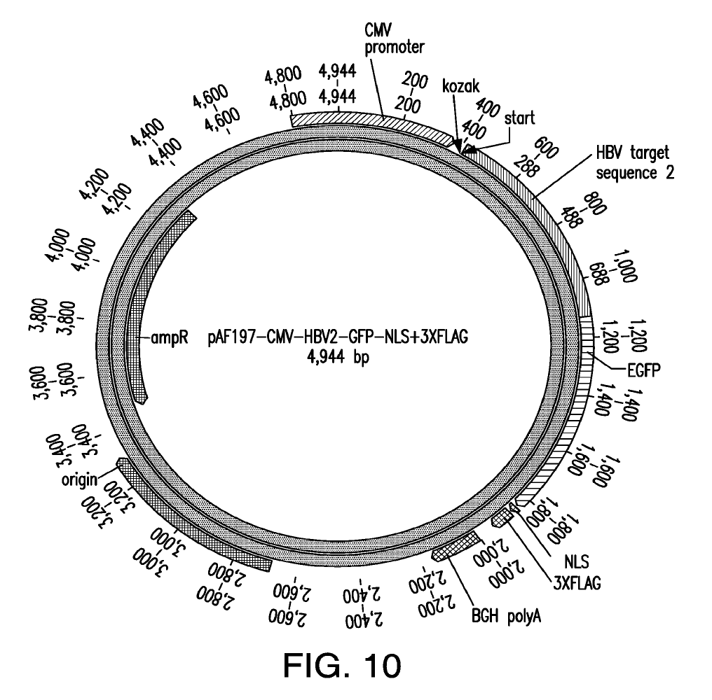

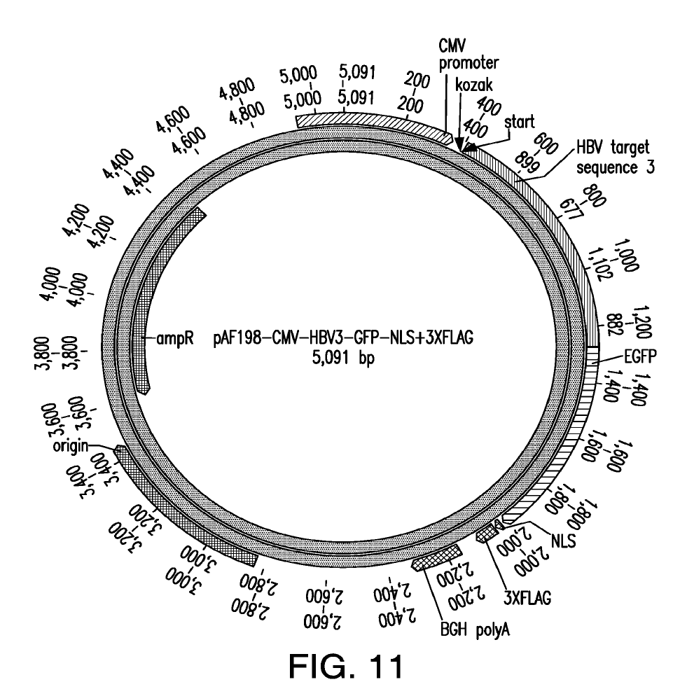

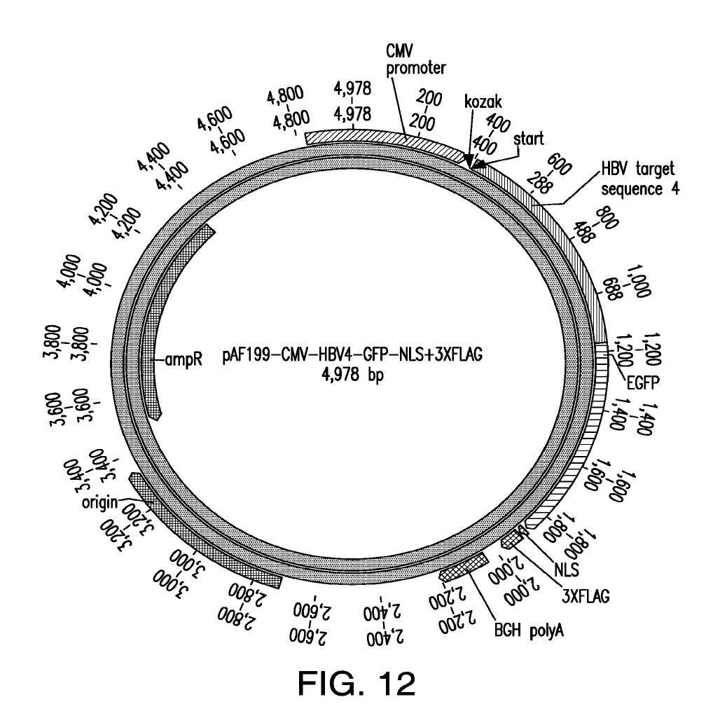

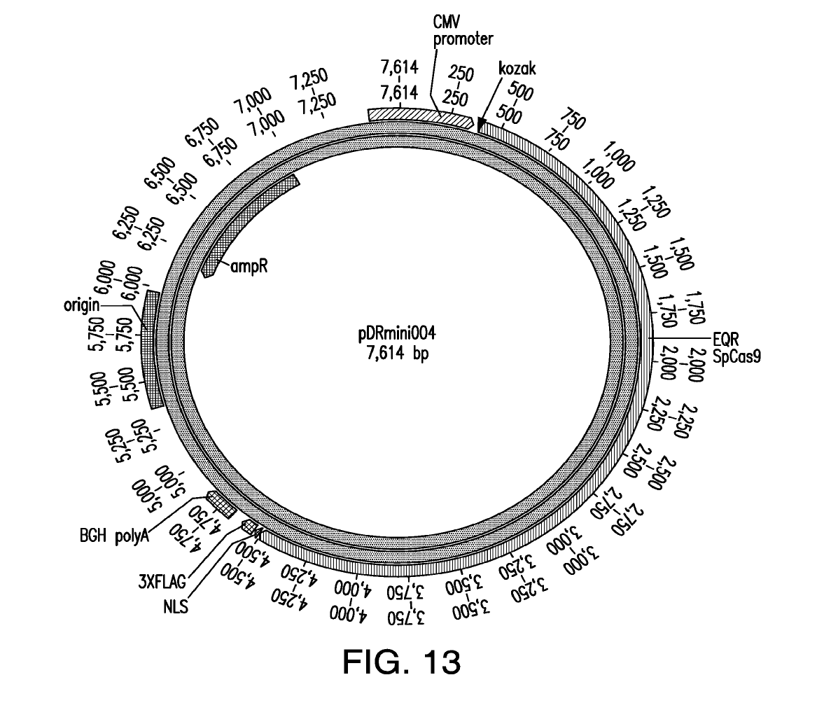

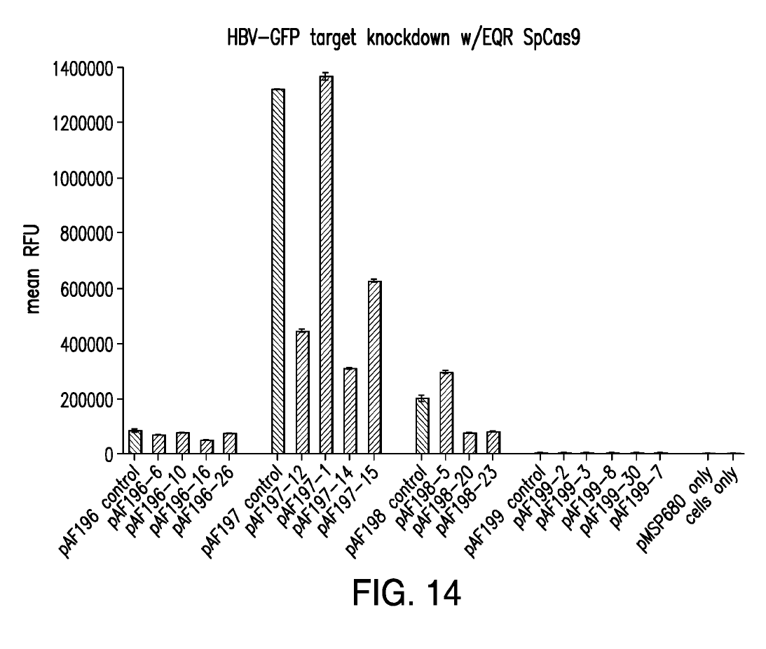

FIGS. 9-13 show the plasmid maps for pAF196-199 and pDRmini004. The nucleotide sequences of plasmids pAF196, pAF197, pAF198, pAF199 and pDRmini004 are set forth in SEQ ID NO: 210, SEQ ID NO: 211, SEQ ID NO: 212, SEQ ID NO: 213 and SEQ ID NO: 214, respectively. FIG. 14 shows the reduction in GFP expression as measured by mean fluorescence (or relative fluorescence units, RFU) of the transfected cell population due to Cas9-mediated cleavage of the HBV target sequences in plasmids pAF196-199.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein.

Such equivalents are intended to be encompassed by the following claims.

What is claimed is:

1. A genome editing system comprising:
a gRNA molecule comprising a targeting domain that is complementary with a target sequence of a Hepatitis B virus (HBV) viral gene selected from the group consisting of PreC gene, C gene, X gene, PreS1 gene, PreS2 gene, S gene, P gene and SP gene; and
a Cas9 molecule.

2. The genome editing system of claim 1, wherein said targeting domain is configured to form a double strand break or a single strand break within about 500 bp, about 450 bp, about 400 bp, about 350 bp, about 300 bp, about 250 bp, about 200 bp, about 150 bp, about 100 bp, about 50 bp, about 25 bp, or about 10 bp of an HBV target position, thereby altering said HBV viral gene.

3. The genome editing system of claim 2, wherein said altering said HBV viral gene comprises knockout of said HBV viral gene, knockdown of said HBV viral gene, or concomitant knockout and knockdown of said HBV viral gene.

4. The genome editing system of claim 1, wherein said targeting domain is configured to target a coding region or a non-coding region of said HBV viral gene, wherein said non-coding region comprises a promoter region, an enhancer region, an intron, the 3′ UTR, the 5′ UTR, or a polyadenylation signal region of said HBV viral gene; and said coding region comprises an early coding region of said HBV viral gene.

5. The genome editing system of claim 1, wherein said targeting domain comprises a nucleotide sequence that is identical to, or differs by no more than 3 nucleotides from, a nucleotide sequence selected from SEQ ID NOS: 215 to 141071.

6. The genome editing system of claim 1, wherein said Cas9 molecule is an S. pyogenes Cas9 molecule, and said targeting domain comprises a nucleotide sequence that is identical to, or differs by no more than 3 nucleotides from, a nucleotide sequence selected from the group consisting of:
(a) SEQ ID NOS: 15389-16329;
(b) SEQ ID NOS: 31598-32518;
(c) SEQ ID NOS: 47978-48841;
(d) SEQ ID NOS: 62798-63714;
(e) SEQ ID NOS: 79221-80079;
(f) SEQ ID NOS: 94449-95356;
(g) SEQ ID NOS: 110120-111022; and
(h) SEQ ID NOS: 125842-126712.

7. The genome editing system of claim 6, wherein said S. pyogenes Cas9 molecule recognizes a Protospacer Adjacent Motif (PAM) of NGG, and
(a) the genome editing system targets HBV genotype A (HBV-A), and said targeting domain comprises a nucleotide sequence that is identical to, or differs by no more than 3 nucleotides from, a nucleotide sequence selected from SEQ ID NOS: 15389-16329;
(b) the genome editing system targets HBV genotype B (HBV-B), and said targeting domain comprises a nucleotide sequence that is identical to, or differs by no more than 3 nucleotides from, a nucleotide sequence selected from SEQ ID NOS: 31598-32518;
(c) the genome editing system targets HBV genotype C (HBV-C), and said targeting domain comprises a nucleotide sequence that is identical to, or differs by no more than 3 nucleotides from, a nucleotide sequence selected from SEQ ID NOS: 47978-48841; or
(d) the genome editing system targets HBV genotype D (HBV-D), and said targeting domain comprises a nucleotide sequence that is identical to, or differs by no more than 3 nucleotides from, a nucleotide sequence selected from SEQ ID NOS: 62798-63714.

8. The genome editing system of claim 1, wherein said Cas9 molecule is an S. pyogenes Cas9 EQR variant, and said targeting domain comprises a nucleotide sequence that is identical to, or differs by no more than 3 nucleotides from, a nucleotide sequence selected from the group consisting of:
(a) SEQ ID NOS: 215-1565;
(b) SEQ ID NOS: 2225-3535;
(c) SEQ ID NOS: 4169-5381;
(d) SEQ ID NOS: 5977-7325;
(e) SEQ ID NOS: 7953-9213;
(f) SEQ ID NOS: 9830-11082;
(g) SEQ ID NOS: 11678-12954; and
(h) SEQ ID NOS: 13563-14791.

9. The genome editing system of claim 8, wherein said S. pyogenes Cas9 EQR variant recognizes a PAM selected from the group consisting of NGAG, NGCG, NGGG, NGTG, NGAA, NGAT, and NGAC, and
(a) the genome editing system targets HBV genotype A (HBV-A), and said targeting domain comprises a nucleotide sequence that is identical to, or differs by no more than 3 nucleotides from, a nucleotide sequence selected from SEQ ID NOS: 215-1565;
(b) the genome editing system targets HBV genotype B (HBV-B), and said targeting domain comprises a nucleotide sequence that is identical to, or differs by no more than 3 nucleotides from, a nucleotide sequence selected from SEQ ID NOS: 2225-3535;
(c) the genome editing system targets HBV genotype C (HBV-C), and said targeting domain comprises a nucleotide sequence that is identical to, or differs by no more than 3 nucleotides from, a nucleotide sequence selected from SEQ ID NOS: 4169-5381; or
(d) the genome editing system targets HBV genotype D (HBV-D), and said targeting domain comprises a nucleotide sequence that is identical to, or differs by no more than 3 nucleotides from, a nucleotide sequence selected from SEQ ID NOS: 5977-7325.

10. The genome editing system of claim 1, wherein said Cas9 molecule is an S. pyogenes Cas9 VRER variant, and said targeting domain comprises a nucleotide sequence that is identical to, or differs by no more than 3 nucleotides from, a nucleotide sequence selected from the group consisting of:
(a) SEQ ID NOS: 1566-2224;
(b) SEQ ID NOS: 3536-4168;
(c) SEQ ID NOS: 5382-5976;
(d) SEQ ID NOS: 7326-7952;
(e) SEQ ID NOS: 9214-9829;
(f) SEQ ID NOS: 11083-11677;
(g) SEQ ID NOS: 12955-13562; and
(h) SEQ ID NOS: 14792-15388.

11. The genome editing system of claim 10, wherein said S. pyogenes Cas9 VRER variant recognizes a PAM selected from the group consisting of NGCG, NGCA, NGCT, and NGCC, and
(a) the genome editing system targets HBV genotype A (HBV-A), and said targeting domain comprises a nucleotide sequence that is identical to, or differs by no more than 3 nucleotides from, a nucleotide sequence selected from SEQ ID NOS: 1566-2224;
(b) the genome editing system targets HBV genotype B (HBV-B), and said targeting domain comprises a nucleotide sequence that is identical to, or differs by no more than 3 nucleotides from, a nucleotide sequence selected from SEQ ID NOS: 3536-4168;
(c) the genome editing system targets HBV genotype C (HBV-C), and said targeting domain comprises a nucleotide sequence that is identical to, or differs by no more than 3 nucleotides from, a nucleotide sequence selected from SEQ ID NOS: 5382-5976; or
(d) the genome editing system targets HBV genotype D (HBV-D), and said targeting domain comprises a nucleotide sequence that is identical to, or differs by no more than 3 nucleotides from, a nucleotide sequence selected from SEQ ID NOS: 7326-7952.

12. The genome editing system of claim 1, wherein said Cas9 molecule is an S. aureus Cas9 molecule, and said targeting domain comprises a nucleotide sequence that is identical to, or differs by no more than 3 nucleotides from, a nucleotide sequence selected from the group consisting of:
(a) SEQ ID NOS: 16330-19822;
(b) SEQ ID NOS: 32519-35976;
(c) SEQ ID NOS: 48842-51921;
(d) SEQ ID NOS: 63715-67224;
(e) SEQ ID NOS: 80080-83218;
(f) SEQ ID NOS: 95357-98663;
(g) SEQ ID NOS: 111023-114350; and
(h) SEQ ID NOS: 126713-129862.

13. The genome editing system of claim 12, wherein said S. aureus Cas9 molecule recognizes a PAM of either NNNRRT or NNNRRV, and
(a) the genome editing system targets HBV genotype A (HBV-A), and said targeting domain comprises a nucleotide sequence that is identical to, or differs by no more than 3 nucleotides from, a nucleotide sequence selected from SEQ ID NOS: 16330-19822;
(b) the genome editing system targets HBV genotype B (HBV-B), and said targeting domain comprises a nucleotide sequence that is identical to, or differs by no more than 3 nucleotides from, a nucleotide sequence selected from SEQ ID NOS: 32519-35976;
(c) the genome editing system targets HBV genotype C (HBV-C), and said targeting domain comprises a nucleotide sequence that is identical to, or differs by no more than 3 nucleotides from, a nucleotide sequence selected from SEQ ID NOS: 48842-51921; or
(d) the genome editing system targets HBV genotype D (HBV-D), and said targeting domain comprises a nucleotide sequence that is identical to, or differs by no more than 3 nucleotides from, a nucleotide sequence selected from SEQ ID NOS: 63715-67224.

14. The genome editing system of claim 1, wherein said Cas9 molecule is an S. aureus Cas9 KKH variant, and said targeting domain comprises a nucleotide sequence that is identical to, or differs by no more than 3 nucleotides from, a nucleotide sequence selected from the group consisting of:
(a) SEQ ID NOS: 19823-31597;
(b) SEQ ID NOS: 35977-47977;
(c) SEQ ID NOS: 51922-62797;
(d) SEQ ID NOS: 67225-79220;
(e) SEQ ID NOS: 83219-94448;
(f) SEQ ID NOS: 98664-110119;
(g) SEQ ID NOS: 114351-125841; and
(h) SEQ ID NOS: 129863-141071.

15. The genome editing system of claim 14, wherein said S. aureus Cas9 KKH variant recognizes a PAM of either NNNRRT or NNNRRV, and
(a) the genome editing system targets HBV genotype A (HBV-A), and said targeting domain comprises a nucleotide sequence that is identical to, or differs by no more than 3 nucleotides from, a nucleotide sequence selected from SEQ ID NOS: 19823-31597;
(b) the genome editing system targets HBV genotype B (HBV-B), and said targeting domain comprises a nucleotide sequence that is identical to, or differs by no more than 3 nucleotides from, a nucleotide sequence selected from SEQ ID NOS: 35977-47977;
(c) the genome editing system targets HBV genotype C (HBV-C), and said targeting domain comprises a nucleotide sequence that is identical to, or differs by no more than 3 nucleotides from, a nucleotide sequence selected from SEQ ID NOS: 51922-62797; or
(d) the genome editing system targets HBV genotype D (HBV-D), and said targeting domain comprises a nucleotide sequence that is identical to, or differs by no more than 3 nucleotides from, a nucleotide sequence selected from SEQ ID NOS: 67225-79220.

16. The genome editing system of claim 1, wherein said Cas9 molecule is selected from the group consisting of an enzymatically active Cas9 (eaCas9) molecule, an enzymatically inactive Cas9 (eiCas9) molecule, and an eiCas9 fusion protein.

17. The genome editing system of claim 1, wherein said Cas9 molecule comprises a wild-type Cas9 molecule, a mutant Cas9 molecule, or a combination thereof.

18. The genome editing system of claim 17, wherein said mutant Cas9 molecule comprises a mutation selected from the group consisting of D10, E762, D986, H840, N854, N863, and N580

19. The genome editing system of claim 1, wherein said Cas9 molecule is an S. aureus Cas9 molecule or an S. pyogenes Cas9 molecule.

20. The genome editing system of claim 19, wherein said S. aureus Cas9 molecule is an S. aureus Cas9 variant.

21. The genome editing system of claim 20, wherein said S. aureus Cas9 variant is an S. aureus Cas9 KKH variant.

22. The genome editing system of claim 19, wherein said S. pyogenes Cas9 molecule is an S. pyogenes Cas9 variant.

23. The genome editing system of claim 22, wherein said S. pyogenes Cas9 variant is an S. pyogenes Cas9 EQR variant or an S. pyogenes Cas9 VRER variant.

24. The genome editing system of claim 1, wherein said gRNA is a modular gRNA molecule or a chimeric gRNA molecule.

25. The genome editing system of claim 1, wherein said targeting domain has a length of 16, 17, 18, 19, 20, 21, 22, 23, 24, 25, or 26 nucleotides.

26. The genome editing system of claim 1, wherein said gRNA molecule comprises from 5′ to 3′:
a targeting domain;
a first complementarity domain;
a linking domain;
a second complementarity domain;
a proximal domain; and
a tail domain.

27. The genome editing system of claim 26, wherein said linking domain is no more than 25 nucleotides in length.

28. The genome editing system of claim 26, wherein said proximal and tail domain, taken together, are at least 20, at least 25, at least 30, or at least 40 nucleotides in length.

29. The genome editing system of claim 1, comprising two, three or four gRNA molecules.

30. A composition comprising a gRNA molecule comprising a targeting domain that is complementary with a target sequence of a HBV viral gene selected from the group consisting of PreC gene, C gene, X gene, PreS1 gene, PreS2 gene, S gene, P gene and SP gene.

31. A vector comprising a polynucleotide encoding a gRNA molecule comprising a targeting domain that is complementary with a target sequence of a HBV viral gene selected from the group consisting of PreC gene, C gene, Xgene, PreS1 gene, PreS2 gene, S gene, P gene and SP gene.

32. A method of altering a HBV viral gene selected from the group consisting of PreC gene, C gene, X gene, PreS1 gene, PreS2 gene, S gene, P gene and SP gene in a cell, comprising administering to said cell one of:
(i) a genome editing system comprising a gRNA molecule comprising a targeting domain that is complementary with a target sequence of said HBV viral gene, and at least a Cas9 molecule;
(ii) a vector comprising a polynucleotide encoding a gRNA molecule comprising a targeting domain that is complementary with a target sequence of said HBV viral gene, and a polynucleotide encoding a Cas9 molecule; or
(iii) a composition comprising a gRNA molecule comprising a targeting domain that that is complementary with a target sequence of said HBV viral gene, and at least a Cas9 molecule.

33. A method of treating, preventing and/or reducing HBV infection in a subject, comprising administering to the subject one of:
(i) a genome editing system comprising a gRNA molecule comprising a targeting domain that is complementary with a target sequence of a HBV viral gene, and at least a Cas9 molecule;
(ii) a vector comprising a polynucleotide encoding a gRNA molecule comprising a targeting domain that is complementary with a target sequence of a HBV viral gene, and a polynucleotide encoding a Cas9 molecule; or
(iii) a composition comprising a gRNA molecule comprising a targeting domain that that is complementary with a target sequence of a HBV viral gene, and at least a Cas9 molecule, wherein said HBV viral gene is selected from the group consisting of PreC gene, C gene, Xgene, PreS1 gene, PreS2 gene, S gene, P gene and SP gene.

34. A gRNA molecule comprising a targeting domain which is complementary with a target sequence of a HBV viral gene selected from the group consisting of PreC gene, C gene, Xgene, PreS1 gene, PreS2 gene, S gene, P gene and SP gene in a cell.

35. A cell comprising the genome editing system of claim 1.

36. A cell comprising the composition of claim 30.

37. A cell comprising the vector of claim 31.
